SEQUENCE LISTING (457 sequences)

SEQ ID NO: 1 - protein, 3 aa, Homo sapiens
  misc_feature (1)..(3): integrin-binding peptide segment
RGD

SEQ ID NO: 2 - protein, 43 aa, Homo sapiens
  misc_feature (1)..(43): wild-type CD8 Stalk
TTTPAPRPPTPAPTIASQPLSLRPEACRPAAGGAVHTRGLDFA

SEQ ID NO: 3 - protein, 42 aa, Homo sapiens
  misc_feature (1)..(42): wild-type CD28 Stalk
FCKIEVMYPPPYLDNEKSNGTIIHVKGKHLCPSPLFPGPSKP

SEQ ID NO: 4 - protein, 4 aa, Artificial Sequence
  Synthetic Hinge
CPPC

SEQ ID NO: 5 - protein, 5 aa, Artificial Sequence
  Synthetic Hinge
DKTHT

SEQ ID NO: 6 - protein, 15 aa, Artificial Sequence
  Synthetic Hinge
CPEPKSCDTPPPCPR

SEQ ID NO: 7 - protein, 12 aa, Artificial Sequence
  Synthetic Hinge
ELKTPLGDTTHT

SEQ ID NO: 8 - protein, 10 aa, Artificial Sequence
  Synthetic Hinge
KSCDKTHTCP

SEQ ID NO: 9 - protein, 7 aa, Artificial Sequence
  Synthetic Hinge
KCCVDCP

SEQ ID NO: 10 - protein, 7 aa, Artificial Sequence
  Synthetic Hinge
KYGPPCP

SEQ ID NO: 11 - protein, 15 aa, Artificial Sequence
  Synthetic Hinge
EPKSCDKTHTCPPCP

SEQ ID NO: 12 - protein, 12 aa, Artificial Sequence
  Synthetic Hinge
ERKCCVECPPCP

SEQ ID NO: 13 - protein, 17 aa, Artificial Sequence
  Synthetic Hinge
ELKTPLGDTTHTCPRCP

SEQ ID NO: 14 - protein, 12 aa, Artificial Sequence
  Synthetic Hinge
SPNMVPHAHHAQ

SEQ ID NO: 15 - protein, 15 aa, Artificial Sequence
  Synthetic Hinge
EPKSCDKTYTCPPCP

SEQ ID NO: 16 - protein, 45 aa, Artificial Sequence
  Synthetic Hinge
TTTPAPRPPTPAPTIASQPLSLRPEACRPAAGGAVHTRGLDFACD

SEQ ID NO: 17 - protein, 24 aa, Homo sapiens
  misc_feature (1)..(24): CD* alpha Transmembrane domain
IYIWAPLAGTCGVLLLSLVITLYC

SEQ ID NO: 18 - protein, 23 aa, Homo sapiens
  misc_feature (1)..(23): CD8 beta Transmembrane domain
LGLLVAGVLVLLVSLGVAIHLCC

SEQ ID NO: 19 - protein, 25 aa, Homo sapiens
  misc_feature (1)..(25): CD4 Transmembrane domain
ALIVLGGVAGLLLFIGLGIFFCVRC

SEQ ID NO: 20 - protein, 23 aa, Homo sapiens
  misc_feature (1)..(23): CD3 zeta Transmembrane domain
LCYLLDGILFIYGVILTALFLRV

SEQ ID NO: 21 - protein, 27 aa, Homo sapiens
  misc_feature (1)..(27): CD28 Transmembrane domain
FWVLVVVGGVLACYSLLVTVAFIIFWV

SEQ ID NO: 22 - protein, 26 aa, Homo sapiens
  misc_feature (1)..(26): OX40 Transmembrane domain
VAAILGLGLVLGLLGPLAILLALYLL

SEQ ID NO: 23 - protein, 24 aa, Homo sapiens
  misc_feature (1)..(24): CD7 Transmembrane domain
ALPAALAVISFLLGLGLGVACVLA

SEQ ID NO: 24 - protein, 69 aa, Homo sapiens
  misc_feature (1)..(69): CD8a Stalk and Transmembrane domain
TTTPAPRPPTPAPTIASQPLSLRPEACRPAAGGAVHTRGLDFACDIYIWAPLAGTCGVLLLSLVITLYC

SEQ ID NO: 25 - protein, 66 aa, Homo sapiens
  misc_feature (1)..(66): CD28 Stalk and Transmembrane domain
IEVMYPPPYLDNEKSNGTIIHVKGKHLCPSPLFPGPSKPFWVLVVVGGVLACYSLLVTVAFIIFWV

SEQ ID NO: 26 - protein, 163 aa, Homo sapiens
  misc_feature (1)..(163): CD3Z Activating domain isoform 1
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 164 aa, Homo sapiens
  misc_feature (1)..(164): CD3Z Activating domain isoform 2
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 112 aa, Homo sapiens
  misc_feature (1)..(112): CD3Z Activating domain isoform 3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 113 aa, Homo sapiens
  misc_feature (1)..(113): CD3Z Activating domain isoform
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 21 aa, Homo sapiens
  misc_feature (1)..(21): CD3Z Activating domain isoform 4
NQLYNELNLGRREEYDVLDKR

SEQ ID NO: 31 - protein, 22 aa, Homo sapiens
  misc_feature (1)..(22): CD3Z Activating domain isoform 5
EGLYNELQKDKMAEAYSEIGMK

SEQ ID NO: 32 - protein, 21 aa, Homo sapiens
  misc_feature (1)..(21): CD3Z Activating domain isoform 6
DGLYQGLSTATKDTYDALHMQ

SEQ ID NO: 33 - protein, 171 aa, Homo sapiens
  misc_feature (1)..(171): CD3D Activating domain isoform 1
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 34 - protein, 127 aa, Homo sapiens
  misc_feature (1)..(127): CD3D Activating domain isoform 2
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 35 - protein, 21 aa, Homo sapiens
  misc_feature (1)..(21): CD3D Activating domain isoform 3
DQVYQPLRDRDDAQYSHLGGN

SEQ ID NO: 36 - protein, 206 aa, Homo sapiens
  misc_feature (1)..(206): CD3E Activating domain isoform 1
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDMSVATIVIVDICITGGLLLLVYYWSKNRKAKAKPVTRGAGAGGRQRGQNKERPPPVPNPDYEPIRKGQRDLYSGLNQRRI

SEQ ID NO: 37 - protein, 21 aa, Homo sapiens
  misc_feature (1)..(21): CD3E Activating domain isoform 2
NPDYEPIRKGQRDLYSGLNQR

SEQ ID NO: 38 - protein, 182 aa, Homo sapiens
  misc_feature (1)..(182): CD3G Activating domain isoform 1
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 39 - protein, 21 aa, Homo sapiens
  misc_feature (1)..(21): CD3G Activating domain isoform 2
DQLYQPLKDREDDQYSHLQGN

SEQ ID NO: 40 - protein, 226 aa, Homo sapiens
  misc_feature (1)..(226): CD79A Activating domain isoform 1
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 41 - protein, 188 aa, Homo sapiens
  misc_feature (1)..(188): CD79A Activating domain isoform 2
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 42 - protein, 21 aa, Homo sapiens
  misc_feature (1)..(21): CD79A Activating domain isoform 3
ENLYEGLNLDDCSMYEDISRG

SEQ ID NO: 43 - protein, 113 aa, Homo sapiens
  misc_feature (1)..(113): DAP12 Activating domain isoform 1
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 44 - protein, 107 aa, Homo sapiens
  misc_feature (1)..(107): DAP12 Activating domain isoform 2
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQ

SEQ ID NO: 45 - protein, 102 aa, Homo sapiens
  misc_feature (1)..(102): DAP12 Activating domain isoform 3
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 46 - protein, 101 aa, Homo sapiens
  misc_feature (1)..(101): DAP12 Activating domain isoform 4
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 47 - protein, 21 aa, Homo sapiens
  misc_feature (1)..(21): DAP12 Activating domain isoform 5
ESPYQELQGQRSDVYSDLNTQ

SEQ ID NO: 48 - protein, 86 aa, Homo sapiens
  misc_feature (1)..(86): FCERlG Activating domain isoform 1
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 49 - protein, 21 aa, Homo sapiens
  misc_feature (1)..(21): FCERlG Activating domain isoform 2
DGVYTGLSTRNQETYETLKHE

SEQ ID NO: 50 - protein, 20 aa, Homo sapiens
  misc_feature (1)..(20): DAP10 Activating domain
RPRRSPAQDGKVYINMPGRG

SEQ ID NO: 51 - protein, 68 aa, Homo sapiens
  misc_feature (1)..(68): CD28 Activating domain
FWVLVVVGGVLACYSLLVTVAFIIFWVRSKRSRLLHSDYMNMTPRRPGPTRKHYQPYAPPRDFAAYRS

SEQ ID NO: 52 - protein, 619 aa, Homo sapiens
  misc_feature (1)..(619): ZAP70 Activating domain
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 53 - protein, 42 aa, Homo sapiens
  misc_feature (1)..(42): CD137 Co-stimulatory domain
KRGRKKLLYIFKQPFMRPVQTTQEEDGCSCRFPEEEEGGCEL

SEQ ID NO: 54 - protein, 41 aa, Homo sapiens
  misc_feature (1)..(41): CD28 Co-stimulatory domain
RSKRSRLLHSDYMNMTPRRPGPTRKHYQPYAPPRDFAAYRS

SEQ ID NO: 55 - protein, 41 aa, Homo sapiens
  misc_feature (1)..(41): IC? Co-stimulatory domain
RSKRSRLLHSDYMNMTPRRPGPTRKHYQAYAAARDFAAYRS

SEQ ID NO: 56 - protein, 35 aa, Homo sapiens
  misc_feature (1)..(35): ICOS Co-stimulatory domain
TKKKYSSSVHDPNGEYMFMRAVNTAKKSRLTDVTL

SEQ ID NO: 57 - protein, 37 aa, Homo sapiens
  misc_feature (1)..(37): OX40 Co-stimulatory domain
RRDQRLPPDAHKPPGGGSFRTPIQEEQADAHSTLAKI

SEQ ID NO: 58 - protein, 49 aa, Homo sapiens
  misc_feature (1)..(49): CD27 Co-stimulatory domain
HQRRKYRSNKGESPVEPAEPCRYSCPREEEGSTIPIQEDYRKPEPACSP

SEQ ID NO: 59 - protein, 114 aa, Homo sapiens
  misc_feature (1)..(114): BLTA Co-stimulatory domain
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 60 - protein, 187 aa, Homo sapiens
  misc_feature (1)..(187): CD30 Co-stimulatory domain
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 61 - protein, 54 aa, Homo sapiens
  misc_feature (1)..(54): GITR Co-stimulatory domain
HIWQLRSQCMWPRETQLLLEVPPSTEDARSCQFPEEERGERSAEEKGRLGDLWV

SEQ ID NO: 62 - protein, 60 aa, Homo sapiens
  misc_feature (1)..(60): HVEM Co-stimulatory domain
CVKRRKPRGDVVKVIVSVQRKRQEAEGEATVIEALQAPPDVTTVAVEETIPSFTGRSPNH

SEQ ID NO: 63 - protein, 15 aa, Artificial Sequence
  Synthetic Linker
GGGGSGGGGSGGGGS

SEQ ID NO: 64 - protein, 30 aa, Artificial Sequence
  Synthetic Linker
GGGGSGGGGSGGGGSGGGGSGGGGSGGGGS

SEQ ID NO: 65 - protein, 14 aa, Artificial Sequence
  Synthetic Linker
GGGGSGGGSGGGGS

SEQ ID NO: 66 - protein, 4 aa, Artificial Sequence
  Synthetic Linker
GGSG

SEQ ID NO: 67 - protein, 5 aa, Artificial Sequence
  Synthetic Linker
GGSGG

SEQ ID NO: 68 - protein, 5 aa, Artificial Sequence
  Synthetic Linker
GSGSG

SEQ ID NO: 69 - protein, 5 aa, Artificial Sequence
  Synthetic Linker
GSGGG

SEQ ID NO: 70 - protein, 5 aa, Artificial Sequence
  Synthetic Linker
GGGSG

SEQ ID NO: 71 - protein, 5 aa, Artificial Sequence
  Synthetic Linker
GSSSG

SEQ ID NO: 72 - protein, 21 aa, Homo sapiens
  misc_feature (1)..(21): CD8 Signal peptide
MALPVTALLLPLALLLHAARP

SEQ ID NO: 73 - protein, 9 aa, Artificial Sequence
  Synthetic HA Epitope
YPYDVPDYA

SEQ ID NO: 74 - protein, 8 aa, Artificial Sequence
  Synthetic FLAG epitope
DYKDDDDK

SEQ ID NO: 75 - protein, 10 aa, Artificial Sequence
  Synthetic c-myc Epitope
EQKLISEEDL

SEQ ID NO: 76 - protein, 5 aa, Artificial Sequence
  Synthetic His5 Affinity
HHHHH

SEQ ID NO: 77 - protein, 6 aa, Artificial Sequence
  Synthetic HisX6 Affinity
HHHHHH

SEQ ID NO: 78 - protein, 8 aa, Artificial Sequence
  Synthetic Strep Tag Affinity
WSHPQFEK

SEQ ID NO: 79 - protein, 5 aa, Artificial Sequence
  Synthetic Affinity tag
RYIRS

SEQ ID NO: 80 - protein, 4 aa, Artificial Sequence
  Synthetic Affinity tag
FHHT

SEQ ID NO: 81 - protein, 17 aa, Artificial Sequence
  Synthetic Affinity tag
WEAAAREACCRECCARA

SEQ ID NO: 82 - protein, 357 aa, Homo sapiens
  misc_feature (1)..(357): EGFR Truncation
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 83 - protein, 21 aa, Artificial Sequence
  Synthetic Cleavage signal
GSGEGRGSLLTCGDVEENPGP

SEQ ID NO: 84 - protein, 368 aa, Artificial Sequence
  Synthetic eTAG IL7RA Ins PPCL (interleukin 7 receptor)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPEINNSSGEMDPILLPPCLTISILSFFSVALLVILACVL

SEQ ID NO: 85 - protein, 232 aa, Artificial Sequence
  Synthetic eTAG IL7RA Ins PPCL (interleukin 7 receptor)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQPEINNSSGEMDPILLPPCLTISILSFFSVALLVILACVL

SEQ ID NO: 86 - protein, 194 aa, Artificial Sequence
  Synthetic Myc Tag LMP1 NC_007605_1
MEQKLISEEDLEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 87 - protein, 174 aa, Artificial Sequence
  Synthetic Myc LMP1 NC_007605_1
MEQKLISEEDLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 88 - protein, 184 aa, Artificial Sequence
  Synthetic LMP1 NC_007605_1
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 89 - protein, 162 aa, Artificial Sequence
  Synthetic LMP1 NC_007605_1
MSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM

SEQ ID NO: 90 - protein, 363 aa, Artificial Sequence
  Synthetic eTAG CRLF2 transcript variant 1 NM_022148_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGAETPTPPKPKLSKCILISSLAILLMVSLLLLSLW

SEQ ID NO: 91 - protein, 227 aa, Artificial Sequence
  Synthetic eTAG CRLF2 transcript variant 1 NM_022148_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQAETPTPPKPKLSKCILISSLAILLMVSLLLLSLW

SEQ ID NO: 92 - protein, 354 aa, Artificial Sequence
  Synthetic eTAG CSF2RB NM_000395_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTESVLPMWVLALIEIFLTIAVLLAL

SEQ ID NO: 93 - protein, 218 aa, Artificial Sequence
  Synthetic eTAG CSF2RB NM_000395_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTESVLPMWVLALIEIFLTIAVLLAL

SEQ ID NO: 94 - protein, 360 aa, Artificial Sequence
  Synthetic eTAG CSF3R transcript variant 1 NM_000760_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPEGSELHIILGLFGLLLLLNCLCGTAWLCC

SEQ ID NO: 95 - protein, 224 aa, Artificial Sequence
  Synthetic eTAG CSF3R transcript variant 1 NM_000760_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPEGSELHIILGLFGLLLLLNCLCGTAWLCC

SEQ ID NO: 96 - protein, 359 aa, Artificial Sequence
  Synthetic eTAG EPOR transcript variant 1 NM_000121_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPSDLDPCCLTLSLILVVILVLLTVLALLS

SEQ ID NO: 97 - protein, 223 aa, Artificial Sequence
  Synthetic eTAG EPOR transcript variant 1 NM_000121_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPSDLDPCCLTLSLILVVILVLLTVLALLS

SEQ ID NO: 98 - protein, 368 aa, Artificial Sequence
  Synthetic eTAG GHR transcript variant 1 NM_000163_4
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTLPQMSQFTCCEDFYFPWLLCIIFGIFGLTVMLFVFLFS

SEQ ID NO: 99 - protein, 232 aa, Artificial Sequence
  Synthetic eTAG GHR transcript variant 1 NM_000163_4
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTLPQMSQFTCCEDFYFPWLLCIIFGIFGLTVMLFVFLFS

SEQ ID NO: 100 - protein, 360 aa, Artificial Sequence
  Synthetic eTAG truncated after Fn F523C IL27RA NM_004843_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGHLPDNTLRWKVLPGILCLWGLFLLGCGLSLA

SEQ ID NO: 101 - protein, 224 aa, Artificial Sequence
  Synthetic eTAG truncated after Fn F523C IL27RA NM_004843_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQHLPDNTLRWKVLPGILCLWGLFLLGCGLSLA

SEQ ID NO: 102 - protein, 359 aa, Artificial Sequence
  Synthetic eTAG truncated after Fn S505N MPL NM_005373_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGETATETAWISLVTALHLVLGLNAVLGLLLL

SEQ ID NO: 103 - protein, 223 aa, Artificial Sequence
  Synthetic eTAG truncated after Fn S505N MPL NM_005373_2
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQETATETAWISLVTALHLVLGLNAVLGLLLL

SEQ ID NO: 104 - protein, 368 aa, Artificial Sequence
  Synthetic eTag  0A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKV

SEQ ID NO: 105 - protein, 369 aa, Artificial Sequence
  Synthetic eTag  1A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVA

SEQ ID NO: 106 - protein, 369 aa, Artificial Sequence
  Synthetic eTag  2A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAA

SEQ ID NO: 107 - protein, 371 aa, Artificial Sequence
  Synthetic eTag  3A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAA

SEQ ID NO: 108 - protein, 372 aa, Artificial Sequence
  Synthetic eTag  4A JUN NM_002228_3
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAAA

SEQ ID NO: 109 - protein, 69 aa, Artificial Sequence
  Synthetic Myc Tag 0A JUN NM_002228_3
MTILGTTFGMVFSLLQVVSGEQKLISEEDLLERIARLEEKVKTLKAQNSELASTANMLREQVAQLKQKV

SEQ ID NO: 110 - protein, 70 aa, Artificial Sequence
  Synthetic Myc Tag 1A JUN NM_002228_3
MTILGTTFGMVFSLLQVVSGEQKLISEEDLLERIARLEEKVKTLKAQNSELASTANMLREQVAQLKQKVA

SEQ ID NO: 111 - protein, 71 aa, Artificial Sequence
  Synthetic Myc Tag 2A JUN NM_002228_3
MTILGTTFGMVFSLLQVVSGEQKLISEEDLLERIARLEEKVKTLKAQNSELASTANMLREQVAQLKQKVAA

SEQ ID NO: 112 - protein, 72 aa, Artificial Sequence
  Synthetic Myc Tag 3A JUN NM_002228_3
MTILGTTFGMVFSLLQVVSGEQKLISEEDLLERIARLEEKVKTLKAQNSELASTANMLREQVAQLKQKVAAA

SEQ ID NO: 113 - protein, 73 aa, Artificial Sequence
  Synthetic Myc Tag 4A JUN NM_002228_3
MTILGTTFGMVFSLLQVVSGEQKLISEEDLLERIARLEEKVKTLKAQNSELASTANMLREQVAQLKQKVAAAA

SEQ ID NO: 114 - protein, 23 aa, Artificial Sequence
  Synthetic CD2 transcript variant 1 NM_001328609_1
LIIGICGGGSLLMVFVALLVFYI

SEQ ID NO: 115 - protein, 21 aa, Artificial Sequence
  Synthetic CD3D transcript variant 1 NM_000732_4
GIIVTDVIATLLLALGVFCFA

SEQ ID NO: 116 - protein, 26 aa, Artificial Sequence
  Synthetic CD3E NM_000733_3
VMSVATIVIVDICITGGLLLLVYYWS

SEQ ID NO: 117 - protein, 21 aa, Artificial Sequence
  Synthetic CD3G NM_000073_2
GFLFAEIVSIFVLAVGVYFIA

SEQ ID NO: 118 - protein, 21 aa, Artificial Sequence
  Synthetic CD3Z CD247 transcript variant 1 NM_198053_2
LCYLLDGILFIYGVILTALFL

SEQ ID NO: 119 - protein, 22 aa, Artificial Sequence
  Synthetic CD4 transcript variant 1 and 2 NM_000616_4
MALIVLGGVAGLLLFIGLGIFF

SEQ ID NO: 120 - protein, 21 aa, Artificial Sequence
  Synthetic CD8A transcript variant 1 NM_001768_6
IYIWAPLAGTCGVLLLSLVIT

SEQ ID NO: 121 - protein, 23 aa, Artificial Sequence
  Synthetic CD8B transcript variant 2 NM_172213_3
LGLLVAGVLVLLVSLGVAIHLCC

SEQ ID NO: 122 - protein, 21 aa, Artificial Sequence
  Synthetic CD27 NM_001242_4
ILVIFSGMFLVFTLAGALFLH

SEQ ID NO: 123 - protein, 27 aa, Artificial Sequence
  Synthetic CD28 transcript variant 1 NM_006139_3
FWVLVVVGGVLACYSLLVTVAFIIFWV

SEQ ID NO: 124 - protein, 22 aa, Artificial Sequence
  Synthetic CD40 transcript variant 1 and 6 NM_001250_5
ALVVIPIIFGILFAILLVLVFI

SEQ ID NO: 125 - protein, 22 aa, Artificial Sequence
  Synthetic CD79A transcript variant 1 NM_001783_3
IITAEGIILLFCAVVPGTLLLF

SEQ ID NO: 126 - protein, 22 aa, Artificial Sequence
  Synthetic CD79B transcript variant 3 NM_001039933_2
GIIMIQTLLIILFIIVPIFLLL

SEQ ID NO: 127 - protein, 21 aa, Artificial Sequence
  Synthetic CRLF2 transcript variant 1 NM_022148_3
FILISSLAILLMVSLLLLSLW

SEQ ID NO: 128 - protein, 21 aa, Artificial Sequence
  Synthetic CRLF2 transcript variant 1 NM_022148_3
CILISSLAILLMVSLLLLSLW

SEQ ID NO: 129 - protein, 26 aa, Artificial Sequence
  Synthetic CSF2RA transcript variant 7 and 8 NM_001161529_1
NLGSVYIYVLLIVGTLVCGIVLGFLF

SEQ ID NO: 130 - protein, 19 aa, Artificial Sequence
  Synthetic CSF2RB NM_000395_2
MWVLALIVIFLTIAVLLAL

SEQ ID NO: 131 - protein, 19 aa, Artificial Sequence
  Synthetic CSF2RB NM_000395_2
MWVLALIEIFLTIAVLLAL

SEQ ID NO: 132 - protein, 23 aa, Artificial Sequence
  Synthetic CSF3R transcript variant 1 NM_000760_3
IILGLFGLLLLLTCLCGTAWLCC

SEQ ID NO: 133 - protein, 23 aa, Artificial Sequence
  Synthetic CSF3R transcript variant 1 NM_000760_3
IILGLFGLLLLLNCLCGTAWLCC

SEQ ID NO: 134 - protein, 23 aa, Artificial Sequence
  Synthetic EPOR transcript variant 1 NM_000121_3
LILTLSLILVVILVLLTVLALLS

SEQ ID NO: 135 - protein, 23 aa, Artificial Sequence
  Synthetic EPOR transcript variant 1 NM_000121_3
CCLTLSLILVVILVLLTVLALLS

SEQ ID NO: 136 - protein, 21 aa, Artificial Sequence
  Synthetic FCER1G NM_004106_1
LCYILDAILFLYGIVLTLLYC

SEQ ID NO: 137 - protein, 23 aa, Artificial Sequence
  Synthetic FCGR2C NM_201563_5
IIVAVVTGIAVAAIVAAVVALIY

SEQ ID NO: 138 - protein, 23 aa, Artificial Sequence
  Synthetic FCGRA2 transcript variant 1 NM_001136219_1
IIVAVVIATAVAAIVAAVVALIY

SEQ ID NO: 139 - protein, 24 aa, Artificial Sequence
  Synthetic GHR transcript variant 1 NM_000163_4
FPWLLIIIFGIFGLTVMLFVFLFS

SEQ ID NO: 140 - protein, 24 aa, Artificial Sequence
  Synthetic GHR transcript variant 1 NM_000163_4
FPWLLCIIFGIFGLTVMLFVFLFS

SEQ ID NO: 141 - protein, 21 aa, Artificial Sequence
  Synthetic ICOS NM_012092.3
FWLPIGCAAFVVVCILGCILI

SEQ ID NO: 142 - protein, 21 aa, Artificial Sequence
  Synthetic IFNAR1 NM_000629_2
IWLIVGICIALFALPFVIYAA

SEQ ID NO: 143 - protein, 21 aa, Artificial Sequence
  Synthetic IFNAR2 transcript variant 1 NM_207585_2
IGGIITVFLIALVLTSTIVTL

SEQ ID NO: 144 - protein, 21 aa, Artificial Sequence
  Synthetic IFNGR1 NM_000416_2
SLWIPVVAALLLFLVLSLVFI

SEQ ID NO: 145 - protein, 21 aa, Artificial Sequence
  Synthetic IFNGR2 transcript variant 1 NM_001329128_1
VILISVGTFSLLSVLAGACFF

SEQ ID NO: 146 - protein, 21 aa, Artificial Sequence
  Synthetic IFNLR1 NM_170743_3
FLVLPSLLILLLVIAAGGVIW

SEQ ID NO: 147 - protein, 23 aa, Artificial Sequence
  Synthetic IL1R1 transcript variant 2 NM_001288706_1
HMIGICVTLTVIIVCSVFIYKIF

SEQ ID NO: 148 - protein, 21 aa, Artificial Sequence
  Synthetic IL1RAP transcript variant 1 NM_002182_3
VLLVVILIVVYHVYWLEMVLF

SEQ ID NO: 149 - protein, 21 aa, Artificial Sequence
  Synthetic IL1RL1 transcript variant 1 NM_016232.4
IYCIIAVCSVFLMLINVLVII

SEQ ID NO: 150 - protein, 21 aa, Artificial Sequence
  Synthetic IL1RL2 NM_003854.2
AYLIGGLIALVAVAVSVVYIY

SEQ ID NO: 151 - protein, 19 aa, Artificial Sequence
  Synthetic IL2RA transcript variant 1 NM_000417_2
VAVAGCVFLLISVLLLSGL

SEQ ID NO: 152 - protein, 25 aa, Artificial Sequence
  Synthetic IL2RB transcript variant 1 NM_000878_4
IPWLGHLLVGLSGAFGFIILVYLLI

SEQ ID NO: 153 - protein, 21 aa, Artificial Sequence
  Synthetic IL2RG NM_000206_2
VVISVGSMGLIISLLCVYFWL

SEQ ID NO: 154 - protein, 20 aa, Artificial Sequence
  Synthetic IL3RA transcript variant 1 and 2 NM_002183_3
TSLLIALGTLLALVCVFVIC

SEQ ID NO: 155 - protein, 24 aa, Artificial Sequence
  Synthetic IL4R transcript variant 1 NM_000418_3
LLLGVSVSCIVILAVCLLCYVSIT

SEQ ID NO: 156 - protein, 20 aa, Artificial Sequence
  Synthetic IL5RA transcript variant 1 NM_000564_4
FVIVIMATICFILLILSLIC

SEQ ID NO: 157 - protein, 21 aa, Artificial Sequence
  Synthetic IL6R transcript variant 1 NM_000565_3
TFLVAGGSLAFGTLLCIAIVL

SEQ ID NO: 158 - protein, 22 aa, Artificial Sequence
  Synthetic IL6ST transcript variant 1 and 3 NM_002184_3
AIVVPVCLAFLLTTLLGVLFCF

SEQ ID NO: 159 - protein, 23 aa, Artificial Sequence
  Synthetic IL7RA NM_002185_3
ILLTISILSFFSVALLVILACVL

SEQ ID NO: 160 - protein, 27 aa, Artificial Sequence
  Synthetic IL7RA Ins PPCL (interleukin 7 receptor)
ILLPPCLTISILSFFSVALLVILACVL

SEQ ID NO: 161 - protein, 21 aa, Artificial Sequence
  Synthetic IL9R transcript variant 1 NM_002186_2
GNTLVAVSIFLLLTGPTYLLF

SEQ ID NO: 162 - protein, 21 aa, Artificial Sequence
  Synthetic IL10RA transcript variant 1 NM_001558_3
VIIFFAFVLLLSGALAYCLAL

SEQ ID NO: 163 - protein, 22 aa, Artificial Sequence
  Synthetic IL10RB NM_000628_4
WMVAVILMASVFMVCLALLGCF

SEQ ID NO: 164 - protein, 21 aa, Artificial Sequence
  Synthetic IL11RA NM_001142784_2
SLGILSFLGLVAGALALGLWL

SEQ ID NO: 165 - protein, 25 aa, Artificial Sequence
  Synthetic IL12RB1 transcript variant 1 and 4 NM_005535_2
WLIFFASLGSFLSILLVGVLGYLGL

SEQ ID NO: 166 - protein, 21 aa, Artificial Sequence
  Synthetic IL12RB2 transcript variant 1 and 3 NM_001559_2
WMAFVAPSICIAIIMVGIFST

SEQ ID NO: 167 - protein, 24 aa, Artificial Sequence
  Synthetic IL13RA1 NM_001560_2
LYITMLLIVPVIVAGAIIVLLLYL

SEQ ID NO: 168 - protein, 20 aa, Artificial Sequence
  Synthetic IL13RA2 NM_000640_2
FWLPFGFILILVIFVTGLLL

SEQ ID NO: 169 - protein, 23 aa, Artificial Sequence
  Synthetic IL15RA transcript variant 4 NM_001256765_1
VAISTSTVLLCGLSAVSLLACYL

SEQ ID NO: 170 - protein, 21 aa, Artificial Sequence
  Synthetic IL17RA NM_014339_6
VYWFITGISILLVGSVILLIV

SEQ ID NO: 171 - protein, 21 aa, Artificial Sequence
  Synthetic IL17RB NM_018725_3
LLLLSLLVATWVLVAGIYLMW

SEQ ID NO: 172 - protein, 21 aa, Artificial Sequence
  Synthetic IL17RC transcript variant 1 NM_153460_3
WALVWLACLLFAAALSLILLL

SEQ ID NO: 173 - protein, 21 aa, Artificial Sequence
  Synthetic IL17RD transcript variant 2 NM_017563_4
AVAITVPLVVISAFATLFTVM

SEQ ID NO: 174 - protein, 21 aa, Artificial Sequence
  Synthetic IL17RE transcript variant 1 NM_153480_1
LGLLILALLALLTLLGVVLAL

SEQ ID NO: 175 - protein, 21 aa, Artificial Sequence
  Synthetic IL18R1 transcript variant 1 NM_003855_3
GMIIAVLILVAVVCLVTVCVI

SEQ ID NO: 176 - protein, 21 aa, Artificial Sequence
  Synthetic IL18RAP NM_003853_3
GVVLLYILLGTIGTLVAVLAA

SEQ ID NO: 177 - protein, 21 aa, Artificial Sequence
  Synthetic IL20RA transcript variant 1 NM_014432_3
IIFWYVLPISITVFLFSVMGY

SEQ ID NO: 178 - protein, 21 aa, Artificial Sequence
  Synthetic IL20RB NM_144717_3
VLALFAFVGFMLILVVVPLFV

SEQ ID NO: 179 - protein, 21 aa, Artificial Sequence
  Synthetic IL21R transcript variant 2 NM_181078_2
GWNPHLLLLLLLVIVFIPAFW

SEQ ID NO: 180 - protein, 21 aa, Artificial Sequence
  Synthetic IL22RA1 NM_021258_3
YSFSGAFLFSMGFLVAVLCYL

SEQ ID NO: 181 - protein, 21 aa, Artificial Sequence
  Synthetic IL23R NM_144701_2
LLLGMIVFAVMLSILSLIGIF

SEQ ID NO: 182 - protein, 21 aa, Artificial Sequence
  Synthetic IL27RA NM_004843_3
VLPGILFLWGLFLLGCGLSLA

SEQ ID NO: 183 - protein, 21 aa, Artificial Sequence
  Synthetic IL27RA NM_004843_3
VLPGILCLWGLFLLGCGLSLA

SEQ ID NO: 184 - protein, 24 aa, Artificial Sequence
  Synthetic IL31RA transcript variant 1 NM_139017_5
IILITSLIGGGLLILIILTVAYGL

SEQ ID NO: 185 - protein, 23 aa, Artificial Sequence
  Synthetic LEPR transcript variant 1 NM_002303_5
AGLYVIVPVIISSSILLLGTLLI

SEQ ID NO: 186 - protein, 25 aa, Artificial Sequence
  Synthetic LIFR NM_001127671_1
VGLIIAILIPVAVAVIVGVVTSILC

SEQ ID NO: 187 - protein, 22 aa, Artificial Sequence
  Synthetic MPL NM_005373_2
ISLVTALHLVLGLSAVLGLLLL

SEQ ID NO: 188 - protein, 22 aa, Artificial Sequence
  Synthetic MPL NM_005373_2
ISLVTALHLVLGLNAVLGLLLL

SEQ ID NO: 189 - protein, 21 aa, Artificial Sequence
  Synthetic OSMR transcript variant 4 NM_001323505_1
LIHILLPMVFCVLLIMVMCYL

SEQ ID NO: 190 - protein, 24 aa, Artificial Sequence
  Synthetic PRLR transcript variant 1 NM_000949_6
TTVWISVAVLSAVICLIIVWAVAL

SEQ ID NO: 191 - protein, 21 aa, Artificial Sequence
  Synthetic TNFRSF4 NM_003327_3
VAAILGLGLVLGLLGPLAILL

SEQ ID NO: 192 - protein, 28 aa, Artificial Sequence
  Synthetic TNFRSF8 transcript variant 1 NM_001243_4
PVLDAGPVLFWVILVLVVVVGSSAFLLC

SEQ ID NO: 193 - protein, 27 aa, Artificial Sequence
  Synthetic TNFRSF9 NM_001561_5
IISFFLALTSTALLFLLFFLTLRFSVV

SEQ ID NO: 194 - protein, 21 aa, Artificial Sequence
  Synthetic TNFRSF14 transcript variant 1 NM_003820_3
WWFLSGSLVIVIVCSTVGLII

SEQ ID NO: 195 - protein, 21 aa, Artificial Sequence
  Synthetic TNFRSF18 transcript variant 1 NM_004195_2
LGWLTVVLLAVAACVLLLTSA

SEQ ID NO: 196 - protein, 117 aa, Artificial Sequence
  Synthetic CD2 transcript variant 1 NM_001328609_1
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 197 - protein, 45 aa, Artificial Sequence
  Synthetic CD3D transcript variant 1 NM_000732_4
GHETGRLSGAADTQALLRNDQVYQPLRDRDDAQYSHLGGNWARNK

SEQ ID NO: 198 - protein, 55 aa, Artificial Sequence
  Synthetic CD3E NM_000733_3
KNRKAKAKPVTRGAGAGGRQRGQNKERPPPVPNPDYEPIRKGQRDLYSGLNQRRI

SEQ ID NO: 199 - protein, 45 aa, Artificial Sequence
  Synthetic CD3G NM_000073_2
GQDGVRQSRASDKQTLLPNDQLYQPLKDREDDQYSHLQGNQLRRN

SEQ ID NO: 200 - protein, 40 aa, Artificial Sequence
  Synthetic CD4 transcript variant 1 and 2 NM_000616_4
CVRCRHRRRQAERMSQIKRLLSEKKTCQCPHRFQKTCSPI

SEQ ID NO: 201 - protein, 32 aa, Artificial Sequence
  Synthetic CD8A transcript variant 1 NM_001768_6
LYCNHRNRRRVCKCPRPVVKSGDKPSLSARYV

SEQ ID NO: 202 - protein, 48 aa, Artificial Sequence
  Synthetic CD8B transcript variant 2 NM_172213_3
RRRRARLRFMKQPQGEGISGTFVPQCLHGYYSNTTTSQKLLNPWILKT

SEQ ID NO: 203 - protein, 26 aa, Artificial Sequence
  Synthetic CD8B transcript variant 3 NM_172101_3
RRRRARLRFMKQLRLHPLEKCSRMDY

SEQ ID NO: 204 - protein, 15 aa, Artificial Sequence
  Synthetic CD8B transcript variant 5 NM_004931_4
RRRRARLRFMKQFYK

SEQ ID NO: 205 - protein, 48 aa, Artificial Sequence
  Synthetic CD27 NM_001242_4
QRRKYRSNKGESPVEPAEPCRYSCPREEEGSTIPIQEDYRKPEPACSP

SEQ ID NO: 206 - protein, 41 aa, Artificial Sequence; identical to SEQ ID NO: 55
  Synthetic mutated Delta Lck CD28 transcript variant 1 NM_006139_3

SEQ ID NO: 207 - protein, 41 aa, Artificial Sequence; identical to SEQ ID NO: 54
  Synthetic CD28 transcript variant 1 NM_006139_3

SEQ ID NO: 208 - protein, 62 aa, Artificial Sequence
  Synthetic CD40 transcript variant 1 and 6 NM_001250_5
KKVAKKPTNKAPHPKQEPQEINFPDDLPGSNTAAPVQETLHGCQPVTQEDGKESRISVQERQ

SEQ ID NO: 209 - protein, 66 aa, Artificial Sequence
  Synthetic CD40 transcript variant 5 NM_001322421_1
SESSEKVAKKPTNKAPHPKQEPQEINFPDDLPGSNTAAPVQETLHGCQPVTQEDGKESRISVQERQ

SEQ ID NO: 210 - protein, 61 aa, Artificial Sequence
  Synthetic CD79A transcript variant 1 NM_001783_3
RKRWQNEKLGLDAGDEYEDENLYEGLNLDDCSMYEDISRGLQGTYQDVGSLNIGDVQLEKP

SEQ ID NO: 211 - protein, 49 aa, Artificial Sequence
  Synthetic CD79B transcript variant 3 NM_001039933_2
LDKDDSKAGMEEDHTYEGLDIDQTATYEDIVTLRTGEVKWSVGEHPGQE

SEQ ID NO: 212 - protein, 119 aa, Artificial Sequence
  Synthetic CRLF2 transcript variant 1 NM_022148_3
KLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 213 - protein, 437 aa, Artificial Sequence
  Synthetic CSF2RB NM_000395_2
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 214 - protein, 54 aa, Artificial Sequence
  Synthetic CSF2RA transcript variant 7 and 8 NM_001161529_1
KRFLRIQRLFPPVPQIKDKLNDNHEVEDEIIWEEFTPEEGKGYREEVLTVKEIT

SEQ ID NO: 215 - protein, 64 aa, Artificial Sequence
  Synthetic CSF2RA transcript variant 9 NM_001161531_1
KRFLRIQRLFPPVPQIKDKLNDNHEVEDEMGPQRHHRCGWNLYPTPGPSPGSGSSPRLGSESSL

SEQ ID NO: 216 - protein, 186 aa, Artificial Sequence
  Synthetic CSF3R transcript variant 1 NM_000760_3
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 217 - protein, 213 aa, Artificial Sequence
  Synthetic CSF3R transcript variant 3 NM_156039_3
SPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 218 - protein, 133 aa, Artificial Sequence
  Synthetic CSF3R transcript variant 4 NM_172313_2
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQAGPPRRSAYFKDQIMLHPAPPNGLLCLFPITSVL

SEQ ID NO: 219 - protein, 235 aa, Artificial Sequence
  Synthetic EPOR transcript variant 1 NM_000121_3
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 220 - protein, 235 aa, Artificial Sequence
  Synthetic EPOR transcript variant 1 NM_000121_3
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKFLFLVVSDSGISTDYSSGDSQGAQGGLSDGPYSNPYENSLIPAAEPLPPSYVACS

SEQ ID NO: 221 - protein, 42 aa, Artificial Sequence
  Synthetic FCER1G NM_004106_1
RLKIQVRKAAITSYEKSDGVYTGLSTRNQETYETLKHEKPPQ

SEQ ID NO: 222 - protein, 77 aa, Artificial Sequence
  Synthetic FCGR2C NM_201563_5
CRKKRISANSTDPVKAAQFEPPGRQMIAIRKRQPEETNNDYETADGGYMTLNPRAPTDDDKNIYLTLPPNDHVNSNN

SEQ ID NO: 223 - protein, 77 aa, Artificial Sequence
  Synthetic FCGRA2 transcript variant 1 NM_001136219_1
CRKKRISANSTDPVKAAQFEPPGRQMIAIRKRQLEETNNDYETADGGYMTLNPRAPTDDDKNIYLTLPPNDHVNSNN

SEQ ID NO: 224 - protein, 350 aa, Artificial Sequence
  Synthetic GHR transcript variant 1 NM_000163_4
KQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 225 - protein, 38 aa, Artificial Sequence
  Synthetic ICOS NM_012092.3
CWLTKKKYSSSVHDPNGEYMFMRAVNTAKKSRLTDVTL

SEQ ID NO: 226 - protein, 100 aa, Artificial Sequence
  Synthetic IFNAR1 NM_000629_2
KVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 227 - protein, 251 aa, Artificial Sequence
  Synthetic IFNAR2 transcript variant 1 NM_207585_2
KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 228 - protein, 67 aa, Artificial Sequence
  Synthetic IFNAR2 transcript variant 2 NM_000874_4
KWIGYICLRNSLPKVLRQGLAKGWNAVAIHRCSHNALQSETPELKQSSCLSFPSSWDYKRASLCPSD

SEQ ID NO: 229 - protein, 223 aa, Artificial Sequence
  Synthetic IFNGR1 NM_000416_2
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 230 - protein, 69 aa, Artificial Sequence
  Synthetic IFNGR2 transcript variant 1 NM_001329128_1
LVLKYRGLIKYWFHTPPSIPLQIEEYLKDPTQPILEALDKDSSPKDDVWDSVSIISFPEKEQEDVLQTL

SEQ ID NO: 231 - protein, 271 aa, Artificial Sequence
  Synthetic IFNLR1 NM_170743_3
KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 232 - protein, 242 aa, Artificial Sequence
  Synthetic IFNLR1 transcript variant 2 NM_173064_2
KTLMGNPWFQRAKMPRALELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 233 - protein, 179 aa, Artificial Sequence
  Synthetic IL1R1 transcript variant 2 NM_001288706_1
KIDIVLWYRDSCYDFLPIKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 234 - protein, 210 aa, Artificial Sequence
  Synthetic IL1R1 transcript variant 3 NM_001320978_1
KIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 235 - protein, 182 aa, Artificial Sequence
  Synthetic IL1RAP transcript variant 1 NM_002182_3
YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 236 - protein, 299 aa, Artificial Sequence
  Synthetic IL1RAP transcript variant 6 NM_001167931_1
YRAHFGTDETILDGKEYDIYVSYARNAEEEEFVLLTLRGVLENEFGYKLCIFDRDSLPGGNTVEAVFDFIQRSRRMIVVLSPDYVTEKSISMLEFKLGVMCQNSIATKLIVVEYRPLEHPHPGILQLKESVSFVSWKGEKSKHSGSKFWKALRLALPLRSLSASSGWNESCSSQSDISLDHVQRRRSRLKEPPELQSSERAAGSPPAPGTMSKHRGKSSATCRCCVTYCEGENHLRNKSRAEIHNQPQWETHLCKPVPQESETQWIQNGTRLEPPAPQISALALHHFTDLSNNNDFYIL

SEQ ID NO: 237 - protein, 207 aa, Artificial Sequence
  Synthetic IL1RL1 transcript variant 1 NM_016232.4
LKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 238 - protein, 219 aa, Artificial Sequence
  Synthetic IL1RL2 NM_003854.2
NIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 239 - protein, 13 aa, Artificial Sequence
  Synthetic IL2RA transcript variant 1 NM_000417_2
TWQRRQRKSRRTI

SEQ ID NO: 240 - protein, 286 aa, Artificial Sequence
  Synthetic IL2RB transcript variant 1 NM_000878_4
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 241 - protein, 86 aa, Artificial Sequence
  Synthetic IL2RG NM_000206_2
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 242 - protein, 53 aa, Artificial Sequence
  Synthetic IL3RA transcript variant 1 and 2 NM_002183_3
RRYLVMQRLFPRIPHMKDPIGDSFQNDKLVVWEAGKAGLEECLVTEVQVVQKT

SEQ ID NO: 243 - protein, 569 aa, Artificial Sequence
  Synthetic IL4R transcript variant 1 NM_000418_3
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 244 - protein, 569 aa, Artificial Sequence; identical to SEQ ID NO: 243
  Synthetic IL4R transcript variant 1 NM_000418_3

SEQ ID NO: 245 - protein, 58 aa, Artificial Sequence
  Synthetic IL5RA transcript variant 1 NM_000564_4
KICHLWIKLFPPIPAPKSNIKDLFVTTNYEKAGSSETEIEVICYIEKPGVETLEDSVF

SEQ ID NO: 246 - protein, 82 aa, Artificial Sequence
  Synthetic IL6R transcript variant 1 NM_000565_3
RFKKTWKLRALKEGKTSMHPPYSLGQLVPERPRPTPVLVPLISPPVSPSSLGSDNTSSHNRPDARDPRSPYDISNTDYFFPR

SEQ ID NO: 247 - protein, 277 aa, Artificial Sequence
  Synthetic IL6ST transcript variant 1 and 3 NM_002184_3
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 248 - protein, 196 aa, Artificial Sequence
  Synthetic IL7RA Isoform 1 NM_002185.4
WKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 249 - protein, 35 aa, Artificial Sequence
  Synthetic IL7RA Isoform 3 (C-term deletion) (interleukin 7 receptor)
WKKRIKPIVWPSLPDHKKTLEHLCKKPRKVSVFGA

SEQ ID NO: 250 - protein, 230 aa, Artificial Sequence
  Synthetic IL9R transcript variant 1 NM_002186_2
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 251 - protein, 322 aa, Artificial Sequence
  Synthetic IL10RA transcript variant 1 NM_001558_3
QLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 252 - protein, 83 aa, Artificial Sequence
  Synthetic IL10RB NM_000628_4
ALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 253 - protein, 31 aa, Artificial Sequence
  Synthetic IL11RA NM_001142784_2
RLRRGGKDGSPKPGFLASVIPVDRRPGAPNL

SEQ ID NO: 254 - protein, 92 aa, Artificial Sequence
  Synthetic IL12RB1 transcript variant 1 and 4 NM_005535_2
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 255 - protein, 90 aa, Artificial Sequence
  Synthetic IL12RB1 transcript variant 3 NM_001290023_1
NRAARHLCPPLPTPCASSAIEFPGGKETWQWINPVDFQEEASLQEALVVEMSWDKGERTEPLEKTELPEGAPELALDTELSLEDGDRCDR

SEQ ID NO: 256 - protein, 219 aa, Artificial Sequence
  Synthetic IL12RB2 transcript variant 1 and 3 NM_001559_2
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 257 - protein, 60 aa, Artificial Sequence
  Synthetic IL13RA1 NM_001560_2
KRLKIIIFPPIPDPGKIFKEMFGDQNDDTLHWKKYDIYEKQTKEETDSVVLIENLKKASQ

SEQ ID NO: 258 - protein, 17 aa, Artificial Sequence
  Synthetic IL13RA2 NM_000640_2
RKPNTYPKMIPEFFCDT

SEQ ID NO: 259 - protein, 39 aa, Artificial Sequence
  Synthetic IL15RA transcript variant 4 NM_001256765_1
KSRQTPPLASVEMEAMEALPVTWGTSSRDEDLENCSHHL

SEQ ID NO: 260 - protein, 525 aa, Artificial Sequence
  Synthetic IL17RA NM_014339_6
CMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 261 - protein, 189 aa, Artificial Sequence
  Synthetic IL17RB NM_018725_3
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 262 - protein, 232 aa, Artificial Sequence
  Synthetic IL17RC transcript variant 1 NM_153460_3
KKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 263 - protein, 219 aa, Artificial Sequence
  Synthetic IL17RC transcript variant 4 NM_001203263_1
KKDHAKAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 264 - protein, 419 aa, Artificial Sequence
  Synthetic IL17RD transcript variant 2 NM_017563_4
CRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 265 - protein, 192 aa, Artificial Sequence
  Synthetic IL17RE transcript variant 1 NM_153480_1
TCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 266 - protein, 191 aa, Artificial Sequence
  Synthetic IL18R1 transcript variant 1 NM_003855_3
YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 267 - protein, 222 aa, Artificial Sequence
  Synthetic IL18RAP NM_003853_3
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 268 - protein, 282 aa, Artificial Sequence
  Synthetic IL20RA transcript variant 1 NM_014432_3
SIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 269 - protein, 57 aa, Artificial Sequence
  Synthetic IL20RB NM_144717_3
WKMGRLLQYSCCPVVVLPDTLKITNSPQKLISCRREEVDACATAVMSPEELLRAWIS

SEQ ID NO: 270 - protein, 285 aa, Artificial Sequence
  Synthetic IL21R transcript variant 2 NM_181078_2
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 271 - protein, 325 aa, Artificial Sequence
  Synthetic IL22RA1 NM_021258_3
SYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 272 - protein, 253 aa, Artificial Sequence
  Synthetic IL23R NM_144701_2
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 273 - protein, 99 aa, Artificial Sequence
  Synthetic IL27RA NM_004843_3
TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 274 - protein, 86 aa, Artificial Sequence
  Synthetic IL27RA NM_004843_3
TSWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 275 - protein, 189 aa, Artificial Sequence
  Synthetic IL31RA transcript variant 1 NM_139017_5
KKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 276 - protein, 106 aa, Artificial Sequence
  Synthetic IL31RA transcript variant 4 NM_001242638_1
KKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 277 - protein, 303 aa, Artificial Sequence
  Synthetic LEPR transcript variant 1 NM_002303_5
SHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 278 - protein, 96 aa, Artificial Sequence
  Synthetic LEPR transcript variant 2 NM_001003680_3
SHQRMKKLFWEDVPNPKNCSWAQGLNFQKMLEGSMFVKSHHHSLISSTQGHKHCGRPQGPLHRKTRDLCSLVYLLTLPPLLSYDPAKSPSVRNTQE

SEQ ID NO: 279 - protein, 34 aa, Artificial Sequence
  Synthetic LEPR transcript variant 3 NM_001003679_3
SHQRMKKLFWEDVPNPKNCSWAQGLNFQKRTDIL

SEQ ID NO: 280 - protein, 44 aa, Artificial Sequence
  Synthetic LEPR transcript variant 5 NM_001198688_1
SHQRMKKLFWEDVPNPKNCSWAQGLNFQKKMPGTKELLGGGWLT

SEQ ID NO: 281 - protein, 239 aa, Artificial Sequence
  Synthetic LIFR NM_001127671_1
YRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 282 - protein, 202 aa, Artificial Sequence
  Synthetic LMP1 NC_007605_1
YYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 283 - protein, 122 aa, Artificial Sequence
  Synthetic MPL NM_005373_2
RWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 284 - protein, 304 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRLIPIKYKAMKKEFPSILRFITVCDYTNPCTKSWFWTRLAKALSLP

SEQ ID NO: 285 - protein, 296 aa, Artificial Sequence
  Synthetic MYD88 transcript variant 2 NM_002468_4
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 286 - protein, 251 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 287 - protein, 191 aa, Artificial Sequence
  Synthetic MYD88 transcript variant 4 NM_001172569_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGAAGWWWLSLMITCRARNVTSRPNLHSASLQVPIRSD

SEQ ID NO: 288 - protein, 146 aa, Artificial Sequence
  Synthetic MYD88 transcript variant 5 NM_001172566_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGAAGWWWLSLMITCRARNVTSRPNLHSASLQVPIRSD

SEQ ID NO: 289 - protein, 172 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 290 - protein, 127 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDI

SEQ ID NO: 291 - protein, 304 aa, Artificial Sequence
  Synthetic MYD88 transcript variant 1 NM_001172567_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRPIPIKYKAMKKEFPSILRFITVCDYTNPCTKSWFWTRLAKALSLP

SEQ ID NO: 292 - protein, 296 aa, Artificial Sequence
  Synthetic MYD88 transcript variant 2 NM_002468_4
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRPIPIKYKAMKKEFPSILRFITVCDYTNPCTKSWFWTRLAKALSLP

SEQ ID NO: 293 - protein, 251 aa, Artificial Sequence
  Synthetic MYD88 transcript variant 3 NM_001172568_1
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRPIPIKYKAMKKEFPSILRFITVCDYTNPCTKSWFWTRLAKALSLP

SEQ ID NO: 294 - protein, 218 aa, Artificial Sequence
  Synthetic OSMR transcript variant 4 NM_001323505_1
KSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC

SEQ ID NO: 295 - protein, 364 aa, Artificial Sequence
  Synthetic PRLR transcript variant 1 NM_000949_6
KGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 296 - protein, 42 aa, Artificial Sequence
  Synthetic TNFRSF4 NM_003327_3
ALYLLRRDQRLPPDAHKPPGGGSFRTPIQEEQADAHSTLAKI

SEQ ID NO: 297 - protein, 188 aa, Artificial Sequence
  Synthetic TNFRSF8 transcript variant 1 NM_001243_4
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 298 - protein, 42 aa, Artificial Sequence; identical to SEQ ID NO: 53
  Synthetic TNFRSF9 NM_001561_5

SEQ ID NO: 299 - protein, 60 aa, Artificial Sequence; identical to SEQ ID NO: 62
  Synthetic TNFRSF14 transcript variant 1 NM_003820_3

SEQ ID NO: 300 - protein, 58 aa, Artificial Sequence
  Synthetic TNFRSF18 transcript variant 1 NM_004195_2
QLGLHIWQLRSQCMWPRETQLLLEVPPSTEDARSCQFPEEERGERSAEEKGRLGDLWV

SEQ ID NO: 301 - protein, 51 aa, Artificial Sequence
  Synthetic TNFRSF18 transcript variant 3_ NM_148902_1
QLGLHIWQLRKTQLLLEVPPSTEDARSCQFPEEERGERSAEEKGRLGDLWV

SEQ ID NO: 302 - protein, 23 aa, Artificial Sequence
  Synthetic Linker
GSGGSEGGGSEGGAATAGSGSGS

SEQ ID NO: 303 - protein, 5 aa, Artificial Sequence
  Synthetic TRAF1, TRAF2, and TRAF3 consensus binding sequence
  misc_feature (2)..(2): Xaa can be any naturally occurring amino acid
  misc_feature (4)..(4): Xaa can be any naturally occurring amino acid
PXQXT

SEQ ID NO: 304 - protein, 4 aa, Artificial Sequence
  Synthetic TRAF2 consensus binding sequence
  misc_feature (2)..(3): Xaa can be any naturally occurring amino acid
SXXE

SEQ ID NO: 305 - protein, 6 aa, Artificial Sequence
  Synthetic TRAF6 consensus binding sequence
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
QXPXEX

SEQ ID NO: 306 - protein, 4 aa, Artificial Sequence
  Synthetic Box1 motif
  misc_feature (2)..(3): Xaa can be any naturally occurring amino acid
PXXP

SEQ ID NO: 307 - protein, 4 aa, Artificial Sequence
  Synthetic Shc phosphotyrosine-binding binding motif
  misc_feature (2)..(3): Xaa can be any naturally occurring amino acid
NXXY

SEQ ID NO: 308 - protein, 4 aa, Artificial Sequence
  Synthetic STAT3 consensus binding sequence
  misc_feature (2)..(3): Xaa can be any naturally occurring amino acid
YXXQ

SEQ ID NO: 309 - protein, 4 aa, Artificial Sequence
  Synthetic STAT5 recruitment sequence
YLPL

SEQ ID NO: 310 - protein, 4 aa, Artificial Sequence
  Synthetic STAT5 consensus recruitment sequence
  misc_feature (1)..(1): Xaa is phosphorylated tyrosine
  misc_feature (3)..(3): Xaa can be any naturally occurring amino acid
XLXL

SEQ ID NO: 311 - protein, 570 aa, Influenza virus
  misc_feature (1)..(570): Influenze A HA from H1N1
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRRRKKRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 312 - protein, 470 aa, Influenza virus
  misc_feature (1)..(470): Influenze A NA from H10N7
MNPNQKLFALSGVAIALSILNLLIGISNVGLNVSLHLKGSSDQDKNWTCTSVTQNNTTLIENTYVNNTTVINKGTGTTKQNYLMLNKSLCKVEGWVVVAKDNAIRFGESEQIIVTREPYVSCDPLGCKMYALHQGTTIRNKHSNGTIHDRTAFRGLISTPLGSPPVVSNSDFLCVGWSSTSCHDGIGRMTICVQGNNNNATATVYYDRRLTTTIKTWAGNILRTQESECVCHNGTCVVIMTDGSASSQAHTKVLYFHKGLVIKEEALKGSARHIEECSCYGHNSKVTCVCRDNWQGANRPVIEIDMNAMEHTSQYLCTGVLTDTSRPSDKSMGDCNNPITGSPGAPGVKGFGFLDSDNTWLGRTISPRSRSGFEMLKIPNAGTDPNSRITERQEIVDNNNWSGYSGSFIDYWDESSVCYNPCFYVELIRGRPEEAKYVWWTSNSLVALCGSPISVGSGSFPDGAQIQYFS

SEQ ID NO: 313 - protein, 523 aa, Artificial Sequence
  Synthetic MV(ed)-F-delta-30
MSIMGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 314 - protein, 599 aa, Artificial Sequence
  Synthetic MV(ed)-H-delta-18
MGSRIVINREHLMIDRPYVLLAVLFVMSLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 315 - protein, 593 aa, Artificial Sequence
  Synthetic MV(ed)-H-delta-24
MNREHLMIDRPYVLLAVLFVMSLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 316 - DNA, 477 bp, Artificial Sequence
  Synthetic hGH polyA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctgttgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 317 - DNA, 49 bp, Artificial Sequence
  Synthetic SPA1
aataaaagatctttattttcattagatctgtgtgttggttttttgtgtg

SEQ ID NO: 318 - DNA, 120 bp, Artificial Sequence
  Synthetic SPA2
aataaaatatctcagagctctagacatctgtgtgttggttttttgtgtgtagtaatgaggatctggagatattgaagtatcttccggacgactaacagctgtcattggcggatcttaata

SEQ ID NO: 319 - DNA, 295 bp, Artificial Sequence
  Synthetic b-globin polyA spacer B
atctcaagagtggcagcggtcttgagtggcagcggcggtatacggcagcggcatgtaactagctcctcagtggcagcgatgaggaggcaataaaggaaattgattttcattgcaatagtgtgttggaattttttgtgtctctcaaggttctgttaagtaactgaacccaatgtcgttagtgacgcttagctcttaagaggtcactgacctaacaatctcaagagtggcagcggtcttgagtggcagcggcggtatacggcagcgctatctaagtagtaacaagtagcgtggggca

SEQ ID NO: 320 - DNA, 512 bp, Artificial Sequence
  Synthetic b-globin polyA spacer A
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttaataaaggaaattgattttcattgcaatagtgtgttggaattttttgtgtctctcacacgtagtgggccatcgccctgatagacggtttttcgccctttgacgttggagtccacgttcttcgatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttagaattt

SEQ ID NO: 321 - DNA, 243 bp, Artificial Sequence
  Synthetic 250 cHS4 insulator v1
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacgtggggggatacggggaaaagctt

SEQ ID NO: 322 - DNA, 243 bp, Artificial Sequence
  Synthetic 250 cHS4 insulator v2
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcgtgcagacacgtggggggatacggggaaaagctt

SEQ ID NO: 323 - DNA, 650 bp, Artificial Sequence
  Synthetic 650 cHS4 insulator
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatatt

SEQ ID NO: 324 - DNA, 420 bp, Artificial Sequence
  Synthetic 400 cHS4 insulator
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccac

SEQ ID NO: 325 - DNA, 949 bp, Artificial Sequence
  Synthetic 650 cHS4 insulator and b-globin polyA spacer B
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatattgcgatgccccacgctacttgttactacttagatagcgctgccgtataccgccgctgccactcaagaccgctgccactcttgagattgttaggtcagtgacctcttaagagctaagcgtcactaacgacattgggttcagttacttaacagaaccttgagagacacaaaaaattccaacacactattgcaatgaaaatcaatttcctttattgcctcctcatcgctgccactgaggagctagttacatgccgctgccgtataccgccgctgccactcaagaccgctgccactcttgagat

SEQ ID NO: 326 - DNA, 949 bp, Artificial Sequence
  Synthetic b-globin polyA spacer B and 650 cHS4 insulator
atctcaagagtggcagcggtcttgagtggcagcggcggtatacggcagcggcatgtaactagctcctcagtggcagcgatgaggaggcaataaaggaaattgattttcattgcaatagtgtgttggaattttttgtgtctctcaaggttctgttaagtaactgaacccaatgtcgttagtgacgcttagctcttaagaggtcactgacctaacaatctcaagagtggcagcggtcttgagtggcagcggcggtatacggcagcgctatctaagtagtaacaagtagcgtggggcatcgcgagctcacggggacagcccccccccaaagcccccagggatggtcgtacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcatgcagacacatggggggatacggggaaaaagctttaggctctgcatgtttgatggtgtatggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccagcgtgcccactggccatgtccctcagtgccacatccccacagttcttcatcacctccagggacggtgacccccccacctccgtgggcagctgtgccactgcagcaccgctctttggagaagataaatcttgctaaatccagcccgaccctcccctggcacaacataaggccattatctctcatccaactccaggacggagtcagtgagaatatt

SEQ ID NO: 327 - DNA, 15 bp, Artificial Sequence
  Synthetic Kozak sequence
  misc_feature (1)..(3): nnn, if present, is GCC
  misc_feature (10)..(10): n is A or G
nnngccgccnccatg

SEQ ID NO: 328 - DNA, 9 bp, Artificial Sequence
  Synthetic Kozak sequence
  misc_feature (7)..(7): n is T or U
  misc_feature (9)..(9): n, if present, is G
ccaccangn

SEQ ID NO: 329 - DNA, 9 bp, Artificial Sequence
  Synthetic Kozak-type sequence 2
  misc_feature (7)..(7): n is T or U
  misc_feature (9)..(9): n, if present, is G
ccgccangn

SEQ ID NO: 330 - DNA, 13 bp, Artificial Sequence
  Synthetic Kozak-type sequence 3
  misc_feature (11)..(11): n is T or U
  misc_feature (13)..(13): n, if present, is G
gccgccgccangn

SEQ ID NO: 331 - DNA, 13 bp, Artificial Sequence
  Synthetic Kozak sequence
  misc_feature (11)..(11): n is T or U
  misc_feature (13)..(13): n, if present, is G
gccgccaccangn

SEQ ID NO: 332 - RNA, 12 nt, Artificial Sequence
  Synthetic Kozak sequence
gccgccaccaug

SEQ ID NO: 333 - DNA, 28 bp, Mus musculus
  misc_feature (1)..(28): SIBR (synthetic inhibitory BIC-derived RNA)
ctggaggcttgctgaaggctgtatgctg

SEQ ID NO: 334 - DNA, 45 bp, Mus musculus
  misc_feature (1)..(45): 3? microRNA flanking sequence of miR-155
caggacacaaggcctgttactagcactcacatggaacaaatggcc

SEQ ID NO: 335 - DNA, 19 bp, Artificial Sequence
  Synthetic synthetic DNA encoding stem
gttttggccactgactgac

SEQ ID NO: 336 - protein, 511 aa, Artificial Sequence
  Synthetic VSV-G envelope protein
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 337 - protein, 563 aa, Artificial Sequence
  Synthetic Baboon retroviral envelope glycoprotein
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 338 - protein, 654 aa, Artificial Sequence
  Synthetic MuLV envelope protein
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 339 - protein, 545 aa, Artificial Sequence
  Synthetic Baboon retroviral envelope glycoprotein -delta-R (HA)
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHA

SEQ ID NO: 340 - protein, 546 aa, Artificial Sequence
  Synthetic Baboon retroviral envelope glycoprotein -delta-R (HAM)
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 341 - protein, 905 aa, Artificial Sequence
  Synthetic Fusion of anti-CD3 scFV from UCHT1 to MuLV envelope protein
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 342 - DNA, 223 bp, Artificial Sequence
  Synthetic cCBL miRNA at P1
cggcacctgcgctgccgttggatcggggatgaaagctggcgctggaggcttgctgaaggctgtatgctgtaataactcccaactcactgggttttggccactgactgacccagtgaggggagttattacaggacacaaggcctgttactagcactcacatggaacaaatggcccacattggtgccggatgaagctcttatgttgcgtcccatcgcaggtgcct

SEQ ID NO: 343 - DNA, 204 bp, Artificial Sequence
  Synthetic cCBL miRNA at P2
cctcacctgcttgcgtcccattctggaggcttgctgaaggctgtatgctgtttagtaatccgaaatgtgtcgttttggccactgactgacgacacattggattactaaacaggacacaaggcctgttactagcactcacatggaacaaatggccgttgcctgagtcttggcagcgagagatcactaactgctaagcaggtgctt

SEQ ID NO: 344 - DNA, 204 bp, Artificial Sequence
  Synthetic cCBL miRNA at P3
tgtcacctgcactaactgctaactggaggcttgctgaaggctgtatgctgtaatcattgcaggtcagatcagttttggccactgactgactgatctgatgcaatgattacaggacacaaggcctgttactagcactcacatggaacaaatggccgtgttaattgtccatgtagcgaggcatccttatggcgtgggcaggtgtcc

SEQ ID NO: 345 - DNA, 200 bp, Artificial Sequence
  Synthetic cCBL miRNA at P4
ccttcacctgccttatggcgtggctggaggcttgctgaaggctgtatgctgtttgtgaatgaatttctggaggttttggccactgactgacctccagaatcattcacaaacaggacacaaggcctgttactagcactcacatggaacaaatggccggtgtccgttatcggggaagaaggtcgcgcacatagcaggtgtcc

SEQ ID NO: 346 - DNA, 223 bp, Artificial Sequence
  Synthetic CD3Z miRNA at P1
cggcacctgcgctgccgttggatcggggatgaaagctggcgctggaggcttgctgaaggctgtatgctgacatggtacagttcaatggtggttttggccactgactgaccaccattgctgtaccatgtcaggacacaaggcctgttactagcactcacatggaacaaatggcccacattggtgccggatgaagctcttatgttgcgtcccatcgcaggtgcct

SEQ ID NO: 347 - DNA, 204 bp, Artificial Sequence
  Synthetic CD3Z miRNA at P2
cctcacctgcttgcgtcccattctggaggcttgctgaaggctgtatgctgtcagtctgttcatcttctggcgttttggccactgactgacgccagaaggaacagactgacaggacacaaggcctgttactagcactcacatggaacaaatggccgttgcctgagtcttggcagcgagagatcactaactgctaagcaggtgctt

SEQ ID NO: 348 - DNA, 204 bp, Artificial Sequence
  Synthetic CD3Z miRNA at P3
tgtcacctgcactaactgctaactggaggcttgctgaaggctgtatgctgaagcgtgaagtgaatcaacgggttttggccactgactgacccgttgatacttcacgcttcaggacacaaggcctgttactagcactcacatggaacaaatggccgtgttaattgtccatgtagcgaggcatccttatggcgtgggcaggtgtcc

SEQ ID NO: 349 - DNA, 200 bp, Artificial Sequence
  Synthetic CD3Z miRNA at P4
ccttcacctgccttatggcgtggctggaggcttgctgaaggctgtatgctggcagtatcctagtacattgacgttttggccactgactgacgtcaatgttaggatactgccaggacacaaggcctgttactagcactcacatggaacaaatggccggtgtccgttatcggggaagaaggtcgcgcacatagcaggtgtcc

SEQ ID NO: 350 - DNA, 223 bp, Artificial Sequence
  Synthetic PD1 miRNA at P1
cggcacctgcgctgccgttggatcggggatgaaagctggcgctggaggcttgctgaaggctgtatgctgaatgggttccaaggagagctcgttttggccactgactgacgagctctctggaacccattcaggacacaaggcctgttactagcactcacatggaacaaatggcccacattggtgccggatgaagctcttatgttgcgtcccatcgcaggtgcct

SEQ ID NO: 351 - DNA, 204 bp, Artificial Sequence
  Synthetic PD1 miRNA at P2
cctcacctgcttgcgtcccattctggaggcttgctgaaggctgtatgctgttctctcgccactggaaatccgttttggccactgactgacggatttcctggcgagagaacaggacacaaggcctgttactagcactcacatggaacaaatggccgttgcctgagtcttggcagcgagagatcactaactgctaagcaggtgctt

SEQ ID NO: 352 - DNA, 204 bp, Artificial Sequence
  Synthetic PD1 miRNA at P3
tgtcacctgcactaactgctaactggaggcttgctgaaggctgtatgctgtttagcacgaagctctccgatgttttggccactgactgacatcggagattcgtgctaaacaggacacaaggcctgttactagcactcacatggaacaaatggccgtgttaattgtccatgtagcgaggcatccttatggcgtgggcaggtgtcc

SEQ ID NO: 353 - DNA, 200 bp, Artificial Sequence
  Synthetic PD1 miRNA at P4
ccttcacctgccttatggcgtggctggaggcttgctgaaggctgtatgctgttgtccgtctggttgctgggggttttggccactgactgacccccagcacagacggacaacaggacacaaggcctgttactagcactcacatggaacaaatggccggtgtccgttatcggggaagaaggtcgcgcacatagcaggtgtcc

SEQ ID NO: 354 - DNA, 223 bp, Artificial Sequence
  Synthetic CTLA4 miRNA at P1
cggcacctgcgctgccgttggatcggggatgaaagctggcgctggaggcttgctgaaggctgtatgctgattacataaatctgggttccggttttggccactgactgaccggaacccatttatgtaatcaggacacaaggcctgttactagcactcacatggaacaaatggcccacattggtgccggatgaagctcttatgttgcgtcccatcgcaggtgcct

SEQ ID NO: 355 - DNA, 204 bp, Artificial Sequence
  Synthetic CTLA4 miRNA at P2
cctcacctgcttgcgtcccattctggaggcttgctgaaggctgtatgctgatactcacacacaaagctggcgttttggccactgactgacgccagctttgtgtgagtatcaggacacaaggcctgttactagcactcacatggaacaaatggccgttgcctgagtcttggcagcgagagatcactaactgctaagcaggtgctt

SEQ ID NO: 356 - DNA, 204 bp, Artificial Sequence
  Synthetic CTLA4 miRNA at P3
tgtcacctgcactaactgctaactggaggcttgctgaaggctgtatgctgtaaatctgggttccgttgcctgttttggccactgactgacaggcaacgacccagatttacaggacacaaggcctgttactagcactcacatggaacaaatggccgtgttaattgtccatgtagcgaggcatccttatggcgtgggcaggtgtcc

SEQ ID NO: 357 - DNA, 200 bp, Artificial Sequence
  Synthetic CTLA4 miRNA at P4
ccttcacctgccttatggcgtggctggaggcttgctgaaggctgtatgctgtaacttaattccttgacccacgttttggccactgactgacgtgggtcagaattaagttacaggacacaaggcctgttactagcactcacatggaacaaatggccggtgtccgttatcggggaagaaggtcgcgcacatagcaggtgtcc

SEQ ID NO: 358 - DNA, 223 bp, Artificial Sequence
  Synthetic TIM3 miRNA at P1
cggcacctgcgctgccgttggatcggggatgaaagctggcgctggaggcttgctgaaggctgtatgctgtttgatgaccaacttcaggttgttttggccactgactgacaacctgaatggtcatcaaacaggacacaaggcctgttactagcactcacatggaacaaatggcccacattggtgccggatgaagctcttatgttgcgtcccatcgcaggtgcct

SEQ ID NO: 359 - DNA, 204 bp, Artificial Sequence
  Synthetic TIM3 miRNA at P2
cctcacctgcttgcgtcccattctggaggcttgctgaaggctgtatgctgtttatctgaagtttcatggacgttttggccactgactgacgtccatgacttcagataaacaggacacaaggcctgttactagcactcacatggaacaaatggccgttgcctgagtcttggcagcgagagatcactaactgctaagcaggtgctt

SEQ ID NO: 360 - DNA, 204 bp, Artificial Sequence
  Synthetic TIM3 miRNA at P3
tgtcacctgcactaactgctaactggaggcttgctgaaggctgtatgctgtatagcttcagtttggtccacgttttggccactgactgacgtggaccactgaagctatacaggacacaaggcctgttactagcactcacatggaacaaatggccgtgttaattgtccatgtagcgaggcatccttatggcgtgggcaggtgtcc

SEQ ID NO: 361 - DNA, 200 bp, Artificial Sequence
  Synthetic TIM3 miRNA at P4
ccttcacctgccttatggcgtggctggaggcttgctgaaggctgtatgctgtatgcctgggatttggatccggttttggccactgactgaccggatccatcccaggcatacaggacacaaggcctgttactagcactcacatggaacaaatggccggtgtccgttatcggggaagaaggtcgcgcacatagcaggtgtcc

SEQ ID NO: 362 - DNA, 223 bp, Artificial Sequence
  Synthetic LAG3 miRNA at P1
cggcacctgcgctgccgttggatcggggatgaaagctggcgctggaggcttgctgaaggctgtatgctgttatacatgatggagacgttggttttggccactgactgaccaacgtctatcatgtataacaggacacaaggcctgttactagcactcacatggaacaaatggcccacattggtgccggatgaagctcttatgttgcgtcccatcgcaggtgcct

SEQ ID NO: 363 - DNA, 204 bp, Artificial Sequence
  Synthetic LAG3 miRNA at P2
cctcacctgcttgcgtcccattctggaggcttgctgaaggctgtatgctgtaaagtcgccattgtctccaggttttggccactgactgacctggagactggcgactttacaggacacaaggcctgttactagcactcacatggaacaaatggccgttgcctgagtcttggcagcgagagatcactaactgctaagcaggtgctt

SEQ ID NO: 364 - DNA, 204 bp, Artificial Sequence
  Synthetic LAG3 miRNA at P3
tgtcacctgcactaactgctaactggaggcttgctgaaggctgtatgctgattgccaatgtgacagtggcagttttggccactgactgactgccactgacattggcaatcaggacacaaggcctgttactagcactcacatggaacaaatggccgtgttaattgtccatgtagcgaggcatccttatggcgtgggcaggtgtcc

SEQ ID NO: 365 - DNA, 200 bp, Artificial Sequence
  Synthetic LAG3 miRNA at P4
ccttcacctgccttatggcgtggctggaggcttgctgaaggctgtatgctgattgtctccagtcaccaggaggttttggccactgactgacctcctggtctggagacaatcaggacacaaggcctgttactagcactcacatggaacaaatggccggtgtccgttatcggggaagaaggtcgcgcacatagcaggtgtcc

SEQ ID NO: 366 - DNA, 223 bp, Artificial Sequence
  Synthetic SMAD2 miRNA at P1
cggcacctgcgctgccgttggatcggggatgaaagctggcgctggaggcttgctgaaggctgtatgctgaagattgcactatcacttagggttttggccactgactgaccctaagtgagtgcaatcttcaggacacaaggcctgttactagcactcacatggaacaaatggcccacattggtgccggatgaagctcttatgttgcgtcccatcgcaggtgcct

SEQ ID NO: 367 - DNA, 204 bp, Artificial Sequence
  Synthetic SMAD2 miRNA at P2
cctcacctgcttgcgtcccattctggaggcttgctgaaggctgtatgctgtatgacatgcttgagcaacgcgttttggccactgactgacgcgttgctagcatgtcatacaggacacaaggcctgttactagcactcacatggaacaaatggccgttgcctgagtcttggcagcgagagatcactaactgctaagcaggtgctt

SEQ ID NO: 368 - DNA, 204 bp, Artificial Sequence
  Synthetic SMAD2 miRNA at P3
tgtcacctgcactaactgctaactggaggcttgctgaaggctgtatgctgttaagtagtactgatgtggtggttttggccactgactgaccaccacatgtactacttaacaggacacaaggcctgttactagcactcacatggaacaaatggccgtgttaattgtccatgtagcgaggcatccttatggcgtgggcaggtgtcc

SEQ ID NO: 369 - DNA, 200 bp, Artificial Sequence
  Synthetic SMAD2 miRNA at P4
ccttcacctgccttatggcgtggctggaggcttgctgaaggctgtatgctgttacctaggacatttactctggttttggccactgactgaccagagtaagtcctaggtaacaggacacaaggcctgttactagcactcacatggaacaaatggccggtgtccgttatcggggaagaaggtcgcgcacatagcaggtgtcc

SEQ ID NO: 370 - DNA, 223 bp, Artificial Sequence
  Synthetic TNFRSF10B miRNA at P1
cggcacctgcgctgccgttggatcggggatgaaagctggcgctggaggcttgctgaaggctgtatgctgaaaggaggtcattccagtgaggttttggccactgactgacctcactggtgacctcctttcaggacacaaggcctgttactagcactcacatggaacaaatggcccacattggtgccggatgaagctcttatgttgcgtcccatcgcaggtgcct

SEQ ID NO: 371 - DNA, 204 bp, Artificial Sequence
  Synthetic TNFRSF10B miRNA at P2
cctcacctgcttgcgtcccattctggaggcttgctgaaggctgtatgctgtatgggtgcaaatgagactgcgttttggccactgactgacgcagtctcttgcacccatacaggacacaaggcctgttactagcactcacatggaacaaatggccgttgcctgagtcttggcagcgagagatcactaactgctaagcaggtgctt

SEQ ID NO: 372 - DNA, 204 bp, Artificial Sequence
  Synthetic TNFRSF10B miRNA at P3
tgtcacctgcactaactgctaactggaggcttgctgaaggctgtatgctgtagtcctgtccatatttgcaggttttggccactgactgacctgcaaatggacaggactacaggacacaaggcctgttactagcactcacatggaacaaatggccgtgttaattgtccatgtagcgaggcatccttatggcgtgggcaggtgtcc

SEQ ID NO: 373 - DNA, 200 bp, Artificial Sequence
  Synthetic TNFRSF10B miRNA at P4
ccttcacctgccttatggcgtggctggaggcttgctgaaggctgtatgctgatactcacgatctcattgagggttttggccactgactgaccctcaatgatcgtgagtatcaggacacaaggcctgttactagcactcacatggaacaaatggccggtgtccgttatcggggaagaaggtcgcgcacatagcaggtgtcc

SEQ ID NO: 374 - DNA, 223 bp, Artificial Sequence
  Synthetic PPP2CA miRNA at P1
cggcacctgcgctgccgttggatcggggatgaaagctggcgctggaggcttgctgaaggctgtatgctgaaatcgttactacattccggtgttttggccactgactgacaccggaatagtaacgatttcaggacacaaggcctgttactagcactcacatggaacaaatggcccacattggtgccggatgaagctcttatgttgcgtcccatcgcaggtgcct

SEQ ID NO: 375 - DNA, 204 bp, Artificial Sequence
  Synthetic PPP2CA miRNA at P2
cctcacctgcttgcgtcccattctggaggcttgctgaaggctgtatgctgaataacaaagaacaacatggggttttggccactgactgaccccatgtttctttgttattcaggacacaaggcctgttactagcactcacatggaacaaatggccgttgcctgagtcttggcagcgagagatcactaactgctaagcaggtgctt

SEQ ID NO: 376 - DNA, 204 bp, Artificial Sequence
  Synthetic PPP2CA miRNA at P3
tgtcacctgcactaactgctaactggaggcttgctgaaggctgtatgctgtttgtatctggtgatttgccagttttggccactgactgactggcaaatccagatacaaacaggacacaaggcctgttactagcactcacatggaacaaatggccgtgttaattgtccatgtagcgaggcatccttatggcgtgggcaggtgtcc

SEQ ID NO: 377 - DNA, 200 bp, Artificial Sequence
  Synthetic PPP2CA miRNA at P4
ccttcacctgccttatggcgtggctggaggcttgctgaaggctgtatgctgtttgccaccaattctaaacaggttttggccactgactgacctgtttagttggtggcaaacaggacacaaggcctgttactagcactcacatggaacaaatggccggtgtccgttatcggggaagaaggtcgcgcacatagcaggtgtcc

SEQ ID NO: 378 - DNA, 223 bp, Artificial Sequence
  Synthetic TNFRSF6 miRNA at P1
cggcacctgcgctgccgttggatcggggatgaaagctggcgctggaggcttgctgaaggctgtatgctgtttaatcaatgtgtcatacgcgttttggccactgactgacgcgtatgacattgattaaacaggacacaaggcctgttactagcactcacatggaacaaatggcccacattggtgccggatgaagctcttatgttgcgtcccatcgcaggtgcct

SEQ ID NO: 379 - DNA, 204 bp, Artificial Sequence
  Synthetic TNFRSF6 miRNA at P2
cctcacctgcttgcgtcccattctggaggcttgctgaaggctgtatgctgtttacagccagctattaagaagttttggccactgactgacttcttaatctggctgtaaacaggacacaaggcctgttactagcactcacatggaacaaatggccgttgcctgagtcttggcagcgagagatcactaactgctaagcaggtgctt

SEQ ID NO: 380 - DNA, 204 bp, Artificial Sequence
  Synthetic TNFRSF6 miRNA at P3
tgtcacctgcactaactgctaactggaggcttgctgaaggctgtatgctgtttaacttgacttagtgtcatgttttggccactgactgacatgacactgtcaagttaaacaggacacaaggcctgttactagcactcacatggaacaaatggccgtgttaattgtccatgtagcgaggcatccttatggcgtgggcaggtgtcc

SEQ ID NO: 381 - DNA, 200 bp, Artificial Sequence
  Synthetic TNFRSF6 miRNA at P4
ccttcacctgccttatggcgtggctggaggcttgctgaaggctgtatgctgttagtatctccaaaccaggctgttttggccactgactgacagcctggtggagatactaacaggacacaaggcctgttactagcactcacatggaacaaatggccggtgtccgttatcggggaagaaggtcgcgcacatagcaggtgtcc

SEQ ID NO: 382 - DNA, 223 bp, Artificial Sequence
  Synthetic BTLA miRNA at P1
cggcacctgcgctgccgttggatcggggatgaaagctggcgctggaggcttgctgaaggctgtatgctgtttaaacgttctactattctggttttggccactgactgaccagaataggaacgtttaaacaggacacaaggcctgttactagcactcacatggaacaaatggcccacattggtgccggatgaagctcttatgttgcgtcccatcgcaggtgcct

SEQ ID NO: 383 - DNA, 204 bp, Artificial Sequence
  Synthetic BTLA miRNA at P2
cctcacctgcttgcgtcccattctggaggcttgctgaaggctgtatgctgttaatttcccttcctgctgtggttttggccactgactgaccacagcagagggaaattaacaggacacaaggcctgttactagcactcacatggaacaaatggccgttgcctgagtcttggcagcgagagatcactaactgctaagcaggtgctt

SEQ ID NO: 384 - DNA, 204 bp, Artificial Sequence
  Synthetic BTLA miRNA at P3
tgtcacctgcactaactgctaactggaggcttgctgaaggctgtatgctgaattccagtttctgatagcaggttttggccactgactgacctgctatcaaactggaattcaggacacaaggcctgttactagcactcacatggaacaaatggccgtgttaattgtccatgtagcgaggcatccttatggcgtgggcaggtgtcc

SEQ ID NO: 385 - DNA, 200 bp, Artificial Sequence
  Synthetic BTLA miRNA at P4
ccttcacctgccttatggcgtggctggaggcttgctgaaggctgtatgctgttagaataaacttcagaccctgttttggccactgactgacagggtctggtttattctaacaggacacaaggcctgttactagcactcacatggaacaaatggccggtgtccgttatcggggaagaaggtcgcgcacatagcaggtgtcc

SEQ ID NO: 386 - DNA, 223 bp, Artificial Sequence
  Synthetic TIGIT miRNA at P1
cggcacctgcgctgccgttggatcggggatgaaagctggcgctggaggcttgctgaaggctgtatgctgttaactgtaagttcttgaggggttttggccactgactgacccctcaagcttacagttaacaggacacaaggcctgttactagcactcacatggaacaaatggcccacattggtgccggatgaagctcttatgttgcgtcccatcgcaggtgcct

SEQ ID NO: 387 - DNA, 204 bp, Artificial Sequence
  Synthetic TIGIT miRNA at P2
cctcacctgcttgcgtcccattctggaggcttgctgaaggctgtatgctgtattgtgcctgtcatcattccgttttggccactgactgacggaatgatcaggcacaatacaggacacaaggcctgttactagcactcacatggaacaaatggccgttgcctgagtcttggcagcgagagatcactaactgctaagcaggtgctt

SEQ ID NO: 388 - DNA, 204 bp, Artificial Sequence
  Synthetic TIGIT miRNA at P3
tgtcacctgcactaactgctaactggaggcttgctgaaggctgtatgctgaagtagtcatgcagctcggcagttttggccactgactgactgccgagccatgactacttcaggacacaaggcctgttactagcactcacatggaacaaatggccgtgttaattgtccatgtagcgaggcatccttatggcgtgggcaggtgtcc

SEQ ID NO: 389 - DNA, 200 bp, Artificial Sequence
  Synthetic TIGIT miRNA at P4
ccttcacctgccttatggcgtggctggaggcttgctgaaggctgtatgctgtttcctacaagttccctagacgttttggccactgactgacgtctagggcttgtaggaaacaggacacaaggcctgttactagcactcacatggaacaaatggccggtgtccgttatcggggaagaaggtcgcgcacatagcaggtgtcc

SEQ ID NO: 390 - DNA, 223 bp, Artificial Sequence
  Synthetic A2AR miRNA at P1
cggcacctgcgctgccgttggatcggggatgaaagctggcgctggaggcttgctgaaggctgtatgctgttgttccaacctagcatgggagttttggccactgactgactcccatgcggttggaacaacaggacacaaggcctgttactagcactcacatggaacaaatggcccacattggtgccggatgaagctcttatgttgcgtcccatcgcaggtgcct

SEQ ID NO: 391 - DNA, 204 bp, Artificial Sequence
  Synthetic A2AR miRNA at P2
cctcacctgcttgcgtcccattctggaggcttgctgaaggctgtatgctgtaaagatctccttcccttagggttttggccactgactgaccctaagggggagatctttacaggacacaaggcctgttactagcactcacatggaacaaatggccgttgcctgagtcttggcagcgagagatcactaactgctaagcaggtgctt

SEQ ID NO: 392 - DNA, 204 bp, Artificial Sequence
  Synthetic A2AR miRNA at P3
tgtcacctgcactaactgctaactggaggcttgctgaaggctgtatgctgaatagacacccagcatgagcagttttggccactgactgactgctcatggggtgtctattcaggacacaaggcctgttactagcactcacatggaacaaatggccgtgttaattgtccatgtagcgaggcatccttatggcgtgggcaggtgtcc

SEQ ID NO: 393 - DNA, 200 bp, Artificial Sequence
  Synthetic A2AR miRNA at P4
ccttcacctgccttatggcgtggctggaggcttgctgaaggctgtatgctgaattggtgtgggagaggacgagttttggccactgactgactcgtcctcccacaccaattcaggacacaaggcctgttactagcactcacatggaacaaatggccggtgtccgttatcggggaagaaggtcgcgcacatagcaggtgtcc

SEQ ID NO: 394 - DNA, 223 bp, Artificial Sequence
  Synthetic AHR miRNA at P1
cggcacctgcgctgccgttggatcggggatgaaagctggcgctggaggcttgctgaaggctgtatgctgttaagtcggtctctatgccgcgttttggccactgactgacgcggcatagaccgacttaacaggacacaaggcctgttactagcactcacatggaacaaatggcccacattggtgccggatgaagctcttatgttgcgtcccatcgcaggtgcct

SEQ ID NO: 395 - DNA, 204 bp, Artificial Sequence
  Synthetic AHR miRNA at P2
cctcacctgcttgcgtcccattctggaggcttgctgaaggctgtatgctgttaataccaactttaagcagtgttttggccactgactgacactgcttagttggtattaacaggacacaaggcctgttactagcactcacatggaacaaatggccgttgcctgagtcttggcagcgagagatcactaactgctaagcaggtgctt

SEQ ID NO: 396 - DNA, 204 bp, Artificial Sequence
  Synthetic AHR miRNA at P3
tgtcacctgcactaactgctaactggaggcttgctgaaggctgtatgctgttaatgcaacatcaaagaagcgttttggccactgactgacgcttcttttgttgcattaacaggacacaaggcctgttactagcactcacatggaacaaatggccgtgttaattgtccatgtagcgaggcatccttatggcgtgggcaggtgtcc

SEQ ID NO: 397 - DNA, 200 bp, Artificial Sequence
  Synthetic AHR miRNA at P4
ccttcacctgccttatggcgtggctggaggcttgctgaaggctgtatgctgtttcgtaaatgctctgttcctgttttggccactgactgacaggaacagcatttacgaaacaggacacaaggcctgttactagcactcacatggaacaaatggccggtgtccgttatcggggaagaaggtcgcgcacatagcaggtgtcc

SEQ ID NO: 398 - DNA, 223 bp, Artificial Sequence
  Synthetic EOMES miRNA at P1
cggcacctgcgctgccgttggatcggggatgaaagctggcgctggaggcttgctgaaggctgtatgctgtaatgtcctcacactttatgggttttggccactgactgacccataaagtgaggacattacaggacacaaggcctgttactagcactcacatggaacaaatggcccacattggtgccggatgaagctcttatgttgcgtcccatcgcaggtgcct

SEQ ID NO: 399 - DNA, 204 bp, Artificial Sequence
  Synthetic EOMES miRNA at P2
cctcacctgcttgcgtcccattctggaggcttgctgaaggctgtatgctgaaatgtctccttctgaaacgggttttggccactgactgacccgtttcaaggagacatttcaggacacaaggcctgttactagcactcacatggaacaaatggccgttgcctgagtcttggcagcgagagatcactaactgctaagcaggtgctt

SEQ ID NO: 400 - DNA, 204 bp, Artificial Sequence
  Synthetic EOMES miRNA at P3
tgtcacctgcactaactgctaactggaggcttgctgaaggctgtatgctgtttgcgcctttgttattggtggttttggccactgactgaccaccaataaaaggcgcaaacaggacacaaggcctgttactagcactcacatggaacaaatggccgtgttaattgtccatgtagcgaggcatccttatggcgtgggcaggtgtcc

SEQ ID NO: 401 - DNA, 200 bp, Artificial Sequence
  Synthetic EOMES miRNA at P4
ccttcacctgccttatggcgtggctggaggcttgctgaaggctgtatgctgtttgttggtcccaggttgctggttttggccactgactgaccagcaaccggaccaacaaacaggacacaaggcctgttactagcactcacatggaacaaatggccggtgtccgttatcggggaagaaggtcgcgcacatagcaggtgtcc

SEQ ID NO: 402 - DNA, 223 bp, Artificial Sequence
  Synthetic SMAD3 miRNA at P1
cggcacctgcgctgccgttggatcggggatgaaagctggcgctggaggcttgctgaaggctgtatgctgaaatagcgctgtcactgaggcgttttggccactgactgacgcctcagtcagcgctatttcaggacacaaggcctgttactagcactcacatggaacaaatggcccacattggtgccggatgaagctcttatgttgcgtcccatcgcaggtgcct

SEQ ID NO: 403 - DNA, 204 bp, Artificial Sequence
  Synthetic SMAD3 miRNA at P2
cctcacctgcttgcgtcccattctggaggcttgctgaaggctgtatgctgttcaggtgcagctcaatccaggttttggccactgactgacctggattgctgcacctgaacaggacacaaggcctgttactagcactcacatggaacaaatggccgttgcctgagtcttggcagcgagagatcactaactgctaagcaggtgctt

SEQ ID NO: 404 - DNA, 204 bp, Artificial Sequence
  Synthetic SMAD3 miRNA at P3
tgtcacctgcactaactgctaactggaggcttgctgaaggctgtatgctgtttcagcttgcagaagtgctggttttggccactgactgaccagcacttgcaagctgaaacaggacacaaggcctgttactagcactcacatggaacaaatggccgtgttaattgtccatgtagcgaggcatccttatggcgtgggcaggtgtcc

SEQ ID NO: 405 - DNA, 200 bp, Artificial Sequence
  Synthetic SMAD3 miRNA at P4
ccttcacctgccttatggcgtggctggaggcttgctgaaggctgtatgctgaatactacctgttctgctcacgttttggccactgactgacgtgagcagcaggtagtattcaggacacaaggcctgttactagcactcacatggaacaaatggccggtgtccgttatcggggaagaaggtcgcgcacatagcaggtgtcc

SEQ ID NO: 406 - DNA, 223 bp, Artificial Sequence
  Synthetic SMAD4 miRNA at P1
cggcacctgcgctgccgttggatcggggatgaaagctggcgctggaggcttgctgaaggctgtatgctgaattaggtgtgtatggtgcaggttttggccactgactgacctgcaccacacacctaattcaggacacaaggcctgttactagcactcacatggaacaaatggcccacattggtgccggatgaagctcttatgttgcgtcccatcgcaggtgcct

SEQ ID NO: 407 - DNA, 204 bp, Artificial Sequence
  Synthetic SMAD4 miRNA at P2
cctcacctgcttgcgtcccattctggaggcttgctgaaggctgtatgctgaagtacttcgtctaggagctggttttggccactgactgaccagctcctacgaagtacttcaggacacaaggcctgttactagcactcacatggaacaaatggccgttgcctgagtcttggcagcgagagatcactaactgctaagcaggtgctt

SEQ ID NO: 408 - DNA, 204 bp, Artificial Sequence
  Synthetic SMAD4 miRNA at P3
tgtcacctgcactaactgctaactggaggcttgctgaaggctgtatgctgtatcccacgatctactccccggttttggccactgactgaccggggagtatcgtgggatacaggacacaaggcctgttactagcactcacatggaacaaatggccgtgttaattgtccatgtagcgaggcatccttatggcgtgggcaggtgtcc

SEQ ID NO: 409 - DNA, 200 bp, Artificial Sequence
  Synthetic SMAD4 miRNA at P4
ccttcacctgccttatggcgtggctggaggcttgctgaaggctgtatgctgttatgatggtaagtagctggcgttttggccactgactgacgccagctataccatcataacaggacacaaggcctgttactagcactcacatggaacaaatggccggtgtccgttatcggggaagaaggtcgcgcacatagcaggtgtcc

SEQ ID NO: 410 - DNA, 223 bp, Artificial Sequence
  Synthetic TGFBR2 miRNA at P1
cggcacctgcgctgccgttggatcggggatgaaagctggcgctggaggcttgctgaaggctgtatgctgttattaaccgacttctgaacggttttggccactgactgaccgttcagatcggttaataacaggacacaaggcctgttactagcactcacatggaacaaatggcccacattggtgccggatgaagctcttatgttgcgtcccatcgcaggtgcct

SEQ ID NO: 411 - DNA, 204 bp, Artificial Sequence
  Synthetic TGFBR2 miRNA at P2
cctcacctgcttgcgtcccattctggaggcttgctgaaggctgtatgctgtaaacacgataaagcctagaggttttggccactgactgacctctaggctatcgtgtttacaggacacaaggcctgttactagcactcacatggaacaaatggccgttgcctgagtcttggcagcgagagatcactaactgctaagcaggtgctt

SEQ ID NO: 412 - DNA, 204 bp, Artificial Sequence
  Synthetic TGFBR2 miRNA at P3
tgtcacctgcactaactgctaactggaggcttgctgaaggctgtatgctgattctaggacttctggagccagttttggccactgactgactggctccaagtcctagaatcaggacacaaggcctgttactagcactcacatggaacaaatggccgtgttaattgtccatgtagcgaggcatccttatggcgtgggcaggtgtcc

SEQ ID NO: 413 - DNA, 200 bp, Artificial Sequence
  Synthetic TGFBR2 miRNA at P4
ccttcacctgccttatggcgtggctggaggcttgctgaaggctgtatgctgttcttcacgaggatattggaggttttggccactgactgacctccaatactcgtgaagaacaggacacaaggcctgttactagcactcacatggaacaaatggccggtgtccgttatcggggaagaaggtcgcgcacatagcaggtgtcc

SEQ ID NO: 414 - DNA, 223 bp, Artificial Sequence
  Synthetic PPP2R2D miRNA at P1
cggcacctgcgctgccgttggatcggggatgaaagctggcgctggaggcttgctgaaggctgtatgctgaaatgtccggcttaactatgcgttttggccactgactgacgcatagttgccggacatttcaggacacaaggcctgttactagcactcacatggaacaaatggcccacattggtgccggatgaagctcttatgttgcgtcccatcgcaggtgcct

SEQ ID NO: 415 - DNA, 204 bp, Artificial Sequence
  Synthetic PPP2R2D miRNA at P2
cctcacctgcttgcgtcccattctggaggcttgctgaaggctgtatgctgattaattctcaggtcatctgcgttttggccactgactgacgcagatgatgagaattaatcaggacacaaggcctgttactagcactcacatggaacaaatggccgttgcctgagtcttggcagcgagagatcactaactgctaagcaggtgctt

SEQ ID NO: 416 - DNA, 204 bp, Artificial Sequence
  Synthetic PPP2R2D miRNA at P3
tgtcacctgcactaactgctaactggaggcttgctgaaggctgtatgctgtttgaagctactttaaaccaggttttggccactgactgacctggtttagtagcttcaaacaggacacaaggcctgttactagcactcacatggaacaaatggccgtgttaattgtccatgtagcgaggcatccttatggcgtgggcaggtgtcc

SEQ ID NO: 417 - DNA, 200 bp, Artificial Sequence
  Synthetic PPP2R2D miRNA at P4
ccttcacctgccttatggcgtggctggaggcttgctgaaggctgtatgctgttcaccgacaggtagtctctggttttggccactgactgaccagagactctgtcggtgaacaggacacaaggcctgttactagcactcacatggaacaaatggccggtgtccgttatcggggaagaaggtcgcgcacatagcaggtgtcc

SEQ ID NO: 418 - DNA, 223 bp, Artificial Sequence
  Synthetic TNFSF6 miRNA at P1
cggcacctgcgctgccgttggatcggggatgaaagctggcgctggaggcttgctgaaggctgtatgctgttattatgcaagcctctagtcgttttggccactgactgacgactagagttgcataataacaggacacaaggcctgttactagcactcacatggaacaaatggcccacattggtgccggatgaagctcttatgttgcgtcccatcgcaggtgcct

SEQ ID NO: 419 - DNA, 204 bp, Artificial Sequence
  Synthetic TNFSF6 miRNA at P2
cctcacctgcttgcgtcccattctggaggcttgctgaaggctgtatgctgaaattgaccagagagagctcagttttggccactgactgactgagctctctggtcaatttcaggacacaaggcctgttactagcactcacatggaacaaatggccgttgcctgagtcttggcagcgagagatcactaactgctaagcaggtgctt

SEQ ID NO: 420 - DNA, 204 bp, Artificial Sequence
  Synthetic TNFSF6 miRNA at P3
tgtcacctgcactaactgctaactggaggcttgctgaaggctgtatgctgtattcctccatttgtctggctgttttggccactgactgacagccagacatggaggaatacaggacacaaggcctgttactagcactcacatggaacaaatggccgtgttaattgtccatgtagcgaggcatccttatggcgtgggcaggtgtcc

SEQ ID NO: 421 - DNA, 200 bp, Artificial Sequence
  Synthetic TNFSF6 miRNA at P4
ccttcacctgccttatggcgtggctggaggcttgctgaaggctgtatgctgtttcaatctgcctaaatactcgttttggccactgactgacgagtatttgcagattgaaacaggacacaaggcctgttactagcactcacatggaacaaatggccggtgtccgttatcggggaagaaggtcgcgcacatagcaggtgtcc

SEQ ID NO: 422 - DNA, 223 bp, Artificial Sequence
  Synthetic CASP3 miRNA at P1
cggcacctgcgctgccgttggatcggggatgaaagctggcgctggaggcttgctgaaggctgtatgctgtttcagcatggcacaaagcgagttttggccactgactgactcgctttgccatgctgaaacaggacacaaggcctgttactagcactcacatggaacaaatggcccacattggtgccggatgaagctcttatgttgcgtcccatcgcaggtgcct

SEQ ID NO: 423 - DNA, 204 bp, Artificial Sequence
  Synthetic CASP3 miRNA at P2
cctcacctgcttgcgtcccattctggaggcttgctgaaggctgtatgctgtttgagcctttgaccatgcccgttttggccactgactgacgggcatggaaaggctcaaacaggacacaaggcctgttactagcactcacatggaacaaatggccgttgcctgagtcttggcagcgagagatcactaactgctaagcaggtgctt

SEQ ID NO: 424 - DNA, 204 bp, Artificial Sequence
  Synthetic CASP3 miRNA at P3
tgtcacctgcactaactgctaactggaggcttgctgaaggctgtatgctgttattgcctcaccacctttaggttttggccactgactgacctaaaggttgaggcaataacaggacacaaggcctgttactagcactcacatggaacaaatggccgtgttaattgtccatgtagcgaggcatccttatggcgtgggcaggtgtcc

SEQ ID NO: 425 - DNA, 200 bp, Artificial Sequence
  Synthetic CASP3 miRNA at P4
ccttcacctgccttatggcgtggctggaggcttgctgaaggctgtatgctgaaggactcaaattctgttgccgttttggccactgactgacggcaacagtttgagtccttcaggacacaaggcctgttactagcactcacatggaacaaatggccggtgtccgttatcggggaagaaggtcgcgcacatagcaggtgtcc

SEQ ID NO: 426 - DNA, 223 bp, Artificial Sequence
  Synthetic SOCS2 miRNA at P1
cggcacctgcgctgccgttggatcggggatgaaagctggcgctggaggcttgctgaaggctgtatgctgtaatcaagaaagttccttctggttttggccactgactgaccagaaggatttcttgattacaggacacaaggcctgttactagcactcacatggaacaaatggcccacattggtgccggatgaagctcttatgttgcgtcccatcgcaggtgcct

SEQ ID NO: 427 - DNA, 204 bp, Artificial Sequence
  Synthetic SOCS2 miRNA at P2
cctcacctgcttgcgtcccattctggaggcttgctgaaggctgtatgctgtttagtcttgttggtaaaggcgttttggccactgactgacgcctttacacaagactaaacaggacacaaggcctgttactagcactcacatggaacaaatggccgttgcctgagtcttggcagcgagagatcactaactgctaagcaggtgctt

SEQ ID NO: 428 - DNA, 204 bp, Artificial Sequence
  Synthetic SOCS2 miRNA at P3
tgtcacctgcactaactgctaactggaggcttgctgaaggctgtatgctgatatgatagagtccaatctgagttttggccactgactgactcagattgctctatcatatcaggacacaaggcctgttactagcactcacatggaacaaatggccgtgttaattgtccatgtagcgaggcatccttatggcgtgggcaggtgtcc

SEQ ID NO: 429 - DNA, 200 bp, Artificial Sequence
  Synthetic SOCS2 miRNA at P4
ccttcacctgccttatggcgtggctggaggcttgctgaaggctgtatgctgttagtaggtagtctgaatgcggttttggccactgactgaccgcattcactacctactaacaggacacaaggcctgttactagcactcacatggaacaaatggccggtgtccgttatcggggaagaaggtcgcgcacatagcaggtgtcc

SEQ ID NO: 430 - DNA, 223 bp, Artificial Sequence
  Synthetic TIEG1 miRNA at P1
cggcacctgcgctgccgttggatcggggatgaaagctggcgctggaggcttgctgaaggctgtatgctgatttagcttgctcacttccatgttttggccactgactgacatggaagtgcaagctaaatcaggacacaaggcctgttactagcactcacatggaacaaatggcccacattggtgccggatgaagctcttatgttgcgtcccatcgcaggtgcct

SEQ ID NO: 431 - DNA, 204 bp, Artificial Sequence
  Synthetic TIEG1 miRNA at P2
cctcacctgcttgcgtcccattctggaggcttgctgaaggctgtatgctgatttgacacttgagagggttcgttttggccactgactgacgaaccctcaagtgtcaaatcaggacacaaggcctgttactagcactcacatggaacaaatggccgttgcctgagtcttggcagcgagagatcactaactgctaagcaggtgctt

SEQ ID NO: 432 - DNA, 204 bp, Artificial Sequence
  Synthetic TIEG1 miRNA at P3
tgtcacctgcactaactgctaactggaggcttgctgaaggctgtatgctgaaatcagatactggtgtaacagttttggccactgactgactgttacacgtatctgatttcaggacacaaggcctgttactagcactcacatggaacaaatggccgtgttaattgtccatgtagcgaggcatccttatggcgtgggcaggtgtcc

SEQ ID NO: 433 - DNA, 200 bp, Artificial Sequence
  Synthetic TIEG1 miRNA at P4
ccttcacctgccttatggcgtggctggaggcttgctgaaggctgtatgctgtttctctcacatttggatctggttttggccactgactgaccagatccatgtgagagaaacaggacacaaggcctgttactagcactcacatggaacaaatggccggtgtccgttatcggggaagaaggtcgcgcacatagcaggtgtcc

SEQ ID NO: 434 - DNA, 223 bp, Artificial Sequence
  Synthetic JunB miRNA at P1
cggcacctgcgctgccgttggatcggggatgaaagctggcgctggaggcttgctgaaggctgtatgctggtttcaggagtttgtagtcgtgttttggccactgactgacacgactacactcctgaaaccaggacacaaggcctgttactagcactcacatggaacaaatggcccacattggtgccggatgaagctcttatgttgcgtcccatcgcaggtgcct

SEQ ID NO: 435 - DNA, 204 bp, Artificial Sequence
  Synthetic JunB miRNA at P2
cctcacctgcttgcgtcccattctggaggcttgctgaaggctgtatgctgatatgaatcgagtctgtttccgttttggccactgactgacggaaacagtcgattcatatcaggacacaaggcctgttactagcactcacatggaacaaatggccgttgcctgagtcttggcagcgagagatcactaactgctaagcaggtgctt

SEQ ID NO: 436 - DNA, 204 bp, Artificial Sequence
  Synthetic JunB miRNA at P3
tgtcacctgcactaactgctaactggaggcttgctgaaggctgtatgctgtaaacgtcgaggtggaaggacgttttggccactgactgacgtccttccctcgacgtttacaggacacaaggcctgttactagcactcacatggaacaaatggccgtgttaattgtccatgtagcgaggcatccttatggcgtgggcaggtgtcc

SEQ ID NO: 437 - DNA, 200 bp, Artificial Sequence
  Synthetic JunB miRNA at P4
ccttcacctgccttatggcgtggctggaggcttgctgaaggctgtatgctgttgcgctccactttgatgcgcgttttggccactgactgacgcgcatcagtggagcgcaacaggacacaaggcctgttactagcactcacatggaacaaatggccggtgtccgttatcggggaagaaggtcgcgcacatagcaggtgtcc

SEQ ID NO: 438 - DNA, 223 bp, Artificial Sequence
  Synthetic Cbx3 miRNA at P1
cggcacctgcgctgccgttggatcggggatgaaagctggcgctggaggcttgctgaaggctgtatgctgtaatgactatggacatttcccgttttggccactgactgacgggaaatgcatagtcattacaggacacaaggcctgttactagcactcacatggaacaaatggcccacattggtgccggatgaagctcttatgttgcgtcccatcgcaggtgcct

SEQ ID NO: 439 - DNA, 204 bp, Artificial Sequence
  Synthetic Cbx3 miRNA at P2
cctcacctgcttgcgtcccattctggaggcttgctgaaggctgtatgctgataatccaatgagtgtgggcagttttggccactgactgactgcccacacattggattatcaggacacaaggcctgttactagcactcacatggaacaaatggccgttgcctgagtcttggcagcgagagatcactaactgctaagcaggtgctt

SEQ ID NO: 440 - DNA, 204 bp, Artificial Sequence
  Synthetic Cbx3 miRNA at P3
tgtcacctgcactaactgctaactggaggcttgctgaaggctgtatgctgaaatcctcttggtttgtcagcgttttggccactgactgacgctgacaacaagaggatttcaggacacaaggcctgttactagcactcacatggaacaaatggccgtgttaattgtccatgtagcgaggcatccttatggcgtgggcaggtgtcc

SEQ ID NO: 441 - DNA, 200 bp, Artificial Sequence
  Synthetic Cbx3 miRNA at P4
ccttcacctgccttatggcgtggctggaggcttgctgaaggctgtatgctgttcatatttgcctctttcgccgttttggccactgactgacggcgaaaggcaaatatgaacaggacacaaggcctgttactagcactcacatggaacaaatggccggtgtccgttatcggggaagaaggtcgcgcacatagcaggtgtcc

SEQ ID NO: 442 - DNA, 223 bp, Artificial Sequence
  Synthetic Tet2 miRNA at P1
cggcacctgcgctgccgttggatcggggatgaaagctggcgctggaggcttgctgaaggctgtatgctgaaataacgacttggcgtgaaagttttggccactgactgactttcacgcagtcgttatttcaggacacaaggcctgttactagcactcacatggaacaaatggcccacattggtgccggatgaagctcttatgttgcgtcccatcgcaggtgcct

SEQ ID NO: 443 - DNA, 204 bp, Artificial Sequence
  Synthetic Tet2 miRNA at P2
cctcacctgcttgcgtcccattctggaggcttgctgaaggctgtatgctgtttcatggtctgactataagggttttggccactgactgacccttatagagaccatgaaacaggacacaaggcctgttactagcactcacatggaacaaatggccgttgcctgagtcttggcagcgagagatcactaactgctaagcaggtgctt

SEQ ID NO: 444 - DNA, 204 bp, Artificial Sequence
  Synthetic Tet2 miRNA at P3
tgtcacctgcactaactgctaactggaggcttgctgaaggctgtatgctgataccctctactttcttgtgtgttttggccactgactgacacacaagagtagagggtatcaggacacaaggcctgttactagcactcacatggaacaaatggccgtgttaattgtccatgtagcgaggcatccttatggcgtgggcaggtgtcc

SEQ ID NO: 445 - DNA, 200 bp, Artificial Sequence
  Synthetic Tet2 miRNA at P4
ccttcacctgccttatggcgtggctggaggcttgctgaaggctgtatgctgttgtcctgtagctctccactggttttggccactgactgaccagtggagctacaggacaacaggacacaaggcctgttactagcactcacatggaacaaatggccggtgtccgttatcggggaagaaggtcgcgcacatagcaggtgtcc

SEQ ID NO: 446 - DNA, 223 bp, Artificial Sequence
  Synthetic HK2 miRNA at P1
cggcacctgcgctgccgttggatcggggatgaaagctggcgctggaggcttgctgaaggctgtatgctgaatactactgactgccctaaggttttggccactgactgaccttagggctcagtagtattcaggacacaaggcctgttactagcactcacatggaacaaatggcccacattggtgccggatgaagctcttatgttgcgtcccatcgcaggtgcct

SEQ ID NO: 447 - DNA, 204 bp, Artificial Sequence
  Synthetic HK2 miRNA at P2
cctcacctgcttgcgtcccattctggaggcttgctgaaggctgtatgctgaaatcgatgagaatgttacgggttttggccactgactgacccgtaacactcatcgatttcaggacacaaggcctgttactagcactcacatggaacaaatggccgttgcctgagtcttggcagcgagagatcactaactgctaagcaggtgctt

SEQ ID NO: 448 - DNA, 204 bp, Artificial Sequence
  Synthetic HK2 miRNA at P3
tgtcacctgcactaactgctaactggaggcttgctgaaggctgtatgctgcaatgtcgatatcaaagtcccgttttggccactgactgacgggacttttatcgacattgcaggacacaaggcctgttactagcactcacatggaacaaatggccgtgttaattgtccatgtagcgaggcatccttatggcgtgggcaggtgtcc

SEQ ID NO: 449 - DNA, 200 bp, Artificial Sequence
  Synthetic HK2 miRNA at P4
ccttcacctgccttatggcgtggctggaggcttgctgaaggctgtatgctgttatccatgaagttagccagggttttggccactgactgaccctggctattcatggataacaggacacaaggcctgttactagcactcacatggaacaaatggccggtgtccgttatcggggaagaaggtcgcgcacatagcaggtgtcc

SEQ ID NO: 450 - DNA, 9 bp, Artificial Sequence
  Synthetic Kozak-typesequence
gccgccacc

SEQ ID NO: 451 - DNA, 9 bp, Artificial Sequence
  Synthetic triple stopsequence
taatagtga

SEQ ID NO: 452 - DNA, 191 bp, Artificial Sequence
  Synthetic WPRE
gtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 453 - protein, 654 aa, Artificial Sequence
  Synthetic MuLVSUx
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 454 - protein, 905 aa, Artificial Sequence
  Synthetic UMuLVSUx
MARSTLSKPPQDKINPWKPLIVMGVLLGVGDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSAAAIEGRMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTIEGREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 455 - protein, 770 aa, Artificial Sequence
  Synthetic UCHT1-(G4S)3-VSVG
MKCLLYLAFLFIGVNCDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 456 - protein, 767 aa, Artificial Sequence
  Synthetic UCHT1-hinge-VSVG
MKCLLYLAFLFIGVNCDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSEPKSCDKTHTCPKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 457 - DNA, 1823 bp, Artificial Sequence
  Synthetic synthetic EF-1a promoter with miRs
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttcctggaggcttgctgaaggctgtatgctgacatggtacagttcaatggtggttttggccactgactgaccaccattgctgtaccatgtcaggacacaaggcctgttactagcactcacatggaacaaatggcccacattggtgccggatgaagctcttatgttgcacggtcatctggaggcttgctgaaggctgtatgctgtcagtctgttcatcttctggcgttttggccactgactgacgccagaaggaacagactgacaggacacaaggcctgttactagcactcacatggaacaaatggccgttgccggagtcttggcagcgagagatcactatcaactaactggaggcttgctgaaggctgtatgctgaagcgtgaagtgaatcaacgggttttggccactgactgacccgttgatacttcacgcttcaggacacaaggcctgttactagcactcacatggaacaaatggccgtgttaattgtccatgtagcgaggcatccttatggcgtggctggaggcttgctgaaggctgtatgctggcagtatcctagtacattgacgttttggccactgactgacgtcaatgttaggatactgccaggacacaaggcctgttactagcactcacatggaacaaatggccgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

(19) United States
(12) Patent Application Publication
(10) Pub. No.: US 2021/0317408 A1
(43) Pub. Date: Oct. 14, 2021

(54) METHODS AND COMPOSITIONS FOR GENETICALLY MODIFYING LYMPHOCYTES IN BLOOD OR IN ENRICHED PBMCS

(71) Applicant: Exuma Biotech Corp., West Palm Beach, FL (US)

(72) Inventors: Gregory Ian Frost, West Palm Beach, FL (US); James Joseph Onuffer, Alameda, CA (US); Farzad Haerizadeh, San Diego, CA (US); Frederic Vigant, West-Bay, Grand Cayman (KY); Anirban Kundu, West-Bay, Grand Cayman (KY)

(73) Assignee: Exuma Biotech Corp., West Palm Beach, FL (US)

(21) Appl. No.: 17/272,631

(22) PCT Filed: Sep. 2, 2019
(86) PCT No.: PCT/US2019/049259

§ 371 Date: Mar. 1, 2021

Related U.S. Application Data
(60) Provisional application No. 62/894,853, filed on Sep. 1, 2019.
(60) Provisional application No. 62/821,434, filed on Mar. 20, 2019.
(60) Provisional application No. 62/732,528, filed on Sep. 17, 2018.
(60) Provisional application No. 62/728,056, filed on Sep. 6, 2018.
(60) Provisional application No. 62/726,294, filed on Sep. 2, 2018.
(60) Provisional application No. 62/726,293, filed on Sep. 2, 2018.

(51) Int. Cl. C12N 5/0783; C07K 16/28; C07K 14/47; C12N 15/11; C07K 14/725; C12N 7/00

(52) U.S. Cl. CPC C12N 5/0636; C07K 16/2809; C07K 14/4702; C12N 15/11; C07K 14/7051; C12N 2501/91; C12N 2510/00; C12N 2740/15022; C07K 2319/035; C07K 2317/622; C07K 2319/02; C12N 7/00

(57) ABSTRACT

The present disclosure provides methods and compositions for genetically modifying lymphocytes, for example T cells and/or NK cells, in shorter times than previously and/or in whole blood or a component thereof. In some embodiments a lymphodepletion filter assembly is used before or after forming a reaction mixture where lymphocytes are contacted with recombinant retroviral particles in a closed system, to genetically modify the lymphocytes.

### CROSS-REFERENCE TO RELATED APPLICATIONS

This application is a continuation-in-part of International Application No. PCT/US2018/051392 filed Sep. 17, 2018; and claims the benefit of U.S. Provisional Application No. 62/726,293, filed Sep. 2, 2018; U.S. Provisional Application No. 62/726,294, filed Sep. 2, 2018; U.S. Provisional Application No. 62/728,056 filed Sep. 6, 2018; U.S. Provisional Application No. 62/732,528, filed Sep. 17, 2018; U.S. Provisional Application No. 62/821,434, filed Mar. 20, 2019; and U.S. Provisional Application No. 62/894,853, filed Sep. 1, 2019; and International Application No. PCT/US2018/051392 is a continuation-in-part of International Application No. PCT/US2018/020818, filed Mar. 3, 2018; and claims the benefit of U.S. Provisional Application No. 62/560,176, filed Sep. 18, 2017; U.S. Provisional Application No. 62/564,253, filed Sep. 27, 2017; U.S. Provisional Application No. 62/564,991, filed Sep. 28, 2017; and U.S. Provisional Application No. 62/728,056, filed Sep. 6, 2018; International Application No. PCT/US2018/020818 is a continuation-in-part of International Application No. PCT/US2017/023112 filed Mar. 19, 2017; a continuation-in-part of International Application No. PCT/US2017/041277 filed Jul. 8, 2017; a continuation-in-part of U.S. application Ser. No. 15/462,855 filed Mar. 19, 2017; and a continuation-in-part of U.S. application Ser. No. 15/644,778 filed Jul. 8, 2017; and claims the benefit of U.S. Provisional Application No. 62/467,039 filed Mar. 3, 2017; U.S. Provisional Application No. 62/560,176 filed Sep. 18, 2017; U.S. Provisional Application No. 62/564,253 filed Sep. 27, 2017; and U.S. Provisional Application No. 62/564,991 filed Sep. 28, 2017; International Application No. PCT/US2017/023112 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; International Application No. PCT/US2017/041277 claims the benefit of International Application No. PCT/US2017/023112, filed Mar. 19, 2017; U.S. patent application Ser. No. 15/462,855, filed Mar. 19, 2017; U.S. Provisional Application No. 62/360,041, filed Jul. 8, 2016; and U.S. Provisional Application No. 62/467,039, filed Mar. 3, 2017; U.S. application Ser. No. 15/462,855 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; and U.S. application Ser. No. 15/644,778 is a continuation-in-part of International Application No. PCT/US2017/023112, filed Mar. 19, 2017; and a continuation-in-part of U.S. patent application Ser. No. 15/462,855, filed Mar. 19, 2017; and claims the benefit of U.S. Provisional Application No. 62/360,041, filed Jul. 8, 2016, and U.S. Provisional Application No. 62/467,039, filed Mar. 3, 2017. These applications are incorporated by reference herein in their entireties.

### SEQUENCE LISTING

This application hereby incorporates by reference the material of the electronic Sequencing Listing filed concurrently herewith. The materials in the electronic Sequence Listing is submitted as a text (.txt) file entitled “F1_001_WO_05_Sequence_Listing_September_02_2019.txt” created on Sep. 2, 2019, which has a file size of 450 KB, and is herein incorporated by reference in its entirety.

### FIELD OF INVENTION

This disclosure relates to the field of immunology, or more specifically, to the genetic modification of T lymphocytes or other immune cells, and methods of controlling proliferation of such cells.

### BACKGROUND OF THE DISCLOSURE

Lymphocytes isolated from a subject (e.g. patient) can be activated in vitro and genetically modified to express synthetic proteins that enable redirected engagement with other cells and environments based upon the genetic programs incorporated. Examples of such synthetic proteins include recombinant T cell receptors (TCRs) and chimeric antigen receptors (CARs). One CAR that is currently used is a fusion of an extracellular recognition domain (e.g., an antigen-binding domain), a transmembrane domain, and one or more intracellular signaling domains encoded by a replication incompetent recombinant retrovirus.

While recombinant retroviruses have shown efficacy in infecting non-dividing cells, resting CD4 and CD8 lymphocytes are refractory to genetic transduction by these vectors. To overcome this difficulty, these cells are typically activated in vitro using stimulation reagents before genetic modification with the CAR gene vector can occur. Following stimulation and transduction, the genetically modified cells are expanded in vitro and subsequently reintroduced into a lymphodepleted patient. Upon antigen engagement in vivo, the intracellular signaling portion of the CAR can initiate an activation-related response in an immune cell and release of cytolytic molecules to induce target cell death.

Such current methods require extensive manipulation and manufacturing of proliferating T cells outside the body prior to their reinfusion into the patient, as well as lymphodepleting chemotherapy to free cytokines and deplete competing receptors to facilitate T cell engraftment. Such CAR therapies further cannot be controlled for propagation rate in vivo once introduced into the body, nor safely directed towards targets that are also expressed outside the tumor. As a result, CAR therapies today are typically infused from cells expanded ex vivo from 12 to 28 days using doses from 1×10⁵ to 1×10⁸ cells/kg and are directed towards targets, for example tumor targets, for which off tumor on target toxicity is generally acceptable. These relatively long ex vivo expansion times create issues of cell viability and sterility, as well as sample identity in addition to challenges of scalability. Thus, there are significant needs for a safer, more effective scalable T cell or NK cell therapy.

Since our understanding of processes that drive transduction, proliferation and survival of lymphocytes is central to various potential commercial uses that involve immunological processes, there is a need for improved methods and compositions for studying lymphocytes. For example, it would be helpful to identify methods and compositions that can be used to better characterize and understand how lymphocytes can be genetically modified and the factors that influence their survival and proliferation. Furthermore, it would be helpful to identify compositions that drive lymphocyte proliferation and survival. Such compositions could be used to study the regulation of such processes. In addition to methods and compositions for studying lymphocytes, there is a need for improved viral packaging cell lines and methods of making and using the same. For example, such cell lines and methods would be useful in analyzing different components of recombinant viruses, such as recombinant retroviral particles, and for methods that use packaging cells lines for the production of recombinant retroviral particles.

More recent methods have been developed that can be performed without pre-activation and ex vivo expansion. However, further reduction in the complexity and time required for such methods would be highly desirable, especially if such methods allow a subject to have their blood collected, for example within an infusion center, and then reintroduced into the subject that same day. Furthermore, simpler and quicker methods alone or methods that require fewer specialized instruments, could democratize these cell therapy processes, which are currently performed regularly only at highly specialized medical centers.

Some groups have attempted to simplify ex-vivo processing for cell therapy by eliminating ex-vivo transduction expansion, by infusion viral particles intravenously, to transduce cells in vivo. However, such methods require large quantities of vector and the methods have the risk of inactivation of the retroviral particles by clotting factors, and/or other enzymes present in vivo. Finally, such methods risk a high level of transduction of non-target cells/organs.

### SUMMARY

Provided herein are methods, compositions, and kits that help overcome issues related to the effectiveness and safety of methods for transducing and/or genetically modifying lymphocytes such as T cells and/or NK cells. Certain embodiments of such methods are useful for performing adoptive cell therapy with these cells. Accordingly, in some aspects, provided herein are methods, compositions, and kits for genetically modifying lymphocytes, especially T cell and/or NK cells, and/or for regulating the activity of transduced and/or genetically modified T cells and/or NK cells. Such methods, compositions, and kits provide improved efficacy and safety over current technologies, especially with respect to T cells and/or NK cells that express recombinant T cell receptors (TCRs), chimeric antigen receptors (CARs), and in illustrative embodiments microenvironment restricted biologic (“MRB”) CARs. Transduced and/or genetically modified T cells and/or NK cells that are produced by and/or used in methods provided herein, include functionality and combinations of functionality, in illustrative embodiments delivered from retroviral (e.g. lentiviral) genomes via retroviral (e.g. lentiviral) particles, that provide improved features for such cells and for methods that utilize such cells, such as research methods, commercial production methods, and adoptive cellular therapy. For example, such cells can be produced in less time ex vivo, and that have improved growth properties that can be better regulated.

In some aspects, methods are provided for transducing and/or genetically modifying lymphocytes such as T cells and/or NK cells, and in illustrative embodiments, ex vivo methods for transducing and/or genetically modifying resting T cells and/or NK cells. Some of these aspects can be performed much more quickly than previous methods, which can facilitate more efficient research, more effective commercial production, and improved methods of patient care. Methods, compositions, and kits provided herein, can be used as research tools, in commercial production, and in adoptive cellular therapy with transduced and/or genetically modified T cells and/or NK cells expressing a TCR or a CAR.

With respect to methods, uses and compositions provided herein that relate to transduction of lymphocytes such as T cells and/or NK cells, methods, and associated uses and compositions, are provide herein that include transduction reactions of enriched PBMCs or transduction reactions without prior PBMC enrichment, such as in whole blood that are simplified and quicker methods for performing ex-vivo cell processing, for example for CAR-T therapy. Such methods require less specialized instrumentation and training. Furthermore, such methods reduce the risk of non-targeted cell transduction compared to in vivo transduction methods. Furthermore, provided herein are methods, uses, and compositions, including embodiments of the methods immediately above, that include certain target inhibitory RNAs, polypeptide lymphoproliferative elements, and pseudotyping elements that can be optionally be combined with any other aspects provided herein to provide powerful methods, uses, and compositions for driving expansion of lymphocytes, especially T cells and/or NK cells in vitro, ex vivo, and in vivo.

Further details regarding aspects and embodiments of the present disclosure are provided throughout this patent application. Sections and section headers are for ease of reading and are not intended to limit combinations of disclosure, such as methods, compositions, and kits or functional elements therein across sections.

### BRIEF DESCRIPTION OF THE DRAWINGS

FIGS. 1A-1B are flowcharts of non-limiting exemplary cell processing workflows. FIG. 1A is a flow chart of a process that uses a system with PBMC enrichment before the contacting of T cells and NK cells in the PBMCs with retroviral particles. FIG. 1B is a flow chart of a process in which no blood cell fractionation or enrichment is performed before T cells and NK cells in the whole blood are contacted with retroviral particles, and a PBMC enrichment is performed after transduction.

FIG. 2 is a diagram of a non-limiting exemplary leukodepletion filter assembly (200) with associated blood processing bags, tubes, valves, and filter enclosure (210) comprising a leukodepletion filter set.

FIGS. 3A and 3B show histograms of experimental results with different pseudotyping elements. FIG. 3A shows a histogram of the total number of live cells per well on Day 6 following transduction. FIG. 3B shows a histogram of the percent of CD3+ cells transduced as measured by eTAG expression.

FIGS. 4A and 4B show histograms of experimental results with transduction reaction mixtures that include whole blood, lentiviral particles, and anti-coagulants EDTA or heparin, without PBMC enrichment before the reaction mixture was formed. The process was performed by contacting whole blood for 4 hours with the indicated lentiviral particle F1-3-23G or F1-3-23GU followed by a density gradient centrifugation-based PBMC enrichment procedure. FIG. 4A shows a histogram of the absolute cell number per uL of the live lymphocyte population. FIG. 4B shows a histogram of the percentage (%) CD3+eTag+ cells in the live lymphocyte population at Day 6 post-transduction.

FIG. 5 is a histogram showing the CD3+FLAG+ cell number per μl of culture at Day 6 after transduction of unstimulated PBMCs by the different recombinant lentiviral particles at an MOI of 1 for the indicated period of time. F1-3-253 encoded an anti-CD19 CAR and F1-3-451 encoded a CLE in addition to the same CAR. The lentiviral particles were pseudotyped with VSV-G [VSV-G] and optionally displayed UCHT1ScFvFc-GPI [VSV-G+U] as indicated. Samples were treated with dapivirine, an inhibitor of reverse transcription (RT inb) or dolutegravir, an inhibitor to integration (INT Inb), as indicated.

FIG. 6 is a schematic of a non-limiting, exemplary transgene expression cassette containing a polynucleotide sequence encoding a CAR and a candidate CLE of Libraries analyzed in Example 6.

### DEFINITIONS

As used herein, the term “chimeric antigen receptor” or “CAR” or “CARs” refers to engineered receptors, which graft an antigen specificity onto cells, for example T cells, NK cells, macrophages, and stem cells. The CARs of the invention include at least one antigen-specific targeting region (ASTR), a transmembrane domain (TM), and an intracellular activating domain (IAD) and can include a stalk, and one or more co-stimulatory domains (CSDs). In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

As used herein, the term “microenvironment” means any portion or region of a tissue or body that has constant or temporal, physical, or chemical differences from other regions of the tissue or regions of the body. For example, a “tumor microenvironment” as used herein refers to the environment in which a tumor exists, which is the non-cellular area within the tumor and the area directly outside the tumorous tissue but does not pertain to the intracellular compartment of the cancer cell itself. The tumor microenvironment can refer to any and all conditions of the tumor milieu including conditions that create a structural and or functional environment for the malignant process to survive and/or expand and/or spread. For example, the tumor microenvironment can include alterations in conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions a skilled artisan will understand.

As used interchangeably herein, the terms “polynucleotide” and “nucleic acid” refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “antibody” includes polyclonal and monoclonal antibodies, including intact antibodies and fragments of antibodies which retain specific binding to antigen. The antibody fragments can be, but are not limited to, fragment antigen binding (Fab) fragments, Fab′ fragments, F(ab′)₂ fragments, Fv fragments, Fab′-SH fragments, (Fab′)₂ Fv fragments, Fd fragments, recombinant IgG (rIgG) fragments, single-chain antibody fragments, including single-chain variable fragments (scFv), divalent scFv's, trivalent scFv's, and single domain antibody fragments (e.g., sdAb, sdFv, nanobody). The term includes genetically engineered and/or otherwise modified forms of immunoglobulins, such as intrabodies, peptibodies, chimeric antibodies, single-chain antibodies, fully human antibodies, humanized antibodies, fusion proteins including an antigen-specific targeting region of an antibody and a non-antibody protein, heteroconjugate antibodies, multispecific, e.g., bispecific, antibodies, diabodies, triabodies, and tetrabodies, tandem di-scFv's, and tandem tri-scFv's. Unless otherwise stated, the term “antibody” should be understood to include functional antibody fragments thereof. The term also includes intact or full-length antibodies, including antibodies of any class or sub-class, including IgG and sub-classes thereof, IgM, IgE, IgA, and IgD.

As used herein, the term “antibody fragment” includes a portion of an intact antibody, for example, the antigen binding or variable region of an intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

As used interchangeably herein, the terms “single-chain Fv,” “scFv,” or “sFv” antibody fragments include the V_{H} and V_{L} domains of antibody, wherein these domains are present in a single polypeptide chain. In some embodiments, the Fv polypeptide further includes a polypeptide linker or spacer between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, “naturally occurring” VH and VL domains refer to VH and VL domains that have been isolated from a host without further molecular evolution to change their affinities when generated in an scFv format under specific conditions such as those disclosed in U.S. Pat. No. 8,709,755 B2 and application WO/2016/033331A1.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

As used herein, the term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, reference to a “cell surface expression system” or “cell surface display system” refers to the display or expression of a protein or portion thereof on the surface of a cell. Typically, a cell is generated that expresses proteins of interest fused to a cell-surface protein. For example, a protein is expressed as a fusion protein with a transmembrane domain.

As used herein, the term “element” includes polypeptides, including fusions of polypeptides, regions of polypeptides, and functional mutants or fragments thereof and polynucleotides, including microRNAs and shRNAs, and functional mutants or fragments thereof.

As used herein, the term “region” is any segment of a polypeptide or polynucleotide.

As used herein, a “domain” is a region of a polypeptide or polynucleotide with a functional and/or structural property.

As used herein, the terms “stalk” or “stalk domain” refer to a flexible polypeptide connector region providing structural flexibility and spacing to flanking polypeptide regions and can consist of natural or synthetic polypeptides. A stalk can be derived from a hinge or hinge region of an immunoglobulin (e.g., IgG1) that is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The stalk may be of natural occurrence or non-natural occurrence, including but not limited to an altered hinge region, as disclosed in U.S. Pat. No. 5,677,425. The stalk can include a complete hinge region derived from an antibody of any class or subclass. The stalk can also include regions derived from CD8, CD28, or other receptors that provide a similar function in providing flexibility and spacing to flanking regions.

As used herein, the term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

As used herein, a “polypeptide” is a single chain of amino acid residues linked by peptide bonds. A polypeptide does not fold into a fixed structure nor does it have any posttranslational modification. A “protein” is a polypeptide that folds into a fixed structure. “Polypeptides” and “proteins” are used interchangeably herein.

As used herein, a polypeptide may be “purified” to remove contaminant components of a polypeptide's natural environment, e.g. materials that would interfere with diagnostic or therapeutic uses for the polypeptide such as, for example, enzymes, hormones, and other proteinaceous or nonproteinaceous solutes. A polypeptide can be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, “T cell” includes all types of immune cells expressing CD3 including T-helper cells (CD4⁺ cells), cytotoxic T cells (CD8⁺ cells), T-regulatory cells (Treg) and gamma-delta T cells.

As used herein, a “cytotoxic cell” includes CD8⁺ T cells, natural-killer (NK) cells, NK-T cells, γδ T cells, a subpopulation of CD4⁺ cells, and neutrophils, which are cells capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used interchangeably herein, the terms “individual”, “subject”, “host”, and “patient” refer to a mammal, including, but not limited to, humans, murines (e.g., rats, mice), lagomorphs (e.g., rabbits), non-human primates, humans, canines, felines, ungulates (e.g., equines, bovines, ovines, porcines, caprines), etc.

As used herein, the terms “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

As used herein, the term “evolution” or “evolving” refers to using one or more methods of mutagenesis to generate a different polynucleotide encoding a different polypeptide, which is itself an improved biological molecule and/or contributes to the generation of another improved biological molecule. “Physiological” or “normal” or “normal physiological” conditions are conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions, that would be considered within a normal range at the site of administration, or at the tissue or organ at the site of action, to a subject.

As used herein, a “genetically modified cell” is a cell that contain an exogenous nucleic acid(s) regardless of whether the exogenous nucleic acid(s) is integrated into the genome of the cell. As used herein, a “transduced cell” is a cell that contains an exogenous nucleic acid(s) that is integrated into the genome of the cell.

A “polypeptide” as used herein can include part of or an entire protein molecule as well as any posttranslational or other modifications.

A pseudotyping element as used herein can include a “binding polypeptide” that includes one or more polypeptides, typically glycoproteins, that identify and bind the target host cell, and one or more “fusogenic polypeptides” that mediate fusion of the retroviral and target host cell membranes, thereby allowing a retroviral genome to enter the target host cell. The “binding polypeptide” as used herein, can also be referred to as a “T cell and/or NK cell binding polypeptide” or a “target engagement element,” and the “fusogenic polypeptide” can also be referred to as a “fusogenic element”.

A “resting” lymphocyte, such as for example, a resting T cell, is a lymphocyte in the GO stage of the cell cycle that does not express activation markers such as Ki-67. Resting lymphocytes can include naïve T cells that have never encountered specific antigen and memory T cells that have been altered by a previous encounter with an antigen. A “resting” lymphocyte can also be referred to as a “quiescent” lymphocyte.

As used herein, “lymphodepletion” involves methods that reduce the number of lymphocytes in a subject, for example by administration of a lymphodepletion agent. Lymphodepletion can also be attained by partial body or whole body fractioned radiation therapy. A lymphodepletion agent can be a chemical compound or composition capable of decreasing the number of functional lymphocytes in a mammal when administered to the mammal. One example of such an agent is one or more chemotherapeutic agents. Such agents and dosages are known, and can be selected by a treating physician depending on the subject to be treated. Examples of lymphodepletion agents include, but are not limited to, fludarabine, cyclophosphamide, cladribine, denileukin diftitox, or combinations thereof.

RNA interference (RNAi) is a biological process in which RNA molecules inhibit gene expression or translation by neutralizing targeted RNA molecules. The RNA target may be mRNA, or it may be any other RNA susceptible to functional inhibition by RNAi. As used herein, an “inhibitory RNA molecule” refers to an RNA molecule whose presence within a cell results in RNAi and leads to reduced expression of a transcript to which the inhibitory RNA molecule is targeted. An inhibitory RNA molecule as used herein has a 5′ stem and a 3′ stem that is capable of forming an RNA duplex. The inhibitory RNA molecule can be, for example, a miRNA (either endogenous or artificial) or a shRNA, a precursor of a miRNA (i.e. a Pri-miRNA or Pre-miRNA) or shRNA, or a dsRNA that is either transcribed or introduced directly as an isolated nucleic acid, to a cell or subject.

As used herein, “double stranded RNA” or “dsRNA” or “RNA duplex” refers to RNA molecules that are comprised of two strands. Double-stranded molecules include those comprised of two RNA strands that hybridize to form the duplex RNA structure or a single RNA strand that doubles back on itself to form a duplex structure. Most, but not necessarily all of the bases in the duplex regions are base-paired. The duplex region comprises a sequence complementary to a target RNA. The sequence complementary to a target RNA is an antisense sequence, and is frequently from 18 to 29, from 19 to 29, from 19 to 21, or from 25 to 28 nucleotides long, or in some embodiments between 18, 19, 20, 21, 22, 23, 24, 25 on the low end and 21, 22, 23, 24, 25, 26, 27, 28 29, or 30 on the high end, where a given range always has a low end lower than a high end. Such structures typically include a 5′ stem, a loop, and a 3′ stem connected by a loop which is contiguous with each stem and which is not part of the duplex. The loop comprises, in certain embodiments, at least 3, 4, 5, 6, 7, 8, 9, or 10 nucleotides. In other embodiments the loop comprises from 2 to 40, from 3 to 40, from 3 to 21, or from 19 to 21 nucleotides, or in some embodiments between 2, 3, 4, 5, 6, 7, 8, 9, 10, 11, 12, 13, 14, 15, 16, 17, 18, 19, or 20 on the low end and 10, 11, 12, 13, 14, 15, 16, 17, 18, 19, 20, 25, 30, 35, or 40 on the high end, where a given range always has a low end lower than a high end.

The term “microRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure.

The term “linker” when used in reference to a multiplex inhibitory RNA molecule refers to a connecting means that joins two inhibitory RNA molecules.

As used herein, a “recombinant retrovirus” refers to a non-replicable, or “replication incompetent”, retrovirus unless it is explicitly noted as a replicable retrovirus. The terms “recombinant retrovirus” and “recombinant retroviral particle” are used interchangeably herein. Such retrovirus/retroviral particle can be any type of retroviral particle including, for example, gamma retrovirus, and in illustrative embodiments, lentivirus. As is known, such retroviral particles, for example lentiviral particles, typically are formed in packaging cells by transfecting the packing cells with plasmids that include packaging components such as Gag, Pol and Rev, an envelope or pseudotyping plasmid that encodes a pseudotyping element, and a transfer, genomic, or retroviral (e.g. lentiviral) expression vector, which is typically a plasmid on which a gene(s) or other coding sequence of interest is encoded. Accordingly, a retroviral (e.g. lentiviral) expression vector includes sequences (e.g. a 5′ LTR and a 3′ LTR flanking e.g. a psi packaging element and a target heterologous coding sequence) that promote expression and packaging after transfection into a cell. The terms “lentivirus” and “lentiviral particle” are used interchangeably herein.

A “framework” of a miRNA consists of “5′ microRNA flanking sequence” and/or “3′ microRNA flanking sequence” surrounding a miRNA and, in some cases, a loop sequence that separates the stems of a stem-loop structure in a miRNA. In some examples, the “framework” is derived from naturally occurring miRNAs, such as, for example, miR-155. The terms “5′ microRNA flanking sequence” and “5′ arm” are used interchangeably herein. The terms “3′ microRNA flanking sequence” and “3′ arm” are used interchangeably herein.

As used herein, the term “miRNA precursor” refers to an RNA molecule of any length which can be enzymatically processed into an miRNA, such as a primary RNA transcript, a pri-miRNA, or a pre-miRNA.

As used herein, the term “construct” refers to an isolated polypeptide or an isolated polynucleotide encoding a polypeptide. A polynucleotide construct can encode a polypeptide, for example, a lymphoproliferative element. A skilled artisan will understand whether a construct refers to an isolated polynucleotide or an isolated polypeptide depending on the context.

As used herein, “MOI”, refers to Multiplicity of Infection ratio where the MOI is equal to the ratio of the number of virus particles used for infection per number of cells. Functional titering of the number of virus particles can be performed using FACS and reporter expression.

“Peripheral blood mononuclear cells” (PBMCs) include peripheral blood cells having a round nucleus and include lymphocytes (e.g. T cells, NK cells, and B cells) and monocytes. Some blood cell types that are not PBMCs include red blood cells, platelets and granulocytes (i.e. neutrophils, eosinophils, and basophils).

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. When multiple low and multiple high values for ranges are given that overlap, a skilled artisan will recognize that a selected range will include a low value that is less than the high value. All headings in this specification are for the convenience of the reader and are not limiting.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

The present disclosure overcomes prior art challenges by providing improved methods and compositions for genetically modifying lymphocytes, for example NK cells and in illustrative embodiments, T cells. Some of the methods and compositions herein, provide simplified and more rapid processes for transducing lymphocytes that avoid some steps that require specialized devices. Furthermore, the methods provide better control of post-transduction processing since any such processing is done ex vivo, which therefore allows the option of removing various unwanted cells. Thus, the methods provide an important step toward democratization of cell therapy methods.

Illustrative methods and compositions for genetically modifying lymphocytes, for example NK cells and in illustrative embodiments, T cells, are performed in less time than prior methods. Furthermore, compositions that have many uses, including their use in these improved methods, are provided. Some of these compositions are genetically modified lymphocytes that have improved proliferative and survival qualities, including in in vitro culturing, for example in the absence of growth factors. Such genetically modified lymphocytes will have utility for example, as research tools to better understand factors that influence T cell proliferation and survival, and for commercial production, for example for the production of certain factors, such as growth factors and immunomodulatory agents, that can be harvested and tested or used in commercial products.

Methods for Transducing and/or Genetically Modifying Lymphocytes

Provided herein in certain aspects, is a method of transducing and/or genetically modifying a lymphocyte, such as a (typically a population of) peripheral blood mononuclear cell (PBMC), typically a T cell and/or an NK cell, and in certain illustrative embodiments a resting T cell and/or resting NK cell, that includes contacting the lymphocyte with a (typically a population of) replication incompetent recombinant retroviral particle, wherein the replication incompetent recombinant retroviral particle typically comprises a pseudotyping element on its surface, wherein said contacting (and incubation under contacting conditions) facilitates membrane association, membrane fusion, and optionally transduction of the resting T cell and/or NK cell by the replication incompetent recombinant retroviral particle, thereby producing the genetically modified T cell and/or NK cell. In illustrative embodiments, pre-activation of the T cell and/or NK cell is not required, and an activation element, which can be any activation element provided herein, is present in a reaction mixture in which the contacting takes place. In further illustrative embodiments, the activation element is present on a surface of the replication incompetent recombinant retroviral particle. In illustrative embodiments, the activation element is anti-CD3, such as anti-CD3 scFv, or anti-CD3 scFvFc.

In some embodiments, the contacting step and an optional incubation thereafter, which includes a step to remove retroviral particles not associated with cells, in a method provided herein of transducing and/or genetically modifying a PBMC or a lymphocyte, typically a T cell and/or an NK cell, can be performed (or can occur), for 72, 48, or 24 hours or less or for any of the contacting time ranges provided herein. However, in illustrative embodiments, the contacting is performed for less than 2 hours, less than 1 hour, less than 30 minutes or less than 15 minutes, but in each case there is at least an initial contacting step in which retroviral particles and cells are brought into contact in suspension in a transduction reaction mixture. This contacting typically includes an initial step in which retroviral particles that are not associated with a cell of the reaction mixture are separated from the cells, which are then further processed. Such suspension can include allowing cells and retroviral particles to settle or causing such settling through application of a force, such as a centrifugal force, to the bottom of a vessel or chamber, as discussed in further detail herein. In illustrative embodiments, such g force is lower than the g forces used successfully in spinoculation procedures. Further contacting times and discussions regarding contacting and the optional incubation, are discussed further herein. In further illustrative embodiments, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture, which can be a step of separating free retroviral particles in a reaction mixture from those associated with cells.

Various embodiments of this method, as well as other aspects, such as use and NK cells and T cells made by such a method, are disclosed in detail herein. Furthermore, various elements or steps of such method aspects for transducing and/or genetically modifying a PBMC, lymphocyte, T cell and/or NK cell, are provided herein, for example in this section and the Exemplary Embodiments section, and such methods include embodiments that are provided throughout this specification, as further discussed herein, For example, embodiments of any of the aspects for transducing and/or genetically modifying a PBMC or a lymphocyte, for example an NK cell or in illustrative embodiments, a T cell, provided for example in this section and in the Exemplary Embodiments section, can include any of the embodiments of replication incompetent recombinant retroviral particles provided herein, including those that include one or more lymphoproliferative element, CAR, pseudotyping element, riboswitch, activation element, membrane-bound cytokine, miRNA, Kozak-type sequence, WPRE element, triple stop codon, and/or other element disclosed herein, and can be combined with methods herein for producing retroviral particles using a packaging cell. In certain illustrative embodiments, the retroviral particle is a lentiviral particle. Such a method for genetically modifying and/or transducing a PBMC or a lymphocyte, such as a T cell and/or NK cell can be performed in vitro or ex vivo. A skilled artisan will recognize that details provided herein for transducing and/or genetically modifying PBMCs or lymphocytes, such as T cell(s) and/or NK cell(s) can apply to any aspect that includes such step(s).

In certain illustrative embodiments, the cell is genetically modified and/or transduced without requiring prior activation or stimulation, whether in vivo, in vitro, or ex vivo. In certain illustrative embodiments, the cell is activated during the contacting and is not activated at all or for more than 15 minutes, 30 minutes, 1, 2, 4, or 8 hours before the contacting. In certain illustrative embodiments, activation by elements that are not present on the retroviral particle surface is not required for genetically modifying and/or transducing the cell. Accordingly, such activation or stimulation elements are not required other than on the retroviral particle, before, during, or after the contacting. Thus, as discussed in more detail herein, these illustrative embodiments that do not require pre-activation or stimulation provide the ability to rapidly perform in vitro experiments aimed at better understanding T cells and the biologicals mechanisms, therein. Furthermore, such methods provide for much more efficient commercial production of biological products produced using PBMCs, lymphocytes, T cells, or NK cells, and development of such commercial production methods. Finally, such methods provide for more rapid ex vivo processing of PBMCs for adoptive cell therapy, fundamentally simplifying the delivery of such therapies, for example by providing point of care methods.

### Compositions and Methods for Transducing Lymphocytes in Whole Bloodlymphocytes in Whole Blood

Provided herein in certain aspects, is a method of transducing and/or genetically modifying peripheral blood mononuclear cells (PBMCs), or lymphocytes, typically T cells and/or NK cells, and in certain illustrative embodiments resting T cells and/or resting NK cells, in a reaction mixture comprising blood, or a component thereof, and/or an anticoagulant, that includes contacting the lymphocytes with replication incompetent recombinant retroviral particles in the reaction mixture that itself represents a separate aspect provided herein, The reaction mixture in illustrative embodiments comprises the lymphocytes and the replication incompetent recombinant retroviral particles, a T cell activation element and one or more additional blood components set out below that in illustrative embodiments are present because the reaction mixture comprises at least 10% whole blood, wherein the replication incompetent recombinant retroviral particles typically comprises a pseudotyping element on its surface. In such methods, the contacting (and incubation under contacting conditions) facilitates association of the lymphocytes with the replication incompetent recombinant retroviral particles, wherein the recombinant retroviral particles genetically modify and/or transduce the lymphocytes. The reaction mixture of this aspect comprises at least 10% whole blood (e.g. at least 10%, 20%, 25%, 50%, 60%, 70%, 80%, 90%, 95%, or 99% whole blood) and optionally an effective amount of an anticoagulant, or the reaction mixture further comprises at least one additional blood or blood preparation component that is not a PBMC, for example the reaction mixture comprises an effective amount of an anti-coagulant and one or more blood preparation component that is not a PBMC. In illustrative embodiments such blood or blood preparation component that is not a PBMC is one or more (e.g. at least one, two, three, four, or five) or all of the following additional components:
a) erythrocytes, wherein the erythrocytes comprise between 1 and 60% of the volume of the reaction mixture;
b) neutrophils, wherein the neutrophils comprise at least 10% of the white blood cells in the reaction mixture, or wherein the reaction mixture comprises at least 10% as many neutrophils as T cells;
c) basophils, wherein the basophils comprise at least 0.05% of the white blood cells in the reaction mixture;
d) eosinophils, wherein the reaction mixture comprises at least 0.1% of the white blood cells in the reaction mixture;
e) plasma, wherein the plasma comprises at least 1% of the volume of the reaction mixture; and
f) an anti-coagulant
(such blood or blood preparation components a-f above referred to herein as (“Noteworthy Non-PBMC Blood or Blood Preparation Components”)).

The one or more additional blood components are present in certain illustrative embodiments of the reaction mixture (including related use, genetically modified T cell or NK cell, or method for genetically modifying T cells and/or NK cells aspects provided herein) because in these illustrative embodiments the reaction mixture comprises at least 10% whole blood, and in certain illustrative embodiments, at least 25%, 50%, 75%, 90%, or 95% whole blood, or for example between 25% and 95% whole blood. In these illustrative embodiments, such reaction mixtures are formed by combining whole blood with an anticoagulant (for example by collecting whole blood into a blood collection tube comprising an anti-coagulant), and adding a solution of recombinant retroviruses to the blood with anticoagulant. Thus, in illustrative embodiments, the reaction mixture comprises an anti-coagulant as set out in more detail herein. In some embodiments, the whole blood is not, or does not comprise, cord blood.

The reaction mixture in these aspects, typically does not include a PBMC enrichment procedure before the transduction reaction mixture is formed. Thus, typically such reaction mixtures include additional components listed in a)-f) above, which are not PBMCs. Furthermore, in illustrative embodiments, the reaction mixture comprises all of the additional components listed in a) to e) above, because the reaction mixture comprises substantially whole blood, or whole blood. “Substantially whole blood” is blood that was isolated from an individual(s), has not been subjected to a PBMC enrichment procedure, and is diluted by less than 50% with other solutions. For example, this dilution can be from addition of an anti-coagulant as well as addition of a volume of fluid comprising retroviral particles. Further reaction mixture embodiments for methods and compositions that relate to transducing lymphocytes in whole blood, are provided herein.

In another aspect, provided herein are genetically modified lymphocytes, in illustrative embodiments genetically modified T cells and/or NK cells made by the above method of transducing and/or genetically modifying lymphocytes in whole blood. In yet another aspect provided herein, is use of replication incompetent recombinant retroviral particles in the manufacture of a kit for genetically modifying lymphocytes, in illustrative embodiments T cells and/or NK cells of a subject, wherein the use of the kit comprises the above method of transducing and/or genetically modifying lymphocytes in whole blood. In another aspect, provided herein are methods for administering genetically modified lymphocytes to a subject, wherein the genetically modified lymphocytes are produced by the above method of transducing and/or genetically modifying lymphocytes in whole blood. Aspects provided herein that include such methods of transducing and/or genetically modifying lymphocytes in whole blood, uses of such a method in the manufacture of a kit, reaction mixtures formed in such a method, genetically modified lymphocytes made by such a method, and methods for administering a genetically modified lymphocyte made by such a method, are referred to herein as “composition and method aspects for transducing lymphocytes in whole blood.” It should be noted that although illustrative embodiments for such aspects involve contacting T cells and/or NK cells with retroviral particles in whole blood, such aspects also include other embodiments, where one or more of additional components a-f above, are present in transduction reaction mixtures at higher concentrations than is typical after a PBMC enrichment procedure.

Various elements or steps of such method aspects for transducing lymphocytes in whole blood, are provided herein, for example in this section and the Exemplary Embodiments section, and such methods include embodiments that are provided throughout this specification, as further discussed herein. A skilled artisan will recognize that many embodiments provided herein anywhere in this specification can be applied to any of the aspects of the composition and method aspects for transducing lymphocytes in whole blood. For example, embodiments of any of the composition and method aspects for transducing lymphocytes in whole blood provided for example in this section and/or in the Exemplary Embodiments section, can include any of the embodiments of replication incompetent recombinant retroviral particles provided herein, including those that include one or more polypeptide lymphoproliferative element, inhibitory RNA, CAR, pseudotyping element, riboswitch, activation element, membrane-bound cytokine, miRNA, Kozak-type sequence, WPRE element, triple stop codon, and/or other element disclosed herein, and can be combined with methods herein for producing retroviral particles using a packaging cell.

As non-limiting examples of embodiments that can be used in many aspects herein, as discussed in more detail herein, the pseudotyping element is typically capable of binding lymphocytes (e.g. T cells and/or NK cells) in illustrative embodiments resting T cells and/or resting NK cells and facilitating membrane fusion on its own or in conjunction with other protein(s) of the replication incompetent recombinant retroviral particles. In certain illustrative embodiments, the retroviral particle is a lentiviral particle. Such a method for genetically modifying a lymphocyte, such as a T cell and/or NK cell in whole blood, can be performed in vitro or ex vivo.

Anticoagulants are included in reaction mixtures for certain embodiments of the composition and method aspects for transducing lymphocytes in whole blood provided herein. In some illustrative embodiments, blood is collected with the anti-coagulant present in the collection vessel (e.g. tube or bag), for example using standard blood collection protocols known in the art. Anticoagulants that can be used in composition and method aspects for transducing lymphocytes in whole blood provided herein include compounds or biologics that block or limit the thrombin blood clotting cascade. The anti-coagulants include: metal chelating agents, preferably calcium ion chelating agents, such as citrate (e.g. containing free citrate ion), including solutions of citrate that contain one or more components such as citric acid, sodium citrate, phosphate, adenine and mono or polysaccharides, for example dextrose, oxalate, and EDTA; heparin and heparin analogues, such as unfractionated heparin, low molecular weight heparins, and other synthetic saccharides; and vitamin K antagonists such as coumarins. Exemplary citrate compositions include: acid citrate dextrose (ACD) (also called anticoagulant citrate dextrose solution A and solution B (United States Pharmacopeia 26, 2002, pp 158)); and a citrate phosphate dextrose (CPD) solution, which can also be prepared as CPD-A1 as is known in the art. Accordingly, the anticoagulant composition may also include phosphate ions or monobasic phosphate ion, adenine, and mono or polysaccharides.

Such anti-coagulants can be present in a reaction mixture at concentrations that are effective for preventing coagulation of blood (i.e. effective amounts) as known in the art, or at a concentration that is, for example, 2 times, 1.5 times, 1.25 times, 1.2 times, 1.1 times, or 9/10, ⅘, 7/10, ⅗, ½, ⅖, 3/10, ⅕, or 1/10 the effective concentration. The effective concentrations of many different anticoagulants is known and can be readily determined empirically by analyzing different concentrations for their ability to prevent blood coagulation, which can be physically observed. Numerous coagulometers are available commercially that measure coagulation, and various sensor technologies can be used, for example QCM sensors (See e.g., Yao et al., “Blood Coagulation Testing Smartphone Platform Using Quartz Crystal Microbalance Dissipation Method,” Sensors (Basel). 2018 September; 18(9): 3073). The effective concentration includes the concentration of any commercially available anti-coagulant in a commercially available tube or bag after the anti-coagulant is diluted in the volume of blood intended for the tube or bag. For example, the concentration of acid citrate dextrose (ACD) in a reaction mixture in certain embodiments of the composition and method aspects for transducing lymphocytes in whole blood provided herein, can be between 0.1 and 5×, or between 0.25 and 2.5×, between 0.5 and 2×, between 0.75 and 1.5×, between 0.8 and 1.2×, between 0.9 and 1.1×, about 1×, or 1× the concentration of ACD in a commercially available ACD blood collection tube or bag. For example, in a standard process, blood can be collected into tubes or bags containing 3.2% (109 mM) sodium citrate (109 mM) at a ratio of 9 parts blood and 1 part anticoagulant. Thus, in certain illustrative embodiments with a reaction mixture made by adding 1-2 parts of a retroviral particle solution to this mixture of 1 part anticoagulant to 9 parts blood, the citrate concentration can be between for example, 0.25% to 0.4%, or 0.30% to 0.35%. In an illustrative standard blood collection embodiment, 15 mls of ACD Solution A are present in a blood bag for collecting 100 mL of blood. The ACD before addition of blood contains Citric acid (anhydrous) 7.3 g/L (0.73%), Sodium citrate (dihydrate) 22.0 g/L (2.2%), and Dextrose (monohydrate) 24.5 g/L [USP] (2.4%). After addition of 100 ml of blood to the bag that contains ACD, a volume of for example, between 5 and 20 mis of the genetically modified retroviral particles is added. Thus, in some embodiments, the concentration of ACD components in a reaction mixture can be between 0.05 and 0.1%, or 0.06 and 0.08% Citric acid (anhydrous), 0.17 and 0.27, or 0.20 and 0.24 Sodium citrate (dihydrate), 0.2 and 0.3, or 0.20 and 0.28, or 0.22 and 0.26% Dextrose (monohydrate). In certain embodiments, sodium citrate is used at a concentration of between 0.001 and 0.02 M in the reaction mixture.

In some embodiments, heparin is present in the reaction mixtures, for example at a concentration between 0.1 and 5×, or between 0.25 and 2.5×, between 0.5 and 2×, between 0.75 and 1.5×, between 0.8 and 1.2×, between 0.9 and 1.1×, about 1×, or 1× the concentration of heparin in a commercially available heparin blood collection tube. Heparin is a glycosaminoglycan anticoagulant with a molecular weight ranging from 5,000-30,000 daltons. In some embodiments, heparin is used at a concentration of about 1.5 to 45, 5 to 30, 10 to 20, or 15 USP units/ml of reaction mixture. In some embodiments, the effective concentration for EDTA, for example as K₂EDTA, in the reaction mixtures herein can be between 0.15 and 5 mg/ml, between 1 and 3 mg/ml between 1.5-2.2 mg/ml of blood, or between 1 and 2 mg/ml, or about 1.5 mg/ml. The reaction mixtures in composition and method aspects for transducing lymphocytes in whole blood provided herein, can include two or more anticoagulants whose combined effective dose prevents coagulation of the blood prior to formation of the reaction mixture and/or of the reaction mixture itself.

In some embodiments, the anti-coagulant can be administered to a subject before blood is collected from the subject for ex vivo transduction, such that coagulation of the blood when it is collected in inhibited, at least partially and at least through a contacting step and optional incubation period thereafter. In such embodiments, for example acid citrate dextrose can be administered to the subject at between 80 mg/kg/day and 5 mg/kg/day (mg refer to the mg of citric acid and kg applies to the mammal to be treated). Heparin, can be delivered for example, at a dose of between 5 units/kg/hr to 30 units/kg/hr.

In addition to, or instead of an anti-coagulant, composition and method aspects for transducing lymphocytes in whole blood provided herein, can include at least one additional component selected from one or more of the following components:
a) erythrocytes, wherein the erythrocytes comprise between 0.1 and 75% of the volume of the reaction mixture;
b) neutrophils, wherein the neutrophils comprise at least 10% of the white blood cells in the reaction mixture, or wherein the reaction mixture comprises at least 10% as many neutrophils as T cells;
c) basophils, wherein the basophils comprise at least 0.05% of the white blood cells in the reaction mixture;
d) eosinophils, wherein the reaction mixture comprises at least 0.1% of the white blood cells in the reaction mixture;
e) plasma, wherein the plasma comprises at least 1% of the volume of the reaction mixture; and
f) platelets, wherein the platelets comprise at least 1×10⁶ platelets/liter of the reaction mixture.

With respect to erythrocytes, in some embodiments, erythrocytes can comprise between 0.1, 0.5, 1, 5, 10, 25, 35 or 40% of the volume of the reaction mixture on the low end of the range, and between 25, 50, 60, or 75% of the volume of the reaction mixture on the high end of the range. In illustrative embodiments, erythrocytes comprise between 1 and 60%, between 10 and 60%, between 20 and 60%, between 30 and 60%, between 40 and 60%, between 40 and 50%, between 42 and 48%, between 44 and 46%, about 45% or 45%.

With respect to neutrophils, in some embodiments, neutrophils can comprise between 0.1, 0.5, 1, 5, 10, 20, 25, 35 or 40% of the white blood cells of the reaction mixture on the low end of the range, and between 25, 50, 60, 70, 75 and 80% of the white blood cells of the reaction mixture on the high end of the range, for example between 25% and 70%, or between 30% and 60%, or between 40% and 60% of the white blood cells of the reaction mixture. In some embodiments, more neutrophils are present than T cells and/or NK cells, in reaction mixtures herein.

With respect to eosinophils in some embodiments, eosinophils can comprise between 0.05, 0.1, 0.2, 0.4, 0.6, 0.8, 1.0, 1.2, 1.4, 1.6, and 1.8% of the white blood cells of the reaction mixture on the low end of the range, and between 2.0, 2.2, 2.4, 2.6, 2.8, 3.0, 3.5, 4, 5, 6, 8 and 10% of the white blood cells of the reaction mixture on the high end of the range. In illustrative embodiments, eosinophils comprise between 0.05 and 10.0%, between 0.1 and 9%, between 0.2 and 8%, between 0.2 and 6%, between 0.5 and 4%, between 0.8 and 4%, or between 1 and 4% of the white blood cells of the reaction mixture.

With respect to basophils in some embodiments, basophils can comprise between 0.05, 0.1, 0.2, 0.4, 0.45 and 0.5% of the white blood cells of the reaction mixture on the low end of the range, and between 0.8, 0.9, 1.0, 1.1, 1.2, 1.5 and 2.0% of the white blood cells of the reaction mixture on the high end of the range. In illustrative embodiments, basophils comprise between 0.05 and 1.4%, between 0.1 and 1.4%, between 0.2 and 1.4%, between 0.3 and 1.4%, between 0.4 and 1.4%, between 0.5 and 1.4%, between 0.5 and 1.2%, between 0.5 and 1.1%, or between 0.5 and 1.0% of the white blood cells of the reaction mixture.

With respect to plasma, in some embodiments, plasma can comprise between 0.1, 0.5, 1, 5, 10, 25, 35 or 45% of the volume of the reaction mixture on the low end of the range, and between 25, 50, 60, 70 and 80% of the volume of the reaction mixture on the high end of the range. In illustrative embodiments, plasma comprise between 0.1 and 80%, between 1 and 80%, between 5 and 80%, between 10 and 80%, between 30 and 80%, between 40 and 80%, between 45 and 70%, between 50 and 60%, between 52 and 58%, between 54 and 56%, about 55% or 55% of the reaction mixture.

With respect to platelets, in some embodiments, platelets can comprise between 1×10⁵, 1×10⁶, 1×10⁷, or 1×10⁸ platelets/mL of the reaction mixture on the low end of the range, and between 1×10⁹, 1×10¹⁰, 1×10¹¹, 1×10¹², 2×10¹³, or 2×10¹⁴ platelets/mL of the reaction mixture on the high end of the range. In illustrative embodiments, platelets comprise between 1×10⁵ and 1×10¹² platelets, between 1×10⁶ and 1×10¹¹ platelets, between 1×10⁷ and 1×10¹⁰ platelets, between 1×10⁸, and 1×10⁹ platelets/mL, or between 1×10⁸ and 5×10⁸ platelets/ml of the reaction mixture.

Illustrative Cell Processing Methods for Genetically Modifying T Cells and/or NK Cells in the Presence of Blood, or a Component Thereof

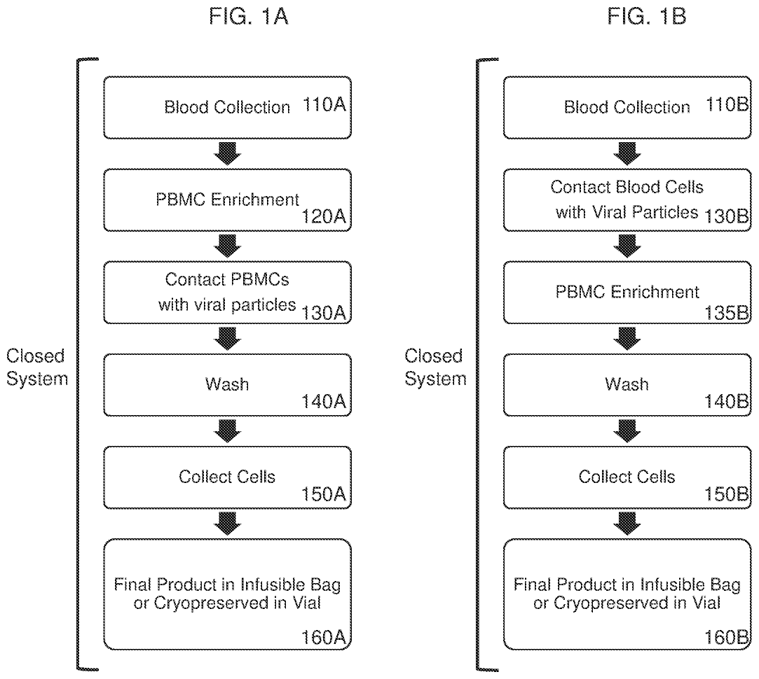

It is noteworthy that some embodiments of methods for genetically modifying provided herein do not include a step of collecting blood from a subject. However, as shown in FIG. 1, some of the methods provided herein include a step where blood is collected (110) from a subject. Blood can be collected or obtained from a subject by any suitable method known in the art as discussed in more detail herein. For example, the blood can be collected by venipuncture or any other blood collection method by which a sample of blood is collected. In some embodiments, the volume of blood collected is between 25 ml and 250 ml, for example, between 25 ml and 60 ml, between 50 ml and 90 ml, between 75 ml and 125 ml, or between 90 ml and 120 ml, or between 95 and 110 ml.

Regardless of whether blood is collected from a subject, in any of the method aspects provided herein for genetically modifying lymphocytes (e.g. T cells and/or NK cells), the lymphocytes are contacted with replication incompetent retroviral particles in a reaction mixture. In illustrative embodiments, this contacting, and the reaction mixture in which the contacting occurs, takes place within a closed cell processing system, as discussed in more detail herein. In traditional closed cell processing methods that involve genetic modification and/or transductions of lymphocytes ex vivo, especially in methods for autologous cell therapy, many steps occur over days, such as PBMC enrichment(s), washing(s), cell activation, transduction, expansion, collection, and optionally reintroduction. In more recent methods (See FIG. 1A), some of the steps and time involved in this ex vivo cell processing have been reduced (See e.g. WO2019/055946). These more recent methods (as well as the further improved cell processing methods provided herein), furthermore use a rapid ex vivo transduction process, for example that includes no or minimal preactivation (e.g. less than 30, 15, 10, or 5 minutes of contacting lymphocytes such as T cells and/or NK cells with an activation agent before they are contacted with retroviral particles). In certain embodiments of such methods, a T cell and/or NK cell activation element is present in the reaction mixture in which the contacting step occurs. In illustrative embodiments, the T cell and/or NK cell activation element is associated with surfaces of retroviral particles present in the reaction mixture. In illustrative embodiments, such a method is used in a point of care autologous cell therapy method. However, such more recent methods still involve a PBMC enrichment step/procedure (120), which typically takes at least around 1 hour within the closed system, followed by cell counting, transfer and media addition, which takes at least around 45 additional minutes before lymphocytes are contacted with retroviral particles to form a transduction reaction mixture (130A). Following the “viral transduction” step, which typically is a contacting step with incubating as discussed in detail herein, lymphocytes are typically washed away from retroviral particles that remain in suspension (140A), for example using a Sepax, and collected (150A), with the final product typically in an infusion bag for reinfusion or cryopreservation vial for storage (160A). As discussed in further detail herein, traditional PBMC enrichment procedures typically involve ficoll density gradients and centrifugal (e.g. centrifugation) or centripetal (e.g. Sepax) forces or use leukophoresis to enrich PBMCs.

As demonstrated in the Examples provided herein, it was surprisingly found that lymphocytes (e.g. T cells and/or NK cells) can be contacted with replication incompetent retroviral particles in a reaction mixture of whole blood that contains an anti-coagulant, and a significant percentage of the lymphocytes can be genetically modified and transduced. Thus, it was discovered that effective genetic modification of lymphocytes by recombinant retroviral particles can be carried out in the presence of blood components and blood cells in addition to PBMCs. Furthermore, based on the surprising finding discussed immediately above regarding effective genetic modification of T cells and optionally NK cells by retroviral particles even when contacting is performed in whole blood, provided herein in an illustrative embodiment, is a further simplified method in which lymphocytes are genetically modified and/or transduced by adding replication incompetent retroviral particles directly to whole blood to form a reaction mixture (130B), and cells in the whole blood are contacted by the replication incompetent retroviral particles for contacting times with optional incubations provided herein. Such a further improved method in this illustrative embodiment, thus includes no lymphocyte enrichment steps before lymphocytes in whole blood, typically containing an anti-coagulant, are contacted with retroviral particles. This further improved method, like other cell processing methods herein, is typically carried out within a closed cell processing system and can include no or minimal preactivation before lymphocytes are contacted with retroviral particles. In these further simplified methods lymphocytes in whole blood can be contacted with retroviral particles directly in a blood bag. After the contacting step (130B) in such methods, lymphocytes that were contacted with retroviral particles, are washed and concentrated using a PBMC enrichment procedure (135B), which also reduces neutrophils to facilitate reintroduction into a subject. Thus, in such embodiments, no PBMC enrichment procedure and no lymphocyte-enriching filtration is performed before cells in whole blood, and typically comprising an anticoagulant, are contacted with recombinant retroviral particles. However, in the embodiment of FIG. 1B, such a PBMC enrichment method is performed (135B) for example using a Sepax with a ficoll gradient, after the contacting with optional incubation (130B) is carried out. Following the PBMC enrichment, lymphocytes optionally can be washed further away from any retroviral particles that remain (140B), for example using a Sepax, and collected (150B), with the final product typically in an infusion bag for reinfusion or cryopreservation vial for storage (160B).

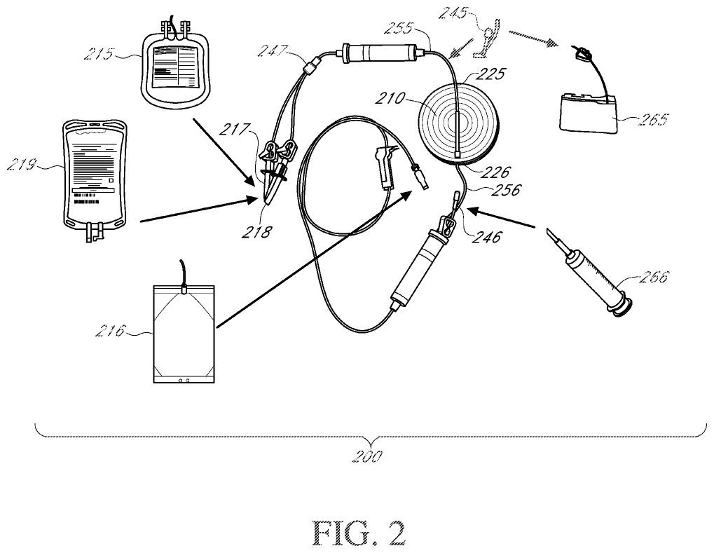

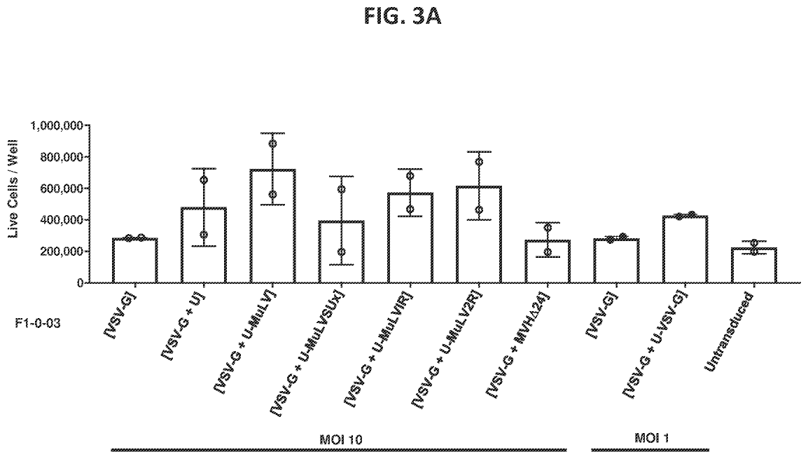

FIG. 2 provides a non-limiting illustrative example of a cell processing leukodepletion filtration assembly (200) that enriches nucleated cells that can be used as the leukodepletion filter in the methods of FIG. 1. The illustrative leukodepletion filtration assembly (200), which in illustrative embodiments is a single-use filtration assembly, comprises a leukocyte depletion media (e.g. filter set) within a filter enclosure (210), that has an inlet (225), and an outlet (226), and a configuration of bags, valves and/or channels/tubes that provide the ability to concentrate, enrich, wash and collect retained white blood cells or nucleated blood cells using perfusion and reverse perfusion (see e.g. EP2602315A1, incorporated by reference herein, in its entirety). In an illustrative embodiment, the leukodepletion filtration assembly (200) is a commercially available HemaTrate filter (Cook Regenetec, Indianapolis, Ind.). Leukodepletion filtration assemblies can be used, to concentrate total nucleated cells (TNC) including granulocytes, which are removed in PBMC enrichment procedures in a closed cell processing system. Since a filter assembly comprising leukocyte depletion media of EP2602315A1 such as a HemaTrate filter and the illustrative leukodepletion filter assembly of FIG. 2 do not remove granulocytes, they are not considered PBMC enrichment assemblies or filters herein, and methods that incorporate them are not considered PBMC enrichment procedures or steps herein.

The leukodepletion filter assembly (200) of FIG. 2 is a single-use sterile assembly that includes various tubes and valves, typically needle-free valves, that allow isolation of white blood cells from whole blood and blood cell preparations that include leukocytes, as well as rapid washing and concentrating of white blood cells. In this illustrative assembly, a blood bag (215), for example a 500 ml PVC bag containing about 120 ml of a transduction/contacting reaction mixture comprising whole blood, an anti-coagulant, and retroviral particles is connected to the assembly (200) at a first assembly opening (217) of an inlet tubing (255), after the reaction mixture is subjected to a contacting step with optional incubation, as disclosed in detail herein. Lymphocytes, including some T cells and/or NK cells with associated retroviral particles, and some that could be genetically modified at this point, as well as other blood cells and components in the whole blood reaction mixture as well as the anti-coagulant enter the inlet tubing (255) through the first assembly opening (217) by gravitational force when a clamp on the first inlet tubing (255) is released. The genetically modified T cells and/or NK cells pass through a inlet valve (247) and a collection valve (245), to enter a filter enclosure (210) through a filter enclosure inlet (225) to contact a leukodepletion IV filter set (e.g. SKU J1472A Jorgensen Labs) within the filter enclosure (210). Nucleated blood cells including leukocytes are retained by the filter, but other blood components pass through the filter and out the filter enclosure outlet (226) into the outlet tubing (256), then through an outlet valve (247) and are collected in a waste collection bag (216), which for example can be a 2 L PVC waste collection bag.

An optional buffer wash step can be performed by switching inlet valve (247) to a wash position. In this optional wash step, a buffer bag (219), for example a 500 ml saline wash bag, is connected to a second assembly opening (218) of inlet tubing (255). The buffer moves into the inlet tubing (255) through the second assembly opening (218) by gravitational force when a clamp on the inlet tubing (255) is released. The buffer passes through inlet valve (247) and collection valve (245), to enter filter enclosure (210) through the filter enclosure inlet (225) and passes through the leukodepletion filter set within the filter enclosure (210) to rinse the lymphocytes retained on the filter. The buffer moves out the filter enclosure outlet (226) into the outlet tubing (256), then through an outlet valve (247) and is collected in a waste collection bag (216), which can be the same waste collection bag as used to collect reaction mixture components that passed through the filter in the previous step, or a new waste collection bag swapped in place of the first waste collection bag before the buffer was allowed to enter the second assembly opening (218). The optional wash step can be optionally performed multiple times by repeating the above process with additional buffer.

Once the entire or substantially the entire volume of the reaction mixture in the blood bag (215) passes over the filter (210), and the optional washing step(s) is optionally performed, a reverse perfusion process is initiated to move fluid in an opposite direction in the assembly (200) to collect lymphocytes retained on the filter set within the filter enclosure (210). Illustrative embodiments of leukodepletion filter assemblies herein are adaptable for reperfusion. Before initiating the reverse perfusion process in the illustrative assembly (200), the outlet valve (247) is switched to a reperfusion position and the collection valve (245) is switched to a collection position. To initiate reperfusion, a buffer (e.g. PBS) in syringe (266), which for example can be a 25 ml syringe, is passed into outlet tubing (256) by injection using syringe (266). The buffer then enters the filter enclosure (210) through the filter enclosure outlet (226) and moves lymphocytes retained on the filter set out of the filter enclosure (210) through the filter enclosure inlet (225) and into the inlet tubing (255). Then lymphocytes, including some T cells and/or NK cells with associated retroviral particles, some of which could be genetically modified and/or transduced at this point, are collected in a cell sample collection bag (265), which for example can be a 25 ml cryopreservation bag, after the pass through the collection valve (245).

In some aspects, provided herein is a kit for genetically modifying NK cells and/or in illustrative embodiments, T cells. The kit includes a leukodepletion filtration assembly and any of the replication incompetent retroviral vector embodiments disclosed herein, typically contained in a tube or vial. The leukodepletion filtration assembly in such a kit typically includes a leukodepletion filter or a leukodepletion filter set, typically within a filter enclosure, as exemplified by the illustrative assembly of FIG. 2, as well as a plurality of connected sterile tubes and a plurality of valves connected thereto, that are adapted for use in a single-use closed blood processing system. Such a kit optionally includes a blood collection bag, in illustrative embodiments comprising an anti-coagulant, a blood processing buffer bag, a blood processing waste collection bag, a blood processing cell sample collection bag, and a sterile syringe. In illustrative embodiments, the kit includes a T cell activation element as disclosed in detail herein, for example anti-CD3. Such activation element can be provided in solution in the tube or vial containing the retroviral particle, or in a separate tube or vial. In illustrative embodiments, the activation element is an anti-CD3 associated with a surface of the replication incompetent retroviral particle. In illustrative embodiments, the replication incompetent recombinant retroviral particles in the kit comprise a polynucleotide comprising one or more transcriptional units operatively linked to a promoter active in T cells and/or NK cells, wherein the one or more transcriptional units encode a first polypeptide comprising a chimeric antigen receptor (CAR) and optionally a lymphoproliferative element, according to any of the embodiments provided herein.

### Steps and Reaction Mixtures for Methods for Genetically Modifying Lymphocytes

Some embodiments of any methods used in any aspects provided herein, which are typically methods for genetically modifying lymphocytes, PBMCs, and in illustrative embodiments NK cells and/or in further illustrative embodiments, T cells, can include a step of collecting blood from a subject. The blood includes blood components including blood cells such as lymphocytes (e.g. T cells and NK cells) that can be used in methods and compositions provided herein. In certain illustrative embodiments, the subject is a human subject afflicted with cancer (i.e. a human cancer subject). It is noteworthy that certain embodiments, do not include such a step. However, in embodiments that include collecting blood from a subject, blood can be collected or obtained from a subject by any suitable method known in the art as discussed in more detail herein. For example, the blood can be collected by venipuncture or any other blood collection method by which a sample of blood is collected. In some embodiments, the volume of blood collected is between 50 ml and 250 ml, for example, between 75 ml and 125 ml, or between 90 ml and 120 ml, or between 95 and 110 ml. In some embodiments, the volume of blood collected can be between 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml on the low end of the range and 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml or 1 L on the high end of the range. In some embodiments, lymphocytes (e.g. T cells and/or NK cells) can be obtained by apheresis. In some embodiments, the volume of blood taken and processed during apheresis can be between 0.5, 0.6, 0.7, 0.75, 0.8, 0.9, 1, 1.25, or 1.5 total blood volumes of a subject on the low end of the range and 0.6, 0.7, 0.75, 0.8, 0.9, 1, 1.25, 1.5 1.75, 2, 2.25, or 2.5 total blood volumes of a subject on the high end of the range. The total blood volume of a human typically ranges from 4.5 to 6 L and thus much more blood is taken and processed during apheresis than if the blood is collected and then lymphocytes therein are genetically modified and/or transduced, as in illustrative embodiments herein.

Regardless of whether blood is collected from a subject, in any of the method aspects provided herein for genetically modifying lymphocytes (e.g. T cells and/or NK cells), the lymphocytes are contacted with replication incompetent retroviral particles in a reaction mixture. The contacting in any embodiment provided herein, can be performed for example in a chamber of a closed system adapted for processing of blood cells, for example within a blood bag, as discussed in more detail herein. The transduction reaction mixture can include one or more buffers, ions, and a culture media. With respect to retroviral particles, and in illustrative embodiments, lentiviral particles, in certain exemplary reaction mixtures provided herein, between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15, multiplicity of infection (MOI); or at least 1 and less than 6, 11, or 51 MOI; or in some embodiments, between 5 and 10 MOI units of replication incompetent recombinant retroviral particles are present. In some embodiments, the MOI can be at least 0.1, 0.5, 1, 2, 2.5, 3, 5, 10 or 15. With respect to composition and method for transducing lymphocytes in blood, in certain embodiments higher MOI can be used than in methods wherein PBMCs are isolated and used in the reaction mixtures. For example, illustrative embodiments of compositions and methods for transducing lymphocytes in whole blood, assuming 1×10⁶ PBMCs/ml of blood, can use retroviral particles with an MOI of between 1 and 50, 2 and 25, 2.5 and 20, 2.5 and 10, 4 and 6, or about 5, and in some embodiments between 5 and 20, 5 and 15, 10 and 20, or 10 and 15.

In illustrative embodiments, this contacting, and the reaction mixture in which the contacting occurs, takes place within a closed cell processing system, as discussed in more detail herein. A packaging cell, and in illustrative embodiments a packaging cell line, and in particularly illustrative embodiments a packaging cell provided in certain aspects herein, can be used to produce the replication incompetent recombinant retroviral particles. The lymphocytes in the reaction mixture can be PBMCs, or in aspects herein that provide compositions and methods for transducing lymphocytes in whole blood, an anti-coagulant and/or an additional blood component, including additional types of blood cells that are not PBMCs, as discussed herein. In fact, in illustrative embodiments of these composition and method aspects for transducing lymphocytes in whole blood, the reaction mixture can essentially be whole blood, and typically an anti-coagulant, retroviral particles, and a small amount of the solution in which the retroviral particles were delivered to the whole blood.

In some reaction mixture provided herein, T-cells can be present for example, between 10, 20, 30, or 40% of the lymphocytes of the reaction mixture on the low end of the range, and between 40, 50, 60, 70, 80, or 90% of the lymphocytes of the reaction mixture on the high end of the range. In illustrative embodiments, T-cells comprise between 10 and 90%, between 20 and 90%, between 30 and 90%, between 40 and 90%, between 40 and 80%, between 45% to 75% or of the lymphocytes. In such embodiments, for example, NK cells can be present at between 1, 2, 3, 4, or 5% of the lymphocytes of the reaction mixture on the low end of the range, and between 5, 6, 7, 8, 9, 10, 11, 12, 13, or 14% of the lymphocytes of the reaction mixture on the high end of the range. In illustrative embodiments, T-cells comprise between 1 and 14%, between 2 and 14%, between 3 and 14%, between 4 and 14%, between 5 and 14%, between 5 to 13%, between 5 to 12%, between 5 to 11% or, between 5 to 10% of the lymphocytes of the reaction mixture.

In reaction mixtures that relate to composition and method aspects for genetically modifying lymphocytes in whole blood provided herein, lymphocytes, including NK cells and T cells, can be present at a lower percent of blood cells, and at a lower percentage of white blood cells, in the reaction mixture than methods that involve a PBMC enrichment procedure before forming the reaction mixture. For example, in some embodiments of these aspects, more granulocytes or neutrophils are present in the reaction mixture than NK cells or even T cells. Details regarding compositions of anti-coagulants and one or more additional blood components present in the reaction mixtures of aspects for genetically modifying lymphocytes in whole blood, are provided in detail in other sections herein.

As disclosed herein, composition and method aspects for transducing lymphocytes in whole blood typically do not involve a PBMC enrichment step of a blood sample, before lymphocytes from the blood sample are contacted with retroviral particles in the reaction mixtures disclosed herein for those aspects. However, in some embodiments, neutrophils/granulocytes are separated away from other blood cells before the cells are contacted with replication incompetent recombinant retroviral particles. In some embodiments, peripheral blood mononuclear cells (PBMCs) including peripheral blood lymphocytes (PBLs) such as T cell and/or NK cells, are isolated away from other components of a blood sample using for example, a PBMC enrichment procedure, before they are combined into a reaction mixture with retroviral particles.

A PBMC enrichment procedure is a procedure in which PBMCs are enriched at least 25-fold, and typically at least 50-fold from other blood cell types. For example, it is believed that PBMCs make up less than 1% of blood cells in whole blood. After a PBMC enrichment procedure, at least 30%, and in some examples as many as 70% of cells isolated in the PBMC fraction are PBMCs. It is possible that even higher enrichment of PBMCs is achieved using some PBMC enrichment procedures. Various different PBMC enrichment procedures are known in the art. For example, a PBMC enrichment procedure is a ficoll density gradient centrifugation process that separates the main cell populations, such as lymphocytes, monocytes, granulocytes, and red blood cells, throughout a density gradient medium. In such a method the aqueous medium includes ficoll, a hydrophilic polysaccharide that forms the high density solution. Layering of whole blood over or under a density medium without mixing of the two layers followed by centrifugation will disperse the cells according to their densities with the PBMC fraction forming a thin white layer at the interface between the plasma and the density gradient medium (see e.g. Panda and Ravindran (2013) Isolation of Human PBMCs. BioProtoc. Vol. 3(3)). Furthermore, centripetal forces can be used to separate PBMCs from other blood components, in ficoll using the spinning force of a Sepax cell processing system.

In another PBMC enrichment method, an automated leukapheresis collection system (such as SPECTRA OPTIA® APHERESIS SYSTEM form TERUMO BCT, INC. Lakewood Colo. 80215, USA) is used to separate the inflow of whole blood from the target PBMC fraction using high-speed centrifugation while typically returning the outflow material, such as plasma, red blood cells, and granulocytes, back to the donor, although this returning would be optional in methods provided herein. Further processing may be necessary to remove residual red blood cells and granulocytes. Both methods include a time intensive purification of the PBMCs, and the leukapheresis method requires the presence and participation of the patient during the PBMC enrichment step.

As further non-limiting examples of PBMC enrichment procedures, in some embodiments for methods of transducing or genetically modifying herein, PBMCs are isolated using a Sepax or Sepax 2 cell processing system (BioSafe). In some embodiments, the PBMCs are isolated using a CliniMACS Prodigy cell processor (Miltenyi Biotec). In some embodiments, an automated apheresis separator is used which takes blood from the subject, passes the blood through an apparatus that sorts out a particular cell type (such as, for example, PBMCs), and returns the remainder back into the subject. Density gradient centrifugation can be performed after apheresis. In some embodiments, the PBMCs are isolated using a leukodepletion filter assembly. In some embodiments, magnetic bead activated cell sorting is then used for purifying a specific cell population from PBMCs, such as, for example, PBLs or a subset thereof, according to a cellular phenotype (i.e. positive selection), before they are used in a reaction mixture herein.

Other methods for purification can also be used, such as, for example, substrate adhesion, which utilizes a substrate that mimics the environment that a T cell encounters during recruitment, to purify T cells before adding them to a reaction mixture, or negative selection can be used, in which unwanted cells are targeted for removal with antibody complexes that target the unwanted cells for removal before a reaction mixture for a contacting step is formed. In some embodiments, red blood cell rosetting can be used to remove red blood cells before forming a reaction mixture. In other embodiments, hematopoietic stem cells can be removed before a contacting step, and thus in these embodiments, are not present during the contacting step. In some embodiments herein, especially for compositions and methods for transducing lymphocytes in whole blood, an ABC transporter inhibitor and/or substrate is not present before, during, or both before and during the contacting (i.e. not present in the reaction mixture in which contacting takes place) with or without optional incubating, or any step of the method.

In certain illustrative embodiments for any aspects provided herein, lymphocytes are genetically modified and/or transduced without prior activation or stimulation, and/or without requiring prior activation or stimulation, whether in vivo, in vitro, or ex-vivo; and/or furthermore, in some embodiments, without ex vivo or in vitro activation or stimulation after an initial contacting with or without an optional incubation, or without requiring ex vivo or in vitro activation or stimulation after an initial contacting with or without an optional incubation. Thus, in illustrative embodiments, some, most, at least 25%, 50%, 60%, 70%, 75%, 80%, 90%, at least 95%, at least 99%, or all of the lymphocytes are resting when they are combined with retroviral particles to form a reaction mixture, and typically are resting when they are contacted with retroviral viral particles in a reaction mixture. In methods for genetically modifying lymphocytes such as T cells and/or NK cells in blood or a component thereof, lymphocytes can be contacted in the typically resting state they were in when present in the collected blood in vivo immediately before collection. In some embodiments, the T cells and/or NK cells consist of between 95 and 100% resting cells (Ki-67-). In some embodiments, the T cell and/or NK cells that are contacted by replication incompetent recombinant retroviral particles include between 90, 91, 92, 93, 94, and 95% resting cells on the low end of the range and 96, 97, 98, 99, or 100% resting cells on the high end of the range. In some embodiments, the T cells and/or NK cells include naïve cells. In some illustrative embodiments, the subembodiments in this paragraph are included in composition and method aspects for transducing lymphocytes in whole blood.

Contact between the T cells and/or NK cells and the replication incompetent recombinant retroviral particles can facilitate transduction of the T cells and/or NK cells by the replication incompetent recombinant retroviral particles. Not to be limited by theory, during the period of contact, the replication incompetent recombinant retroviral particles identify and bind to T cells and/or NK cells at which point the retroviral and host cell membranes start to fuse. Then, as a next step in the process of transduction, genetic material from the replication incompetent recombinant retroviral particles enters the T cells and/or NK cells at which time the T cells and/or NK cells are “genetically modified” as the phrase is used herein. It is noteworthy that such process might occur hours or even days after the contacting is initiated, and even after non-associated retroviral particles are rinsed away. Then the genetic material is typically integrated into the genomic DNA of the T cells and/or NK cells, at which time the T cells and/or NK cells are now “transduced” as the term is used herein. Accordingly, in illustrative embodiments, any method for genetically modifying lymphocytes (e.g. T cells and/or NK cells) herein, is a method for transducing lymphocytes (e.g. T cells and/or NK cells). It is believed that by day 6 in vivo or ex vivo, after contacting is initiated, the vast majority of genetically modified cells have been transduced. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. Throughout this disclosure, a transduced T cell and/or NK cell includes progeny of ex vivo transduced cells that retain at least some of the nucleic acids or polynucleotides that are incorporated into the genome of a cell during the ex vivo transduction. In methods herein that recite “reintroducing” a transduced cell, it will be understood that such cell is typically not in a transduced state when it is collected from the blood of a subject.

Many of the methods provided herein include genetic modification and transduction of T cells and/or NK cells. Methods are known in the art for genetically modifying and transducing T cells and/or NK cells ex vivo with replication incompetent recombinant retroviral particles, such as replication incompetent recombinant lentiviral particles. Methods provided herein, in illustrative embodiments, do not require ex vivo stimulation or activation. Thus, this common step in prior methods can be avoided in the present method, although ex vivo stimulatory molecule(s) such as anti-CD3 and/or anti-CD28 beads, can be present during the contacting and optional incubation thereafter. However, with illustrative methods provided herein, ex vivo stimulation is not required.

In certain illustrative embodiments for any aspects herein, the blood cells, such as lymphocytes, and especially T cells and/or NK cells are activated during the contacting or an optional incubation thereafter, and are not activated at all or for more than 15 minutes, 30 minutes, 1, 2, 4, or 8 hours before the contacting. In certain illustrative embodiments, activation by elements that are not present on the retroviral particle surface is not required for genetically modifying the lymphocytes. Accordingly, such activation or stimulation elements are not required other than on the retroviral particle, before, during, or after the contacting. Thus, as discussed in more detail herein, these illustrative embodiments that do not require pre-activation or stimulation provide the ability to rapidly perform in vitro experiments aimed at better understanding T cells and the biologicals mechanisms, therein. Furthermore, such methods provide for much more efficient commercial production of biological products produced using PBMCs, lymphocytes, T cells, or NK cells, and development of such commercial production methods. Finally, such methods provide for more rapid ex vivo processing of lymphocytes (e.g. NK cells and especially T cells) for adoptive cell therapy, fundamentally simplifying the delivery of such therapies, for example by providing point of care methods.

Although in illustrative embodiments, T cells and/or NK cells are not activated prior to being contacted with a recombinant retrovirus in methods herein, a T cell activation element in illustrative embodiments is present in the reaction mixture where initial contacting of a recombinant retrovirus and lymphocytes occurs. For example, such T cell activation element can be in solution in the reaction mixture. For example, soluble anti-CD3 antibodies can be present in the reaction mixture during the contacting and optional incubation thereafter, at 25-200, 50-150, 75-125, or 100 ng/ml. In illustrative embodiments, the T cell activation element is associated with the retroviral surface. The T cell activation element can be any T cell activation element provided herein. In illustrative embodiments, the T cell activation element can be anti-CD3, such as anti-CD3 scFv, or anti-CD3 scFvFc. Accordingly, in some embodiments, the replication incompetent recombinant retroviral particle can further include a T cell activation element, which in further illustrative examples is associated with the external side of the surface of the retrovirus.

The contacting step of a method for transducing and/or a method for genetically modifying lymphocytes in whole blood, provided herein, typically includes an initial step in which the retroviral particle, typically a population of retroviral particles, are brought into contact with blood cells, typically a population of blood cells that includes an anti-coagulant and/or additional blood components other than PBMCs, that are not present after a PBMC enrichment procedure, while in suspension in a liquid buffer and/or media to form a transduction reaction mixture. This contacting, as in other aspects provided herein, can be followed by an optional incubating period in this reaction mixture that includes the retroviral particles and the blood cells comprising lymphocytes (e.g. T cells and/or NK cells) in suspension. In methods for genetically modifying T cells and/or NK cells in blood or a component thereof, the reaction mixture can include at least one, two, three, four, five, or all additional blood components as disclosed herein, and in illustrative embodiments includes one or more anticoagulants.

The transduction reaction mixture in any of the aspects provided herein can be incubated at between 23 and 39° C., and in some illustrative embodiments at 37° C., in an optional incubation step after the initial contacting of retroviral particles and lymphocytes. In certain embodiments, the transduction reaction can be carried out at 37-39° C. for faster fusion/transduction. The cells and retroviral particles when brought into contact in the transduction reaction mixture can be immediately processed to remove the retroviral particles that remain free in suspension and not associated with cells, from the cells. Optionally, the cells in suspension and retroviral particles whether free in suspension or associated with the cells in suspension, can be incubated for various lengths of time, as provided herein for a contacting step in a method provided herein. Before further steps, a wash can be performed, regardless of whether such cells will be studied in vitro, ex vivo or introduced into a subject.

Illustrative methods are disclosed herein for genetically modifying lymphocytes, especially NK cells and in illustrative embodiments, T cells, that are much shorter and simpler than prior methods. Accordingly, in some embodiments, the contacting step in any method provided herein of transducing and/or genetically modifying a PBMC or a lymphocyte, typically a T cell and/or an NK cell, can be performed (or can occur) for any of the time periods provided in this specification, included, but not limited to those provided in the Exemplary Embodiments section. For example, said contacting can be for less than 24 hours, for example, less than 12 hours, less than 8 hours, less than 4 hours, less than 2 hours, less than 1 hour, less than 30 minutes or less than 15 minutes, but in each case there is at least an initial contacting step in which retroviral particles and cells come into contact in suspension in a transduction reaction mixture before retroviral particles that remain in suspension not associated with a cell, are separated from cells and typically discarded, as discussed in further detail herein. It should be noted, but not intending to be limited by theory, that it is believed that contacting begins at the time that retroviral particles and lymphocytes are combined together, typically by adding a solution containing the retroviral particles into a solution containing lymphocytes (e.g. T cells and/or NK cells).

After such initial contacting, in some embodiments there is an incubating of the reaction mixture containing cells and retroviral particles in suspension for a specified time period without removing retroviral particles that remain free in solution and not associated with cells. This incubating is sometimes referred to herein as an optional incubation. Thus, In illustrative embodiments, the contacting (including initial contacting and optional incubation) can be performed (or can occur) (where as indicated in general herein the low end of a selected range is less than the high end of the selected range) for between 30 seconds or 1, 2, 5, 10, 15, 30 or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. In certain illustrative embodiments, the contacting step can be performed for between 30 seconds, 1 minute, 5 minutes, 10 minutes, 15 minutes, or 30 minutes on the low end of the range and 1 hour, 2 hours, 4 hours, 6 hours, 8 hours, 10 hours, or 12 hours on the high end of the range. In some embodiments, the contacting step is performed for between 30 seconds, 1 minute, and 5 minutes on the low end of the range, and 10 minutes, 15 minutes, 30 minutes, 1 hour, 2 hours, 4 hours, or 8 hours on the high end of the range. Thus, in some embodiments, after the time when a reaction mixture is formed by adding retroviral particles to lymphocytes, the reaction mixture can be incubated for between 5 minutes and 12 hours, between 5 minutes and 10 hours, between 5 minutes and 8 hours, between 5 minutes and 6 hours, between 5 minutes and 4 hours, between 5 minutes and 2 hours, between 5 minutes and 1 hour, between 5 minutes and 30 minutes, or between 5 minutes and 15 minutes. In other embodiments, the reaction mixture can be incubated for between 15 minutes and 12 hours, between 15 minutes and 10 hours, between 15 minutes and 8 hours, between 15 minutes and 6 hours, between 15 minutes and 4 hours, between 15 minutes and 2 hours, between 15 minutes and 1 hour, between 15 minutes and 45 minutes, or between 15 minutes and 30 minutes. In other embodiments, the reaction mixture can be incubated for between 30 minutes and 12 hours, between 30 minutes and 10 hours, between 30 minutes and 8 hours, between 30 minutes and 6 hours, between 30 minutes and 4 hours, between 30 minutes and 2 hours, between 30 minutes and 1 hour, between 30 minutes and 45 minutes. In other embodiments, the reaction mixture can be incubated for between 1 hour and 12 hours, between 1 hour and 8 hours, between 1 hour and 4 hours, or between 1 hour and 2 hours. In another illustrative embodiment, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture.

After the indicated time period for the initial contacting and optional incubation that can be part of the contacting step, blood cells or a T cell and/or NK cell-containing fraction thereof in the reaction mixture, are separated from retroviral particles that are not associated with such cells. For example, this can be performed using a PBMC enrichment procedure (e.g. a Ficoll gradient in a Sepax unit), or in certain illustrative embodiments provided herein, by filtering the reaction mixture over a leukocyte depletion filter set assembly, and then collecting the leukocytes, which include T cells and NK cells. In another embodiment, this can be performed by centrifugation of the reaction mixture at a relative centrifugal force less than 500 g, for example 400 g, or between 300 and 490 g, or between 350 and 450 g. Such centrifugation to separate retroviral particles from cells can be performed for example, for between 5 minutes and 15 minutes, or between 5 minutes and 10 minutes. In illustrative embodiments where centrifugal force is used to separate cells from retroviral particles that are not associated with cells, such g force is typically lower than the g forces used successfully in spinoculation procedures.

In some illustrative embodiments, a method provided herein in any aspect, does not involve performing a spinoculation. In some embodiments, spinoculation is included as part of a contacting step. In illustrative embodiments, when spinoculation is performed there is no additional incubating as part of the contacting, as the time of the spinoculation provides the incubation time of the optional incubation discussed above. In other embodiments, there is an additional incubation after the spinoculating of between 15 minutes and 4 hours, or between 15 minutes and 2 hours, or between 15 minutes and 1 hour. The spinoculation can be performed for example, for 30 minutes to 120 minutes, typically for at least 60 minutes, for example for 60 minutes to 180 minutes, or 60 minutes to 90 minutes. The spinoculation is typically performed in a centrifuge with a relative centrifugal force of at least 800 g, and more typically at least 1,200 g, for example between 800 g and 2400 g, or between 800 g and 1800 g, or between 1200 g and 2400 g, or between 1200 g and 1800 g. After the spinoculation, such methods typically involve an additional step of resuspending the pelleted cells and retroviral particles, and then removing retroviral particles that are not associated with cells according to steps discussed above when spinoculation is not performed.

The contacting step including the optional incubation therein, and the spinoculation, in embodiments that include spinoculation, can be performed at between 4 C and 42 C, or between 20 C and 37 C. In certain illustrative embodiments, spinoculation is not performed and the contacting and associated optional incubation are carried out at 20-25 C for 4 hours or less, 2 hours or less, 1 hour or less, 30 minutes or less, 15 minutes or less, or 15 minutes to 2 hours, 15 minutes to 1 hour, or 15 minutes to 30 minutes.

In some embodiments of the methods and compositions disclosed herein, between 5% and 85% of the total lymphocytes collected from the blood are genetically modified. In some embodiments, the percent of lymphocytes that are genetically modified and/or transduced is between 1, 5, and 10% on the low end of the range, and 15, 20, 25, 30, 40, 50, 60, 70, 80, and 85% on the high end of the range. In some embodiments, the percent of T cells and NK cells that are genetically modified and/or transduced is at least 5%, at least 10%, at least 15%, or at least 20%. As illustrated in the Examples herein, in exemplary methods provided herein for transducing lymphocytes in whole blood, between 1% and 20%, or between 1% and 15%, or between 5% and 15%, or between 7% and 12% or about 10% of lymphocytes are genetically modified and/or transduced.

Methods of genetically modifying lymphocytes provided according to any method herein, typically include insertion into the cell, of a polynucleotide comprising one or more transcriptional units encoding a CAR or a lymphoproliferative element, or in illustrative embodiments encoding both a CAR and a lymphoproliferative element according to any of the CAR and lymphoproliferative element embodiments provided herein. Such CAR and lymphoproliferative elements can be provided to support the shorter and more simplified methods provided herein, which can support expansion of genetically modified and/or transduced T cells and/or NK cells after the contacting and optional incubation. Accordingly, in exemplary embodiments of any methods provided herein, lymphoproliferative elements can be delivered from the genome of the retroviral particles inside genetically modified and/or transduced T cells and/or NK cells, such that those cells have the characteristics of increased proliferation and/or survival disclosed in the Lymphoproliferative Elements section herein. In exemplary embodiments of any methods provided herein, the genetically modified T cell or NK cell is capable of engraftment in vivo in mice and/or enrichment in vivo in mice for at least 7, 14, or 28 days. A skilled artisan will recognize that such mice may be treated or otherwise genetically modified so that any immunological differences between the genetically modified T cell and/or NK cell do not result in an immune response being elicited in the mice against any component of the lymphocyte transduced by the replication incompetent recombinant retroviral particle.

Media that can be included in a contacting step, for example when the cells and retroviral particles are initially brought into contact, or in any aspects provided herein, during optional incubation periods with the reaction mixture thereafter that include retroviral particles and cells in suspension in the media, or media that can be used during cell culturing and/or during various wash steps in any aspects provided herein, can include base media such as commercially available media for ex vivo T cell and/or NK cell culture. Non-limiting examples of such media include, X-VIVO™ 15 Chemically Defined, Serum-free Hematopoietic Cell Medium (Lonza) (2018 catalog numbers BE02-060F, BE02-00Q, BE-02-061Q, 04-744Q, or 04-418Q), ImmunoCult™-XF T Cell Expansion Medium (STEMCELL Technologies) (2018 catalog number 10981), PRIME-XV® T Cell Expansion XSFM (Irvine Scientific) (2018 catalog number 91141), AIM V® Medium CTS™ (Therapeutic Grade) (Thermo Fisher Scientific (Referred to herein as “Thermo Fisher”), or CTS™ Optimizer™ media (Thermo Fisher) (2018 catalog numbers A10221-01 (basal media (bottle)), and A10484-02 (supplement), A10221-03 (basal media (bag)), A1048501 (basal media and supplement kit (bottle)) and, A1048503 (basal media and supplement kit (bag)). Such media can be a chemically defined, serum-free formulation manufactured in compliance with cGMP. The media can be xeno-free and complete. In some embodiments, the base media has been cleared by regulatory agencies for use in ex vivo cell processing, such as an FDA 510(k) cleared device. In some embodiments, the media is the basal media with or without the supplied T cell expansion supplement of 2018 catalog number A1048501 (CTS™ OpTmizer™ T Cell Expansion SFM, bottle format) or A1048503 (CTS™ OpTmizer™ T Cell Expansion SFM, bag format) both available from Thermo Fisher (Waltham, Mass.). Additives such as human serum albumin, human AB+ serum, and/or serum derived from the subject can be added to the transduction reaction mixture. Supportive cytokines can be added to the transduction reaction mixture, such as IL2, IL7, or IL15, or those found in human sera. dGTP can be added to the transduction reaction in certain embodiments.

In some embodiments of any method herein that includes a step of genetically modifying lymphocytes (e.g. T cells and/or NK cells), the cells can be contacted with a retroviral particle without prior activation. In some embodiments of any method herein that includes a step of genetically modifying T cells and/or NK cells, the T cells and/or NK cells have not been incubated on a substrate that adheres to monocytes for more than 4 hours in one embodiment, or for more than 6, hours in another embodiment, or for more than 8 hours in another embodiment before the transduction. In one illustrative embodiment, the T cells and/or NK cells have been incubated overnight on an adherent substrate to remove monocytes before the transduction. In another embodiment, the method can include incubating the T cells and/or NK cells on an adherent substrate that binds monocytes for no more than 30 minutes, 1 hour, or 2 hours before the transduction. In another embodiment, the T cells and/or NK cells are exposed to no step of removing monocytes by an incubation on an adherent substrate before said transduction step. In another embodiment, the T cells and/or NK cells are not incubated with or exposed to a bovine serum, such as a cell culturing bovine serum, for example fetal bovine serum before or during a contacting step and/or a genetically modifying and/or transduction step.

Some or all of the steps of the methods for genetically modifying provided herein, or uses of such methods, are performed in a closed system. Thus, reaction mixtures formed in such methods, and genetically modified and/or transduced lymphocytes (e.g. T cells and/or NK cells) made by such methods, can be contained within such a closed system. A closed system is a cell processing system that is generally closed or fully closed to an environment, such as an environment within a room or even the environment within a hood, outside of the conduits such as tubes, and chambers, of the system in which cells are processed and/or transported. One of the greatest risks to safety and regulatory control in the cell processing procedure is the risk of contamination through frequent exposure to the environment as is found in traditional open cell culture systems. To mitigate this risk, particularly in the absence of antibiotics, some commercial processes have been developed that focus on the use of disposable (single-use) equipment. However, even with their use under aseptic conditions, there is always a risk of contamination from the opening of flasks to sample or add additional growth media. To overcome this problem, methods provided herein, which are typically ex vivo methods, are typically performed within a closed-system. Such a process is designed and can be operated such that the product is not exposed to the outside environment. Material transfer occurs via sterile connections, such as sterile tubing and sterile welded connections. Air for gas exchange can occur via a gas permeable membrane, via 0.2 μm filter to prevent environmental exposure. In some illustrative embodiments, the methods are performed on T cells, for example to provide genetically modified T cells.

Such closed system methods can be performed with commercially available devices. Different closed system devices can be used at different steps within a method and the cells can be transferred between these devices using tubing and connections such as welded, luer, spike, or clave ports to prevent exposure of the cells or media to the environment. For example, blood can be collected into an IV bag or syringe, optionally including an anti-coagulant, and transferred to a Sepax 2 device (Biosafe) for PBMC enrichment and isolation. In other embodiments, whole blood can be filtered to collect leukocytes using a leukodepletion filter assembly. The isolated PBMCs or isolated leukocytes can be transferred to a chamber of a G-Rex device for an optional activation, a transduction and optional expansion. Alternatively, collected blood can be transduced in a blood bag, for example, the bag in which it was collected. Finally, the cells can be harvested and collected into another bag using a Sepax 2 device. The methods can be carried out in any device or combination of devices adapted for closed system T cell and/or NK cell production. Non-limiting examples of such devices include G-Rex devices (Wilson Wolf), GatheRex (Wilson Wolf), Sepax 2 (Biosafe), WAVE Bioreactors (General Electric), a CultiLife Cell Culture bag (Takara), a PermaLife bag (OriGen), CliniMACS Prodigy (Miltenyi Biotec), and VueLife bags (Saint-Gobain). In illustrative embodiments, the optional activating, the transducing and optional expanding can be performed in the same chamber or vessel in the closed system. For example, in illustrative embodiments, the chamber can be a chamber of a G-Rex device and PBMCs or leukocytes can be transferred to the chamber of the G-Rex device after they are enriched and isolated, and can remain in the same chamber of the G-Rex device until harvesting.

Methods provided herein can include transferring blood and cells therein and/or fractions thereof, as well as lymphocytes before or after they are contacted with retroviral particles, between vessels within a closed system, which thus is without environmental exposure. Vessels used in the closed system, for example, can be a tube, bag, syringe, or other container. In some embodiments, the vessel is a vessel that is used in a research facility. In some embodiments, the vessel is a vessel used in commercial production. In other embodiments, the vessel can be a collection vessel used in a blood collection process. Methods for genetically modifying herein, typically involve a contacting step wherein lymphocytes are contacted with a replication incompetent recombinant retroviral particle. The contacting in some embodiments, can be performed in the vessel, for example, within a blood bag. Blood and various lymphocyte-containing fractions thereof, can be transferred from the vessel to another vessel (for example from a first vessel to a second vessel) within the closed system for the contacting. The second vessel can be a cell processing compartment of a closed device, such as a G-Rex device. In some embodiments, after the contacting the genetically modified (e.g. transduced) cells can be transferred to a different vessel within the closed system (i.e. without exposure to the environment). Either before or after this transfer the cells are typically washed within the closed system to remove substantially all or all of the retroviral particles. In some embodiments, a process disclosed herein, from collection of blood, to contacting (e.g. transduction), optional incubating, and post-incubation isolation and optional washing, is performed for between 15 minutes, 30 minutes, or 1, 2, 3, or 4 hours on the low end of the range, and 4, 8, 10, or 12 hours on the high end of the range.

Not to be limited by theory, in non-limiting illustrative methods, the delivery of a polynucleotide encoding a lymphoproliferative element, to a resting T cell and/or NK cell ex vivo, which can integrate into the genome of the T cell or NK cell, provides that cell with a driver for in vivo expansion without the need for lymphodepleting the host. Thus, in illustrative embodiments, the subject is not exposed to a lymphodepleting agent within 1, 2, 3, 4, 5, 6, 7, 10, 14, 21, or 28 days, or within 1 month, 2 months, 3 months or 6 months of performing the contacting, during the contacting, and/or within 1, 2, 3, 4, 5, 6, 7, 10, 14, 21, or 28 days, or within 1 month, 2 months, 3 months or 6 months after the modified T cells and/or NK cells are reintroduced back into the subject. Furthermore, in non-limiting illustrative embodiments, methods provided herein can be performed without exposing the subject to a lymphodepleting agent during a step wherein a replication incompetent recombinant retroviral particle is in contact with resting T cells and/or resting NK cells of the subject and/or during the entire ex vivo method. Hence, methods of expanding genetically modified T cells and/or NK cells in a subject in vivo is a feature of some embodiments of the present disclosure. In illustrative embodiments, such methods are ex vivo propagation-free or substantially propagation-free.

This entire method/process from blood draw from a subject to reintroduction of blood back into the subject after ex vivo transduction of T cells and/or NK cells, in non-limiting illustrative embodiments of any aspects provided herein, can occur over a time period less than 48 hours, less than 36 hours, less than 24 hours, less than 12 hours, less than 11 hours, less than 10 hours, less than 9 hours, less than 8 hours, less than 7 hours, less than 6 hours, less than 5 hours, less than 4 hours, less than 3 hours, 2 hours, or less than 2 hours. In other embodiments, the entire method/process from blood draw/collection from a subject to reintroduction of blood back into the subject after ex vivo transduction of T cells and/or NK cells, in non-limiting illustrative embodiments herein, occurs over a time period between 1 hour and 12 hours, or between 2 hours and 8 hours, or between 1 hour and 3 hours, or between 2 hours and 4 hours, or between 2 hours and 6 hours, or between 4 hours and 12 hours, or between 4 hours and 24 hours, or between 8 hours and 24 hours, or between 8 hours and 36 hours, or between 8 hours and 48 hours, or between 12 hours and 24 hours, or between 12 hours and 36 hours, or between 12 hours and 48 hours, or over a time period between 15, 30, 60, 90, 120, 180, and 240 minutes on the low end of the range, and 120, 180, and 240, 300, 360, 420, and 480 minutes on the high end of the range. In other embodiments, the entire method/process from blood draw/collection from a subject to reintroduction of blood back into the subject after ex vivo transduction of T cells and/or NK cells, occurs over a time period between 1, 2, 3, 4, 6, 8, 10, and 12 hours on the low end of the range, and 8, 9, 10, 11, 12, 18, 24, 36, or 48 hours on the high end of the range. In some embodiments, the genetically modified T cells and/or NK cells are separated from the replication incompetent recombinant retroviral particles after the time period in which contact occurs.

Because methods provided herein for genetically modifying lymphocytes, and associated methods for performing adoptive cell therapy can be performed in significantly less time than prior methods, fundamental improvements in patient care and safety as well as product manufacturability are made possible. Therefore, such processes are expected to be favorable in the view of regulatory agencies responsible for approving such processes when carried out in vivo for therapeutic purposes. For example, the subject in non-limiting examples of any aspects provided herein that include a subject, can remain in the same building (e.g. infusion clinic) or room as the instrument processing their blood or sample for the entire time that the sample is being processed before modified T cells and/or NK cells are reintroduced into the patient. In non-limiting illustrative embodiments, a subject remains within line of site and/or within 100, 50, 25, or 12 feet or arm's distance of their blood or cells that are being processed, for the entire method/process from blood draw/collection from the subject to reintroduction of blood to the subject after ex vivo transduction of T cells and/or NK cells. In other non-limiting illustrative embodiments, a subject remains awake and/or at least one person can continue to monitor the blood or cells of the subject that are being processed, throughout and/or continuously for the entire method/process from blood draw/collection from the subject to reintroduction of blood to the subject after ex vivo transduction of T cells and/or NK cells. Because of improvements provided herein, the entire method/process for adoptive cell therapy and/or for transducing resting T cells and/or NK cells from blood draw/collection from the subject to reintroduction of blood to the subject after ex vivo transduction of T cells and/or NK cells can be performed with continuous monitoring by a human. In other non-limiting illustrative embodiments, at no point the entire method/process from blood draw/collection from the subject to reintroduction of blood to the subject after ex vivo transduction of T cells and/or NK cells, are blood cells incubated in a room that does not have a person present. In other non-limiting illustrative embodiments, the entire method/process from blood draw/collection from the subject to reintroduction of blood to the subject after ex vivo transduction of T cells and/or NK cells, is performed next to the subject and/or in the same room as the subject and/or next to the bed or chair of the subject. Thus, sample identity mix-ups can be avoided, as well as long and expensive incubations over periods of days or weeks. This is further provided by the fact that methods provided herein are readily adaptable to closed and automated blood processing systems, where a blood sample and its components that will be reintroduced into the subject, only make contact with disposable, single-use components.

Methods for genetically modifying and/or transducing lymphocytes such as T cells and/or NK cells provided herein, can be part of a method for performing adoptive cell therapy. Typically, methods for performing adoptive cell therapy include steps of collecting blood from a subject, and returning genetically modified and/or transduced lymphocytes (e.g T cells and/or NK cells) to the subject. The present disclosure provides various treatment methods using a CAR. A CAR of the present disclosure, when present in a T lymphocyte or an NK cell, can mediate cytotoxicity toward a target cell. A CAR of the present disclosure binds to an antigen present on a target cell, thereby mediating killing of a target cell by a T lymphocyte or an NK cell genetically modified to produce the CAR. The ASTR of the CAR binds to an antigen present on the surface of a target cell. The present disclosure provides methods of killing, or inhibiting the growth of, a target cell, the method involving contacting a cytotoxic immune effector cell (e.g., a cytotoxic T cell, or an NK cell) that is genetically modified to produce a subject CAR, such that the T lymphocyte or NK cell recognizes an antigen present on the surface of a target cell, and mediates killing of the target cell. The target cell can be a cancer cell, for example, and autologous cell therapy methods herein, can be methods for treating cancer, in some illustrative embodiments. In these embodiments, the subject can be a an animal or human suspected of having cancer, or more typically, a subject that is known to have cancer.

In some embodiments of any of the methods provided herein for genetically modifying lymphocytes (e.g. T cells and/or NK cells), and aspects directed to use of replication incompetent recombinant retroviral particles in the manufacture of a kit for genetically modifying T cells and/or NK cells of a subject, the genetically modified and/or transduced lymphocyte (e.g. T cell and/or NK cell) or population thereof, are introduced or reintroduced into the subject. Introduction or reintroduction of the genetically modified lymphocytes into a subject can be via any route known in the art. For example, introduction or reintroduction can be delivery via infusion into a blood vessel of the subject. In some embodiments, the genetically modified and/or transduced lymphocyte (e.g. T cell and/or NK cell) or population thereof, undergo 4 or fewer cell divisions ex vivo prior to being introduced or reintroduced into the subject. In some embodiments, the lymphocyte(s) used in such a method are resting T cells and/or resting NK cells that are in contact with the replication incompetent recombinant retroviral particles for between 1 hour and 12 hours. In some embodiments, no more than 12 hours, 10 hours, 8 hours, 6 hours, 4 hours, 2 hours, or 1 hour pass(es) between the time blood is collected from the subject and the time the genetically modified T cells and/or NK cells are reintroduced into the subject. In some embodiments, all steps after the blood is collected and before the blood is reintroduced, are performed in a closed system in which a person monitors the closed system throughout the processing.

In some embodiments of the methods and compositions disclosed herein, the genetically modified T cells and/or NK cells are introduced back, reintroduced, reinfused or otherwise delivered into the subject without additional ex vivo manipulation, such as stimulation and/or activation of T cells and/or NKs. In the prior art methods, ex vivo manipulation is used for stimulation/activation of T cells and/or NK cells and for expansion of genetically modified T cells and/or NK cells prior to introducing the genetically modified T cells and/or NK cells into the subject. In prior art methods, this generally takes days or weeks and requires a subject to return to a clinic for a blood infusion days or weeks after an initial blood draw. In some embodiments of the methods and compositions disclosed herein, T cells and/or NK cells are not stimulated ex vivo by exposure to anti-CD3/anti-CD28 solid supports such as, for example, beads coated with anti-CD3/anti-CD28, prior to contacting the T cells and/or NK cells with the replication incompetent recombinant retroviral particles. As such provided herein is an ex vivo propagation-free method. In other embodiments, genetically modified T cells and/or NK cells are not expanded ex vivo, or only expanded for a small number of cell divisions (e.g. 1, 2, 3, 4, 5, 6, 7, 8, 9, or 10 rounds of cell division), but are rather expanded, or predominantly expanded, in vivo, i.e. within the subject. In some embodiments, no additional media is added to allow for further expansion of the cells. In some embodiments, no cell manufacturing of the primary blood lymphocytes (PBLs) occurs while the PBLs are contacted with the replication incompetent recombinant retroviral particles. In illustrative embodiments, no cell manufacturing of the PBLs occurs while the PBLs are ex vivo. In traditional methods of adoptive cell therapy, subjects are lymphodepleted prior to reinfusion with genetically modified T cells and or NK cells. In some embodiments, patients or subjects are not lymphodepleted prior to blood being withdrawn. In some embodiments, patients or subjects are not lymphodepleted prior to reinfusion with genetically modified T cells and or NK cells. However, the embodiments of the methods and compositions disclosed herein can be used on pre-activated or pre-stimulated T cells and/or NK cells as well. In some embodiments, T cells and/or NK cells can be stimulated ex vivo by exposure to anti-CD3/anti-CD28 solid supports prior to contacting the T cells and/or NK cells with the replication incompetent recombinant retroviral particles. In some embodiments, the T cells and/or NK cells can be exposed to anti-CD3/anti-CD28 solid supports for less than 1, 2, 3, 4, 6, 8, 10, 12, 14, 16, 18, or 24 hours, including no exposure, before the T cells and/or NK cells are contacted the replication incompetent recombinant retroviral particles. In illustrative embodiments, the T cells and/or NK cells can be exposed to anti-CD3/anti-CD28 solid supports for less than 1, 2, 3, 4, 6, or 8 hours before the T cells and/or NK cells are contacted the replication incompetent recombinant retroviral particles.

In some illustrative embodiments, cells are introduced or reintroduced into the subject by infusion into a vein or artery. In any of the embodiments disclosed herein, the number of T cells and/or NK cells to be reinfused into a subject can be between 1×10³, 2.5×10³, 5×10³, 1×10⁴, 2.5×10⁴, 5×10⁴, 1×10⁵, 2.5×10⁵, 5×10⁵, 1×10⁶, 2.5×10⁶, 5×10⁶, and 1×10⁷ cells/kg on the low end of the range and 5×10⁴, 1×10⁵, 2.5×10⁵, 5×10⁵, 1×10⁶, 2.5×10⁶, 5×10⁶, 1×10⁷, 2.5×10⁷, 5×10⁷, and 1×10⁸ cells/kg on the high end of the range. In illustrative embodiments, the number of T cells and/or NK cells to be reinfused or otherwise delivered into a subject can be between 1×10⁴, 2.5×10⁴, 5×10⁴, and 1×10⁶ cells/kg on the low end of the range and 2.5×10⁴, 5×10⁴, 1×10⁵, 2.5×10⁴, 5×10⁵, and 1×10⁶ cells/kg on the high end of the range. In some embodiments, the number of PBLs to be reinfused or otherwise delivered into a subject can be fewer than 5×10⁵, 1×10⁶, 2.5×10⁶, 5×10⁶, 1×10⁷, 2.5×10⁷, 5×10⁷, and 1×10⁸ cells and the low end of the range and 2.5×10⁶, 5×10⁶, 1×10⁷, 2.5×10⁷, 5×10⁷, 1×10⁸, 2.5×10⁸, 5×10⁸, and 1×10⁹ cells on the high end of the range. In some embodiments, the number of T cells and/or NK cells available for infusion or reinfusion into a 70 kg subject or patient is between 7×10⁵ and 2.5×10⁸ cells. In other embodiments, the number of T cells and/or NK cells available for transduction is approximately 7×10⁶ plus or minus 10%.

### Engineered Signaling Polypeptide(s)

In some embodiments, the replication incompetent recombinant retroviral particles used to contact T cells and/or NK cells have a polynucleotide or nucleic acid having one or more transcriptional units that encode one or more engineered signaling polypeptides. In some embodiments, an engineered signaling polypeptide includes any combination of an extracellular domain (e.g. an antigen-specific targeting region or ASTR), a stalk and a transmembrane domain, combined with one or more intracellular activating domains, optionally one or more modulatory domains (such as a co-stimulatory domain), and optionally one or more T cell survival motifs. In illustrative embodiments, at least one, two, or all of the engineered signaling polypeptides is a chimeric antigen receptor (CAR) or a lymphoproliferative element (LE) such as a chimeric lymphoproliferative element (CLE). In some embodiments, at least one, two, or all of the engineered signaling polypeptides is a recombinant T cell receptor (TCR). In some embodiments, when two signaling polypeptides are utilized, one encodes a lymphoproliferative element and the other encodes a chimeric antigen receptor (CAR) that includes an antigen-specific targeting region (ASTR), a transmembrane domain, and an intracellular activating domain. For any domain of an engineered signaling polypeptide disclosed herein, exemplary sequences can be found in WO2019/055946, incorporated herein in its entirety by reference. A skilled artisan will recognize that such engineered polypeptides can also be referred to as recombinant polypeptides. The engineered signaling polypeptides, such as CARs, recombinant TCRs, LEs, and CLEs provided herein, are typically transgenes with respect to lymphocytes, especially T cells and NK cells, and most especially T cells and/or NK cells that are engineered using methods and compositions provided herein, to express such signaling polypeptides.

### Extracellular Domain

In some embodiments, an engineered signaling polypeptide includes an extracellular domain that is a member of a specific binding pair. For example, in some embodiments, the extracellular domain can be the extracellular domain of a cytokine receptor, or a mutant thereof, or a hormone receptor, or a mutant thereof. Such mutant extracellular domains in some embodiments have been reported to be constitutively active when expressed at least in some cell types. In illustrative embodiments, such extracellular and transmembrane domains do not include a ligand binding region. It is believed that such domains do not bind a ligand when present in an engineered signaling polypeptide and expressed in B cells, T cells, and/or NK cells. Mutations in such receptor mutants can occur in the extracellular juxtamembrane region. Not to be limited by theory, a mutation in at least some extracellular domains (and some extracellular-transmembrane domains) of engineered signaling polypeptides provided herein, are responsible for signaling of the engineered signaling polypeptide in the absence of ligand, by bringing activating chains together that are not normally together. Further embodiments regarding extracellular domains that comprise mutations in extracellular domains can be found, for example, in the Lymphoproliferative Element section herein.

In certain illustrative embodiments, the extracellular domain comprises a dimerizing motif. In an illustrative embodiment the dimerizing motif comprises a leucine zipper. In some embodiments, the leucine zipper is from a jun polypeptide, for example c-jun. Further embodiments regarding extracellular domains that comprise a dimerizing motif can be found, for example, in the Lymphoproliferative Element section herein.

In certain embodiments, the extracellular domain is an antigen-specific targeting region (ASTR), sometimes called an antigen binding domain herein. Specific binding pairs include, but are not limited to, antigen-antibody binding pairs; ligand-receptor binding pairs; and the like. Thus, a member of a specific binding pair suitable for use in an engineered signaling polypeptide of the present disclosure includes an ASTR that is an antibody, an antigen, a ligand, a receptor binding domain of a ligand, a receptor, a ligand binding domain of a receptor, and an affibody.

An ASTR suitable for use in an engineered signaling polypeptide of the present disclosure can be any antigen-binding polypeptide. In certain embodiments, the ASTR is an antibody such as a full-length antibody, a single-chain antibody, an Fab fragment, an Fab′ fragment, an (Fab′)2 fragment, an Fv fragment, and a divalent single-chain antibody or a diabody.

In some embodiments, the ASTR is a single chain Fv (scFv). In some embodiments, the heavy chain is positioned N-terminal of the light chain in the engineered signaling polypeptide. In other embodiments, the light chain is positioned N-terminal of the heavy chain in the engineered signaling polypeptide. In any of the disclosed embodiments, the heavy and light chains can be separated by a linker as discussed in more detail herein. In any of the disclosed embodiments, the heavy or light chain can be at the N-terminus of the engineered signaling polypeptide and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the engineered signaling polypeptides and methods using the engineered signaling polypeptides of the present disclosure. In some instances, T cell receptor (TCR) based recognition domains.

Certain embodiments for any aspect or embodiment herein that includes a CAR, include CARs having extracellular domains engineered to co-opt the endogenous TCR signaling complex and CD3Z signaling pathway. In one embodiment, a chimeric antigen receptor ASTR is fused to one of the endogenous TCR complex chains (e.g. TCR alpha, CD3E etc) to promote incorporation into the TCR complex and signaling through the endogenous CD3Z chains. In other embodiments, a CAR contains a first scFv or protein that binds to the TCR complex and a second scFv or protein that binds to the target antigen (e.g. tumor antigen). In another embodiment, the TCR can be a single chain TCR (scTv, single chain two-domain TCR containing VαVβ). Finally, scFv's may also be generated to recognize the specific MHC/peptide complex, thereby acting as a surrogate TCR. Such peptide/MHC scFv-binders may be used in many similar configurations as CAR's.

In some embodiments, the ASTR can be multispecific, e.g. bispecific antibodies. Multispecific antibodies have binding specificities for at least two different sites. In certain embodiments, one of the binding specificities is for one target antigen and the other is for another target antigen. In certain embodiments, bispecific antibodies may bind to two different epitopes of a target antigen. Bispecific antibodies may also be used to localize cytotoxic agents to cells which express a target antigen. Bispecific antibodies can be prepared as full-length antibodies or antibody fragments.

An ASTR suitable for use in an engineered signaling polypeptide of the present disclosure can have a variety of antigen-binding specificities. In some cases, the antigen-binding domain is specific for an epitope present in an antigen that is expressed by (synthesized by) a target cell. In one example, the target cell is a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which an ASTR of an engineered signaling polypeptide can bind include, e.g., CD19, CD20, CD38, CD30, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-Al, IL-13R-a2, GD2, Axl, Ror2, and the like.

In some embodiments, a member of a specific binding pair suitable for use in an engineered signaling polypeptide is an ASTR that is a ligand for a receptor. Ligands include, but are not limited to, hormones (e.g. erythropoietin, growth hormone, leptin, etc.); cytokines (e.g., interferons, interleukins, certain hormones, etc.); growth factors (e.g., heregulin; vascular endothelial growth factor (VEGF); and the like); an integrin-binding peptide (e.g., a peptide comprising the sequence Arg-Gly-Asp (SEQ ID NO:1); and the like.

Where the member of a specific binding pair in an engineered signaling polypeptide is a ligand, the engineered signaling polypeptide can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some cases, the member of a specific binding pair that is included in an engineered signaling polypeptide is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

In certain embodiments of any of the aspects provided herein that include an ASTR, the ASTR can be directed to an intermediate protein that links the ASTR with a target molecule expressed on a target cell. The intermediate protein may be endogenously expressed or introduced exogenously and may be natural, engineered, or chemically modified. In certain embodiments the ASTR can be an anti-tag ASTR such that at least one tagged intermediate, typically an antibody-tag conjugate, is included between a tag recognized by the ASTR and a target molecule, typically a protein target, expressed on a target cell. Accordingly, in such embodiments, the ASTR binds a tag and the tag is conjugated to an antibody directed against an antigen on a target cell, such as a cancer cell. Non-limiting examples of tags include fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkaline phosphatase, glucose oxidase, and maltose binding protein. As such, the ASTR comprises a molecule that binds the tag.

### Stalk

In some embodiments, the engineered signaling polypeptide includes a stalk which is located in the portion of the engineered signaling polypeptide lying outside the cell and interposed between the ASTR and the transmembrane domain. In some embodiments, the stalk has at least 85, 90, 95, 96, 97, 98, 99, or 100% identity to a wild-type CD8 stalk region (TTTPAPRPPTPAPTIASQPLSLRPEACRPAAGG AVHTRGLDFA (SEQ ID NO:2), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO:3), or has at least 85, 90, 95, 96, 97, 98, 99, or 100% identity to a wild-type immunoglobulin heavy chain stalk region. In an engineered signaling polypeptide, the stalk employed allows the antigen-specific targeting region, and typically the entire engineered signaling polypeptide, to retain increased binding to a target antigen.

The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some embodiments, the stalk of an engineered signaling polypeptide includes at least one cysteine. For example, In some embodiments, the stalk can include the sequence Cys-Pro-Pro-Cys (SEQ ID NO:4). If present, a cysteine in the stalk of a first engineered signaling polypeptide can be available to form a disulfide bond with a stalk in a second engineered signaling polypeptide.

Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:5); CPPC (SEQ ID NO:4); CPEPKSCDTPPPCPR (SEQ ID NO:6) (see, e.g., Glaser et al. (2005) J. Biol. Chem. 280:41494); ELKTPLGDTTHT (SEQ ID NO:7); KSCDKTHTCP (SEQ ID NO:8); KCCVDCP (SEQ ID NO:9); KYGPPCP (SEQ ID NO:10); EPKSCDKTHTCPPCP (SEQ ID NO:11) (human IgG1 hinge); ERKCCVECPPCP (SEQ ID NO:12) (human IgG2 hinge); ELKTPLGDTTHTCPRCP (SEQ ID NO:13) (human IgG3 hinge); SPNMVPHAHHAQ (SEQ ID NO:14) (human IgG4 hinge); and the like. The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (SEQ ID NO:15), (see, e.g., Yan et al. (2012) J. Biol. Chem. 287:5891). The stalk can include an amino acid sequence derived from human CD8; e.g., the stalk can include the amino acid sequence: TTTPAPRPPTPAPTIASQPLSLRPEACRPAAGGAVHTRGLDFACD (SEQ ID NO:16), or a variant thereof.

### Transmembrane Domain

An engineered signaling polypeptide of the present disclosure can include transmembrane domains for insertion into a eukaryotic cell membrane. The transmembrane domain can be interposed between the ASTR and the co-stimulatory domain. The transmembrane domain can be interposed between the stalk and the co-stimulatory domain, such that the chimeric antigen receptor includes, in order from the amino terminus (N-terminus) to the carboxyl terminus (C-terminus): an ASTR; a stalk; a transmembrane domain; and an activating domain.

Any transmembrane (TM) domain that provides for insertion of a polypeptide into the cell membrane of a eukaryotic (e.g., mammalian) cell is suitable for use in aspects and embodiments disclosed herein.

Non-limiting examples of TM domains suitable for any of the aspects or embodiments provided herein, include a domain with at least 50, 60, 70, 75, 80, 85, 90, 95, 96, 97, 98, 99 or 100% sequence identity to a stretch of at least 10, 15, 20, or all of the amino acids of any of the following TM domains or combined stalk and TM domains: a) CD8 alpha TM (SEQ ID NO:17); b) CD8 beta TM (SEQ ID NO:18); c) CD4 stalk (SEQ ID NO:19); d) CD3Z TM (SEQ ID NO:20); e) CD28 TM (SEQ ID NO:21); f) CD134 (OX40) TM: (SEQ ID NO:22); g) CD7 TM (SEQ ID NO:23); h) CD8 stalk and TM (SEQ ID NO:24); and i) CD28 stalk and TM (SEQ ID NO:25).

As non-limiting examples, a transmembrane domain of an aspect of the invention can have at least 80%, 90%, or 95% or can have 100% sequence identity to the SEQ ID NO:17 transmembrane domain, or can have 100% sequence identity to any of the transmembrane domains from the following genes respectively: the CD8 beta transmembrane domain, the CD4 transmembrane domain, the CD3 zeta transmembrane domain, the CD28 transmembrane domain, the CD134 transmembrane domain, or the CD7 transmembrane domain.

### Intracellular Activating Domain

Intracellular activating domains suitable for use in an engineered signaling polypeptide of the present disclosure when activated, typically induce the production of one or more cytokines; increase cell death; and/or increase proliferation of CD8⁺ T cells, CD4⁺ T cells, NKT cells, γδ T cells, and/or neutrophils. Activating domains can also be referred to as activation domains herein. Activating domains can be used in CARs or in lymphoproliferative elements provided herein.

In some embodiments, the intracellular activating domain includes at least one (e.g., one, two, three, four, five, six, etc.) ITAM motifs as described below. In some embodiments, an intracellular activating domain of an aspect of the invention can have at least 80%, 90%, or 95% or can have 100% sequence identity to the CD3Z, CD3D, CD3E, CD3G, CD79A, CD79B, DAP12, FCERIG, FCGR2A, FCGR2C, DAP10/CD28, or ZAP70 domains as described below.

Intracellular activating domains suitable for use in an engineered signaling polypeptide of the present disclosure include immunoreceptor tyrosine-based activation motif (ITAM)-containing intracellular signaling polypeptides. An ITAM motif is YX₁X₂L/I, where X₁ and X₂ are independently any amino acid. In some embodiments, the intracellular activating domain of an engineered signaling polypeptide includes 1, 2, 3, 4, or 5 ITAM motifs. In some embodiments, an ITAM motif is repeated twice in an intracellular activating domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some embodiments, the intracellular activating domain of an engineered signaling polypeptide includes 3 ITAM motifs.

A suitable intracellular activating domain can be an ITAM motif-containing portion that is derived from a polypeptide that contains an ITAM motif. For example, a suitable intracellular activating domain can be an ITAM motif-containing domain from any ITAM motif-containing protein. Thus, a suitable intracellular activating domain need not contain the entire sequence of the entire protein from which it is derived. Examples of suitable ITAM motif-containing polypeptides include, but are not limited to: CD3Z (CD3 zeta); CD3D (CD3 delta); CD3E (CD3 epsilon); CD3G (CD3 gamma); CD79A (antigen receptor complex-associated protein alpha chain); CD79B (antigen receptor complex-associated protein beta chain)DAP12; and FCERIG (Fc epsilon receptor I gamma chain).

In some embodiments, the intracellular activating domain is derived from T cell surface glycoprotein CD3 zeta chain (also known as CD3Z, T cell receptor T3 zeta chain, CD247, CD3-ZETA, CD3H, CD3Q, T3Z, TCRZ, etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms): MKWKALFTAAILQAQLPITEAQSFGLLDPKLCYLLDGILFIYGVILTALFLRVKFSRSADAPAYQQ GQNQLI[YNELNLGRREEYDVL]DKRRGRDPEMGGKPRRKNPQEGLI[YNELQKDKMAEAYSEI]G MKGERRRGKGHDGL[YQGLSTATKDTYDAL]HMQALPPR (SEQ ID NO:26) or MKWKALFTAAILQAQLPITEAQSFGLLDPKLCYLLDGILFIYGVILTALFLRVKFSRSADAPAYQQ GQNQLI[YNELNLGRREEYDVL]DKRRGRDPEMGGKPQRRKNPQEGL[YNELQKDKMAEAYSEI]GMKGERRRGKGHDGL[YQGLSTATKDTYDAL]HMQALPPR (SEQ ID NO:27), where the ITAM motifs are set out with brackets.

Likewise, a suitable intracellular activating domain polypeptide can include an ITAM motif-containing a portion of the full length CD3 zeta amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences: RVKFSRSADAPAYQQGQNQLI[YNELNLGRREEYDVL]DKRRGRDPEMGGKPRRKNPQEGL[YNE LQKDKMAEAYSEI]GMKGERRRGKGHDGL[YQGLSTATKDTYDAL]HMQALPPR (SEQ ID NO:28); RVKFSRSADAPAYQQGQNQLI[YNELNLGRREEYDVL]DKRRGRDPEMGGKPQRRKNPQEGL[YN ELQKDKMAEAYSEI]GMKGERRRGKGHDGL[YQGLSTATKDTYDAL]HMQALPPR (SEQ ID NO:29); NQL[YNELNLGRREEYDVL]DKR SEQ ID NO:30); EGL[YNELQKDKMAEAYSEI]GMK (SEQ ID NO:31); or DGL[YQGLSTATKDTYDAL]HMQ (SEQ ID NO:32), where the ITAM motifs are set out in brackets.

In some embodiments, the intracellular activating domain is derived from T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences: MEHSTFLSGLVLATLLSQVSPFKIPIEELEDRVFVNCNTSITWVEGTVGTLLSDITRLDLGKRILDP RGIYRCNGTDIYKDKESTVQVHYRMCQSCVELDPATVAGIIVTDVIATLLLALGVFCFAGHETGR LSGAADTQALLRNDQV[YQPLRDRDDAQYSHL]GGNWARNK (SEQ ID NO:33) or MEHSTFLSGLVLATLLSQVSPFKIPIEELEDRVFVNCNTSITWVEGTVGTLLSDITRLDLGKRILDP RGIYRCNGTDIYKDKESTVQVHYRTADTQALLRNDQV[YQPLRDRDDAQYSHL]GGNWARNK (SEQ ID NO:34), where the ITAM motifs are set out in brackets.

Likewise, a suitable intracellular activating domain polypeptide can comprise an ITAM motif-containing portion of the full length CD3 delta amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DQV[YQPLRDRDDAQYSHL]GGN (SEQ ID NO:35), where the ITAM motifs are set out in brackets.

In some embodiments, the intracellular activating domain is derived from T cell surface glycoprotein CD3 epsilon chain (also known as CD3e, T cell surface antigen T3/Leu-4 epsilon chain, T cell surface glycoprotein CD3 epsilon chain, A1504783, CD3, CD3epsilon, T3e, etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of the following amino acid sequence: MQSGTHWRVLGLCLLSVGVWGQDGNEEMGGITQTPYKVSISGTTVILTCPQYPGSEILWQHNDK NIGGDEDDKNIGSDEDHLSLKEFSELEQSGYYVCYPRGSKPEDANFYLYLRARVCENCMEMDMS VATIVIVDICITGGLLLLVYYWSKNRKAKAKPVTRGAGAGGRQRGQNKERPPPVPNPD[YEPIRK GQRDLYSGL]NQRRI (SEQ ID NO:36), where the ITAM motifs are set out in brackets.

Likewise, a suitable intracellular activating domain polypeptide can comprise an ITAM motif-containing portion of the full length CD3 epsilon amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: NPD[YEPIRKGQRDLYSGL]NQR (SEQ ID NO:37), where the ITAM motifs are set out in brackets.

In some embodiments, the intracellular activating domain is derived from T cell surface glycoprotein CD3 gamma chain (also known as CD3G, T cell receptor T3 gamma chain, CD3-GAMMA, T3G, gamma polypeptide (TiT3 complex), etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of the following amino acid sequence: MEQGKGLAVLILAIILLQGTLAQSIKGNHLVKVYDYQEDGSVLLTCDAEAKNITWFKDGKMIGF LTEDKKKWNLGSNAKDPRGMYQCKGSQNKSKPLQVYYRMCQNCIELNAATISGFLFAEIVSIFV LAVGVYFIAGQDGVRQSRASDKQTLLPNDQL[YQPLKDREDDQYSHL]QGNQLRRN (SEQ ID NO:38), where the ITAM motifs are set out in brackets.

Likewise, a suitable intracellular activating domain polypeptide can comprise an ITAM motif-containing portion of the full length CD3 gamma amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DQL[YQPLKDREDDQYSHL]QGN (SEQ ID NO:39), where the ITAM motifs are set out in brackets.

In some embodiments, the intracellular activating domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences: MPGGPGVLQALPATIFLLFLLSAVYLGPGCQALWMHKVPASLMVSLGEDAHFQCPHNSSNNAN VTWWRVLHGNYTWPPEFLGPGEDPNGTLIIQNVNKSHGGIYVCRVQEGNESYQQSCGTYLRVR QPPPRPFLDMGEGTKNRIITAEGIILLFCAVVPGTLLLFRKRWQNEKLGLDAGDEYEDENL[YEGL NLDDCSMYEDI]SRGLQGTYQDVGSLNIGDVQLEKP (SEQ ID NO:40) or MPGGPGVLQALPATIFLLFLLSAVYLGPGCQALWMHKVPASLMVSLGEDAHFQCPHNSSNNAN VTWWRVLHGNYTWPPEFLGPGEDPNEPPPRPFLDMGEGTKNRIITAEGIILLFCAVVPGTLLLFRK RWQNEKLGLDAGDEYEDENL[YEGLNLDDCSMYEDI]SRGLQGTYQDVGSLNIGDVQLEKP (SEQ ID NO:41), where the ITAM motifs are set out in brackets.

Likewise, a suitable intracellular activating domain polypeptide can comprise an ITAM motif-containing portion of the full length CD79A amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: ENL[YEGLNLDDCSMYEDI]SRG (SEQ ID NO:42), where the ITAM motifs are set out in brackets.

In some embodiments, the intracellular activating domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (4 isoforms): MGGLEPCSRLLLLPLLLAVSGLRPVQAQAQSDCSCSTVSPGVLAGIVMGDLVLTVLIALAVYFLG RLVPRGRGAAEAATRKQRITETESP[YQELQGQRSDVYSDL]NTQRPYYK (SEQ ID NO:43), MGGLEPCSRLLLLPLLLAVSGLRPVQAQAQSDCSCSTVSPGVLAGIVMGDLVLTVLIALAVYFLG RLVPRGRGAAEATRKQRITETESP[YQELQGQRSDVYSDL]NTQ (SEQ ID NO:44), MGGLEPCSRLLLLPLLLAVSDCSCSTVSPGVLAGIVMGDLVLTVLIALAVYFLGRLVPRGRGAAE AATRKQRITETESP[YQELQGQRSDVYSDL]NTQRPYYK (SEQ ID NO:45), or MGGLEPCSRLLLLPLLLAVSDCSCSTVSPGVLAGIVMGDLVLTVLIALAVYFLGRLVPRGRGAAE ATRKQRITETESP[YQELQGQRSDVYSDL]NTQRPYYK (SEQ ID NO:46), where the ITAM motifs are set out in brackets.

Likewise, a suitable intracellular activating domain polypeptide can comprise an ITAM motif-containing portion of the full length DAP12 amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: ESP[YQELQGQRSDVYSDL]NTQ (SEQ ID NO:47), where the ITAM motifs are set out in brackets.

In some embodiments, the intracellular activating domain is derived from FCERIG (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 50 amino acids to about 60 amino acids (aa), from about 60 aa to about 70 aa, from about 70 aa to about 80 aa, or from about 80 aa to about 88 aa, of the following amino acid sequence: MIPAVVLLLLLLVEQAAALGEPQLCYILDAILFLYGIVLTLLYCRLKIQVRKAAITSYEKSDGV[YT GLSTRNQETYETL]KHEKPPQ (SEQ ID NO:48), where the ITAM motifs are set out in brackets.

Likewise, a suitable intracellular activating domain polypeptide can comprise an ITAM motif-containing portion of the full length FCER1G amino acid sequence. Thus, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequence: DGV[YTGLSTRNQETYETL]KHE (SEQ ID NO:49), where the ITAM motifs are set out in brackets.

Intracellular activating domains suitable for use in an engineered signaling polypeptide of the present disclosure include a DAP10/CD28 type signaling chain. An example of a DAP10 signaling chain is the amino acid SEQ ID NO:50. In some embodiments, a suitable intracellular activating domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in SEQ ID NO:50.

An example of a CD28 signaling chain is the amino acid sequence is SEQ ID NO:51. In some embodiments, a suitable intracellular domain includes a domain with at least 50%, 60%, 70%, 75%, 80%, 85%, 90%, 95%, 96%, 97%, 98%, 99% or 100% sequence identity to a stretch of at least 10, 15, 20, or all amino acids of SEQ ID NO:51.

Intracellular activating domains suitable for use in an engineered signaling polypeptide of the present disclosure include a ZAP70 polypeptide, For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 300 amino acids to about 400 amino acids, from about 400 amino acids to about 500 amino acids, or from about 500 amino acids to 619 amino acids, of SEQ ID NO:52.

### Modulatory Domains

Modulatory domains can change the effect of the intracellular activating domain in the engineered signaling polypeptide, including enhancing or dampening the downstream effects of the activating domain or changing the nature of the response. Modulatory domains suitable for use in an engineered signaling polypeptide of the present disclosure include co-stimulatory domains. A modulatory domain suitable for inclusion in the engineered signaling polypeptide can have a length of from about 30 amino acids to about 70 amino acids (aa), e.g., a modulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

Co-stimulatory domains typically enhance and/or change the nature of the response to an activation domain. Co-stimulatory domains suitable for use in an engineered signaling polypeptide of the present disclosure are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). Non-limiting examples of suitable co-stimulatory polypeptides include, but are not limited to, 4-1BB (CD137), CD27, CD28, CD28 deleted for Lck binding (ICΔ), ICOS, OX40, BTLA, CD27, CD30, GITR, and HVEM. For example, a co-stimulatory domain of an aspect of the invention can have at least 80%, 90%, or 95% sequence identity to the co-stimulatory domain of 4-1BB (CD137), CD27, CD28, CD28 deleted for Lck binding (ICΔ), ICOS, OX40, BTLA, CD27, CD30, GITR, or HVEM. For example, a co-stimulatory domain of an aspect of the invention can have at least 80%, 90%, or 95% sequence identity to the co-stimulatory domain of non-limiting examples of suitable co-stimulatory polypeptides include, but are not limited to, 4-1BB (CD137), CD27, CD28, CD28 deleted for Lck binding (ICΔ), ICOS, OX40, BTLA, CD27, CD30, GITR, and HVEM. For example, a co-stimulatory domain of an aspect of the invention can have at least 80%, 90%, or 95% sequence identity to the co-stimulatory domain of 4-1BB (CD137), CD27, CD28, CD28 deleted for Lck binding (ICΔ), ICOS, OX40, BTLA, CD27, CD30, GITR, or HVEM.

A co-stimulatory domain suitable for inclusion in an engineered signaling polypeptide can have a length of from about 30 amino acids to about 70 amino acids (aa), e.g., a co-stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, the co-stimulatory domain can have a length of from about 70 aa to about 100 aa, from about 100 aa to about 200 aa, or greater than 200 aa.

In some embodiments, the co-stimulatory domain is derived from an intracellular portion of the transmembrane protein CD137 (also known as TNFRSF9; CD137; 4-1BB; CDwl37; ILA; etc.). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:53. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some embodiments, the co-stimulatory domain is derived from an intracellular portion of the transmembrane protein CD28 (also known as Tp44). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:54. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some embodiments, the co-stimulatory domain is derived from an intracellular portion of the transmembrane protein CD28 deleted for Lck binding (ICΔ). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:55. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some embodiments, the co-stimulatory domain is derived from an intracellular portion of the transmembrane protein ICOS (also known as AILIM, CD278, and CVIDI). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:56. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some embodiments, the co-stimulatory domain is derived from an intracellular portion of the transmembrane protein OX40 (also known as TNFRSF4, RP5-902P8.3, ACT35, CD134, OX-40, TXGPIL). OX40 contains a p⁸⁵ PI3K binding motif at residues 34-57 and a TRAF binding motif at residues 76-102, each of SEQ ID NO: 296 (of Table 1). In some embodiments, the costimulatory domain can include the p⁸⁵ PI3K binding motif of OX40. In some embodiments, the costimulatory domain can include the TRAF binding motif of OX40. Lysines corresponding to amino acids 17 and 41 of SEQ ID NO: 296 are potentially negative regulatory sites that function as parts of ubiquitin targeting motifs. In some embodiments, one or both of these Lysines in the costimulatory domain of OX40 are mutated Arginines or another amino acid. In some embodiments, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:57. In some of these embodiments, the co-stimulatory domain has a length of from about 20 aa to about 25 aa, about 25 aa to about 30 aa, 30 aa to about 35 aa, from about 35 aa to about 40 aa, from about 40 aa to about 45 aa, or from about 45 aa to about 50 aa. In illustrative embodiments, the co-stimulatory domain has a length of from about 20 aa to about 50 aa, for example 20 aa to 45 aa, or 20 aa to 42 aa.

In some embodiments, the co-stimulatory domain is derived from an intracellular portion of the transmembrane protein CD27 (also known as S 152, T 14, TNFRSF7, and Tp55). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:58. In some of these embodiments, the co-stimulatory domain has a length of from about 30 aa to about 35 aa, from about 35 aa to about 40 aa, from about 40 aa to about 45 aa, or from about 45 aa to about 50 aa.

In some embodiments, the co-stimulatory domain is derived from an intracellular portion of the transmembrane protein BTLA (also known as BTLA1 and CD272). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:59.

In some embodiments, the co-stimulatory domain is derived from an intracellular portion of the transmembrane protein CD30 (also known as TNFRSF8, DlS166E, and Ki-1). For example, a suitable co-stimulatory domain can include a domain with at least 50%, 60%, 70%, 75%, 80%, 85%, 90%, 95%, 96%, 97%, 98%, 99% or 100% sequence identity to a stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa of SEQ ID NO:60.

In some embodiments, the co-stimulatory domain is derived from an intracellular portion of the transmembrane protein GITR (also known as TNFRSF18, RP5-902P8.2, AITR, CD357, and GITR-D). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:61. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some embodiments, the co-stimulatory domain derived from an intracellular portion of the transmembrane protein HVEM (also known as TNFRSF14, RP3-395M20.6, ATAR, CD270, HVEA, HVEM, LIGHTR, and TR2). For example, a suitable co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:62. In some of these embodiments, the co-stimulatory domain of both the first and the second polypeptide has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Linker

In some embodiments, the engineered signaling polypeptide includes a linker between any two adjacent domains. For example, a linker can be between the transmembrane domain and the first co-stimulatory domain. As another example, the ASTR can be an antibody and a linker can be between the heavy chain and the light chain. As another example, a linker can be between the ASTR and the transmembrane domain and a co-stimulatory domain. As another example, a linker can be between the co-stimulatory domain and the intracellular activating domain of the second polypeptide. As another example, the linker can be between the ASTR and the intracellular signaling domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ, GGGSₙ, and GGGGSₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGGGSGGGGSGGGGS (SEQ ID NO:63), GGGGSGGGGSGGGGSGGGGSGGGGSGGGGS (SEQ ID NO:64), GGGGSGGGSGGGGS (SEQ ID NO:65), GGSG (SEQ ID NO:66), GGSGG (SEQ ID NO:67), GSGSG (SEQ ID NO:68), GSGGG (SEQ ID NO:69), GGGSG (SEQ ID NO:70), GSSSG (SEQ ID NO:71), and the like. The ordinarily skilled artisan will recognize that design of a peptide conjugated to any elements described above can include linkers that are all or partially flexible, such that the linker can include a flexible linker as well as one or more portions that confer less flexible structure.

### Combinations

In some embodiments, a polynucleotide provided by the replication incompetent recombinant retroviral particles has one or more transcriptional units that encode certain combinations of the one or more engineered signaling polypeptides. In some methods and compositions provided herein, genetically modified T cells include the combinations of the one or more engineered signaling polypeptides after transduction of T cells by the replication incompetent recombinant retroviral particles. It will be understood that the reference of a first polypeptide, a second polypeptide, a third polypeptide, etc. is for convenience and elements on a “first polypeptide” and those on a “second polypeptide” means that the elements are on different polypeptides that are referenced as first or second for reference and convention only, typically in further elements or steps to that specific polypeptide.

In some embodiments, the first engineered signaling polypeptide includes an extracellular antigen binding domain, which is capable of binding an antigen, and an intracellular signaling domain. In other embodiments, the first engineered signaling polypeptide also includes a T cell survival motif and/or a transmembrane domain. In some embodiments, the first engineered signaling polypeptide does not include a co-stimulatory domain, while in other embodiments, the first engineered signaling polypeptide does include a co-stimulatory domain.

In some embodiments, a second engineered signaling polypeptide includes a lymphoproliferative gene product and optionally an extracellular antigen binding domain. In some embodiments, the second engineered signaling polypeptide also includes one or more of the following: a T cell survival motif, an intracellular signaling domain, and one or more co-stimulatory domains. In other embodiments, when two engineered signaling polypeptides are used, at least one is a CAR.

In one embodiment, the one or more engineered signaling polypeptides are expressed under a T cell specific promoter or a general promoter under the same transcript wherein in the transcript, nucleic acids encoding the engineered signaling polypeptides are separated by nucleic acids that encode one or more internal ribosomal entry sites (IREs) or one or more protease cleavage peptides.

In certain embodiments, the polynucleotide encodes two engineered signaling polypeptides wherein the first engineered signaling polypeptide includes a first extracellular antigen binding domain, which is capable of binding to a first antigen, and a first intracellular signaling domain but not a co-stimulatory domain, and the second polypeptide includes a second extracellular antigen binding domain, which is capable of binding VEGF, and a second intracellular signaling domain, such as for example, the signaling domain of a co-stimulatory molecule. In a certain embodiment, the first antigen is PSCA, PSMA, or BCMA. In a certain embodiment, the first extracellular antigen binding domain comprises an antibody or fragment thereof (e.g., scFv), e.g., an antibody or fragment thereof specific to PSCA, PSMA, or BCMA. In a certain embodiment, the second extracellular antigen binding domain that binds VEGF is a receptor for VEGF, i.e., VEGFR. In certain embodiments, the VEGFR is VEGFR1, VEGFR2, or VEGFR3. In a certain embodiment, the VEGFR is VEGFR2.

In certain embodiments, the polynucleotide encodes two engineered signaling polypeptides wherein the first engineered signaling polypeptide includes an extracellular tumor antigen binding domain and a CD3ζ (signaling domain, and the second engineered signaling polypeptide includes an antigen-binding domain, wherein the antigen is an angiogenic or vasculogenic factor, and one or more co-stimulatory molecule signaling domains. The angiogenic factor can be, e.g., VEGF. The one or more co-stimulatory molecule signaling motifs can comprise, e.g., co-stimulatory signaling domains from each of CD27, CD28, OX40, ICOS, and 4-1BB.

In certain embodiments, the polynucleotide encodes two engineered signaling polypeptides wherein the first engineered signaling polypeptide includes an extracellular tumor antigen-binding domain and a CD3ζ (signaling domain, the second polypeptide comprises an antigen-binding domain, which is capable of binding to VEGF, and co-stimulatory signaling domains from each of CD27, CD28, OX40, ICOS, and 4-1BB. In a further embodiment, the first signaling polypeptide or second signaling polypeptide also has a T cell survival motif. In some embodiments, the T cell survival motif is, or is derived from, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor or the TGFβ decoy receptor (TGF-β-dominant-negative receptor II (DNRII)).

In certain embodiments, the polynucleotide encodes two engineered signaling polypeptides wherein the first engineered signaling polypeptide includes an extracellular tumor antigen-binding domain and a CD3ζ signaling domain, and the second engineered signaling polypeptide includes an antigen-binding domain, which is capable of binding to VEGF, an IL-7 receptor intracellular T cell survival motif, and co-stimulatory signaling domains from each of CD27, CD28, OX40, ICOS, and 4-1BB.

In some embodiments, more than two signaling polypeptides are encoded by the polynucleotide. In certain embodiments, only one of the engineered signaling polypeptides includes an antigen binding domain that binds to a tumor-associated antigen or a tumor-specific antigen; each of the remainder of the engineered signaling polypeptides comprises an antigen binding domain that binds to an antigen that is not a tumor-associated antigen or a tumor-specific antigen. In other embodiments, two or more of the engineered signaling polypeptides include antigen binding domains that bind to one or more tumor-associated antigens or tumor-specific antigens, wherein at least one of the engineered signaling polypeptides comprises an antigen binding domain that does not bind to a tumor-associated antigen or a tumor-specific antigen.

In some embodiments, the tumor-associated antigen or tumor-specific antigen is Her2, prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD19, CD22, CD27, CD30, CD70, GD2 (ganglioside G2), EphA2, CSPG4, CD138, FAP (Fibroblast Activation Protein), CD171, kappa, lambda, 5T4, αvβ6 integrin, integrin αvβ (CD61), galactin, K-Ras (V-Ki-ras2 Kirsten rat sarcoma viral oncogene), Ral-B, B7-H3, B7-H6, CAIX, CD20, CD33, CD44, CD44v6, CD44v7/8, CD123, EGFR, EGP2, EGP40, EpCAM, fetal AchR, FRα, GD3, HLA-A1+MAGE1, HLA-A1+NY-ESO-1, IL-IIRα, IL-13Rα2, Lewis-Y, Muc16, NCAM, NKG2D Ligands, NY-ESO-1, PRAME, ROR1, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein.

In some embodiments, the first engineered signaling polypeptide includes a first extracellular antigen binding domain that binds a first antigen, and a first intracellular signaling domain; and a second engineered signaling polypeptide includes a second extracellular antigen binding domain that binds a second antigen, or a receptor that binds the second antigen; and a second intracellular signaling domain, wherein the second engineered signaling polypeptide does not comprise a co-stimulatory domain. In a certain embodiment, the first antigen-binding domain and the second antigen-binding domain are independently an antigen-binding portion of a receptor or an antigen-binding portion of an antibody. In a certain embodiment, either or both of the first antigen binding domain or the second antigen binding domain are scFv antibody fragments. In certain embodiments, the first engineered signaling polypeptide and/or the second engineered signaling polypeptide additionally comprises a transmembrane domain. In a certain embodiment, the first engineered signaling polypeptide or the second engineered signaling polypeptide comprises a T cell survival motif, e.g., any of the T cell survival motifs described herein.

In another embodiment, the first engineered signaling polypeptide includes a first extracellular antigen binding domain that binds HER2 and the second engineered signaling polypeptide includes a second extracellular antigen binding domain that binds MUC-1.

In another embodiment, the second extracellular antigen binding domain of the second engineered signaling polypeptide binds an interleukin.

In another embodiment, the second extracellular antigen binding domain of the second engineered signaling polypeptide binds a damage associated molecular pattern molecule (DAMP; also known as an alarmin). In other embodiments, a DAMP is a heat shock protein, chromatin-associated protein high mobility group box 1 (HMGB1), S100A8 (also known as MRP8, or calgranulin A), S100A9 (also known as MRP14, or calgranulin B), serum amyloid A (SAA), deoxyribonucleic acid, adenosine triphosphate, uric acid, or heparin sulfate.

In certain embodiments, said second antigen is an antigen on an antibody that binds to an antigen presented by a tumor cell.

In some embodiments, signal transduction activation through the second engineered signaling polypeptide is non-antigenic, but is associated with hypoxia. In certain embodiments, hypoxia is induced by activation of hypoxia-inducible factor-1α (HIF-1α), HIF-1β, HIF-2α, HIF-2β, HIF-3α, or HIF-3β.

In some embodiments, expression of the one or more engineered signaling polypeptides is regulated by a control element, which is disclosed in more detail herein.

### Additional Sequences

The engineered signaling polypeptides, such as CARs, can further include one or more additional polypeptide domains, where such domains include, but are not limited to, a signal sequence; an epitope tag; an affinity domain; and a polypeptide whose presence or activity can be detected (detectable marker), for example by an antibody assay or because it is a polypeptide that produces a detectable signal. Non-limiting examples of additional domains for any of the aspects or embodiments provided herein, include a domain with at least 50%, 60%, 70%, 75%, 80%, 85%, 90%, 95%, 96%, 97%, 98%, 99% or 100% sequence identity to any of the following sequences as described below: a signal sequence, an epitope tag, an affinity domain, or a polypeptide that produces a detectable signal.

Signal sequences that are suitable for use in a subject CAR, e.g., in the first polypeptide of a subject CAR, include any eukaryotic signal sequence, including a naturally-occurring signal sequence, a synthetic (e.g., man-made) signal sequence, etc. In some embodiments, for example, the signal sequence can be the CD8 signal sequence MALPVTALLLPLALLLHAARP (SEQ ID NO:72).

Suitable epitope tags include, but are not limited to, hemagglutinin (HA; e.g., YPYDVPDYA; SEQ ID NO:73); FLAG (e.g., DYKDDDDK; SEQ ID NO:74); c-myc (e.g., EQKLISEEDL; SEQ ID NO:75), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), hemagglutinin, e.g., HA Tag (YPYDVPDYA; SEQ ID NO:73), GST, thioredoxin, cellulose binding domain, RYIRS (SEQ ID NO:79), Phe-His-His-Thr (SEQ ID NO:80), chitin binding domain, S-peptide, T7 peptide, SH2 domain, C-end RNA tag, WEAAAREACCRECCARA (SEQ ID NO:81), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Recognition and/or Elimination Domain

Any of the replication incompetent recombinant retroviral particles provided herein can include nucleic acids that encode a recognition or elimination domain as part of, or separate from, nucleic acids encoding any of the engineered signaling polypeptides provided herein. Thus, any of the engineered signaling polypeptides provided herein, can include a recognition or elimination domain. For example, any of the CARs disclosed herein can include a recognition or elimination domain. Moreover, a recognition or elimination domain can be expressed together with, or even fused with any of the lymphoproliferative elements disclosed herein. The recognition or elimination domains are expressed on the T cell and/or NK cell but are not expressed on the replication incompetent recombinant retroviral particles.

In some embodiments, the recognition or elimination domain can be derived from herpes simplex virus-derived enzyme thymidine kinase (HSV-tk) or inducible caspase-9. In some embodiments, the recognition or elimination domain can include a modified endogenous cell-surface molecule, for example as disclosed in U.S. Pat. No. 8,802,374. The modified endogenous cell-surface molecule can be any cell-surface related receptor, ligand, glycoprotein, cell adhesion molecule, antigen, integrin, or cluster of differentiation (CD) that is modified. In some embodiments, the modified endogenous cell-surface molecule is a truncated tyrosine kinase receptor. In one aspect, the truncated tyrosine kinase receptor is a member of the epidermal growth factor receptor (EGFR) family (e.g., ErbB1, ErbB2, ErbB3, and ErbB4). In some embodiments, the recognition domain can be a polypeptide that is recognized by an antibody that recognizes the extracellular domain of an EGFR member. In some embodiments, the recognition domain can be at least 20 contiguous amino acids of an EGFR family member, or for example, between 20 and 50 contiguous amino acids of an EGFR family member. For example, SEQ ID NO:82, is an exemplary polypeptide that is recognized by, and under the appropriate conditions bound by an antibody that recognizes the extracellular domain of an EGFR member. Such extracellular EGFR epitopes are sometimes referred to herein as eTags. In illustrative embodiments, such epitopes are recognized by commercially available anti-EGFR monoclonal antibodies.

Epidermal growth factor receptor, also known as EGFR, ErbB1 and HER1, is a cell-surface receptor for members of the epidermal growth factor family of extracellular ligands. Alterations in EGFR activity have been implicated in certain cancers. In some embodiments, a gene encoding an EGFR polypeptide including human epidermal growth factor receptor (EGFR) is constructed by removal of nucleic acid sequences that encode polypeptides including the membrane distal EGF-binding domain and the cytoplasmic signaling tail, but retains the extracellular membrane proximal epitope recognized by an anti-EGFR antibody. Preferably, the antibody is a known, commercially available anti-EGFR monoclonal antibody, such as cetuximab, matuzumab, necitumumab or panitumumab.

Others have shown that application of biotinylated-cetuximab to immunomagnetic selection in combination with anti-biotin microbeads successfully enriches T cells that have been lentivirally transduced with EGFRt-containing constructs from as low as 2% of the population to greater than 90% purity without observable toxicity to the cell preparation. Furthermore, others have shown that constitutive expression of this inert EGFR molecule does not affect T cell phenotype or effector function as directed by the coordinately expressed chimeric antigen receptor (CAR), CD19R. In addition, others have shown that through flow cytometric analysis, EGFR was successfully utilized as an in vivo tracking marker for T cell engraftment in mice. Furthermore, EGFR was demonstrated to have suicide gene potential through Erbitux® mediated antibody dependent cellular cytotoxicity (ADCC) pathways. The inventors of the present disclosure have successfully expressed eTag in PBMCs using lentiviral vectors, and have found that expression of eTag in vitro by PBMCs exposed to Cetuximab, provided an effective elimination mechanism for PBMCs. Thus, EGFR may be used as a non-immunogenic selection tool, tracking marker, and suicide gene for transduced T cells that have immunotherapeutic potential. The EGFR nucleic acid may also be detected by means well known in the art.

In some embodiments provided herein, EGFR is expressed as part of a single polypeptide that also includes the CAR or as part of a single polypeptide that includes the lymphoproliferative element. In some embodiments, the amino acid sequence encoding the EGFR recognition domain can be separated from the amino acid sequence encoding the chimeric antigen receptor by a cleavage signal and/or a ribosomal skip sequence. The ribosomal skip and/or cleavage signal can be any ribosomal skip and/or cleavage signal known in the art. Not to be limited by theory, the ribosomal skip sequence can be, for example T2A (also referred to as 2A-1 herein) with amino acid sequence GSGEGRGSLLTCGDVEENPGP (SEQ ID NO:83). Not to be limited by theory, other examples of cleavage signals and ribosomal skip sequences include FMDV 2A (F2A); equine rhinitis A virus 2A (abbreviated as E2A); porcine teschovirus-1 2A (P2A); and Thoseaasigna virus 2A (T2A). In some embodiments, the polynucleotide sequence encoding the recognition domain can be on the same transcript as the CAR or lymphoproliferative element but separated from the polynucleotide sequence encoding the CAR or lymphoproliferative element by an internal ribosome entry site.

In other embodiments as exemplified empirically herein, a recognition domain can be expressed as part of a fusion polypeptide, fused to a lymphoproliferative element. Such constructs provide the advantage, especially in combination with other “space saving” elements provided herein, of taking up less genomic space on an RNA genome compared to separate polypeptides. In one illustrative embodiment, an eTag is expressed as a fusion polypeptide, fused to an IL7Rα mutant, as experimentally demonstrated herein.

### Chimeric Antigen Receptor

In some aspects of the present invention, an engineered signaling polypeptide is a chimeric antigen receptor (CAR) or a polynucleotide encoding a CAR, which, for simplicity, is referred to herein as “CAR.” A CAR of the present disclosure includes: a) at least one antigen-specific targeting region (ASTR); b) a transmembrane domain; and c) an intracellular activating domain. In illustrative embodiments, the antigen-specific targeting region of the CAR is an scFv portion of an antibody to the target antigen. In illustrative embodiments, the intracellular activating domain is from CD3Z, CD3D, CD3E, CD3G, CD79A, CD79B, DAP12, FCERIG, FCGR2A, FCGR2C, DAP10/CD28, or ZAP70, and some further illustrative embodiments, from CD3z. In illustrative embodiments, the CAR further comprises a co-stimulatory domain, for example any of the co-stimulatory domains provided above in the Modulatory Domains section, and in further illustrative embodiments the co-stimulatory domain is the intracellular co-stimulatory domain of 4-1BB (CD137), CD28, ICOS, OX-40, BTLA, CD27, CD30, GITR, and HVEM. In some embodiments, the CAR includes any of the transmembrane domains listed in the Transmembrane Domain section above.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell, an NK-T cell, and a macrophage. When present in the plasma membrane of a eukaryotic cell, a CAR of the present disclosure is active in the presence of one or more target antigens that, in certain conditions, binds the ASTR. The target antigen is the second member of the specific binding pair. The target antigen of the specific binding pair can be a soluble (e.g., not bound to a cell) factor; a factor present on the surface of a cell such as a target cell; a factor presented on a solid surface; a factor present in a lipid bilayer; and the like. Where the ASTR is an antibody, and the second member of the specific binding pair is an antigen, the antigen can be a soluble (e.g., not bound to a cell) antigen; an antigen present on the surface of a cell such as a target cell; an antigen presented on a solid surface; an antigen present in a lipid bilayer; and the like.

In some instances, a CAR of the present disclosure, when present in the plasma membrane of a eukaryotic cell, and when activated by one or more target antigens, increases expression of at least one nucleic acid in the cell. For example, in some cases, a CAR of the present disclosure, when present in the plasma membrane of a eukaryotic cell, and when activated by the one or more target antigens, increases expression of at least one nucleic acid in the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the level of transcription of the nucleic acid in the absence of the one or more target antigens.

As an example, the CAR of the present disclosure can include an immunoreceptor tyrosine-based activation motif (ITAM)-containing intracellular signaling polypeptide.

A CAR of the present disclosure, when present in the plasma membrane of a eukaryotic cell, and when activated by one or more target antigens, can, in some instances, result in increased production of one or more cytokines by the cell. For example, a CAR of the present disclosure, when present in the plasma membrane of a eukaryotic cell, and when activated by the one or more target antigens, can increase production of a cytokine by the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the cell in the absence of the one or more target antigens. Cytokines whose production can be increased include, but are not limited to interferon gamma (IFN-γ), tumor necrosis factor-alpha (TNF-α), IL-2, IL-15, IL-12, IL-4, IL-5, IL-10; a chemokine; a growth factor; and the like.

In some embodiments, a CAR of the present disclosure, when present in the plasma membrane of a eukaryotic cell, and when activated by one or more target antigens, can result in both an increase in transcription of a nucleic acid in the cell and an increase in production of a cytokine by the cell.

In some instances, a CAR of the present disclosure, when present in the plasma membrane of a eukaryotic cell, and when activated by one or more target antigens, results in cytotoxic activity by the cell toward a target cell that expresses on its cell surface an antigen to which the antigen-binding domain of the first polypeptide of the CAR binds. For example, where the eukaryotic cell is a cytotoxic cell (e.g., an NK cell or a cytotoxic T lymphocyte), a CAR of the present disclosure, when present in the plasma membrane of the cell, and when activated by the one or more target antigens, increases cytotoxic activity of the cell toward a target cell that expresses on its cell surface the one or more target antigens. For example, where the eukaryotic cell is an NK cell or a T lymphocyte, a CAR of the present disclosure, when present in the plasma membrane of the cell, and when activated by the one or more target antigens, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of the one or more target antigens.

In some embodiments, a CAR of the present disclosure, when present in the plasma membrane of a eukaryotic cell, and when activated by one or more target antigens, can result in other CAR activation related events such as proliferation and expansion (either due to increased cellular division or anti-apoptotic responses).

In some embodiments, a CAR of the present disclosure, when present in the plasma membrane of a eukaryotic cell, and when activated by one or more target antigens, can result in other CAR activation related events such as intracellular signaling modulation, cellular differentiation, or cell death.

In some embodiments, CARs of the present disclosure are microenvironment restricted. This property is typically the result of the microenvironment restricted nature of the ASTR domain of the CAR. Thus, CARs of the present disclosure can have a lower binding affinity or, in illustrative embodiments, can have a higher binding affinity to one or more target antigens under a condition(s) in a microenvironment than under a condition in a normal physiological environment.

In certain illustrative embodiments, CARs provided herein comprise a co-stimulatory domain in addition to an intracellular activating domain, wherein the co-stimulatory domain is any of the intracellular signaling domains provided herein for lymphoproliferative elements (LEs), such as, for example, intracellular domains of CLEs. In certain illustrative embodiments, the co-stimulatory domains of CARs herein are first intracellular domains (P3 domains) identified herein for CLEs or P4 domains that are shown as effective intracellular signaling domains of CLEs herein in the absence of a P3 domain. Furthermore, in certain illustrative embodiments, co-stimulatory domains of CARs can comprise both a P3 and a P4 intracellular signaling domain identified herein for CLEs. Certain illustrative subembodiments include especially effective P3 and P4 partner intracellular signaling domains as identified herein for CLEs. In illustrative embodiments, the co-stimulatory domain is other than an ITAM-containing intracellular domain of a CAR either as part of the co-stimulatory domain, or in further illustrative embodiments as the only co-stimulatory domain.

In these embodiments that include a CAR with a co-stimulatory domain identified herein as an effective intracellular domain of an LE, the co-stimulatory domain of a CAR can be any intracellular signaling domain in Table 1 provided herein. Active fragments of any of the intracellular domains in Table 1 can be a co-stimulatory domain of a CAR. In illustrative embodiments, the ASTR of the CAR comprises an scFV. In illustrative embodiments, in addition to the c-stimulatory intracellular domain of a CLE, these CARs comprise an intracellular activating domain that in illustrative embodiments is a CD3Z, CD3D, CD3E, CD3G, CD79A, CD79B, DAP12, FCERIG, FCGR2A, FCGR2C. DAP10/CD28, or ZAP70 intracellular activating domain, or in further illustrative embodiments is a CD3z intracellular activating domain.

In these illustrative embodiments, the co-stimulatory domain of a CAR can comprise an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL7RA, IL9R, IL10RA, IL10RB, IL11RA, IL12RB1, IL12RB2, IL13RA1, IL13RA2, IL15RA, IL17RB, IL17RC, IL17RD, IL18R1, IL18RAP, IL20RA, IL20RB, IL21R, IL22RA1, IL23R, IL27RA, IL31RA, LEPR, LIFR, LMP1, MPL, MyD88, OSMR, or PRLR. In some embodiments, the co-stimulatory domain of a CAR can include an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL9R, IL10RA, IL10RB, IL11RA, IL13RA1, IL13RA2, IL17RB, IL17RC, IL17RD, IL18R1, IL18RAP, IL20RA, IL20RB, IL22RA1, IL31RA, LEPR, LIFR, LMP1, MPL, MyD88, OSMR, or PRLR. In some embodiments, the co-stimulatory domain of a CAR can include an intracellular domain or a functional fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL12RB1, IL12RB2, IL13RA2, IL15RA, IL17RD, IL21R, IL23R, IL27RA, IL31RA, LEPR, MPL, MyD88, or OSMR. In some embodiments, the co-stimulatory domain of a CAR can include an intracellular domain or a fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL13RA2, IL17RD, IL31RA, LEPR, MPL, MyD88, or OSMR. In some embodiments, the co-stimulatory domain of a CAR can include an intracellular domain or a functional signaling fragment thereof that includes a signaling domain from CSF2RB, CSF3R, IFNAR1, IFNGR1, IL2RB, IL2RG, IL6ST, IL10RA, IL12RB2, IL17RC, IL17RE, IL18R1, IL27RA, IL31RA, MPL, MyD88, OSMR, or PRLR. In some embodiments, the co-stimulatory domain of a CAR can include an intracellular domain or a functional signaling fragment thereof that includes a signaling domain from CSF2RB, CSF3R, IFNGR1, IL2RB, IL2RG, IL6ST, IL10RA, IL17RE, IL31RA, MPL, or MyD88.

In some embodiments, the co-stimulatory domain of a CAR can include an intracellular domain or a fragment thereof that includes a signaling domain from CSF3R, IL6ST, IL27RA, MPL, and MyD88. In certain illustrative subembodiments, the intracellular activating domain of the CAR is derived from CD3z.

### Recombinant T Cell Receptors (TCRs)

T Cell Receptors (TCRs) recognize specific protein fragments derived from intracellular and well as extracellular proteins. When proteins are broken into peptide fragments, they are presented on the cell surface with another protein called major histocompatibility complex, or MHC, which is called the HLA (human leukocyte antigen) complex in humans. Three different T cell antigen receptors combinations in vertebrates are αβ TCR, γδTCR and pre-TCR. Such combinations are formed by dimerization between members of dimerizing subtypes, such as an α TCR subunit and a β TCR subunit, a γ TCR subunit and a δ TCR subunit, and for pre-TCRs, a pTα subunit and a β TCR subunit. A set of TCR subunits dimerize and recognize a target peptide fragment presented in the context of an MHC. The pre-TCR is expressed only on the surface of immature αβ T cells while the a TCR is expressed on the surface of mature αβ T cells and NK T cells, and γδTCR is expressed on the surface of γδT cells. αβTCRs on the surface of a T cell recognize the peptide presented by MHCI or MHCII and the αβ TCR on the surface of NK T cells recognize lipid antigens presented by CD1. γδTCRs can recognize MHC and MHC-like molecules, and can also recognize non-MHC molecules such as viral glycoproteins. Upon ligand recognition, αβTCRs and γδTCRs transmit activation signals through the CD3zeta chain that stimulate T cell proliferation and cytokine secretion.

TCR molecules belong to the immunoglobulin superfamily with its antigen-specific presence in the V region, where CDR3 has more variability than CDR1 and CDR2, directly determining the antigen binding specificity of the TCR. When the MHC-antigen peptide complex is recognized by a TCR, the CDR1 and CDR2 recognize and bind the sidewall of the MHC molecule antigen binding channel, and the CDR3 binds directly to the antigenic peptide. Recombinant TCRs may thus be engineered that recognize a tumor-specific protein fragment presented on MHC.

Recombinant TCR's such as those derived from human TCRα and TCRβ pairs that recognize specific peptides with common HLAs can thus be generated with specificity to a tumor specific protein (Schmitt, T M et al., 2009). The target of recombinant TCRs may be peptides derived from any of the antigen targets for CAR ASTRs provided herein, but are more commonly derived from intracellular tumor specific proteins such as oncofetal antigens, or mutated variants of normal intracellular proteins or other cancer specific neoepitopes. Libraries of TCR subunits may be screened for their selectivity to a target antigen. Screens of natural and/or recombinant TCR subunits can identify sets of TCR subunits with high avidities and/or reactivities towards a target antigen. Members of such sets of TCR subunits can be selected and cloned to produce one or more polynucleotide encoding the TCR subunit.

Polynucleotides encoding such a set of TCR subunits can be included in a replication incompetent recombinant retroviral particle to genetically modify a lymphocyte, or in illustrative embodiments, a T cell or an NK cell, such that the lymphocyte expresses the recombinant TCR. Accordingly, in any aspect or embodiment provided herein that includes an engineered signaling polypeptide, such as embodiments that include one more CARs and/or lymphoproliferative elements, the engineered signaling polypeptide(s) can include or can be one or more sets of recombinant γδTCR chains, or in illustrative embodiments αβTCR chains. TCR chains that form a set may be co-expressed using a number of different techniques to co-express the two TCR chains as is disclosed herein for expressing two or more other engineered signaling polypeptides such as CARs and lymphoproliferative elements. For example, protease cleavage epitopes such as 2A protease, internal ribosomal entry sites (IRES), and separate promoters may be used.

Several strategies have been employed to reduce the likelihood of mixed TCR dimer formation. In general, this involves modification of the constant (C) domains of the TCRα and TCRβ chains to promote the preferential pairing of the introduced TCR chains with each other, while rendering them less likely to successfully pair with endogenous TCR chains. One approach that has shown some promise in vitro involves replacement of the C domain of human TCRα and TCRβ chains with their mouse counterparts. Another approach involves mutation of the human TCRα common domain and TCRβ chain common regions to promote self-pairing, or the expression of an endogenous TCR alpha and TCR beta miRNA within the viral gene construct. Accordingly, in some embodiments provided herein that include one or more sets of TCR chains as engineered signaling polypeptides, each member of the set of TCR chains, in illustrative embodiments αβTCR chains, comprises a modified constant domain that promotes preferential pairing with each other. In some subembodiments, each member of a set of TCR chains, in illustrative embodiments αβTCR chains, comprises a mouse constant domain from the same TCR chain type, or a constant domain from the same TCR chain subtype with enough sequences derived from a mouse constant domain from the same TCR chain subtype, such that dimerization of the set of TCR chains to each other is preferred over, or occurs to the exclusion of, dimerization with human TCR chains. In other subembodiments, each member of a set of TCR chains, in illustrative embodiments αβTCR chains, comprises corresponding mutations in its constant domain, such that dimerization of the set of TCR chains to each other is preferred over, or occurs to the exclusion of, dimerization with TCR chains that have human constant domains. Such preferred or exclusive dimerization in illustrative embodiments, is under physiological conditions.

### Lymphoproliferative Elements

Peripheral T lymphocyte numbers are maintained at remarkably stable levels throughout adulthood, despite the continuing addition of cells, due to emigration from the thymus and proliferation in response to antigen encounter, and loss of cells owing to the removal of antigen-specific effectors after antigen clearance (Marrak, P. et al. 2000. Nat Immunol 1:107-111; Freitas, A. A. et al. 2000. Annu Rev Immunol 18:83-111). The size of the peripheral T cell compartment is regulated by multiple factors that influence both proliferation and survival. However, in a lymphopenic environment, T lymphocytes divide independently of cognate antigen, due to “acute homeostatic proliferation” mechanisms that maintain the size of the peripheral T cell compartment. Conditions for lymphopenia have been established in subjects or patients during adoptive cell therapy by proliferating T cells in vitro and introducing them into lymphodepleted subjects, resulting in enhanced engraftment and antitumor function of transferred T cells. However, lymphodepletion of a subject is not desirable because it can cause serious side effects, including immune dysfunction and death.

Studies have shown that lymphodepletion removes endogenous lymphocytes functioning as cellular sinks for homeostatic cytokines, thereby freeing cytokines to induce survival and proliferation of adoptively transferred cells. Some cytokines, such as for example, IL-7 and IL-15, are known to mediate antigen-independent proliferation of T cells and are thus capable of eliciting homeostatic proliferation in non-lymphopenic environments. However, these cytokines and their receptors have intrinsic control mechanisms that prevent lymphoproliferative disorders at homeostasis.

Many of the embodiments provided herein include a lymphoproliferative element, or a nucleic acid encoding the same, typically as part of an engineered signaling polypeptide. Accordingly, in some aspects of the present invention, an engineered signaling polypeptide is a lymphoproliferative element (LE) such as a chimeric lymphoproliferative element (CLE). Typically, the LE comprises an extracellular domain, a transmembrane domain, and at least one intracellular signaling domain that drives proliferation, and in illustrative embodiments a second intracellular signaling domain.

In some embodiments, the lymphoproliferative element can include a first and/or second intracellular signaling domain. In some embodiments, the first and/or second intracellular signaling domain can include CD2, CD3D, CD3E, CD3G, CD4, CD8A, CD8B, CD27, mutated Delta Lck CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof. In illustrative embodiments, the first intracellular signaling domain can include MyD88, or a functional mutant and/or fragment thereof. In further illustrative embodiments, the first intracellular signaling domain can include MyD88, or a functional mutant and/or fragment thereof, and the second intracellular signaling domain can include ICOS, TNFRSF4, or TNSFR18, or functional mutants and/or fragments thereof. In some embodiments, the first intracellular domain is MyD88 and the second intracellular domain is an ITAM-containing intracellular domain, for example, an intracellular domain from CD3Z, CD3D, CD3E, CD3G, CD79A, CD79B, DAP12, FCERIG, FCGR2A, FCGR2C, DAP10/CD28, or ZAP70. In some embodiments, the second intracellular signaling domain can include TNFRSF18, or a functional mutant and/or fragment thereof.

In some embodiments, the lymphoproliferative element can include a fusion of an extracellular domain and a transmembrane domain. In some embodiments, the fusion of an extracellular domain and a transmembrane domain can include eTAG IL7RA Ins PPCL (interleukin 7 receptor), Myc LMP1, LMP1, eTAG CRLF2, eTAG CSF2RB, eTAG CSF3R, eTAG EPOR, eTAG GHR, eTAG truncated after Fn F523C IL27RA, or eTAG truncated after Fn S505N MPL, or functional mutants and/or fragments thereof. In some embodiments, the lymphoproliferative element can include an extracellular domain. In some embodiments, the extracellular domain can include eTag with 0, 1, 2, 3, or 4 additional alanines at the carboxy terminus. In some embodiments, the extracellular domain can include Myc with 0, 1, 2, 3, or 4 additional alanines at the carboxy terminus, or functional mutants and/or fragments thereof.

In some embodiments, the lymphoproliferative element can include a transmembrane domain. In some embodiments, the transmembrane domain can include CD2, CD3D, CD3E, CD3G, CD3Z CD247, CD4, CD8A, CD8B, CD27, CD28, CD40, CD79A, CD79B, CRLF2, CSF2RA, CSF2RB, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, MPL, OSMR, PRLR, TNFRSF4, TNFRSF8, TNFRSF9, TNFRSF14, or TNFRSF18, or functional mutants and/or fragments thereof.

CLEs for use in any aspect or embodiment herein can include any CLE disclosed in WO2019/055946 (incorporated by reference herein, in its entirety), the vast majority of which were designed to be and are believed to be constitutively active. As illustrated therein, where there is a first and a second intracellular signaling domain of a CLE, the first intracellular signaling domain is positioned between the membrane associating motif and the second intracellular domain.

In another embodiment, the LE provides, is capable of providing and/or possesses the property of (or a cell genetically modified and/or transduced with the LE is capable of providing, is adapted for, possesses the property of, and/or is modified for) driving T cell expansion in vivo. Methods for performing such an in vivo test are provided in Example 6. For example, as illustrated in Example 6, the in vivo test can utilize a mouse model and measure T cell expansion at 15 to 25 days in vivo, or at 19 to 21 days in vivo, or at approximately 21 days in vivo, after T cells are contacted with lentiviral vectors encoding the LEs, are introduced into the mice.

In some embodiments, the lymphoproliferative element can include any of the sequences listed in Table 1 (SEQ ID NOs: 84-302). Table 1 shows the parts, names (including gene names), and amino acid sequences for domains that were tested in CLEs. Typically, a CLE includes an extracellular domain (denoted P1), a transmembrane domain (denoted P2), a first intracellular domain (denoted P3), and a second intracellular domain (denoted P4). Typically, the lymphoproliferative element includes a first intracellular domain. In illustrative embodiments, the first intracellular domain can include any of the parts listed as S036 to S0216 or in Table 1, or functional mutants and/or fragments thereof. In some embodiments, the lymphoproliferative element can include a second intracellular domain. In illustrative embodiments, the second intracellular domain can include any of the parts listed as S036 to S0216 or in Table 1, or functional mutants and/or fragments thereof. In some embodiments, the lymphoproliferative element can include an extracellular domain. In illustrative embodiments, the extracellular domain can include any of the sequences of parts listed as M001 to M049 or E006 to E015 in Table 1, or functional mutants and/or fragments thereof. In some embodiments, the lymphoproliferative element can include a transmembrane domain. In illustrative embodiments, the transmembrane domain can include any of the parts listed as M001 to M049 or T001 to T082 in Table 1, or functional mutants and/or fragments thereof. In some embodiments, the lymphoproliferative element can be of fusion of an extracellular/transmembrane domain (M001 to M049 in Table 1), a first intracellular domain (5036 to 50216 in Table 1), and a second intracellular domain (5036 to 5216 in Table 1). In some embodiments, the lymphoproliferative element can be a fusion of an extracellular domain (E006 to E015 in Table 1), a transmembrane domain (T001 to T082 in Table 1), a first intracellular domain (S036 to S0216 in Table 1), and a second intracellular domain (S036 to S0216 in Table 1). For example, the lymphoproliferative element can be a fusion of E006, T001, S036, and S216, also written as E006-T001-S036-S216). In illustrative embodiments, the lymphoproliferative element can be the fusion E010-T072-S192-S212, E007-T054-S197-S212, E006-T006-S194-S211, E009-T073-S062-S053, E008-T001-S121-S212, E006-T044-S186-S053, or E006-T016-S186-S050.

In illustrative embodiments, the intracellular domain of an LE, or the first intracellular domain in an LE that has two or more intracellular domains, is other than a functional intracellular activating domain from an ITAM-containing intracellular domain, for example, an intracellular domain from CD3Z, CD3D, CD3E, CD3G, CD79A, CD79B, DAP12, FCERIG, FCGR2A, FCGR2C, DAP10/CD28, or ZAP70, and in a further illustrative subembodiment, CD3z. In illustrative embodiments, a second intracellular domain of an LE is other than a co-stimulatory domain of 4-1BB (CD137), CD28, ICOS, OX-40, BTLA, CD27, CD30, GITR, and HVEM. In illustrative embodiments, the extracellular domain of an LE does not comprise a single-chain variable fragment (scFv). In further illustrative embodiments, the extracellular domain of an LE that upon binding to a binding partner activates an LE, does not comprise a single-chain variable fragment (scFv).

A CLE does not comprise both an ASTR and an activation domain from CD3Z, CD3D, CD3E, CD3G, CD79A, CD79B, DAP12, FCERIG, FCGR2A, FCGR2C, DAP10/CD28, or ZAP70. Not to be limited by theory, the extracellular domain and transmembrane domain are believed to play support roles in LEs, assuring that the intracellular signaling domain(s) is in an effective conformation/orientation/localization for driving proliferation. Thus, the ability of an LE to drive proliferation is believed to be provided by the intracellular domain(s) of the LE, and the extracellular and transmembrane domains are believed to play secondary roles relative to the intracellular domain(s). A lymphoproliferative element includes an intracellular domain that is a signaling polypeptide that is capable of driving proliferation of T cells or NK cells that is associated with a membrane through a membrane-associating motif (e.g. a transmembrane domain) and is oriented in, or capable of being oriented into, an active conformation. The ASTR of an LE in illustrative embodiments, does not include an scFv. Strategies are provided herein for associating an intracellular domain with a membrane, such as by inclusion of a transmembrane domain, a GPI anchor, a myristoylation region, a palmitoylation region, and/or a prenylation region. In some embodiments, a lymphoproliferative element does not include an extracellular domain.

The extracellular domains, transmembrane domains, and intracellular domains of LEs can vary in their respective amino acid lengths. For example, for embodiments that include a replication incompetent retroviral particle, there are limits to the length of a polynucleotide that can be packaged into a retroviral particle so LEs with shorter amino acid sequences can be advantageous in certain illustrative embodiments. In some embodiments, the overall length of the LE can be between 3 and 4000 amino acids, for example between 10 and 3000, 10 and 2000, 50 and 2000, 250 and 2000 amino acids, and, in illustrative embodiments between 50 and 1000, 100 and 1000 or 250 and 1000 amino acids. The extracellular domain, when present to form an extracellular and transmembrane domain, can be between 1 and 1000 amino acids, and is typically between 4 and 400, between 4 and 200, between 4 and 100, between 4 and 50, between 4 and 25, or between 4 and 20 amino acids. In one embodiment, the extracellular region is GGGS for an extracellular and transmembrane domain of this aspect of the invention. The transmembrane domains, or transmembrane regions of extracellular and transmembrane domains, can be between 10 and 250 amino acids, and are more typically at least 15 amino acids in length, and can be, for example, between 15 and 100, 15 and 75, 15 and 50, 15 and 40, or 15 and 30 amino acids in length. The intracellular signaling domains can be, for example, between 10 and 1000, 10 and 750, 10 and 500, 10 and 250, or 10 and 100 amino acids. In illustrative embodiments, the intracellular signaling domain can be at least 30, or between 30 and 500, 30 and 250, 30 and 150, 30 and 100, 50 and 500, 50 and 250, 50 and 150, or 50 and 100 amino acids. In some embodiments, an intracellular signaling domain for a particular gene is at least 90%, 95%, 98%, 99% or 100% identical to at least 10, 25, 30, 40, or 50 amino acids from a sequence of that intracellular signaling domain, such as a sequence provided herein for that intracellular domain, up to the size of the entire intracellular domain sequence, and can include for example, up to an additional 1, 2, 3, 4, 5, 10, 20, or 25 amino acids, provided that such sequence still is capable of providing any of the properties of LEs disclosed herein.

In some embodiments, the lymphoproliferative element is a chimeric cytokine receptor such as but not limited to a cytokine tethered to its receptor that typically constitutively activates the same STAT pathway as a corresponding activated wild-type cytokine receptor such as STAT3, STAT4, and in illustrative embodiments, STAT5. In some embodiments, the chimeric cytokine receptor is an interleukin, or a fragment thereof, tethered to or covalently attached to its cognate receptor, or a fragment thereof, via a linker. In some embodiments, the chimeric cytokine receptor is IL7 tethered to IL7Rα (also known as IL7RA). In other embodiments, the chimeric cytokine receptor is IL-7 tethered to a domain of IL7Rα, such as for example, the extracellular domain of IL-7Rα and/or the transmembrane domain of IL-7Rα. In some embodiments, the lymphoproliferative element is a cytokine receptor that is not tethered to a cytokine, and in fact in illustrative embodiments, provided herein a lymphoproliferative element is a constitutively active cytokine receptor that is not tethered to a cytokine. These chimeric IL-7 receptors typically constitutively activate STAT5 when expressed.

In illustrative embodiments of any of the methods and compositions provided herein that include a lymphoproliferative element, wherein the lymphoproliferative element is a cytokine or cytokine receptor polypeptide, or a fragment thereof comprising a signaling domain, the lymphoproliferative element can comprise an interleukin polypeptide covalently attached to a portion of its cognate interleukin receptor polypeptide via a linker. Typically, this portion of the cognate interleukin receptor includes a functional portion of the extracellular domain capable of binding the interleukin cytokine and the transmembrane domain. In some embodiments, the intracellular domain is an intracellular portion of the cognate interleukin receptor. In some embodiments, the intracellular domain is an intracellular portion of a different cytokine receptor that is capable of promoting lymphocyte proliferation. In some embodiments the lymphoproliferative element is an interleukin polypeptide covalently attached to its full length cognate interleukin receptor polypeptide via a linker.

In illustrative embodiments of any of the methods and compositions provided herein that include a lymphoproliferative element, the intracellular domain can be derived from a portion of the protein IL7RA. The domains, motifs, and point mutations of IL7RA that induce proliferation and/or survival of T cells and/or NK cells are known in the art and a skilled artisan can identify corresponding domains, motifs, and point mutations in IL7RA polypeptides, some of which are discussed in this paragraph. The IL7RA protein has an S region rich in serine residues (359-394 of full-length IL7RA, corresponding to residues 96-133 of SEQ ID NO:248), a T region with three tyrosine residues (residues Y401, Y449, and Y456 of full-length IL7RA, corresponding to residues Y138, Y18, and Y193 of SEQ ID NO:248), and a Box1 motif that can bind the signaling kinase Jak1 (residues 272-280 of full-length IL7RA corresponding to residues 9-17 of SEQ ID NO:248 and 249) (Jiang, Qiong et al. Mol. and Cell. Biol. Vol. 24(14):6501-13 (2004)). In some embodiments, a lymphoproliferative element herein can include one or more, for example all of the domains and motifs of IL7RA disclosed herein or otherwise known to induce proliferation and/or survival of T cells and/or NK cells. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:248 or 249. In some embodiments, the intracellular domain derived from IL7RA has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, or from about 175 aa to about 200 aa. In illustrative embodiments, the intracellular domain derived from IL7RA has a length of from about 30 aa to about 200 aa. In illustrative embodiments of lymphoproliferative elements that include a first intracellular domain derived from IL7RA, the second intracellular domain can be derived from TNFRSF8.

In illustrative embodiments of any of the methods and compositions provided herein that include a lymphoproliferative element, the intracellular domain can be derived from a portion of the protein IL12RB. The domains, motifs, and point mutations of IL12RB that induce proliferation and/or survival of T cells and/or NK cells are known in the art and a skilled artisan can identify corresponding domains, motifs, and point mutations in IL12RB polypeptides, some of which are discussed in this paragraph. Full-length IL12RB contains at least one Box1 motif PXXP (SEQ ID NO:306) where each X can be any amino acid (residues 10⁻¹² of SEQ ID NOs:254 and 255; and residues 107-110 and 139-142 of SEQ ID NO:256) (Presky D H et al. Proc Natl Acad Sci USA. 1996 Nov. 26; 93(24)). In some embodiments, a lymphoproliferative element that includes an IL12RB intracellular domain can include one or more of the above Box1 motifs or other motifs, domains, or mutations of IL12RB known to induce proliferation and/or survival of T cells and/or NK cells. The Box1 motifs of IL12RB are known in the art and a skilled artisan can identify corresponding motifs in IL12RB polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:254-256. In some embodiments, the intracellular domain derived from IL12RB has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, or from about 200 aa to about 219 aa. In illustrative embodiments, the intracellular domain derived from IL12RB has a length of from about 30 aa to about 219 aa, for example, 30 aa to 92 aa, or 30 aa to 90 aa.

In illustrative embodiments of any of the methods and compositions provided herein that include a lymphoproliferative element, the intracellular domain can be derived from a portion of the protein IL31RA. The domains, motifs, and point mutations of IL31RA that induce proliferation and/or survival of T cells and/or NK cells are known in the art and a skilled artisan can identify corresponding domains, motifs, and point mutations in IL31RA polypeptides, some of which are discussed in this paragraph. Full-length IL31RA contains the Box1 motif PXXP (SEQ ID NO:306) where each X can be any amino acid (corresponding to residues 12-15 of SEQ ID NOs:275 and 276) (Cornelissen C et al. Eur J Cell Biol. 2012 June-July; 91(6-7):552-66). In some embodiments, a lymphoproliferative element that includes an IL31RA intracellular domain can include the Box1 motif. Full-length IL31RA also contains three phosphorylatable tyrosine residues that are important for downstream signaling, Y652, Y683, and Y721 (corresponding to residues Y96, Y237, and Y165 of SEQ ID NO:275; these tyrosine residues are not present in SEQ ID NO:276) (Cornelissen C et al. Eur J Cell Biol. 2012 June-July; 91(6-7):552-66). All three tyrosine residues contribute to the activation of STAT1, while Y652 is required for STAT5 activation and Y721 recruits STAT3. In some embodiments, a lymphoproliferative element with an IL31RA intracellular domain includes the Box1 motif and/or the known phosphorylation sites disclosed herein. The Box1 motif and phosphorylatable tyrosines of IL31RA are known in the art and a skilled artisan will be able to identify corresponding motifs and phosphorylatable tyrosines in similar IL31RA polypeptides. In other embodiments, a lymphoproliferative element with an IL31RA intracellular domain does not include the known phosphorylation sites disclosed herein. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:275 or 276. In some embodiments, the intracellular domain derived from IL31RA has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, or from about 175 aa to about 189 aa. In illustrative embodiments, the intracellular domain derived from IL31RA has a length of from about 30 aa to about 200 aa, for example, 30 aa to 189 aa, 30 aa to 106 aa.

In illustrative embodiments of any of the methods and compositions provided herein that include a lymphoproliferative element, the intracellular domain can be derived from an intracellular portion of the transmembrane protein CD40. The domains, motifs, and point mutations of CD40 that induce proliferation and/or survival of T cells and/or NK cells are known in the art and a skilled artisan can identify corresponding domains, motifs, and point mutations in CD40 polypeptides, some of which are discussed in this paragraph. The CD40 protein contains several binding sites for TRAF proteins. Not to be limited by theory, binding sites for TRAF1, TRAF2, and TRAF3 are located at the membrane distal domain of the intracellular portion of CD40 and include the amino acid sequence PXQXT (SEQ ID NO:303) where each X can be any amino acid, (corresponding to amino acids 35-39 of SEQ ID NO:208) (Elgueta et al. Immunol Rev. 2009 May; 229(1):152-72). TRAF2 has also been shown to bind to the consensus sequence SXXE (SEQ ID NO:304) where each X can be any amino acid, (corresponding to amino acids 57-60 of SEQ ID NO:208) (Elgueta et al. Immunol Rev. 2009 May; 229(1):152-72). A distinct binding site for TRAF6 is situated at the membrane proximal domain of intracellular portion of CD40 and includes the consensus sequence QXPXEX (SEQ ID NO:305) where each X can be any amino acid (corresponding to amino acids 16-21 of SEQ ID NO:208) (Lu et al. J Biol Chem. 2003 Nov. 14; 278(46):45414-8). In illustrative embodiments, the intracellular portion of the transmembrane protein CD40 can include all the binding sites for the TRAF proteins. The TRAF binding sites are known in the art and a skilled artisan will be able to identify corresponding TRAF binding sites in similar CD40 polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:208 or SEQ ID NO:209. In some embodiments, the intracellular domain derived from CD40 has a length of from about 30 amino acids (aa) to about 35 aa, from about 35 aa to about 40 aa, from about 40 aa to about 45 aa, from about 45 aa to about 50 aa, from about 50 aa to about 55 aa, from about 55 aa to about 60 aa, or from about 60 aa to about 65 aa. In illustrative embodiments, the intracellular domain derived from CD40 has a length of from about 30 aa to about 66 aa, for example, 30 aa to 65 aa, or 50 aa to 66 aa. In illustrative embodiments of lymphoproliferative elements that include a first intracellular domain derived from CD40, the second intracellular domain can be other than an intracellular domain derived from MyD88, a CD28 family member (e.g. CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, techoic acids, toxins, CD1 1 c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1

In illustrative embodiments of any of the methods and compositions provided herein that include a lymphoproliferative element, the intracellular domain can be derived from an intracellular portion of CD27. The domains, motifs, and point mutations of CD27 that induce proliferation and/or survival of T cells and/or NK cells are known in the art and a skilled artisan can identify corresponding domains, motifs, and point mutations in CD27 polypeptides, some of which are discussed in this paragraph. The serine at amino acid 219 of full-length CD27 (corresponding to the serine at amino acid 6 of SEQ ID NO:205) has been shown to be phosphorylated. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:205. In some embodiments, the intracellular domain derived from CD27 has a length of from about 30 amino acids (aa) to about 35 aa, from about 35 aa to about 40 aa, from about 40 aa to about 45 aa, or from about 45 aa to about 50 aa.

In illustrative embodiments of any of the methods and compositions provided herein that include a lymphoproliferative element, the intracellular domain can be derived from an intracellular portion of CSF2RB. The domains, motifs, and point mutations of CSF2RB that induce proliferation and/or survival of T cells and/or NK cells are known in the art and a skilled artisan can identify corresponding domains, motifs, and point mutations in CSF2RB polypeptides, some of which are discussed in this paragraph. Full-length CSF2RB contains a Box1 motif at amino acids 474-482 (corresponding to amino acids 14-22 of SEQ ID NO:213). The tyrosine at amino acid 766 of full-length CSF2RB (corresponding to the tyrosine at amino acid 306 of SEQ ID NO: 213) has been shown to be phosphorylated. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO: 213. In some embodiments, the intracellular domain derived from CSF2RB has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, from about 300 aa to 350 aa, from about 350 aa to about 400 aa, or from about 400 aa to about 450 aa.

In illustrative embodiments of any of the methods and compositions provided herein that include a lymphoproliferative element, the intracellular domain can be derived from an intracellular portion of IL2RB. The domains, motifs, and point mutations of IL2RB that induce proliferation and/or survival of T cells and/or NK cells are known in the art and a skilled artisan can identify corresponding domains, motifs, and point mutations in IL2RB polypeptides, some of which are discussed in this paragraph. Full-length IL2RB contains a Box1 motif at amino acids 278-286 (corresponding to amino acids 13-21 of SEQ ID NO:240). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:240. In some embodiments, the intracellular domain derived from IL2RB has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, or from about 250 aa to 300 aa.

In illustrative embodiments of any of the methods and compositions provided herein that include a lymphoproliferative element, the intracellular domain can be derived from an intracellular portion of IL6ST. The domains, motifs, and point mutations of IL6ST that induce proliferation and/or survival of T cells and/or NK cells are known in the art and a skilled artisan can identify corresponding domains, motifs, and point mutations in IL6ST polypeptides, some of which are discussed in this paragraph. Full-length IL6ST contains a Box1 motif at amino acids 651-659 (corresponding to amino acids 10-18 of SEQ ID NO:247). The serines at amino acids 661, 667, 782, 789, 829, and 839 of full-length IL6ST (corresponding to serines at amino acids 20, 26, 141, 148, 188, and 198, respectively, of SEQ ID NO:247) have been shown to be phosphorylated. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:246 or SEQ ID NO:247. In some embodiments, the intracellular domain derived from IL6ST has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, or from about 250 aa to 300 aa.

In illustrative embodiments of any of the methods and compositions provided herein that include a lymphoproliferative element, the intracellular domain can be derived from an intracellular portion of IL17RE. The domains, motifs, and point mutations of IL17RE that induce proliferation and/or survival of T cells and/or NK cells are known in the art and a skilled artisan can identify corresponding domains, motifs, and point mutations in IL17RE polypeptides, some of which are discussed in this paragraph. Full-length IL17RE contains a TIR domain at amino acids 372-495 (corresponding to amino acids 13-136 of SEQ ID NO:265). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:265. In some embodiments, the intracellular domain derived from IL17RE has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, or from about 175 aa to about 200 aa.

In illustrative embodiments of any of the methods and compositions provided herein that include a lymphoproliferative element, the intracellular domain can be derived from an intracellular portion of IL2RG. The domains, motifs, and point mutations of IL2RG that induce proliferation and/or survival of T cells and/or NK cells are known in the art and a skilled artisan can identify corresponding domains, motifs, and point mutations in IL2RG polypeptides, some of which are discussed in this paragraph. Full-length IL2RG contains a Box1 motif at amino acids 286-294 (corresponding to amino acids 3-11 of SEQ ID NO:241). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:241. In some embodiments, the intracellular domain derived from IL2RG has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, or from about 70 aa to about 100 aa.

In illustrative embodiments of any of the methods and compositions provided herein that include a lymphoproliferative element, the intracellular domain can be derived from an intracellular portion of IL18R1. The domains, motifs, and point mutations of IL18R1 that induce proliferation and/or survival of T cells and/or NK cells are known in the art and a skilled artisan can identify corresponding domains, motifs, and point mutations in IL18R1 polypeptides, some of which are discussed in this paragraph. Full-length IL18R1 contains a TIR domain at amino acids 222-364 (corresponding to amino acids 28-170 of SEQ ID NO:266). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:266. In some embodiments, the intracellular domain derived from IL18R1 has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, or from about 70 aa to about 100 aa.

In illustrative embodiments of any of the methods and compositions provided herein that include a lymphoproliferative element, the intracellular domain can be derived from an intracellular portion of IL27RA. The domains, motifs, and point mutations of IL27RA that induce proliferation and/or survival of T cells and/or NK cells are known in the art and a skilled artisan can identify corresponding domains, motifs, and point mutations in IL27RA polypeptides, some of which are discussed in this paragraph. Full-length IL27RA contains a Box1 motif at amino acids 554-562 (corresponding to amino acids 17-25 of SEQ ID NO:273). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:273 or SEQ ID NO:274. In some embodiments, the intracellular domain derived from IL27RA has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, or from about 70 aa to about 100 aa.

In illustrative embodiments of any of the methods and compositions provided herein that include a lymphoproliferative element, the intracellular domain can be derived from an intracellular portion of IFNGR2. The domains, motifs, and point mutations of IFNGR2 that induce proliferation and/or survival of T cells and/or NK cells are known in the art and a skilled artisan can identify corresponding domains, motifs, and point mutations in IFNGR2 polypeptides, some of which are discussed in this paragraph. Full-length IFNGR2 contains a dileucine internalization motif at amino acids 276-277 (corresponding to amino acids 8-9 of SEQ ID NO:230). In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:230. In some embodiments, the intracellular domain derived from IFNGR2 has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In illustrative embodiments of any of the methods and compositions provided herein that include a lymphoproliferative element, the intracellular domain can be derived from a portion of the protein MyD88. The domains, motifs, and point mutations of MyD88 that induce proliferation and/or survival of T cells and/or NK cells are known in the art and a skilled artisan can identify corresponding domains, motifs, and point mutations in MyD88 polypeptides, some of which are discussed in this paragraph. The MyD88 protein has an N-terminal death domain that mediates interactions with other death domain-containing proteins (corresponding to amino acids 29-106 of SEQ ID NO:284), an intermediate domain that interacts with IL-1R associated kinase (corresponding to amino acids 107-156 of SEQ ID NO:284), and a C-terminal TIR domain (corresponding to amino acids 160-304 of SEQ ID NO:284) that associates with the TLR-TIR domain (Biol Res. 2007; 40(2):97-112). MyD88 also has canonical nuclear localization and export motifs. Point mutations have been identified in MyD88 and include the loss-of-function mutations L93P and R193C (corresponding to L93P and R196C in SEQ ID NO:284), and the gain-of-function mutation L265P (corresponding to L260P in SEQ ID NO:284) (Deguine and Barton. F1000Prime Rep. 2014 Nov. 4; 6:97). In some embodiments, a lymphoproliferative element herein can include one or more, for example all of the domains and motifs of MyD88 disclosed herein. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:284-293, and in illustrative embodiments includes one or more, in illustrative embodiments all, of the following MyD88 domains/motifs: the death domain, the intermediate domain, the TIR domain, the nuclear localization and export motifs, an amino acid corresponding to position L93, R193, and L265 or P265. In some embodiments, the intracellular domain derived from MyD88 has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, or from about 300 aa to 350 aa. In illustrative embodiments, the intracellular domain derived from MyD88 has a length of from about 30 aa to about 350 aa, for example, 50 aa to 350 aa, or 100 aa to 350 aa, 100 aa to 304 aa, 100 aa to 296 aa, 100 aa to 251 aa, 100 aa to 191 aa, 100 aa to 172 aa, 100 aa to 146 aa, or 100 aa to 127 aa. In illustrative embodiments of lymphoproliferative elements that include a first intracellular domain derived from MyD88, the second intracellular domain can be derived from TNFRSF4 or TNFRSF8. In other illustrative embodiments of lymphoproliferative elements that include a first intracellular domain derived from MyD88, the second intracellular domain can be other than an intracellular domain derived from a CD28 family member (e.g. CD28, ICOS), Pattern Recognition Receptor, a C-reactive protein receptor (i.e., Nodi, Nod2, PtX3-R), a TNF receptor (i.e., CD40, RANK/TRANCE-R, OX40, 4-1BB), an HSP receptor (Lox-1 and CD91), or CD28.

In illustrative embodiments of any of the methods and compositions provided herein that include a lymphoproliferative element, the intracellular domain can be derived from a portion of the transmembrane protein MPL. The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. The transmembrane MPL protein contains the Box1 motif PXXP (SEQ ID NO:306) where each X can be any amino acid (corresponding to amino acids 17-20 in SEQ ID NO:283) and the Box2 motif, a region with increased serine and glutamic acid content (corresponding to amino acids 46-64 in SEQ ID NO:283) (Drachman and Kaushansky. Proc Natl Acad Sci USA. 1997 Mar. 18; 94(6):2350-5). The Box1 and Box2 motifs are involved in binding to JAKs and signal transduction, although the Box2 motif presence is not always required for a proliferative signal (Murakami et al. Proc Natl Acad Sci USA. 1991 Dec. 15; 88(24):11349-53; Fukunaga et al. EMBO J. 1991 October; 10(10):2855-65; and O'Neal and Lee. Lymphokine Cytokine Res. 1993 October; 12(5):309-12). Many cytokine receptors have hydrophobic residues at positions −1, −2, and −6 relative to the Box1 motif (corresponding to amino acids 16, 15, and 11, respectively, of SEQ ID NO:283), that form a “switch motif,” which is required for cytokine-induced JAK2 activation but not for JAK2 binding (Constantinescu et al. Mol Cell. 2001 February; 7(2):377-85; and Huang et al. Mol Cell. 2001 December; 8(6):1327-38). Deletion of the region encompassing amino acids 70-95 in SEQ ID NO:283 was shown to support viral transformation in the context of v-mpl (Benit et al. J Virol. 1994 August; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. In full-length MPL, the lysines K553 (corresponding to K40 of SEQ ID NO: 283) and K573 (corresponding to K60 of SEQ ID NO: 283) have been shown to be negative regulatory sites that function as part of a ubiquitination targeting motif (Saur et al. Blood 2010 Feb. 11; 115(6):1254-63). Thus, in some embodiments herein, a MPL intracellular signaling domain does not comprise these ubiquitination targeting motif residues. In full-length MPL, the tyrosines Y521 (corresponding to Y8 of SEQ ID NO: 283), Y542 (corresponding to Y29 of SEQ ID NO:283), Y591 (corresponding to Y78 of SEQ ID NO: 283), Y626 (corresponding to Y113 of SEQ ID NO: 283), and Y631 (corresponding to Y118 of SEQ ID NO: 283) have been shown to be phosphorylated (Varghese et al. Front Endocrinol (Lausanne). 2017 Mar. 31; 8:59). Y521 and Y591 of full-length MPL are negative regulatory sites that function either as part of a lysosomal targeting motif (Y521) or via an interaction with adaptor protein AP2 (Y591) (Drachman and Kaushansky. Proc Natl Acad Sci USA. 1997 Mar. 18; 94(6):2350-5; and Hitchcock et al. Blood. 2008 Sep. 15; 112(6):2222-31). Y626 and Y631 of full-length MPL are positive regulatory sites (Drachman and Kaushansky. Proc Natl Acad Sci USA. 1997 Mar. 18; 94(6):2350-5) and the murine homolog of Y626 is required for cellular differentiation and the phosphorylation of Shc (Alexander et al. EMBO J. 1996 Dec. 2; 15(23):6531-40) and Y626 is also required for constitutive signaling in MPL with the W515A mutation described below (Pecquet et al. Blood. 2010 Feb. 4; 115(5):1037-48). MPL contains the Shc phosphotyrosine-binding binding motif NXXY (SEQ ID NO:307) where each X can be any amino acid (corresponding to amino acids 110-113 of SEQ ID NO: 283), and this tyrosine is phosphorylated and important for the TPO-dependent phosphorylation of Shc, SHIP, and STAT3 (Laminet et al. J Biol Chem. 1996 Jan. 5; 271(1):264-9; and van der Geer et al. Proc Natl Acad Sci USA. 1996 Feb. 6; 93(3):963-8). MPL also contains the STAT3 consensus binding sequence YXXQ (SEQ ID NO:308) where each X can be any amino acid (corresponding to amino acids 118-121 of SEQ ID NO: 283) (Stahl et al. Science. 1995 Mar. 3; 267(5202):1349-53). The tyrosine of this sequence can be phosphorylated and MPL is capable of partial STAT3 recruitment (Drachman and Kaushansky. Proc Natl Acad Sci USA. 1997 Mar. 18; 94(6):2350-5). MPL also contains the sequence YLPL (SEQ ID NO: 309) (corresponding to amino acid 113-116 of SEQ ID NO: 283), which is similar to the consensus binding site for STAT5 recruitment pYLXL (SEQ ID NO:310) where pY is phosphotyrosine and X can be any amino acid (May et al. FEBS Lett. 1996 Sep. 30; 394(2):221-6). Using computer simulations, Lee et al. found clinically relevant mutations in the transmembrane domain of MPL should activate MPL with the following order of activating effects: W515K (corresponding to the amino acid substitution W2K of SEQ ID NO: 283)>S505A (corresponding to the amino acid substitution S14A of SEQ ID NO:187)>W5151 (corresponding to the amino acid substitution W21 of SEQ ID NO: 283)>S505N (corresponding to the amino acid substitution S14N of SEQ ID NO:187, which was tested in Example 12 as part T075 (SEQ ID NO:188)) (PLoS One. 2011; 6(8):e23396). The simulations predicted these mutations could cause constitutive activation of JAK2, the kinase partner of MPL. In some embodiments, the intracellular portion of MPL can include one or more, or all the domains and motifs described herein that are present in SEQ ID NO: 283. In some embodiments, a transmembrane portion of MPL can include one or more, or all the domains and motifs described herein that are present in SEQ ID NO:187. The domains, motifs, and point mutations of MPL provided herein are known in the art and a skilled artisan would recognize that MPL intracellular signaling domains herein in illustrative embodiments would include one or more corresponding domains, motifs, and point mutations in that have been shown to promote proliferative activity and would not include that that have been shown to inhibit MPLs proliferative activity. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO: 283. In some embodiments, the intracellular domain derived from MPL has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, from about 300 aa to 350 aa, from about 350 aa to about 400 aa, from about 400 aa to about 450 aa, from about 450 aa to about 500 aa, from about 500 aa to about 550 aa, from about 550 aa to about 600 aa, or from about 600 aa to about 635 aa. In illustrative embodiments, the intracellular domain derived from MPL has a length of from about 30 aa to about 200 aa, for example, 30 aa to 150 aa, 30 aa to 119 aa, 30 aa to 121 aa, 30 aa to 122 aa, or 50 aa to 125 aa. In illustrative embodiments of lymphoproliferative elements that include a first intracellular domain derived from MPL, the second intracellular domain can be derived from CD79B.

In illustrative embodiments of any of the methods and compositions provided herein that include a lymphoproliferative element, the intracellular domain can be derived from a portion of the transmembrane protein CD79B, also known as B29; IGB; AGM6. The domains, motifs, and point mutations of CD79B that induce proliferation and/or survival of T cells and/or NK cells are known in the art and a skilled artisan can identify corresponding domains, motifs, and point mutations in CD79B polypeptides, some of which are discussed in this paragraph. CD79B contains an ITAM motif at residues 193-212 (corresponding to amino acids 16-30 of SEQ ID NO:211). CD79B has two tyrosines that are known to be phosphorylated, Y196 and Y207 (corresponding to Y16 and Y27 of SEQ ID NO: 211). In some embodiments, the intracellular portion of the transmembrane protein CD79B includes the ITAM motif and/or the known phosphorylation sites disclosed herein. The motif and phosphorylatable tyrosines of CD79B are known in the art and a skilled artisan will be able to identify corresponding motifs and phosphorylatable tyrosines in similar CD79B polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO: 211. In some embodiments, the intracellular domain derived from CD79B has a length of from about 30 aa to about 35 aa, from about 35 aa to about 40 aa, from about 40 aa to about 45 aa, or from about 45 aa to about 50 aa). In illustrative embodiments, the intracellular domain derived from CD79B has a length of from about 30 aa to about 50 aa. For example, a suitable CD79B intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids of the following sequence: LDKDDSKAGMEEDHT[YEGLDIDQTATYEDI]VTLRTGEVKWSVGEHPGQE (SEQ ID NO: 211), where the ITAM motif is set out in brackets. In illustrative embodiments of lymphoproliferative elements that include a second intracellular domain derived from CD79B, the first intracellular domain can be derived from CSF3R.

In illustrative embodiments of any of the methods and compositions provided herein that include a lymphoproliferative element, the intracellular domain can be derived from a portion of the transmembrane protein OSMR. The domains, motifs, and point mutations of OSMR that induce proliferation and/or survival of T cells and/or NK cells are known in the art and a skilled artisan can identify corresponding domains, motifs, and point mutations in OSMR polypeptides, some of which are discussed in this paragraph. OSMR contains a Box1 motif at amino acids 771-779 of isoform 3 (corresponding to amino acids 16-30 of SEQ ID NO:294). OSMR has two serines at amino acids 829 and 890 of isoform 3 that are known to be phosphorylated (serines at amino acids 65 and 128 of SEQ ID NO:294). In some embodiments, the intracellular portion of the protein OSMR can include the Box1 motif and the known phosphorylation sites disclosed herein. The motif and phosphorylatable serines of OSMR are known in the art and a skilled artisan will be able to identify corresponding motifs and phosphorylatable serines in similar OSMR polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:294. In some embodiments, the intracellular domain derived from OSMR has a length of from about 30 aa to about 35 aa, from about 35 aa to about 40 aa, from about 40 aa to about 45 aa, from about 45 aa to about 50 aa., from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, or from about 200 aa to about 250 aa.

In illustrative embodiments of any of the methods and compositions provided herein that include a lymphoproliferative element, the intracellular domain can be derived from a portion of the transmembrane protein PRLR. The domains, motifs, and point mutations of PRLR that induce proliferation and/or survival of T cells and/or NK cells are known in the art and a skilled artisan can identify corresponding domains, motifs, and point mutations in PRLR polypeptides, some of which are discussed in this paragraph. PRLR contains a growth hormone receptor binding domain at amino acids 185-261 of isoform 6 (corresponding to amino acids 28-104 of SEQ ID NO:295). The growth hormone receptor binding domain of PRLR is known in the art and a skilled artisan will be able to identify corresponding domain in similar PRLR polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:295. In some embodiments, the intracellular domain derived from PRLR has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, from about 300 aa to 350 aa, or from about 350 aa to about 400 aa.

In some embodiments, an intracellular domain of a lymphoproliferative element is derived from an intracellular portion of the transmembrane protein CD30 (also known as TNFRSF8, DlS166E, and Ki-1).

In illustrative embodiments of any of the methods and compositions provided herein that include a lymphoproliferative element, the intracellular domain can be derived from a portion of the protein CD28. The domains, motifs, and point mutations of CD28 that induce proliferation and/or survival of T cells and/or NK cells are known in the art and a skilled artisan can identify corresponding domains, motifs, and point mutations in CD28 polypeptides, some of which are discussed in this paragraph. Full-length CD28 contains a PI3-K- and Grb2-binding motif that corresponds to residues 12-15 of SEQ ID NOs:206 and 207 (Harada et al. J Exp Med. 2003 Jan. 20; 197(2):257-62). In some embodiments, a lymphoproliferative element that includes a CD28 intracellular domain can include the PI3-K- and Grb2-binding motif. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:206 or 207. In some embodiments, the intracellular domain derived from CD28 has a length of from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, or from about 35 aa to about 42 aa.

In illustrative embodiments of any of the methods and compositions provided herein that include a lymphoproliferative element, the intracellular domain can be derived from a portion of the protein ICOS. The domains, motifs, and point mutations of ICOS that induce proliferation and/or survival of T cells and/or NK cells are known in the art and a skilled artisan can identify corresponding domains, motifs, and point mutations in ICOS polypeptides, some of which are discussed in this paragraph. Unlike CD28, ICOS binds PI3-K and not Grb2. The PI3-K-binding motif of full-length ICOS corresponds to residues 19-22 of SEQ ID NO:225. A single amino acid substitution in this motif can lead to Grb2 binding by ICOS and increased IL-2 production (Harada et al. J Exp Med. 2003 Jan. 20; 197(2):257-62). This mutation corresponds to mutating phenylalanine 21 of SEQ ID NO:225 to an asparagine. A skilled artisan will understand how to mutate this residue in SEQ ID NO:225 and generate an ICOS intracellular domain that binds Grb2 in addition to PI3-K. In some embodiments, a lymphoproliferative element that includes an ICOS intracellular domain can include the PI3-K-binding motif. In some embodiments, a lymphoproliferative element that includes an ICOS intracellular domain can include the PI3-K-binding motif that has been mutated to additionally bind Grb2. ICOS also contains a membrane proximal motif in the cytoplasmic tail that is essential for ICOS-assisted calcium signaling (Leconte et al. Mol Immunol. 2016 November; 79:38-46). This calcium signaling-motif corresponds to residues 5-8 of SEQ ID NO:225. In some embodiments, a lymphoproliferative element that includes an ICOS intracellular domain can include the calcium-signaling motif. Two other conserved motifs have been identified in full-length ICOS. A first conserved motif at residues 170-179 (corresponding to residues 9-18 of SEQ ID NO:225) and a second conserved motif at residues 185-191 (corresponding to residues 24-30 of SEQ ID NO:225) (Pedros et al. Nat Immunol. 2016 July; 17(7):825-33). These two conserved motifs might have important function(s) in mediating downstream ICOS signaling. In some embodiments, a lymphoproliferative element that includes an ICOS intracellular domain can include at least one of the first or second conserved motifs. In some embodiments, a lymphoproliferative element that includes an ICOS intracellular domain does not include the first conserved motif, does not include the second conserved motif, or does not include the first and second conserved motifs. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:225. In some embodiments, the intracellular domain derived from ICOS has a length of from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, or from about 35 aa to about 38 aa.

In some embodiments, an intracellular domain of a chimeric lymphoproliferative element is derived from an intracellular portion of the transmembrane protein OX40 (also known as TNFRSF4, RP5-902P8.3, ACT35, CD134, OX-40, TXGPIL). The domains, motifs, and point mutations of OX40 that induce proliferation and/or survival of T cells and/or NK cells are known in the art and a skilled artisan can identify corresponding domains, motifs, and point mutations in OX40 polypeptides, some of which are discussed in this paragraph. OX40 contains a TRAF binding motif at residues 256-263 of full-length OX40 (corresponding to residues 20-27 of SEQ ID NO:296) that are important for binding TRAF1, TRAF2, TRAF3, and TRAF5 (Kawamata, S, et al. J Biol Chem. 1998 Mar. 6; 273(10):5808-14; Hori, T. Int J Hematol. 2006 January; 83(1):17-22). Full-length OX40 also contains a p⁸⁵ PI3K binding motif at residues 34-57. In some embodiments, when OX40 is present as an intracellular domain of a lymphoproliferative element, it includes the p85 PI3K binding motif of OX40. In some embodiments, an intracellular domain of OX40 can include the TRAF binding motif of OX40. In some embodiments, an intracellular domain of OX40 can bind TRAF1, TRAF2, TRAF3, and TRAF5. Lysines corresponding to amino acids 17 and 41 of SEQ ID NO: 296 are potentially negative regulatory sites that function as parts of ubiquitin targeting motifs. In some embodiments, one or both of these lysines in the intracellular domain of OX40 are mutated arginines or another amino acid. In some embodiments, a suitable intracellular domain of a lymphoproliferative element can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:57. In some of these embodiments, the intracellular domain of OX40 has a length of from about 20 aa to about 25 aa, about 25 aa to about 30 aa, 30 aa to about 35 aa, from about 35 aa to about 40 aa, from about 40 aa to about 45 aa, or from about 45 aa to about 50 aa. In illustrative embodiments, the intracellular domain of OX40 has a length of from about 20 aa to about 50 aa, for example 20 aa to 45 aa, or 20 aa to 42 aa.

In some embodiments, an intracellular domain of a chimeric lymphoproliferative element is derived from an intracellular portion of the transmembrane protein IFNAR2. The domains, motifs, and point mutations of IFNAR2 that induce proliferation and/or survival of T cells and/or NK cells are known in the art and a skilled artisan can identify corresponding domains, motifs, and point mutations in IFNAR2 polypeptides, some of which are discussed in this paragraph. Full-length IFNAR2 contains a Box1 motif and two Box2 motifs (known as Box2A and Box2B). (Usacheva A et al. J Biol Chem. 2002 Dec. 13; 277(50):48220-6). In some embodiments, a lymphoproliferative element that includes a IFNAR2 intracellular domain can include one or more of the Box1 or Box2 motifs. In illustrative embodiments, the IFNAR2 intracellular domain can include one or more of the Box1, Box2A, or Box2B motifs. IFNAR2 contains a JAK1-binding site (Gauzzi M C et al. Proc Natl Acad Sci USA. 1997 Oct. 28; 94(22):11839-44; Schindler et al. J Biol Chem. 2007 Jul. 13; 282(28):20059-63). In some embodiments, a lymphoproliferative element that includes a IFNAR2 intracellular domain can include the JAK1-binding site. In some embodiments, a suitable intracellular domain of a lymphoproliferative element can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:227 or 228. In some of these embodiments, the intracellular domain of IFNAR2 has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, or from about 200 aa to about 251 aa. In illustrative embodiments, the intracellular domain of OX40 has a length of from about 30 aa to about 251 aa, for example 30 aa to 67 aa.

In some embodiments, an intracellular domain of a chimeric lymphoproliferative element is derived from an intracellular portion of the transmembrane protein CSF3R. The domains, motifs, and point mutations of CSF3R that induce proliferation and/or survival of T cells and/or NK cells are known in the art and a skilled artisan can identify corresponding domains, motifs, and point mutations in CSF3R polypeptides, some of which are discussed in this paragraph. Full-length CSF3R contains a Box1 and Box2 motif as well as a Box3 motif (Nguyen-Jackson H T et al. G-CSF Receptor Structure, Function, and Intracellular Signal Transduction. Twenty Years of G-CSF, (2011) 83-105). In some embodiments, a lymphoproliferative element that includes a CSF3R intracellular domain can include one or more of the Box1, Box2, or Box3 motifs. CSF3R contains four tyrosine residues, Y704, Y729, Y744, and Y764 in full-length CSF3R, that are important for binding STAT3 (Y704 and Y744), SOCS3 (Y729), and Grb2 and p21Ras (Y764). In some embodiments, a lymphoproliferative element that includes a CSF3R intracellular domain can include one, two, three, or all of the tyrosine residues corresponding to Y704, Y729, Y744, and Y764 of full-length CSF3R. CSF3R contains two threonine residues, T615 and T618 in full-length CSF3R, that can increase receptor dimerization and activity when mutated to alanine and isoleucine, respectively (T615A and T618I) (Maxson et al. J Biol Chem. 2014 Feb. 28; 289(9):5820-7). In some embodiments, a lymphoproliferative element that includes a CSF3R intracellular domain can include one or more of the mutations corresponding to T615A and T618I. In some embodiments, a suitable intracellular domain of a lymphoproliferative element can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:216, 217, or 218. In some of these embodiments, the intracellular domain of CSF3R has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, or from about 200 aa to about 213 aa. In illustrative embodiments, the intracellular domain of CSF3R has a length of from about 30 aa to about 213 aa, for example from about 30 aa to about 186 or from about 30 aa to about 133 aa.

In some embodiments, an intracellular domain of a chimeric lymphoproliferative element is derived from an intracellular portion of the transmembrane protein EPOR. The domains, motifs, and point mutations of EPOR that induce proliferation and/or survival of T cells and/or NK cells are known in the art and a skilled artisan can identify corresponding domains, motifs, and point mutations in EPOR polypeptides, some of which are discussed in this paragraph. EPOR contains a Box1 (residues 257-264 of full-length EPOR) and Box2 (residues 303-313 of full-length EPOR) motif (Constantinescu SN. Trends Endocrinol Metab. 1999 December; 10(1):18-23). EPOR also contains an extended Box2 motif (residues 329-372) important for binding tyrosine kinase receptor KIT (Constantinescu SN. Trends Endocrinol Metab. 1999 December; 10(1):18-23). In some embodiments, a lymphoproliferative element that includes an EPOR intracellular domain can include one or more of the Box1, Box2, or extended Box2 motifs. EPOR also contains a short segment important for EPOR internalization (residues 267-276 of full-length EPOR). In some embodiments, a lymphoproliferative element that includes an EPOR intracellular domain does not include the internalization segment. In some embodiments, a suitable intracellular domain of a lymphoproliferative element can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NOs:219 or 220. In some of these embodiments, the intracellular domain of EPOR has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, or from about 200 aa to about 235 aa. In illustrative embodiments, the intracellular domain of EPOR has a length of from about 30 aa to about 235 aa.

In some embodiments, an intracellular domain of a chimeric lymphoproliferative element is derived from an intracellular portion of the transmembrane protein CD3G. The domains, motifs, and point mutations of CD3G that induce proliferation and/or survival of T cells and/or NK cells are known in the art and a skilled artisan can identify corresponding domains, motifs, and point mutations in CD3G polypeptides, some of which are discussed in this paragraph. Two serine residues, S123 and S126 of full-length CD3G have been shown to be phosphorylated in T cells in response to ionomycin (Davies et al. J Biol Chem. 1987 Aug. 15; 262(23):10918-21). In some embodiments, a lymphoproliferative element that includes a CD3G intracellular domain can include one or more of the serine residues corresponding to full-length S123 and S126. Furthermore, phosphorylation at S126 but not S123 was shown to be required for PKC-mediated down-regulation (Dietrich J et al. EMBO J. 1994 May 1; 13(9):2156-66). In some embodiments, a lymphoproliferative element that includes a CD3G intracellular domain can include the serine residue corresponding to full-length S123 and not include serine residue corresponding to full-length S126. In some embodiments, a lymphoproliferative element that includes a CD3G intracellular domain can include a non-phosphorylatable amino acid substitution at the serine residue corresponding to full-length S126. In illustrative embodiments, the amino acid substitution can be a serine to alanine mutation. Additionally, leucine to alanine mutations of either leucine of a di-leucine motif, L131 and L132 in full-length CD3G, was shown to prevent PKC-mediated down-regulation (Dietrich J et al. EMBO J. 1994 May 1; 13(9):2156-66). In some embodiments, a lymphoproliferative element that includes a CD3G intracellular domain can include at least one amino acid substitution at the leucine residues corresponding to L131 or L132 of full-length CD3G. In illustrative embodiments, the amino acid substitution can be a leucine to alanine mutation. In some embodiments, a suitable intracellular domain of a lymphoproliferative element can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:199. In some of these embodiments, the intracellular domain of CD3G has a length of from about 20 aa to about 25 aa, from about 25 aa to about 30 aa, from about 30 aa to about 35 aa, from about 35 aa to about 40 aa, or from about 40 aa to about 45 aa. In illustrative embodiments, the intracellular domain of CD3D has a length of from about 30 aa to about 45 aa.

The cytoplasmic domains of TNF receptors (TNFRs), which in illustrative embodiments can be TNFRSF4, TNFRSF8, TNFRSF9, TNFRSF14, or TNFRSF18, can recruit signaling molecules, including TRAFs (TNF receptor-associated factors) and/or “death domain” (DD) molecules. The domains, motifs, and point mutations of TNFRs that induce proliferation and/or survival of T cells and/or NK cells are known in the art and a skilled artisan can identify corresponding domains, motifs, and point mutations in TNFR polypeptides, some of which are discussed in this paragraph. In mammals, there are at least six TRAF molecules and a number of nonreceptor DD molecules. Receptors and adaptor proteins that bind to TRAFs share short consensus TRAF-binding motifs that are known in the art (Meads et al. J Immunol. 2010 Aug. 1; 185(3):1606-15). The DD-binding motif is a roughly 60 amino acid globular bundle of 6 conserved α-helices that is also known in the art (Locksley R M et al. Cell. 2001 Feb. 23; 104(4):487-501). A skilled artisan will be able to identify the TRAF- and/or DD-binding motif in the different TNFR families using, for example, sequence alignments to known binding motifs. TNFRs can recruit TRADD and TRAF2, resulting in the activation of NF-κB, MAPK, and JNK (Sedger and McDermott. Cytokine Growth Factor Rev. 2014 August; 25(4):453-72). In some embodiments, a lymphoproliferative element that includes a TNFR intracellular domain can include one or more TRAF-binding motifs. In some embodiments, a lymphoproliferative element that includes a TNFR intracellular domain does not include a DD-binding motif, or has one or more DD-binding motifs deleted or mutated within the intracellular domain. In some embodiments, a lymphoproliferative element that includes a TNFR intracellular domain can recruit TRADD and/or TRAF2. TNFRs also include cysteine-rich domains (CRDs) that are important for ligand binding (Locksley R M et al. Cell. 2001 Feb. 23; 104(4):487-501). In some embodiments, a lymphoproliferative element that includes a TNFR intracellular domain does not include a TNFR CRD.

Lymphoproliferative elements and CLEs that can be included in any of the aspects disclosed herein, can be any of the LEs or CLEs disclosed in WO2019/055946. CLEs were disclosed therein that promoted proliferation in cell culture of PBMCs that were transduced with lentiviral particles encoding the CLEs between day 7 and day 21, 28, 35 and/or 42 after transduction. Furthermore, CLEs were identified therein, that promoted proliferation in vivo in mice in the presence or absence of an antigen recognized by a CAR, wherein T cells expressing one of the CLEs and the CAR were introduced into the mice. As exemplified therein, tests and/or criteria can be used to identify whether any test polypeptide, including LEs, or test domains of an LE, such as a first intracellular domain, or a second intracellular domain, or both a first and second intracellular domain, are indeed LEs or effective intracellular domains of LEs, or especially effective LEs or intracellular domains of LEs. Thus, in certain embodiments, any aspect or other embodiment provided herein that includes an LE or a polynucleotide or nucleic acid encoding an LE can recite that the LE meets, or provides the property of, or is capable of providing and/or possesses the property of, any one or more of the identified tests or criteria for identifying an LE provided herein, or that a cell genetically modified and/or transduced with a retroviral particle, such as a lentiviral particle encoding the LE, is capable of providing, is adapted for, possesses the property of, and/or is modified for achieving the results of one or more of the recited tests. In one embodiment, the LE provides, is capable of providing and/or possesses the property of, (or a cell genetically modified and/or transduced with a retroviral particle encoding the LE is capable of providing, is adapted for, possesses the property of, and/or is modified for) improved expansion to pre-activated PBMCs transduced with a lentivirus comprising a nucleic acid encoding the LE and an anti-CD19 CAR comprising a CD3 zeta intracellular activating domain but no co-stimulatory domain, between day 7 and day 21, 28, 35, and/or 42 of in vitro culturing post-transduction in the absence of exogenously added cytokines, compared to a control retroviral particle, e.g. lentiviral particle under identical conditions. In some embodiments, a lymphoproliferative element test for improved or enhanced survival, expansion, and/or proliferation of cells transduced with a retroviral particle (e.g. lentiviral particle) having a genome encoding a test construct encoding a putative LE (test cells) can be performed based on a comparison to control cells, which can be, for example, either untransduced cells or cells transduced with a control retroviral (e.g. lentiviral) particle identical to the lentiviral particle comprising the nucleic acid encoding the lymphoproliferative element, but lacking the lymphoproliferative element, or lacking the intracellular domain or domains of the test polypeptide construct but comprising the same extracellular domain, if present, and the same transmembrane region or membrane targeting region of the respective test polypeptide construct. In some embodiments control cells are transduced with a retroviral particle (e.g. lentiviral particle) having a genome encoding a lymphoproliferative element or intracellular domain(s) thereof, identified herein as exemplifying a lymphoproliferative element. In such an embodiment, the test criteria can include that there is at least as much enrichment, survival and/or expansion, or no statistical difference of enrichment, survival, and/or expansion when the test is performed using a retroviral particle (e.g. lentiviral particle) having a genome encoding a test construct versus encoding the control lymphoproliferative element, typically by analyzing cells transcribed therewith. Exemplary or illustrative embodiments of lymphoproliferative elements herein, in some embodiments, are illustrative embodiments of control lymphoproliferative elements for such a test.

In some embodiments, this test for an improved property of a putative or test lymphoproliferative element is performed by performing replicates and/or performing a statistical test. A skilled artisan will recognize that many statistical tests can be used for such a lymphoproliferative element test. Contemplated for such a test in these embodiments would be any such test known in the art. In some embodiments, the statistical test can be a T-test or a Mann-Whitney-Wilcoxon test. In some embodiments, the normalized enrichment level of a test construct is significant at a p-value of less than 0.1, or less than 0.05, or less than 0.01.

In another embodiment, the LE provides, is capable of providing and/or possesses the property of (or a cell genetically modified and/or transduced with the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold expansion, or between 1.5 fold and 25-fold expansion, or between 2-fold and 20-fold expansion, or between 2-fold and 15-fold expansion, or between 5-fold and 25-fold expansion, or between 5-fold and 20-fold expansion, or between 5-fold and 15-fold expansion, of pre-activated PBMCs transduced with a nucleic acid encoding the LE when transduced along with an anti-CD19 CAR comprising a CD3 zeta intracellular activating domain but no co-stimulatory domain, between day 7 and day 21, 28, 35, and/or 42 of in vitro culturing in the absence of exogenously added cytokines. In some embodiments, the test is performed in the presence of PBMCs, for example at a 1:1 ratio of transduced cells to PBMCs, which can be for example, from a matched donor, and in some embodiments, the test is performed in the absence of PBMCs. In some embodiments, the analysis of expansion for any of these tests is performed as illustrated in WO2019/055946. In some embodiments, the test can include a further statistical test and a cut-off such as a P value below 0.1, 0.05, or 0.01, wherein a test polypeptide or nucleic acid encoding the same, needs to meet one or both thresholds (i.e. fold expansion and statistical cutoff).

For any of the lymphoproliferative element tests provided herein, the number of test cells and the number of control cells can be compared between day 7 and day 14, 21, 28, 35, 42 or 60 post-transduction. In some embodiments, the numbers of test and control cells can be determined by sequencing DNA and counting the occurrences of unique identifiers present in each construct. In some embodiments, the numbers of test and control cells can be counted directly, for example with a hemocytometer or a cell counter. In some embodiments, all the test cells and control cells can be grown within the same vessel, well or flask. In some embodiments, the test cells can be seeded in one or more wells, flasks or vessels, and the control cells can be seeded in one or more flasks or vessels. In some embodiments, test and control cells can be seeded individually into wells or flasks, e.g., one cell per well. In some embodiments, the numbers of test cells and control cells can be compared using enrichment levels. In some embodiments, the enrichment level for a test or control construct can be calculated by dividing the number of cells at a later time point (day 14, 21, 28, 35, or day 45) by the number of cells at day 7 for each construct. In some embodiments, the enrichment level for a test or control construct can be calculated by dividing the number of cells at a time point (day 14, 21, 28, 35, or day 45) by the number of cells at that time point for untransduced cells. In some embodiments, the enrichment level of each test construct can be normalized to the enrichment level of the respective control construct to generate a normalized enrichment level. In some embodiments, a LE encoded in the test construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level. Enrichment can be measured, for example, by direct cell counting. Cutoff values can be based on a single test, or two, three, four, or five repeats, or based on many repeats. The cutoff can be met when a lymphoproliferative element meets one or more repeat tests, or meets or exceeds a cutoff for all repeats. In some embodiments, the enrichment is measured as log₂((normalized count data on the test day+1)/(normalized count data on day 7+1)).

As illustrated in WO2019/055946, CLEs were identified from libraries of constructs that included constructs that encoded test chimeric polypeptides that were designed to comprise an intracellular domain believed to induce proliferation and/or survival of lymphoid or myeloid cells, and an anti-CD19 CAR that comprised an intracellular activating domain but not a co-stimulatory domain. Preactivation, which was performed overnight at 37° C., was performed in a preactivation reaction mixture comprising PBMCs, a commercial media for lymphocytes (Complete OpTmizer™ CTS™ T-Cell Expansion SFM), recombinant human interleukin-2 (100IU/ml) and anti-CD3 Ab (OKT3) (50 ng/ml). Following preactivation, transduction was performed overnight at 37° C. after addition of test and control lentiviral particles to the preactivation reaction mixtures at a multiplicity of infection (MOI) of 5. Some control lentiviral particles contained constructs encoding polypeptides with extracellular and transmembrane domains but no intracellular domains. In contrast, the test lentiviral particles contained constructs encoding polypeptides with extracellular and transmembrane domains and either one or two intracellular domains. Following transduction, Complete OpTmizer™ CTS™ T-Cell Expansion SFM was added to dilute the reaction mixture 5- to 20-fold and the cells were cultured for up to 45 days at 37° C. After day 7 post-transduction, cultures were either “fed” additional untransduced donor matched PBMCs or not (“unfed”). No additional cytokines (e.g. IL-2, IL-7, or IL-15 and no other lymphoid mitogenic agent) were added to these cultures that were not present in the commercial media, after the transduction reaction mixtures were initially formed. Expansion was measured by analyzing enrichment of cell counts actually counted as nucleic acid sequence counts of unique identifiers for each construct in the mixed cultured PBMC cell populations, such that enrichment was positive as calculated as the logarithm in base 2 of the ratio between normalized count at the last day for analysis plus one to the count at day 7 plus one. Additional details regarding the tests performed to identify the LEs are illustrated in WO2019/055946, including experimental conditions.

As illustrated in WO2019/055946, test constructs were identified as CLEs because the CLEs induced proliferation/expansion in these fed or unfed cultures without added cytokines such as IL-2 between days 7 and day 21, 28, 35, and/or 42. For example, as illustrated in WO2019/055946, effective CLEs were identified by identifying test CLEs that provided increased expansion of these in vitro cultures, whether fed or unfed with untransduced PBMCs, between day 7 and day 21, 28, 35, and/or 42 post-transduction, compared to control constructs that did not include any intracellular domains. WO2019/055946 discloses that at least one and typically more than one test CLE that included an intracellular domain from a test gene provided more expansion than every control construct that was present at day 7 post-transduction, that did not include an intracellular domain. WO2019/055946 further provides a statistical method that was used to identify exceptionally effective genes with respect to a first intracellular domain, and one or more exemplary intracellular domain(s) from these genes. The method used a Mann-Whitney-Wilcoxon test and a false discovery cutoff rate of less than 0.1 or less than 0.05. WO2019/055946 identified especially effective genes for the first intracellular domain or the second intracellular domain, for example, by analyzing scores for genes calculated as combined score for all constructs with that gene. Such analysis can use a cutoff of greater than 1, or greater than negative control constructs without any intracellular domains, or greater than 2, as shown for some of the tests disclosed in WO2019/055946.

In another embodiment, the LE provides, is capable of providing and/or possesses the property of (or a cell genetically modified and/or transduced with the LE is capable of providing, is adapted for, possesses the property of, and/or is modified for) driving T cell expansion in vivo. For example, the in vivo test can utilize a mouse model and measure T cell expansion at 15 to 25 days in vivo, or at 19 to 21 days in vivo, or at approximately 21 days in vivo, after T cells are contacted with lentiviral vectors encoding the LEs, are introduced into the mice, as disclosed in WO2019/055946,

In exemplary aspects and embodiments that include a LE, which typically include a CAR, such as methods provided herein for genetically modifying, genetically modified and/or transduced cells, and uses thereof, the genetically modified cell is modified so as to possess new properties not previously possessed by the cell before genetic modification and/or transduction. Such a property can be provided by genetic modification with a nucleic acid encoding a CAR or a LE, and in illustrative embodiments both a CAR and a LE. For example, in certain embodiments, the genetically modified and/or transduced cell is capable of, is adapted for, possesses the property of, and/or is modified for survival and/or proliferation in ex vivo culture for at least 7, 14, 21, 28, 35, 42, or 60 days or from between day 7 and day 14, 21, 28, 35, 42 or 60 post-transduction, in the absence of added IL-2 or in the absence of added cytokines such as IL-2, IL-15, or IL-7, and in certain illustrative embodiments, in the presence of the antigen recognized by the CAR where the method comprises genetically modifying using a retroviral particle having a pseudotyping element and optionally a separate or fused activation domain on its surface and typically does not require pre-activation.

By capable of enhanced survival and/or proliferation in certain embodiments, it is meant that the genetically modified and/or transduced cell exhibits, is capable of, is adapted for, possesses the property of, and/or is modified for improved survival or expansion in ex vivo or in vitro culture in culture media in the absence of one or more added cytokines such as IL-2, IL-15, or IL-7, or added lymphocyte mitogenic agent, compared to a control cell(s) identical to the genetically modified and/or transduced cell(s) before it was genetically modified and/or transduced or to a control cell that was transduced with a retroviral particle identical to an on-test retroviral particle that comprises an LE or a putative LE, but without the LE or the intracellular domains of the LE, wherein said survival or proliferation of said control cell(s) is promoted by adding said one or more cytokines, such as IL-2, IL-15, or IL-7, or said lymphocyte mitogenic agent to the culture media. By added cytokine or lymphocyte mitogenic agent, it is meant that cytokine or lymphocyte mitogenic agent is added from an exogenous source to a culture media such that the concentration of said cytokine or lymphocyte mitogenic agent is increased in the culture media during culturing of the cell(s) compared to the initial culture media, and in some embodiments can be absent from the initial culture media before said adding. By “added” or “exogenously added”, it is meant that such cytokine or lymphocyte mitogenic agent is added to a lymphocyte media used to culture the genetically modified and/or transduced cell after the genetically modifying, where the culture media may or may not already possess the cytokine or lymphocyte mitogenic agent. All or a portion of the media that includes a mixture of multiple media components is typically stored and in illustrative embodiments has been shipped to a site where the culturing takes place, without the exogenously added cytokine(s) or lymphocyte mitogenic agent(s). The lymphocyte media in some embodiments is purchased from a supplier, and a user such as a technician not employed by the supplier and not located within a supplier facility, adds the exogenously added cytokine or lymphocyte mitogenic agent to the lymphocyte media and then the genetically modified and/or transduced cells are cultured in the presence or absence of such exogenously added cytokine or lymphocyte mitogenic agent.

In some embodiments, improved or enhanced survival, expansion, and/or proliferation can be shown as an increase in the number of cells determined by sequencing DNA from cells transduced with retroviral particle (e.g. lentiviral particle) having a genome encoding CLEs and counting the occurrences of sequences present in unique identifiers from each CLE. In some embodiments, improved survival and/or improved expansion can be determined by counting the cells directly, for example with a hemocytometer or a cell counter, at each time point. In some embodiments, improved survival and/or improved expansion and/or enrichment can be calculated by dividing the number of cells at the later time point (day 21, 28, 35, and/or day 45) by the number of cells at day 7 for each construct. In some embodiments, the cells can be counted by hemocytometer or cell counters. In some embodiments, the enrichment level determined using the nucleic acid counts or the cell counts of each specific test construct can be normalized to the enrichment level of the respective control construct, i.e., the construct with the same extracellular domain and transmembrane domain but lacking the intracellular domains present in the test construct. In these embodiments, the LE encoded in the construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level.

In illustrative embodiments of any of the methods, uses, genetically modified T cells and/or NK cells, and other composition aspects provided herein that include a lymphoproliferative element, the lymphoproliferative element can include an intracellular domain or a fragment thereof that includes an intracellular signaling domain from any of the genes having a P3 signaling domain with or without a P4 domain, or from any of the genes having a P4 domain wherein the P3 domain was a linker, in the CLEs identified in Tables 4 to 8 herein, which promote T cell, e.g. CAR-T cell, expansion in vivo. In illustrative embodiments of any of the methods, uses, and composition aspects provided herein that include a lymphoproliferative element having a P3 and P4 domain, the lymphoproliferative element can include at the P4 position, an intracellular domain or a fragment thereof that includes a signaling domain from any of the genes having a P4 signaling domain in constructs having a P3 and a P4 signaling domain in the CLEs identified in Tables 4 to 8 herein, which promote T cell, e.g. CAR-T cell, expansion in vivo. In illustrative embodiments of any of the methods, uses, and composition aspects provided herein that include a lymphoproliferative element, the lymphoproliferative element can include an intracellular domain or a fragment thereof that includes a signaling domain from any of the genes having a P3 signaling domain and a signaling domain from any of the genes having a P4 domain in the same CLE, in illustrative embodiments in the P3 and P4 positions respectively, in any of the CLEs identified in Tables 4 to 8 herein, which promote T cell, e.g. CAR-T cell, expansion in vivo. In any of the CLEs of embodiments provided in this paragraph, the P2 domain can be from any of the genes identified as having a P2 part in CLEs found in Tables 4 to 8 herein. Furthermore, the CLEs can include in some illustrative embodiments a P1 domain from Tables 4 to 8.

In illustrative embodiments of any of the methods, uses, genetically modified T cells and/or NK cells, and other composition aspects provided herein that include a lymphoproliferative element, the lymphoproliferative element can include a P3 signaling domain from any of the CLEs identified in Tables 4 to 8 herein, which promote T cell, e.g. CAR-T cell, expansion in vivo, or a P4 signaling domain in a construct having no P3 signaling domain, from any of the CLEs identified in Tables 4 to 8 herein, which promote T cell, e.g. CAR-T cell, expansion in vivo. In illustrative embodiments of any of the methods, uses, and composition aspects provided herein that include a lymphoproliferative element having a P3 and P4 domain, the lymphoproliferative element can include at the P4 position, a P4 signaling domain in constructs having a P3 and a P4 signaling domain in the CLEs identified in Tables 4 to 8 herein, which promote T cell, e.g. CAR-T cell, expansion in vivo. In illustrative embodiments of any of the methods, uses, and composition aspects provided herein that include a lymphoproliferative element, the lymphoproliferative element can include a P3 signaling domain and a P4 signaling domain in the P3 and P4 positions respectively, from any one of the CLEs identified in Tables 4 to 8 herein, which promote T cell, e.g. CAR-T cell, expansion in vivo. Furthermore, the CLEs can include in some illustrative embodiments, a P1 domain from Tables 4 to 8. In any of the CLEs of embodiments provided in this paragraph, the P2 domain can comprise or be any P2 domain from a CLE found in Tables 4 to 8 herein, or in illustrative embodiments, a lymphoproliferative element can include a P2 domain, P3 domain and P4 domain, and optionally P1 domain, all from the same CLE identified in Tables 4 to 8 herein. In certain illustrative embodiments of any of the methods, uses, genetically modified T cells and/or NK cells, and other composition aspects provided herein that include a lymphoproliferative element, the lymphoproliferative element can have P3 and P4 domains S121-S212 or S186-S053, or P2, P3, and P4 domains T001-S121-S212 or T044-S186-S053 optionally with a P1 domain E008 or E006.

In some embodiments, the lymphoproliferative element can include a cytokine receptor or a fragment that includes a signaling domain thereof. In some embodiments, the cytokine receptor can be CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGFβ decoy receptor, TNFRSF4, TNFRSF8, TNFRSF9, TNFRSF14, or TNFRSF18. In some embodiments, the cytokine receptor can be CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL13RA1, IL13RA2, IL15RA, IL17RA, IL17RB, IL17RC, IL17RE, IL18R1, IL18RAP, IL20RA, IL20RB, IL22RA1, IL27RA, IL31RA, LEPR, LIFR, MPL, OSMR, PRLR, TNFRSF4, TNFRSF8, TNFRSF9, TNFRSF14, or TNFRSF18.

In illustrative embodiments, the lymphoproliferative element can comprise an intracellular domain from the cytokine receptors CD27, CD40, CRLF2, CSF2RA, CSF3R, EPOR, GHR, IFNAR1, IFNAR2, IFNGR2, IL1R1, IL1RL1, IL2RA, IL2RG, IL3RA, IL5RA, IL6R, IL7R, IL9R, IL10RB, IL11RA, IL12RB1, IL13RA1, IL13RA2, IL15RA, IL17RB, IL18R1, IL18RAP, IL20RB, IL22RA1, IL27RA, IL31RA, LEPR, MPL, OSMR, PRLR, TNFRSF4, TNFRSF8, TNFRSF9, TNFRSF14, or TNFRSF18In illustrative embodiments, the intracellular domain in a lymphoproliferative element comprises a domain from CD40, CRLF2, CSF2RA, CSF3R, EPOR, FCGR2A, IFNAR2, IFNGR2, IL1R1, IL3RA, IL7R, IL10RB, IL11RA, IL12RB1, IL13RA2, IL18RAP, IL31RA, MPL, MYD88, TNFRSF14, or TNFRSF18, which were present in constructs that showed particularly noteworthy enrichments in an initial screen and a repeated screen as disclosed in WO2019/055946.

In illustrative embodiments, the lymphoproliferative element can comprise a costimulatory domain from CD27, CD28, OX40 (also referred to as TNFRSF4), GITR (also referred to as TNFRSF18), or HVEM (also referred to as TNFRSF14). In some embodiments, a lymphoproliferative element comprising a costimulatory domain from OX40 does not comprise an intracellular domain from CD3Z, CD28, 4-1BB, ICOS, CD27, BTLA, CD30, GITR, or HVEM. In some embodiments, a lymphoproliferative element comprising a costimulatory domain from GITR does not comprise an intracellular domain from CD3Z, CD28, 4-1BB, ICOS, CD27, BTLA, CD30, or HVEM. In some embodiments, a lymphoproliferative element comprising a costimulatory domain from CD28 does not comprise an intracellular domain from CD3Z, 4-1BB, ICOS, CD27, BTLA, CD30, or HVEM. In some embodiments, a lymphoproliferative element comprising a costimulatory domain from OX40, CD3Z, CD28, 4-1BB, ICOS, CD27, BTLA, CD30, GITR, or HVEM does not comprise a coiled-coil spacer domain N-terminal of the transmembrane domain. In some embodiments, a lymphoproliferative element comprising a costimulatory domain from GITR does not comprise an intracellular domain from CD3Z that is N-terminal of the costimulatory domain of GITR.

In certain illustrative embodiments, the lymphoproliferative element comprises an intracellular domain of CD40, MPL and IL2Rb. In some embodiments, the lymphoproliferative element can be other than a cytokine receptor. In some embodiments, the lymphoproliferative element other than a cytokine receptor can include an intracellular signaling domain from CD2, CD3D, CD3G, CD3Z, CD4, CD8RA, CD8RB, CD28, CD79A, CD79B, FCER1G, FCGR2A, FCGR2C, or ICOS.

In some embodiments, a lymphoproliferative element, including a CLE, comprises an intracellular activating domain as disclosed hereinabove. In some illustrative embodiments a lymphoproliferative element is a CLE comprising an intracellular activating domain comprising an ITAM-containing domain, as such, the CLE can comprise an intracellular activating domain having at least 80%, 90%, 95%, 98%, or 100% sequence identity to the CD3Z, CD3D, CD3E, CD3G, CD79A, CD79B, DAP12, FCERIG, FCGR2A, FCGR2C, DAP10/CD28, or ZAP70 domains provided herein wherein the CLE does not comprise an ASTR. In certain illustrative embodiments, the intracellular activating domain is an ITAM-containing domain from CD3D, CD3G, CD3Z, CD79A, CD79B, FCER1G, FCGR2A, or FCGR2C. CLEs comprising these intracellular activating domains are illustrated in WO2019/055946, as being effective at promoting proliferation of PBMCs ex vivo in cultures in the absence of exogenous cytokines such as exogenous IL-2. In some embodiments, provided herein are CLEs comprising an intracellular domain from CD3D, CD3G, CD3Z, CD79A, FCER1G.

In some embodiments, one or more domains of a lymphoproliferative element is fused to a modulatory domain, such as a co-stimulatory domain, and/or an intracellular activating domain of a CAR. In some embodiments of the composition and method aspects for transducing lymphocytes in whole blood, one or more intracellular domains of a lymphoproliferative element can be part of the same polypeptide as a CAR or can be fused and optionally functionally connected to some components of CARs. In still other embodiments, an engineered signaling polypeptide can include an ASTR, an intracellular activation domain (such as a CD3 zeta signaling domain), a co-stimulatory domain, and a lymphoproliferative domain. Further details regarding co-stimulatory domains, intracellular activating domains, ASTRs and other CAR domains, are disclosed elsewhere herein.

In some embodiments, the lymphoproliferative element is not a polypeptide, but rather comprises an inhibitory RNA. In some embodiments, methods, uses, compositions, and products of processes according to any aspect herein include both a lymphoproliferative element comprising an inhibitory RNA and a lymphoproliferative element that is an engineered signaling polypeptide. In embodiments where a lymphoproliferative element is or includes an inhibitory RNA, or multiple inhibitory RNAs, the inhibitory RNA or multiple inhibitory RNAs, can have any of the structures identified elsewhere herein, for example in the Inhibitory RNA Molecules section herein. In some embodiments, the inhibitory RNA can be a miRNA that stimulates the STAT5 pathway typically by potentiating activation of STAT5 by degrading or causing down-regulation of a negative regulator in the SOCS pathway. Inhibitory RNA lymphoproliferative elements can target any of the mRNAs identified in the Inhibitory RNA Molecules section herein or elsewhere herein.

In illustrative embodiments, as exemplified herein, such inhibitory RNA (e.g. miRNAs) can be located in introns in packaging cells and/or a replication incompetent recombinant retroviral particle genome and/or a retroviral vector, typically with expression driven by a promoter that is active in a T cell and/or NK cell. Not to be limited by theory, inclusion of introns in transcription units are believed to result in higher expression and/or stability of transcripts. As such, the ability to place miRNAs within introns of a retroviral genome adds to the teachings of the present disclosure that overcome challenges in the prior art of trying to get maximum activities into the size restrictions of a retroviral, such as a lentivirus genome. In some embodiments, 1, 2, 3, 4, 5, 6, 7, 8, 9, or 10 miRNAs, in illustrative embodiments between 2 and 5, for example 4 miRNAs, one or more of which each bind nucleic acids encoding one or more of the targets disclosed herein, can be included in the recombinant retroviral genome and delivered to a target cell, for example T cells and/or NK cells, using methods provided herein. In fact, as provided herein 1, 2, 3, or 4 miRNAs can be delivered in a single intron such as the EF1-a intron.

In some embodiments, the lymphoproliferative element comprises MPL, or is MPL, or a variant and/or fragment thereof, including a variant and/or fragment that includes at least 75, 80, 85, 90, 95, 96, 97, 98, 99, or 100% of the intracellular domain of MPL, with or without a transmembrane and/or extracellular domain of MPL, and/or has at least 75, 80, 85, 90, 95, 96, 97, 98, 99, or 100% sequence identity to the intracellular domain of MPL, with or without a transmembrane and/or extracellular domain of MPL, wherein the variant and/or fragment retains the ability to promote cell proliferation of PBMCs, and in some embodiments T cells. In illustrative embodiments, the lymphoproliferative element comprises an intracellular domain of MPL, or a variant or fragment thereof that includes at least 75, 80, 85, 90, 95, 96, 97, 98, 99, or 100% of the intracellular domain of MPL, and the lymphoproliferative element does not comprise a transmembrane domain of MPL. In some embodiments, the lymphoproliferative element comprises an intracellular domain of MPL, or a variant or fragment thereof that includes at least 75, 80, 85, 90, 95, 96, 97, 98, 99, or 100% of the intracellular domain of MPL, and the lymphoproliferative element comprises a transmembrane domain of MPL. In some embodiments, a cell expressing the lymphoproliferative element comprising an intracellular and transmembrane domain of MPL can be contacted with, exposed to, or treated with eltrombopag. Not to be limited by theory, eltrombopag binds to the transmembrane domain of MPL and induces the activation of the intracellular domain of MPL. In some embodiments, an MPL fragment included in the compositions and methods herein has and/or retains a JAK-2 binding domain. In some embodiments, an MPL fragment included herein has or retains the ability to activate a STAT. The full intracellular domain of MPL is SEQ ID NO:283 (part S186 as illustrated in WO2019/055946). MPL is the receptor for thrombopoietin. Several cytokines such as thrombopoietin and EPO are referred to in the literature and herein as either a hormone or a cytokine.

In some embodiments, which provide separate aspects of the present disclosure, provided herein are chimeric polypeptides that are chimeric lymphoproliferative elements (CLEs), as well as isolated polynucleotides and nucleic acid sequences that encode the same. CLEs can include any of the domains and/or domains derived from specific genes discussed in the section. Similarly, the isolated polynucleotides and nucleic acid sequences encoding CLEs can encode as part of the CLE any of the domains and/or domains derived from specific genes discussed in this section.

Lymphoproliferative elements provided herein typically include a transmembrane domain. For example, the transmembrane domain can have 80%, 85%, 90%, 95%, 97%, 98%, 99% or 100% sequence identity to any one of the transmembrane domains from the following genes and representative sequences disclosed in WO2019/055946: CD8 beta, CD4, CD3 zeta, CD28, CD134, CD7, CD2, CD3D, CD3E, CD3G, CD3Z, CD4, CD8A CD8B, CD27, CD28, CD40, CD79A, CD79B, CRLF2, CRLF2, CSF2RA, CSF2RB, CSF2RB, CSF3R, EPOR, FCER1G, FCGR2C, FCGRA2, GHR, GHR, ICOS, IFNAR, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL27RA, IL31RA, LEPR, LIFR, MPL, OSMR, PRLR, TNFRSF4, TNFRSF8, TNFRSF9, TNFRSF14, and TNFRSF18. Transmembrane (TM) domains suitable for use in any engineered signaling polypeptide include, but are not limited to, constitutively active cytokine receptors, the TM domain from LMP1, and TM domains from type 1 TM proteins comprising a dimerizing motif, as discussed in more detail herein. In any of the aspects disclosed herein containing the transmembrane domain from a type I transmembrane protein, the transmembrane domain can be a Type I growth factor receptor, a hormone receptor, a T cell receptor, or a TNF-family receptor.

Eltrombopag is a small molecule activator of the thrombopoietin receptor MPL (also known as TPOR). In some aspects a cell expressing an LE comprising a MPL transmembrane domain, can be exposed to or contacted with eltrombopag, or a patient or subject to which such a cell has been infused, can be treated with eltrombopag. Upon said contacting or treating, the proliferative and/or survival properties of the LE are activated and provided to the cell, thereby increasing survival and/or proliferation of the cell compared to the absence of the eltrombopag. Not to be limited by theory, binding of eltrombopag occurs in the transmembrane domain and can activate one or more intracellular domains that are part of the same polypeptide. A skilled artisan will understand the amount of eltrombopag to be used to activate a CLE comprising a MPL transmembrane domain.

In some embodiments, CLEs include both an extracellular portion and a transmembrane portion that is from the same protein, in illustrative embodiments the same receptor, either of which in illustrative embodiments is a mutant, thus forming an extracellular and transmembrane domain. These domains can be from a cytokine receptor, or a mutant thereof, or a hormone receptor, or a mutant thereof in some embodiments that have been reported to be constitutively active when expressed at least in some cell types. In illustrative embodiments, such extracellular and transmembrane domains do not include a ligand binding region. It is believed that such domains do not bind a ligand when present in CLEs and expressed in B cells, T cells, and/or NK cells. Mutations in such receptor mutants can occur in the transmembrane region or in the extracellular juxtamembrane region. Not to be limited by theory, a mutation in at least some extracellular-transmembrane domains of CLEs provided herein, are responsible for signaling of the CLE in the absence of ligand, by bringing activating chains together that are not normally together, or by changing the confirmation of a linked transmembrane and/or intracellular domain.

Exemplary extracellular and transmembrane domains for CLEs of embodiments that include such domains, in illustrative embodiments, are extracellular regions, typically less than 30 amino acids of the membrane-proximal extracellular domains along with transmembrane domains from mutant receptors that have been reported to be constitutive, that is not require ligand binding for activation of an associated intracellular domain. In illustrative embodiments, such extracellular and transmembrane domains include IL7RA Ins PPCL, CRLF2 F232C, CSF2RB V449E, CSF3R T640N, EPOR L251C I252C, GHR E260C I270C, IL27RA F523C, and MPL S505N. In some embodiments, the extracellular and transmembrane domain does not comprise more than 10, 20, 25 30 or 50 consecutive amino acids that are identical in sequence to a portion of the extracellular and/or transmembrane domain of IL7RA, or a mutant thereof. In some embodiments, the extracellular and transmembrane domain is other than IL7RA Ins PPCL. In some embodiments, the extracellular and transmembrane does not comprise more than 10, 20, 25, 30, or 50 consecutive amino acids that are identical in sequence to a portion of the extracellular and/or transmembrane domain of IL15R.

In one embodiment of this aspect, an LE provided herein comprises an extracellular domain, and in illustrative embodiments, the extracellular domain comprises a dimerizing motif. In illustrative embodiments of this aspect, the extracellular domain comprises a leucine zipper. In some embodiments, the leucine zipper is from a jun polypeptide, for example c-jun. In certain embodiments the c-jun polypeptide is the c-jun polypeptide region of ECD-11.

In embodiments of any of these aspects and embodiments wherein the transmembrane domain is a type I transmembrane protein, the transmembrane domain can be a Type I growth factor receptor, a hormone receptor, a T cell receptor, or a TNF-family receptor. In an embodiment of any of the aspects and embodiments wherein the chimeric polypeptide comprises an extracellular domain and wherein the extracellular domain comprises a dimerizing motif, the transmembrane domain can be a Type I cytokine receptor, a hormone receptor, a T cell receptor, or a TNF-family receptor.

Exemplary transmembrane domains include any transmembrane domain that was illustrated in WO2019/055946. In some embodiments, the transmembrane domain is from CD4, CD8RB, CD40, CRLF2, CSF2RA, CSF3R, EPOR, FCGR2C, GHR, ICOS, IFNAR1, IFNGR1, IFNGR2, IL1R1, IL1RAP, IL2RG, IL3RA, IL5RA, IL6ST, IL7RA, IL10RB, IL11RA, IL13RA2, IL17RA, IL17RB, IL17RC, IL17RE, IL18R1, IL18RAP, IL20RA, IL22RA1, IL31RA, LEPR, PRLR, and TNFRSF8, or mutants thereof that are known to promote signaling activity in certain cell types if such mutants are present in the constructs provided in WO2019/055946. In some embodiments, the transmembrane domain is from CD40, ICOS, FCGR2C, PRLR, IL3RA, or IL6ST.

In some embodiments, the extracellular and transmembrane domain is the viral protein LMP1, or a mutant and/or fragment thereof. LMP1 is a multispan transmembrane protein that is known to activate cell signaling independent of ligand when targeted to lipid rafts or when fused to CD40 (Kaykas et al. EMBO J. 20: 2641 (2001)). A fragment of LMP1 is typically long enough to span a plasma membrane and to activate a linked intracellular domain(s). For example, the LMP1 can be between 15 and 386, 15 and 200, 15 and 150, 15 and 100, 18 and 50, 18 and 30, 20 and 200, 20 and 150, 20 and 50, 20 and 30, 20 and 100, 20 and 40, or 20 and 25 amino acids. A mutant and/or fragment of LMP1 when included in a CLE provided herein, retains its ability to activate an intracellular domain. Furthermore, if present, the extracellular domain includes at least 1, but typically at least 4 amino acids and is typically linked to another functional polypeptide, such as a clearance domain, for example, an eTag. In some embodiments, the lymphoproliferative element comprises an LMP1 transmembrane domain. In illustrative embodiments, the lymphoproliferative element comprises an LMP1 transmembrane domain and the one or more intracellular domains do not comprise an intracellular domain from TNFRSF proteins (i.e. CD40, 4-IBB, RANK, TACI, OX40, CD27, GITR, LTR, and BAFFR), TLR1 to TLR13, integrins, FcγRIII, Dectin1, Dectin2, NOD1, NOD2, CD16, IL-2R, Type III interferon receptor, chemokine receptors such as CCR5 and CCR7, G-protein coupled receptors, TREM1, CD79A, CD79B, Ig-alpha, IPS-1, MyD88, RIG-1, MDA5, CD3Z, MyD88ΔTIR, TRIF, TRAM, TIRAP, MAL, BTK, RTK, RAC1, SYK, NALP3 (NLRP3), NALP3ΔLRR, NALP1, CARD9, DAI, IPAG, STING, Zap70, or LAT.

In other embodiments of CLEs provided herein, the extracellular domain includes a dimerizing moiety. Many different dimerizing moieties disclosed herein can be used for these embodiments. In illustrative embodiments, the dimerizing moieties are capable of homodimerizing. Not to be limited by theory, dimerizing moieties can provide an activating function on intracellular domains connected thereto via transmembrane domains. Such activation can be provided, for example, upon dimerization of a dimerizing moiety, which can cause a change in orientation of intracellular domains connected thereto via a transmembrane domain, or which can cause intracellular domains to come into proximity. An extracellular domain with a dimerizing moiety can also serve a function of connecting a recognition tag to a cell expressing a CLE. In some embodiments, the dimerizing agent can be located intracellularly rather than extracellularly. In some embodiments, more than one or multiples of dimerizing domains can be used.

Extracellular domains for embodiments where extracellular domains have a dimerizing motif, are long enough to form dimers, such as leucine zipper dimers. As such, extracellular domains that include a dimerizing moiety can be from 15 to 100, 20 to 50, 30 to 45, or 35 to 40 amino acids, of in illustrative embodiments is a c-Jun portion of a c-Jun extracellular domain. Extracellular domains of polypeptides that include a dimerizing moiety, may not retain other functionalities. For example, for leucine zippers embodiments, such leucine zippers are capable of forming dimers because they retain a motif of leucines spaced 7 residues apart along an alpha helix. However, leucine zipper moieties of certain embodiments of CLEs provided herein, may or may not retain their DNA binding function.

A spacer of between 1 and 4 alanine residues can be included in CLEs between the extracellular domain that has a dimerizing moiety, and the transmembrane domain. Not to be limited by theory, it is believed that the alanine spacer affects signaling of intracellular domains connected to the leucine zipper extracellular region via the transmembrane domain, by changing the orientation of the intracellular domains.

The first and optional second intracellular domains of CLEs provided herein, are intracellular signaling domains of genes that are known in at least some cell types, to promote proliferation, survival (anti-apoptotic), and/or provide a co-stimulatory signal that enhances proliferative potential or resistance to cell death. As such, these intracellular domains can be intracellular domains from lymphoproliferative elements and co-stimulatory domains provided herein. Some of the intracellular domains of candidate chimeric polypeptides are known to activate JAK1/JAK2, JAK3, STAT1, STAT2, STAT3, STAT4, STAT5, and STAT6 signaling. Conserved motifs that are found in intracellular domains of cytokine receptors that are responsible for this signaling are known (see e.g., Morris et al., “The molecular details of cytokine signaling via the JAK/STAT pathway,” Protein Science (2018) 27:1984-2009). The Box1 and Box2 motifs are involved in binding to JAKs and signal transduction, although the Box2 motif presence is not always required for a proliferative signal (Murakami et al. Proc Natl Acad Sci USA. 1991 Dec. 15; 88(24):11349-53; Fukunaga et al. EMBO J. 1991 October; 10(10):2855-65; and O'Neal and Lee. Lymphokine Cytokine Res. 1993 October; 12(5):309-12). Accordingly, in some embodiments a lymphoproliferative element herein is a transgenic BOX1-containing cytokine receptor that includes an intracellular domain of a cytokine receptor comprising a Box1 Janus kinase (JAK)-binding motif, optionally a Box2 JAK-binding motif, and a Signal Transducer and Activator of Transcription (STAT) binding motif comprising a tyrosine residue. Many cytokine receptors have hydrophobic residues at positions −1, −2, and −6 relative to the Box1 motif, that form a “switch motif,” which is required for cytokine-induced JAK2 activation but not for JAK2 binding (Constantinescu et al. Mol Cell. 2001 February; 7(2):377-85; and Huang et al. Mol Cell. 2001 December; 8(6):1327-38). Accordingly, in certain embodiments of the transgenic BOX1-containing cytokine receptor lymphoproliferative element has a switch motif, which in illustrative embodiments has one or more, and preferably all hydrophobic residues at positions −1, −2, and −6 relative to the Box1 motif. In certain embodiments, the Box1 motif an ICD of a lymphoproliferative element is located proximal to the transmembrane (TM) domain (for example between 5 and 15 or about 10 residues downstream from the TM domain) relative to the Box2 motif, which is located proximal to the transmembrane domain (for example between 10 and 50 residues downstream from the TM domain) relative to the STAT binding motif. The STAT binding motif typically comprising a tyrosine residue, the phosphorylation of which affects binding of a STAT to the STAT binding motif of the lymphoproliferative element. In some embodiments, the ICDs comprising multiple STAT binding motifs where multiple STAT binding motifs are present in a native ICD (e.g. EPO receptor and IL-6 receptor signaling chain (gp130).

Intracellular domains from IFNAR1, IFNGR1, IFNLR1, IL2RB, IL4R, IL5RB, IL6R, IL6ST, IL7RA, IL9R, IL10RA, IL21R, IL27R, IL31RA, LIFR, and OSMR are known in the art to activate JAK1 signaling. Intracellular domains from CRLF2, CSF2RA, CSF2RB, CSF3R, EPOR, GHR, IFNGR2, IL3RA, IL5RA, IL6ST, IL20RA, IL20RB, IL23R, IL27R, LEPR, MPL, and PRLR are known in the art to activate JAK2. Intracellular domains from IL2RG are known in the art to activate JAK3. Intracellular domains from GHR, IFNAR1, IFNAR2, IFNGR1, IFNGR2, IL2RB, IL2RG, IL4R, IL5RA, IL5RB, IL7RA, IL9R, IL21R, IL22RA1, IL31RA, LIFR, MPL, and OSMR are known in the art to activate STAT1. Intracellular domains from IFNAR1 and IFNAR2 are known in the art to activate STAT2. Intracellular domains from GHR, IL2RB, IL2RG, IL6R, IL7RA, IL9R, IL10RA, IL10RB, IL21R, IL22RA1, IL23R, IL27R, IL31RA, LEPR, LIFR, MPL, and OSMR are known in the art to activate STAT3. Intracellular domains from IL12RB1 are known in the art to activate STAT4. Intracellular domains from CSF2RA, CSF2RB, CSF3R, EPOR, GHR, IL2RB, IL2RG, IL3RA, IL4R, IL5RA, IL5RB, IL7RA, IL9R, IL15RA, IL20RA, IL20RB, IL21R, IL22RA1, IL31RA, LIFR, MPL, OSMR, and PRLR are known in the art to activate STAT5. Intracellular domains from IL4R and OSMR are known in the art to activate STAT6. The genes and intracellular domains thereof that are found in a first intracellular domain are the same as the optional second intracellular domain, except that if the first and second intracellular domain are identical, then at least one, and typically both the transmembrane domain and the extracellular domain are not from the same gene.

In some embodiments, all domains of a CLE are other than an IL-7 receptor, or a mutant thereof, and/or a fragment thereof that has at least 10, 15, 20, or 25 contiguous amino acids of IL-7 receptor, or other than an IL-15 receptor, or a mutant thereof, and/or a fragment thereof that has at least 10, 15, 20, or 25 contiguous amino acids of IL-15 receptor. In some embodiments, a CLE does not comprise a combination of first intracellular domain and second intracellular domain of CD40 and MyD88.

In illustrative embodiments, CLEs include a recognition and/or elimination domain. Details regarding recognition and/or elimination domains are provided in other sections herein. Any of the recognition and/or elimination domains provided herein can be part of a CLE. Typically the recognition domain is linked to the N terminus of the extracellular domain. Not to be limited by theory, in some embodiments, the extracellular domain includes the function of providing a linker, in illustrative embodiments a flexible linker, linking a recognition domain to a cell that expresses the CLE.

Furthermore, polynucleotides that include a nucleic acid sequence encoding a CLE provided herein, also typically comprise a signal sequence to direct expression to the plasma membrane. Exemplary signal sequences are provided herein in other sections. Elements can be provided on the transcript such that both a CAR and CLE are expressed from the same transcript in certain embodiments.

In any aspects or embodiments wherein the extracellular domain of a CLE comprises a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize. In any of the aspects and embodiments herein wherein the extracellular domain of a CLE comprises a dimerizing motif, the dimerizing motif can require a dimerizing agent, and the dimerizing motif and associated dimerizing agent can be selected from the group consisting of: FKBP and rapamycin or analogs thereof, GyrB and coumermycin or analogs thereof, DHFR and methotrexate or analogs thereof, or DmrB and AP20187 or analogs thereof, as well as mutants and/or active fragments of the recited dimerizing proteins that retain the ability to dimerize. In some aspects and illustrative embodiments, a lymphoproliferative element is constitutively active, and is other than a lymphoproliferative element that requires a dimerizing agent for activation.

In illustrative embodiments of any aspects or embodiments herein wherein the extracellular domain of a CLE comprises a dimerizing motif, the extracellular domain can comprise a leucine zipper motif. In some embodiments, the leucine zipper motif is from a jun polypeptide, for example c-jun. In certain embodiments the c-jun polypeptide is the c-jun polypeptide region of ECD-11.

Internally dimerizing and/or multimerizing lymphoproliferative elements in one embodiment are an integral part of a system that uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner. This provides the system with temporal control, reversibility using monomeric drug analogs, and enhanced specificity. The high affinity of third-generation AP20187/AP1903 dimerizer drugs for their binding domain, FKBP12 permits specific activation of the recombinant receptor in vivo without the induction of non-specific side effects through endogenous FKBP12. FKBP12 variants having amino acid substitutions and deletions, such as FKBP12V36, that bind to a dimerizer drug, may also be used. In addition, the synthetic ligands are resistant to protease degradation, making them more efficient at activating receptors in vivo than most delivered protein agents.

### Pseudotyping Elements

Many of the methods and compositions provided herein include pseudotyping elements. The pseudotyping of replication incompetent recombinant retroviral particles with heterologous envelope glycoproteins typically alters the tropism of a virus and facilitates the transduction of host cells. A pseudotyping element as used herein can include a “binding polypeptide” that includes one or more polypeptides, typically glycoproteins, that identify and bind the target host cell, and one or more “fusogenic polypeptides” that mediate fusion of the retroviral and target host cell membranes, thereby allowing a retroviral genome to enter the target host cell. In some embodiments provided herein, pseudotyping elements are provided as polypeptide(s)/protein(s), or as nucleic acid sequences encoding the polypeptide(s)/protein(s).

In some embodiments, the pseudotyping element is the feline endogenous virus (RD114) envelope protein, an oncoretroviral amphotropic envelope protein, an oncoretroviral ecotropic envelope protein, the vesicular stomatitis virus envelope protein (VSV-G) (SEQ ID NO: 336), the baboon retroviral envelope glycoprotein (BaEV) (SEQ ID NO: 337), the murine leukemia envelope protein (MuLV) (SEQ ID NO: 338), the influenza glycoprotein HA surface glycoprotein (HA), the influenza glycoprotein neurominidase (NA), the paramyxovirus Measles envelope protein H, the paramyxovirus Measles envelope protein F, and/or functional variants or fragments of any of these envelope proteins.

In some embodiments, the pseudotyping element can be wild-type BaEV. Not to be limited by theory, BaEV contains an R peptide that has been shown to inhibit transduction. In some embodiments, the BaEV can contain a deletion of the R peptide. In some embodiments, the BaEV can contain a deletion of the inhibitory R peptide after the nucleotides encoding the amino acid sequence HA, referred to herein as BaEVΔR (HA) (SEQ ID NO: 339). In some embodiments, the BaEV can contain a deletion of the inhibitory R peptide after the nucleotides encoding the amino acid sequence HAM, referred to herein as BaEVΔR (HAM) (SEQ ID NO: 340).

In some embodiments, the pseudotyping element can be wild-type MuLV. In some embodiments, the MuLV can contain one or more mutations to remove the furin-mediated cleavage site located between the transmembrane (TM) and surface (SU) subunits of the envelope glycoprotein. In some embodiments the MuLV contains the SUx mutation (MuLVSUx) (SEQ ID NO: 453) which inhibits furin-mediated cleavage of MuLV envelope protein in packaging cells. In certain embodiments the C-terminus of the cytoplasmic tail of the MuLV or MuLVSUx protein is truncated by 4 to 31 amino acids. In certain embodiments the C-terminus of the cytoplasmic tail of the MuLV or MuLVSUx protein is truncated by 4, 8, 12, 16, 20, 24, 28, or 31 amino acids.

In some embodiments, the pseudotyping elements include a binding polypeptide and a fusogenic polypeptide derived from different proteins. For example, the replication incompetent recombinant retroviral particles of the methods and compositions disclosed herein can be pseudotyped with the fusion (F) and/or hemagglutinin (H) polypeptides of the measles virus (MV), as non-limiting examples, clinical wildtype strains of MV, and vaccine strains including the Edmonston strain (MV-Edm) (GenBank; AF266288.2) or fragments thereof. Not to be limited by theory, both hemagglutinin (H) and fusion (F) polypeptides are believed to play a role in entry into host cells wherein the H protein binds MV to receptors CD46, SLAM, and Nectin-4 on target cells and F mediates fusion of the retroviral and host cell membranes. In an illustrative embodiment, especially where the target cell is a T cell and/or NK cell, the binding polypeptide is a Measles Virus H polypeptide and the fusogenic polypeptide is a Measles Virus F polypeptide.

In some studies, lentiviral particles pseudotyped with truncated F and H polypeptides had a significant increase in titers and transduction efficiency (Funke et al. 2008. Molecular Therapy. 16(8):1427-1436), (Frecha et al. 2008. Blood. 112(13):4843-4852). The highest titers were obtained when the F cytoplasmic tail was truncated by 30 residues (referred to as MV(Ed)-FΔ30 (SEQ ID NO:313)). For the H variants, optimal truncation occurred when 18 or 19 residues were deleted (MV(Ed)-HΔ18 (SEQ ID NO:314) or MV(Ed)-HΔ19), although variants with a truncation of 24 residues with and without replacement of deleted residues with alanine (MV(Ed)-HΔ24 (SEQ ID NO:315) and MV(Ed)-HΔ24+A) also resulted in optimal titers. Accordingly, in some embodiments, including those directed to transducing T cells and/or NK cells, the replication incompetent recombinant retroviral particles of the methods and compositions disclosed herein are pseudotyped with mutated or variant versions of the measles virus fusion (F) and hemagglutinin (H) polypeptides, in illustrative examples, cytoplasmic domain deletion variants of measles virus F and H polypeptides. In some embodiments, the mutated F and H polypeptides are “truncated H” or “truncated F” polypeptides, whose cytoplasmic portion has been truncated, i.e. amino acid residues (or coding nucleic acids of the corresponding nucleic acid molecule encoding the protein) have been deleted. “HΔY” and “FΔX” designate such truncated H and F polypeptide, respectively, wherein “Y” refers to 1-34 residues that have been deleted from the amino termini and “X” refers to 1-35 residues that have been deleted from the carboxy termini of the cytoplasmic domains. In a further embodiment, the “truncated F polypeptide” is FΔ24 or FΔ30 and/or the “truncated H protein” is selected from the group consisting of HΔ14, HΔ15, HΔ16, HΔ17, HΔ18, HΔ19, HΔ20, HΔ21+A, HΔ24 and HΔ24+4A, more preferably HΔ18 or HΔ24. In an illustrative embodiment, the truncated F polypeptide is MV(Ed)-FΔ30 and the truncated H polypeptide is MV(Ed)-HΔ18.

In some embodiments, the pseudotyping element includes polypeptides derived from different proteins. For example, the pseudotyping element can comprise an influenza protein hemagglutinin HA and/or a neuraminidase (NA). In certain embodiments the HA is from influenza A virus subtype HIN1. In illustrative embodiments the HA is from H1N1 PR8 1934 in which the monobasic trypsin-dependent cleavage site has been mutated to a more promiscuous multibasic sequence (SEQ ID NO:311). In certain embodiments the NA is from influenza A virus subtype H10N7. In illustrative embodiments the NA is from H10N7-HKWF446C-07 (SEQ ID NO:312).

In some embodiments, the viral particles are copseudotyped with envelope glycoproteins from 2 or more heterologous viruses. In some embodiments, the viral particles are copseudotyped with VSV-G, or a functional variant or fragment thereof, and an envelope protein from RD114, BaEV, MuLV, influenza virus, measles virus, and/or a functional variant or fragment thereof. In some embodiments, the viral particles are copseudotyped with VSV-G and the MV(Ed)-H glycoprotein or the MV(Ed)-H glycoprotein with a truncated cytoplasmic domain. In illustrative embodiments, the viral particles are copseudotyped with VSV-G and MV(Ed)-HΔ24. In certain embodiments, VSV-G is copseudotyped with MuLV or MuLV with a truncated cytoplasmic domain. In other embodiments, VSV-G is copseudotyped with MuLVSUx or MuLVSUx with a truncated cytoplasmic domain. In further illustrative embodiments, VSV-G is copseudotyped with a fusion of an antiCD3scFv to MuLV.

In some embodiments, the fusogenic polypeptide includes multiple elements expressed as one polypeptide. In some embodiments, the binding polypeptide and fusogenic polypeptide are translated from the same transcript but from separate ribosome binding sites; in other embodiments, the binding polypeptide and fusogenic polypeptide are separated by a cleavage peptide site, which not to be bound by theory, is cleaved after translation, as is common in the literature, or a ribosomal skip sequence. In some embodiments, the translation of the binding polypeptide and fusogenic polypeptide from separate ribosome binding sites results in a higher amount of the fusogenic polypeptide as compared to the binding polypeptide. In some embodiments, the ratio of the fusogenic polypeptide to the binding polypeptide is at least 2:1, at least 3:1, at least 4:1, at least 5:1, at least 6:1, at least 7:1, or at least 8:1. In some embodiments, the ratio of the fusogenic polypeptide to the binding polypeptide is between 1.5:1, 2:1, or 3:1, on the low end of the range, and 3:1, 4:1, 5:1, 6:1, 7:1, 8:1, 9:1 or 10:1 on the high end of the range.

### Activation Elements

Many of the methods and composition aspects of the present disclosure include an activation element, also referred to herein as a T cell activation element, or a nucleic acid encoding an activation element. The restrictions associated with lentiviral (LV) transduction into resting T cells are attributed to a series of pre-entry and post-entry barriers as well as cellular restrictive factors (Strebel et al 2009. BMC Medicine 7:48). One restriction is the inability for the envelope pseudotyped-LV particles to recognize potential receptors and mediate fusion with the cellular membrane. However, under certain conditions, the transduction of resting T cells with HIV-1-based lentiviral vectors is possible mostly upon T cell receptor (TCR) CD3 complex and CD28 co-stimulation (Korin & Zack. 1998. Journal of Virology. 72:3161-8, Maurice et al. 2002. Blood 99:2342-50), as well as through exposure to cytokines (Cavalieri et al. 2003).

Cells of the immune system such as T lymphocytes recognize and interact with specific antigens through receptors or receptor complexes which, upon recognition or an interaction with such antigens, cause activation of the cell and expansion in the body. An example of such a receptor is the antigen-specific T lymphocyte receptor complex (TCR/CD3). The T cell receptor (TCR) is expressed on the surface of T lymphocytes. One component, CD3, is responsible for intracellular signaling following occupancy of the TCR by ligand. The T lymphocyte receptor for antigen-CD3 complex (TCR/CD3) recognizes antigenic peptides that are presented to it by the proteins of the major histocompatibility complex (MHC). Complexes of MHC and peptide are expressed on the surface of antigen presenting cells and other T lymphocyte targets. Stimulation of the TCR/CD3 complex results in activation of the T lymphocyte and a consequent antigen-specific immune response. The TCR/CD3 complex plays a central role in the effector function and regulation of the immune system. Thus, activation elements provided herein, activate T cells by binding to one or more components of the T cell receptor associated complex, for example by binding to CD3. In some embodiments, the activation element can activate alone. In other cases, the activation requires activation through the TCR receptor complex in order to further activate cells.

T lymphocytes also require a second, co-stimulatory signal to become fully active in vivo. Without such a signal, T lymphocytes are either non-responsive to antigen binding to the TCR, or become anergic. However, the second, co-stimulatory signal is not required for the transduction and expansion of T cells. Such a co-stimulatory signal, for example, is provided by CD28, a T lymphocyte protein, which interacts with CD80 and CD86 on antigen-producing cells. As used herein, a functional extracellular fragment of CD80 retains its ability to interact with CD28. OX40, 4-1BB, and ICOS (Inducible COStimulator), other T lymphocyte proteins, and provides a co-stimulatory signal when bound to one or more of its respective ligands: OX40L, 4-1BBL, and ICOSLG.

Activation of the T cell receptor (TCR) CD3 complex and co-stimulation with CD28 can occur by ex vivo exposure to solid surfaces (e.g. beads) coated with anti-CD3 and anti-CD28. In some embodiments of the methods and compositions disclosed herein, resting T cells are activated by exposure to solid surfaces coated with anti-CD3 and anti-CD28 ex vivo. In other embodiments, resting T cells or NK cells, and in illustrative embodiments resting T cells, are activated by exposure to soluble anti-CD3 antibodies (e.g. at 50-150, or 75-125, or 100 ng/ml). In such embodiments, which can be part of methods for genetically modifying or transducing, in illustrative embodiments without prior activation, such activation and/or contacting can be carried out by including anti-CD3 in a transduction reaction mixture and contacting with optional incubating for any of the times provided herein. Furthermore, such activation with soluble anti-CD3 can occur by incubating lymphocytes, such as PBMCs, and in illustrative embodiments NK cells and in more illustrative embodiments, T cells, after they are contacted with retroviral particles in a media containing an anti-CD3. Such incubation can be for example, for between 5, 10, 15, 30, 45, 60, or 120 minutes on the low end of the range, and 15, 30, 45, 60, 120, 180, or 240 minutes on the high end of the range, for example, between 15 and 1 hours or 2 hours.

In certain illustrative embodiments of the methods and compositions provided herein, polypeptides that are capable of binding to an activating T cell surface protein are presented as “activation elements” on the surface of replication incompetent recombinant retroviral particles of the methods and compositions disclosed herein, which are also aspects of the invention. In illustrative embodiments, the activation elements on the surfaces of the replication incompetent recombinant retroviral particles can include one or more polypeptides capable of binding CD3. In illustrative embodiments, the activation elements on the surfaces of the replication incompetent recombinant retroviral particles can include one or more polypeptides capable of binding the epsilon chain of CD3 (CD3 epsilon). In other embodiments, the activation element on the surfaces of the replication incompetent recombinant retroviral particles can include one or more polypeptides capable of binding CD28, OX40, 4-1BB, ICOS, CD9, CD53, CD63, CD81, and/or CD82 and optionally one or more polypeptides capable of binding CD3. In illustrative embodiments, the activation element can be a T cell surface protein agonist. The activation element can include a polypeptide that acts as a ligand for a T cell surface protein. In some embodiments, the polypeptide that acts as a ligand for a T cell surface protein is, or includes, one or more of OX40L, 4-1BBL, or ICOSLG.

In some embodiments, one or typically more copies of one or more of these activation elements can be expressed on the surfaces of the replication incompetent recombinant retroviral particles as polypeptides separate and distinct from the pseudotyping elements. In some embodiments, the activation elements can be expressed on the surfaces of the replication incompetent recombinant retroviral particles as fusion polypeptides. In illustrative embodiments, the fusion polypeptides include one or more activation elements and one or more pseudotyping elements. In further illustrative embodiments, the fusion polypeptide includes anti-CD3, for example an anti-CD3scFv, or an anti-CD3scFvFc, and a viral envelope protein. In one example the fusion polypeptide is the OKT-3scFv fused to the amino terminal end of a viral envelope protein such as the MuLV envelope protein, as shown in Maurice et al. (2002). In some embodiments, the fusion polypeptide is UCHT1scFv fused to a viral envelope protein, for example the MuLV envelope protein (SEQ ID NO:341), the MuLVSUx envelope protein (SEQ ID NO:454), VSV-G (SEQ ID NO:455 or SEQ ID NO:456), or functional variants or fragments thereof, including any of the membrane protein truncations provided herein. In such fusion constructs, and any other constructs wherein an activation element is tethered to the surface of a retroviral particle, illustrative embodiments especially for compositions and methods herein for transducing lymphocytes in whole blood, do not include any blood protein (e.g. blood Factor (e.g. Factor X)) cleavage sites in the portion of the fusion protein that resides outside the retroviral particle. In some embodiments, the fusion constructs do not include any furin cleavage sites. Furin is a membrane bound protease expressed in all mammalian cells examined, some of which is secreted and active in blood plasma (See e.g. C. Fernandez et al. J. Internal. Medicine (2018) 284; 377-387). Mutations can be made to fusion constructs using known methods to remove such protease cleavage sites.

Polypeptides that bind CD3, CD28, OX40, 4-1BB, or ICOS are referred to as activation elements because of their ability to activate resting T cells. In certain embodiments, nucleic acids encoding such an activating element are found in the genome of a replication incompetent recombinant retroviral particle that contains the activating element on its surface. In other embodiments, nucleic acids encoding an activating element are not found in the replication incompetent recombinant retroviral particle genome. In still other embodiments, the nucleic acids encoding an activating element are found in the genome of a virus packaging cell.

In some embodiments, the activation element is a polypeptide capable of binding to CD3. In certain embodiments the polypeptide capable of binding to CD3, binds to CD3D, CD3E, CD3G, or CD3Z. In illustrative embodiments the activation element is a polypeptide capable of binding to CD3E. In some embodiments, the polypeptide capable of binding to CD3 is an anti-CD3 antibody, or a fragment thereof that retains the ability to bind to CD3. In illustrative embodiments, the anti-CD3 antibody or fragment thereof is a single chain anti-CD3 antibody, such as but not limited to, an anti-CD3 scFv. In another illustrative embodiment, the polypeptide capable of binding to CD3 is anti-CD3scFvFc.

A number of anti-human CD3 monoclonal antibodies and antibody fragments thereof are available, and can be used in the present invention, including but not limited to UCHT1, OKT-3, HIT3A, TRX4, X35-3, VIT3, BMA030 (BW264/56), CLB-T3/3, CRIS7, YTH12.5, F111409, CLB-T3.4.2, TR-66, WT31, WT32, SPv-T3b, 11D8, XIII-141, XIII46, XIII-87, 12F6, T3/RW2-8C8, T3/RW24B6, OKT3D, M-T301, SMC2 and F101.01.

In some embodiments, the activation element is a polypeptide capable of binding to CD28. In some embodiments, the polypeptide capable of binding to CD28 is an anti-CD28 antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an external fragment of CD80. In some aspects herein, an external fragment of CD80 means a fragment that is typically present on the outside of a cell in the normal cellular location of CD80, that retains the ability to bind to CD28. In illustrative embodiments, the anti-CD28 antibody or fragment thereof is a single chain anti-CD28 antibody, such as, but not limited to, an anti-CD28 scFv. In another illustrative embodiment, the polypeptide capable of binding to CD28 is CD80, or a fragment of CD80 such as an external fragment of CD80.

Anti-CD28 antibodies are known in the art and can include, as non-limiting examples, monoclonal antibody 9.3, an IgG2a antibody (Dr. Jeffery Ledbetter, Bristol Myers Squibb Corporation, Seattle, Wash.), monoclonal antibody KOLT-2, an IgG1 antibody, 15E8, an IgG1 antibody, 248.23.2, an IgM antibody and EX5.3D10, an IgG2a antibody.

In an illustrative embodiment, an activation element includes two polypeptides, a polypeptide capable of binding to CD3 and a polypeptide capable of binding to CD28.

In certain embodiments, the polypeptide capable of binding to CD3 or CD28 is an antibody, a single chain monoclonal antibody or an antibody fragment, for example a single chain antibody fragment. Accordingly, the antibody fragment can be, for example, a single chain fragment variable region (scFv), an antibody binding (Fab) fragment of an antibody, a single chain antigen-binding fragment (scFab), a single chain antigen-binding fragment without cysteines (scFabΔC), a fragment variable region (Fv), a construct specific to adjacent epitopes of an antigen (CRAb), or a single domain antibody (VH or VL).

In any of the embodiments disclosed herein, an activation element, or a nucleic acid encoding the same, can include a dimerizing or higher order multimerizing motif. Dimerizing and multimerizing motifs are well-known in the art and a skilled artisan will understand how to incorporate them into the polypeptides for effective dimerization or multimerization. For example, in some embodiments, the activation element that includes a dimerizing motif can be one or more polypeptides capable of binding to CD3 and/or CD28. In some embodiments, the polypeptide capable of binding to CD3 is an anti-CD3 antibody, or a fragment thereof that retains the ability to bind to CD3. In illustrative embodiments, the anti-CD3 antibody or fragment thereof is a single chain anti-CD3 antibody, such as but not limited to, an anti-CD3 scFv. In another illustrative embodiment, the polypeptide capable of binding to CD3 is anti-CD3scFvFc, which in some embodiments is considered an anti-CD3 with a dimerizing motif without any additional dimerizing motif, since anti-CD3scFvFc constructs are known to be capable of dimerizing without the need for a separate dimerizing motif.

In some embodiments, the dimerizing or multimerizing motif, or a nucleic acid sequence encoding the same, can be an amino acid sequence from transmembrane polypeptides that naturally exist as homodimers or multimers. In some embodiments, the dimerizing or multimerizing motif, or a nucleic acid sequence encoding the same, can be an amino acid sequence from a fragment of a natural protein or an engineered protein. In one embodiment, the homodimeric polypeptide is a leucine zipper motif-containing polypeptide (leucine zipper polypeptide). For example, a leucine zipper polypeptide derived from c-JUN, non-limiting examples of which are disclosed related to chimeric lymphoproliferative elements (CLEs) herein.

In some embodiments, these transmembrane homodimeric polypeptides can include early activation antigen CD69 (CD69), Transferrin receptor protein 1 (CD71), B-cell differentiation antigen (CD72), T-cell surface protein tactile (CD96), Endoglin (Cd105), Killer cell lectin-like receptor subfamily B member 1 (Cd161), P-selectin glycoprotein ligand 1 (Cd162), Glutamyl aminopeptidase (Cd249), Tumor necrosis factor receptor superfamily member 16 (CD271), Cadherin-1 (E-Cadherin) (Cd324), or active fragments thereof. In some embodiments, the dimerizing motif, and nucleic acid encoding the same, can include an amino acid sequence from transmembrane proteins that dimerize upon ligand (also referred to herein as a dimerizer or dimerizing agent) binding. In some embodiments, the dimerizing motif and dimerizer can include (where the dimerizer is in parentheses following the dimerizer-binding pair): FKBP and FKBP (rapamycin); GyrB and GyrB (coumermycin); DHFR and DHFR (methotrexate); or DmrB and DmrB (AP20187). As noted above, rapamycin can serve as a dimerizer. Alternatively, a rapamycin derivative or analog can be used (see, e.g., WO96/41865; WO 99/36553; WO 01/14387; and Ye et al (1999) Science 283:88-91). For example, analogs, homologs, derivatives, and other compounds related structurally to rapamycin (“rapalogs”) include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Additional information is presented in, e.g., U.S. Pat. Nos. 5,525,610; 5,310,903 5,362,718; and 5,527,907. Selective epimerization of the C-28 hydroxyl group has been described (see, e.g., WO 01/14387). Additional synthetic dimerizing agents suitable for use as an alternative to rapamycin include those described in U.S. Patent Publication No. 2012/0130076. As noted above, coumermycin can serve as a dimerizing agent. Alternatively, a coumermycin analog can be used (see, e.g., Farrar et al. (1996) Nature 383:178-181; and U.S. Pat. No. 6,916,846). As noted above, in some cases, the dimerizing agent is methotrexate, e.g., a non-cytotoxic, homo-bifunctional methotrexate dimer (see, e.g., U.S. Pat. No. 8,236,925). Although some embodiments of lymphoproliferative elements include a dimerizing agent, in some aspects and illustrative embodiments, a lymphoproliferative element is constitutively active, and is other than a lymphoproliferative element that requires a dimerizing agent for activation.

In some embodiments, when present on the surface of replication incompetent recombinant retroviral particles, an activation element including a dimerizing motif can be active in the absence of a dimerizing agent. For example, activation elements including a dimerizing motif from transmembrane homodimeric polypeptides including CD69, CD71, CD72, CD96, Cd105, Cd161, Cd162, Cd249, CD271, Cd324, active mutants thereof, and/or active fragments thereof can be active in the absence a dimerizing agent. In some embodiments, the activation element can be an anti-CD3 single chain fragment and include a dimerizing motif selected from the group consisting of CD69, CD71, CD72, CD96, Cd105, Cd161, Cd162, Cd249, CD271, Cd324, active mutants thereof, and/or active fragments thereof. In some embodiments, when present on the surface of replication incompetent recombinant retroviral particles, an activation element including a dimerizing motif can be active in the presence of a dimerizing agent. For example, activation elements including a dimerizing motif from FKBP, GyrB, DHFR, or DmrB can be active in the presence of the respective dimerizing agents or analogs thereof, e.g. rapamycin, coumermycin, methotrexate, and AP20187, respectively. In some embodiments, the activation element can be a single chain antibody fragment against anti-CD3 or anti-CD28, or another molecule that binds CD3 or CD28, and the dimerizing motif and dimerizing agent can be selected from the group consisting of FKBP and rapamycin or analogs thereof, GyrB and coumermycin or analogs thereof, DHFR and methotrexate or analogs thereof, or DmrB and AP20187 or analogs thereof.

In some embodiments, an activation element is fused to a heterologous signal sequence and/or a heterologous membrane attachment sequence or a membrane bound protein, all of which help direct the activation element to the membrane. The heterologous signal sequence targets the activation element to the endoplasmic reticulum, where the heterologous membrane attachment sequence covalently attaches to one or several fatty acids (also known as posttranslational lipid modification) such that the activation elements that are fused to the heterologous membrane attachment sequence are anchored in the lipid rafts of the plasma membrane. In some embodiments, posttranslational lipid modification can occur via myristoylation, palmitoylation, or GPI anchorage. Myristoylation is a post-translational protein modification which corresponds to the covalent linkage of a 14-carbon saturated fatty acid, the myristic acid, to the N-terminal glycine of a eukaryotic or viral protein. Palmitoylation is a post-translational protein modification which corresponds to the covalent linkage of a C16 acyl chain to cysteines, and less frequently to serine and threonine residues, of proteins. GPI anchorage refers to the attachment of glycosylphosphatidylinositol, or GPI, to the C-terminus of a protein during posttranslational modification.

In some embodiments, the heterologous membrane attachment sequence is a GPI anchor attachment sequence. The heterologous GPI anchor attachment sequence can be derived from any known GPI-anchored protein (reviewed in Ferguson M A J, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (N.Y.): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In illustrative embodiments, the heterologous GPI anchor attachment sequence is derived from Fc receptor FcγRIIIb (CD16b) or decay accelerating factor (DAF), otherwise known as complement decay-accelerating factor or CD55.

In some embodiments, one or both of the activation elements include a heterologous signal sequence to help direct expression of the activation element to the cell membrane. Any signal sequence that is active in the packaging cell line can be used. In some embodiments, the signal sequence is a DAF signal sequence. In illustrative embodiments, an activation element is fused to a DAF signal sequence at its N terminus and a GPI anchor attachment sequence at its C terminus.

In an illustrative embodiment, the activation element includes anti-CD3 scFvFc fused to a GPI anchor attachment sequence derived from CD14 and CD80 fused to a GPI anchor attachment sequence derived from CD16b; and both are expressed on the surface of a replication incompetent recombinant retroviral particle provided herein. In some embodiments, the anti-CD3 scFvFc is fused to a DAF signal sequence at its N terminus and a GPI anchor attachment sequence derived from CD14 at its C terminus and the CD80 is fused to a DAF signal sequence at its N terminus and a GPI anchor attachment sequence derived from CD16b at its C terminus; and both are expressed on the surface of a replication incompetent recombinant retroviral particle provided herein. In some embodiments, the DAF signal sequence includes amino acid residues 1-30 of the DAF protein.

### Membrane-Bound Cytokines

Some embodiments of the method and composition aspects provided herein, include a membrane-bound cytokine, or polynucleotides encoding a membrane-bound cytokine. Cytokines are typically, but not always, secreted proteins. Cytokines that are naturally secreted can be engineered as fusion proteins to be membrane-bound. Membrane-bound cytokine fusion polypeptides are included in methods and compositions disclosed herein, and are also an aspect of the invention. In some embodiments, replication incompetent recombinant retroviral particles have a membrane-bound cytokine fusion polypeptide on their surface that is capable of binding a T cell and/or NK cell and promoting proliferation and/or survival thereof. Typically, membrane-bound polypeptides are incorporated into the membranes of replication incompetent recombinant retroviral particles, and when a cell is transduced by the replication incompetent recombinant retroviral particles, the fusion of the retroviral and host cell membranes results in the polypeptide being bound to the membrane of the transduced cell.

In some embodiments, the cytokine fusion polypeptide includes IL-2, IL-7, IL-15, or an active fragment thereof. The membrane-bound cytokine fusion polypeptides are typically a cytokine fused to heterologous signal sequence and/or a heterologous membrane attachment sequence. In some embodiments, the heterologous membrane attachment sequence is a GPI anchor attachment sequence. The heterologous GPI anchor attachment sequence can be derived from any known GPI-anchored protein (reviewed in Ferguson M A J, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (N.Y.): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF.

In illustrative embodiments, the membrane-bound cytokine is a fusion polypeptide of a cytokine fused to DAF. DAF is known to accumulate in lipid rafts that are incorporated into the membranes of replication incompetent recombinant retroviral particles budding from packaging cells. Accordingly, not to be limited by theory, it is believed that DAF fusion proteins are preferentially targeted to portions of membranes of packaging cells that will become part of a recombinant retroviral membrane.

In non-limiting illustrative embodiments, the cytokine fusion polypeptide is an IL-7, or an active fragment thereof, fused to DAF. In a specific non-limiting illustrative embodiment, the fusion cytokine polypeptide includes in order: the DAF signal sequence (residues 1-31 of DAF), IL-7 without its signal sequence, and residues 36-525 of DAF.

### Packaging Cell Lines/Methods of Making Recombinant Retroviral Particles

The present disclosure provides mammalian packaging cells and packaging cell lines that produce replication incompetent recombinant retroviral particles. The cell lines that produce replication incompetent recombinant retroviral particles are also referred to herein as packaging cell lines. A non-limiting example of such method is illustrated in WO2019/055946. Further exemplary methods for making retroviral particles are provided herein, for example in the Examples section herein. Such methods include, for example, a 4 plasmid system or a 5 plasmid system when a nucleic acid encoding an additional membrane bound protein, such as a T cell activation element that is not a fusion with the viral envelope, such as a GPI-linked anti-CD3, is included (See WO2019/05546). In an illustrative embodiment, provided herein is a 4 plasmid system in which a T cell activation element, such as a GPI-linked anti-CD3, is encoded on one of the packaging plasmids such as the plasmid encoding the viral envelope or the plasmid encoding REV, and optionally a second viral membrane-associated transgene such as a membrane bound cytokine can be encoded on the other packaging plasmid. In each case the nucleic acid encoding the viral protein is separated from the transgene by an IRES or a ribosomal skip sequence such as P2A or T2A. Such 4 plasmid system and associated polynucleotides as stated in the Examples, provided increased titers as compared to a 5 vector system in transient transfections, and thus provide illustrative embodiments herein. The present disclosure provides packaging cells and mammalian cell lines that are packaging cell lines that produce replication incompetent recombinant retroviral particles that genetically modify target mammalian cells and the target mammalian cells themselves. In illustrative embodiments, the packaging cell comprises nucleic acid sequences encoding a packageable RNA genome of the replication incompetent retroviral particle, a REV protein, a gag polypeptide, a pol polypeptide, and a pseudotyping element.

The cells of the packaging cell line can be adherent or suspension cells. Exemplary cell types are provided hereinbelow. In illustrative embodiments, the packaging cell line can be a suspension cell line, i.e. a cell line that does not adhere to a surface during growth. The cells can be grown in a chemically-defined media and/or a serum-free media. In some embodiments, the packaging cell line can be a suspension cell line derived from an adherent cell line, for example, the HEK293 cell line can be grown in conditions to generate a suspension-adapted HEK293 cell line according to methods known in the art. The packaging cell line is typically grown in a chemically defined media. In some embodiments, the packaging cell line media can include serum. In some embodiments, the packaging cell line media can include a serum replacement, as known in the art. In illustrative embodiments, the packaging cell line media can be serum-free media. Such media can be a chemically defined, serum-free formulation manufactured in compliance with Current Good Manufacturing Practice (CGMP) regulations of the US Food and Drug Administration (FDA). The packaging cell line media can be xeno-free and complete. In some embodiments, the packaging cell line media has been cleared by regulatory agencies for use in ex vivo cell processing, such as an FDA 510(k) cleared device.

Accordingly, in one aspect, provided herein is a method of making a replication incompetent recombinant retroviral particle including: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; and B. harvesting the replication incompetent recombinant retroviral particle from the serum-free media. In another aspect, provided herein is a method of transducing a lymphocyte with a replication incompetent recombinant retroviral particle comprising: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; B. harvesting the replication incompetent recombinant retroviral particle from the serum-free media; and C. contacting the lymphocyte with the replication incompetent recombinant retroviral particle, wherein the contacting is performed for less than 24 hours, 20 hours, 18 hours, 12 hours, 8 hours, 4 hours, 2 hours, 1 hour, 30 minutes, or 15 minutes (or between contacting and no incubation, or 15 minutes, 30 minutes, 1, 2, 3, or 4 hours on the low end of the range and 1, 2, 3, 4, 6, 8, 12, 18, 20, or 24 hours on the high end of the range), thereby transducing the lymphocyte.

The packageable RNA genome, in certain illustrative embodiments, is designed to express one or more target polypeptides, including as a non-limiting example, any of the engineered signaling polypeptides disclosed herein and/or one or more (e.g. two or more) inhibitory RNA molecules in opposite orientation (e.g., encoding on the opposite strand and in the opposite orientation), from retroviral components such as gag and pol. For example, the packageable RNA genome can include from 5′ to 3′: a 5′ long terminal repeat, or active truncated fragment thereof; a nucleic acid sequence encoding a retroviral cis-acting RNA packaging element; a nucleic acid sequence encoding a first and optionally second target polypeptide, such as, but not limited to, an engineered signaling polypeptide(s) in opposite orientation, which can be driven off a promoter in this opposite orientation with respect to the 5′ long terminal repeat and the cis-acting RNA packaging element, which in some embodiments is called a “fourth” promoter for convenience only (and sometimes referred to herein as the promoter active in T cells and/or NK cells), which is active in a target cell such as a T cell and/or an NK cell but in illustrative examples is not active in the packaging cell or is only inducibly or minimally active in the packaging cell; and a 3′ long terminal repeat, or active truncated fragment thereof. In some embodiments, the packageable RNA genome can include a central polypurine tract (cPPT)/central termination sequence (CTS) element. In some embodiments, the retroviral cis-acting RNA packaging element can be HIV Psi. In some embodiments, the retroviral cis-acting RNA packaging element can be the Rev Response Element. The engineered signaling polypeptide driven by the promoter in the opposite orientation from the 5′ long terminal repeat, in illustrative embodiments, is one or more of the engineered signaling polypeptides disclosed herein and can optionally express one or more inhibitory RNA molecules as disclosed in more detail herein and in WO2017/165245A2, WO2018/009923A1, and WO2018/161064A1.

It will be understood that promoter number, such as a first, second, third, fourth, etc. promoter is for convenience only. A promoter that is called a “fourth” promoter should not be taken to imply that there are any additional promoters, such as first, second or third promoters, unless such other promoters are explicitly recited. It should be noted that each of the promoters are capable of driving expression of a transcript in an appropriate cell type and such transcript forms a transcription unit.

In some embodiments, the engineered signaling polypeptide can include a first lymphoproliferative element. Suitable lymphoproliferative elements are disclosed in other sections herein. As a non-limiting example, the lymphoproliferative element can be expressed as a fusion with a recognition domain, such as an eTag, as disclosed herein. In some embodiments, the packageable RNA genome can further include a nucleic acid sequence encoding a second engineered polypeptide including a chimeric antigen receptor, encoding any CAR embodiment provided herein. For example, the second engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments where the target cell is a T cell, the promoter that is active in a target cell is active in a T cell, as disclosed elsewhere herein.

In some embodiments, the engineered signaling polypeptide can include a CAR, and the nucleic acid sequence can encode any CAR embodiment provided herein. For example, the engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments, the packageable RNA genome can further include a nucleic acid sequence encoding a second engineered polypeptide. In some embodiments, the second engineered polypeptide can be a lymphoproliferative element. In some embodiments where the target cell is a T cell or NK cell, the promoter that is active in a target cell is active in a T cell or NK cell, as disclosed elsewhere herein.

In some embodiments, the packageable RNA genome included in any of the aspects provided herein, can further include a riboswitch, as discussed in WO2017/165245A2, WO2018/009923A1, and WO2018/161064A1. In some embodiments, the nucleic acid sequence encoding the engineered signaling polypeptide can be in a reverse orientation with respect to the 5′ to 3′ orientation established by the 5′ LTR and the 3′ LTR. In further embodiments, the packageable RNA genome can further include a riboswitch and, optionally, the riboswitch can be in reverse orientation. In any of the embodiments disclosed herein, a polynucleotide including any of the elements can include a primer binding site. In illustrative embodiments, insulators and/or polyadenylation sequences can be placed before, after, between, or near genes to prevent or reduce unregulated transcription. In some embodiments, the insulator can be chicken HS4 insulator, Kaiso insulator, SAR/MAR elements, chimeric chicken insulator-SAR elements, CTCF insulator, the gypsy insulator, or the β-globin insulator or fragments thereof known in the art. In some embodiments, the insulator and/or polyadenylation sequence can be hGH polyA (SEQ ID NO:316), SPA1 (SEQ ID NO:317), SPA2 (SEQ ID NO:318), b-globin polyA spacer B (SEQ ID NO:319), b-globin polyA spacer A (SEQ ID NO:320), 250 cHS4 insulator v1 (SEQ ID NO:321), 250 cHS4 insulator v2 (SEQ ID NO:322), 650 cHS4 insulator (SEQ ID NO:323), 400 cHS4 insulator (SEQ ID NO:324), 650 cHS4 insulator and b-globin polyA spacer B (SEQ ID NO:325), or b-globin polyA spacer B and 650 cHS4 insulator (SEQ ID NO:326).

In any of the embodiments disclosed herein, a nucleic acid sequence encoding Vpx can be on the second or an optional third transcriptional unit, or on an additional transcriptional unit that is operably linked to the first inducible promoter.

Some aspects of the present disclosure include or are cells, in illustrative examples, mammalian cells, that are used as packaging cells to make replication incompetent recombinant retroviral particles, such as lentiviruses, for transduction of T cells and/or NK cells.

Any of a wide variety of cells can be selected for in vitro production of a virus or virus particle, such as a redirected recombinant retroviral particle, according to the invention. Eukaryotic cells are typically used, particularly mammalian cells including human, simian, canine, feline, equine and rodent cells. In illustrative examples, the cells are human cells. In further illustrative embodiments, the cells reproduce indefinitely, and are therefore immortal. Examples of cells that can be advantageously used in the present invention include NIH 3T3 cells, COS cells, Madin-Darby canine kidney cells, human embryonic 293T cells and any cells derived from such cells, such as gpnlslacZ φNX cells, which are derived from 293T cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, Hut-78, Jurkat, HL-60, and the like.

### Retroviral Genome Size

In the methods and compositions provided herein, the recombinant retroviral genomes, in non-limiting illustrative examples, lentiviral genomes, have a limitation to the number of polynucleotides that can be packaged into the viral particle. In some embodiments provided herein, the polypeptides encoded by the polynucleotide encoding region can be truncations or other deletions that retain a functional activity such that the polynucleotide encoding region is encoded by less nucleotides than the polynucleotide encoding region for the wild-type polypeptide. In some embodiments, the polypeptides encoded by the polynucleotide encoding region can be fusion polypeptides that can be expressed from one promoter. In some embodiments, the fusion polypeptide can have a cleavage signal to generate two or more functional polypeptides from one fusion polypeptide and one promoter. Furthermore, some functions that are not required after initial ex vivo transduction are not included in the retroviral genome, but rather are present on the surface of the replication incompetent recombinant retroviral particles via the packaging cell membrane. These various strategies are used herein to maximize the functional elements that are packaged within the replication incompetent recombinant retroviral particles.

In some embodiments, the recombinant retroviral genome to be packaged can be between 1,000, 2,000, 3,000, 4,000, 5,000, 6,000, 7,000, and 8,000 nucleotides on the low end of the range and 2,000, 3,000, 4,000, 5,000, 6,000, 7,000, 8,000, 9,000, 10,000, and 11,000 nucleotides on the high end of the range. The retroviral genome to be packaged includes one or more polynucleotide regions encoding a first and second engineering signaling polypeptide as disclosed in detail herein. In some embodiments, the recombinant retroviral genome to be packaged can be less than 5,000, 6,000, 7,000, 8,000, 9,000, 10,000, or 11,000 nucleotides. Functions discussed elsewhere herein that can be packaged include required retroviral sequences for retroviral assembly and packaging, such as a retroviral rev, gag, and pol coding regions, as well as a 5′ LTR and a 3′ LTR, or an active truncated fragment thereof, a nucleic acid sequence encoding a retroviral cis-acting RNA packaging element, and a cPPT/CTS element. Furthermore, in illustrative embodiments a replication incompetent recombinant retroviral particle herein can include any one or more or all of the following, in some embodiments in reverse orientation with respect to a 5′ to 3′ orientation established by the retroviral 5′ LTR and 3′ LTR (as illustrated in WO2019/055946 as a non-limiting example): one or more polynucleotide regions encoding a first and second engineering signaling polypeptide, at least one of which includes at least one lymphoproliferative element; a second engineered signaling polypeptide that can include a chimeric antigen receptor; an miRNA, a control element, such as a riboswitch, which typically regulates expression of the first and/or the second engineering signaling polypeptide; a recognition domain, an intron, a promoter that is active in a target cell, such as a T cell, a 2A cleavage signal and/or an IRES.

### Recombinant Retroviral Particles

Recombinant retroviral particles are disclosed in methods and compositions provided herein, for example, to transduce T cells and/or NK cells to make genetically modified T cells and/or NK cells. The recombinant retroviral particles are themselves aspects of the present invention. Typically, the recombinant retroviral particles included in aspects provided herein, are replication incompetent, meaning that a recombinant retroviral particle cannot replicate once it leaves the packaging cell. In illustrative embodiments, the recombinant retroviral particles are lentiviral particles.

Provided herein in some aspects are replication incompetent recombinant retroviral particles for use in transducing cells, typically lymphocytes and illustrative embodiments T cells and/or NK cells. The replication incompetent recombinant retroviral particles can include any of the pseudotyping elements discussed elsewhere herein. In some embodiments, the replication incompetent recombinant retroviral particles can include any of the activation elements discussed elsewhere herein. In one aspect, provided herein is a replication incompetent recombinant retroviral particle including a polynucleotide including: A. one or more transcriptional units operatively linked to a promoter active in T cells and/or NK cells, wherein the one or more transcriptional units encode a chimeric antigen receptor (CAR); and B. a pseudotyping element and a T cell activation element on its surface, wherein the T cell activation element is not encoded by a polynucleotide in the replication incompetent recombinant retroviral particle. In some embodiments, the T cell activation element can be any of the activation elements discussed elsewhere herein. In illustrative embodiments, the T cell activation element can be anti-CD3 scFvFc. In another aspect, provided herein is a replication incompetent recombinant retroviral particle, including a polynucleotide including one or more transcriptional units operatively linked to a promoter active in T cells and/or NK cells, wherein the one or more transcriptional units encode a first polypeptide including a chimeric antigen receptor (CAR) and a second polypeptide including a lymphoproliferative element. In some embodiments, the lymphoproliferative element can be a chimeric lymphoproliferative element. In illustrative embodiments, the lymphoproliferative element does not comprise IL-7 tethered to the IL-7 receptor alpha chain or a fragment thereof. In some embodiments the lymphoproliferative element does not comprise IL-15 tethered to the IL-2/IL-15 receptor beta chain.

In some aspects, provided herein is a replication incompetent recombinant retroviral particle, comprising a polynucleotide comprising one or more transcriptional units operatively linked to a promoter active in T cells and/or NK cells, wherein the one or more transcriptional units encode a first polypeptide comprising a chimeric antigen receptor (CAR) and a second polypeptide comprising a chimeric lymphoproliferative element, for example a constitutively active chimeric lymphoproliferative element. In illustrative embodiments, the chimeric lymphoproliferative element does not comprise a cytokine tethered to its cognate receptor or tethered to a fragment of its cognate receptor.

Provided herein in some aspects, is a recombinant retroviral particle that includes (i) a pseudotyping element capable of binding to a T cell and/or NK cell and facilitating membrane fusion of the recombinant retroviral particle thereto; (ii) a polynucleotide having one or more transcriptional units operatively linked to a promoter active in T cells and/or NK cells, wherein the one or more transcriptional units encode a first engineered signaling polypeptide having a chimeric antigen receptor that includes an antigen-specific targeting region, a transmembrane domain, and an intracellular activating domain, and a second engineered signaling polypeptide that includes at least one lymphoproliferative element; wherein expression of the first engineered signaling polypeptide and/or the second engineered signaling polypeptide are regulated by an in vivo control element; and (iii) an activation element on its surface, wherein the activation element is capable of binding to a T cell and/or NK cell and is not encoded by a polynucleotide in the recombinant retroviral particle. In some embodiments, the promoter active in T cells and/or NK cells is not active in the packaging cell line or is only active in the packaging cell line in an inducible manner. In any of the embodiments disclosed herein, either of the first and second engineered signaling polypeptides can have a chimeric antigen receptor and the other engineered signaling polypeptide can have at least one lymphoproliferative element.

Various elements and combinations of elements that are included in replication incompetent, recombinant retroviral particles are provided throughout this disclosure, such as, for example, pseudotyping elements, activation elements, and membrane bound cytokines, as well as nucleic acid sequences that are included in a genome of a replication incompetent, recombinant retroviral particle such as, but not limited to, a nucleic acid encoding a CAR; a nucleic acid encoding a lymphoproliferative element; a nucleic acid encoding a control element, such as a riboswitch; a promoter, especially a promoter that is constitutively active or inducible in a T cell; and a nucleic acid encoding an inhibitory RNA molecule. Furthermore, various aspects provided herein, such as methods of making recombinant retroviral particles, methods for performing adoptive cell therapy, and methods for transducing T cells, produce and/or include replication incompetent, recombinant retroviral particles. Replication incompetent recombinant retroviruses that are produced and/or included in such methods themselves form separate aspects of the present invention as replication incompetent, recombinant retroviral particle compositions, which can be in an isolated form. Such compositions can be in dried down (e.g. lyophilized) form or can be in a suitable solution or medium known in the art for storage and use of retroviral particles.

Accordingly, as a non-limiting example, provided herein in another aspect, is a replication incompetent recombinant retroviral particle having in its genome a polynucleotide having one or more nucleic acid sequences operatively linked to a promoter active in T cells and/or NK cells that in some instances, includes a first nucleic acid sequence that encodes one or more (e.g. two or more) inhibitory RNA molecules directed against one or more RNA targets and a second nucleic acid sequence that encodes a chimeric antigen receptor, or CAR, as described herein. In other embodiments, a third nucleic acid sequence is present that encodes at least one lymphoproliferative element described previously herein that is not an inhibitory RNA molecule. In certain embodiments, the polynucleotide incudes one or more riboswitches as presented herein, operably linked to the first nucleic acid sequence, the second nucleic acid sequence, and/or the third nucleic acid sequence, if present. In such a construct, expression of one or more inhibitory RNAs, the CAR, and/or one or more lymphoproliferative elements that are not inhibitory RNAs is controlled by the riboswitch. In some embodiments, two to 10 inhibitory RNA molecules are encoded by the first nucleic acid sequence. In further embodiments, two to six inhibitory RNA molecules are encoded by the first nucleic acid sequence. In illustrative embodiments, 4 inhibitory RNA molecules are encoded by the first nucleic acid sequence. In some embodiments, the first nucleic acid sequence encodes one or more inhibitory RNA molecules and is located within an intron. In certain embodiments, the intron includes all or a portion of a promoter. The promoter can be a Pol I, Pol II, or Pol III promoter. In some illustrative embodiments, the promoter is a Pol II promoter. In some embodiments, the intron is adjacent to and downstream of the promoter active in a T cell and/or NK cell. In some embodiments, the intron is EF1-α intron A.

Recombinant retroviral particle embodiments herein include those wherein the retroviral particle comprises a genome that includes one or more nucleic acids encoding one or more inhibitory RNA molecules. Various alternative embodiments of such nucleic acids that encode inhibitory RNA molecules that can be included in a genome of a retroviral particle, including combinations of such nucleic acids with other nucleic acids that encode a CAR or a lymphoproliferative element other than an inhibitory RNA molecule, are included for example, in the inhibitory RNA section provided herein, as well as in various other paragraphs that combine these embodiments. Furthermore, various alternatives of such replication incompetent recombinant retroviruses can be identified by exemplary nucleic acids that are disclosed within packaging cell line aspects disclosed herein. A skilled artisan will recognize that disclosure in this section of a recombinant retroviral particle that includes a genome that encodes one or more (e.g. two or more) inhibitory RNA molecules, can be combined with various alternatives for such nucleic acids encoding inhibitory RNA molecules provided in other sections herein. Furthermore, a skilled artisan will recognize that such nucleic acids encoding one or more inhibitory RNA molecules can be combined with various other functional nucleic acid elements provided herein, as for example, disclosed in the section herein that focuses on inhibitory RNA molecules and nucleic acid encoding these molecules. In addition, the various embodiments of specific inhibitory RNA molecules provided herein in other sections can be used in recombinant retroviral particle aspects of the present disclosure.

Necessary elements of recombinant retroviral vectors, such as lentiviral vectors, are known in the art. These elements are included in the packaging cell line section and in details for making replication incompetent, recombinant retroviral particles provided in the Examples section and as illustrated in WO2019/055946. For example, lentiviral particles typically include packaging elements REV, GAG and POL, which can be delivered to packaging cell lines via one or more packaging plasmids, a pseudotyping element, various examples which are provided herein, which can be delivered to a packaging cell line via a pseudotyping plasmid, and a genome, which is produced by a polynucleotide that is delivered to a host cell via a transfer plasmid. This polynucleotide typically includes the viral LTRs and a psi packaging signal. The 5′ LTR can be a chimeric 5′ LTR fused to a heterologous promoter, which includes 5′ LTRs that are not dependent on Tat transactivation. The transfer plasmid can be self-inactivating, for example, by removing a U3 region of the 3′ LTR. In some non-limiting embodiments, Vpu, such as a polypeptide comprising Vpu (sometimes called a “Vpu polypeptide” herein) including but not limited to, Src-FLAG-Vpu, is packaged within the retroviral particle for any composition or method aspect and embodiment provided herein that includes a retroviral particle. In some non-limiting embodiments, Vpx, such as Src-FLAG-Vpx, is packaged within the retroviral particle. Not to be limited by theory, upon transduction of a T cells, Vpx enters the cytosol of the cells and promotes the degradation of SAMHD1, resulting in an increased pool of cytoplasmic dNTPs available for reverse transcription. In some non-limiting embodiments, Vpu and Vpx is packaged within the retroviral particle for any composition or method aspect and embodiment that includes a retroviral particle provided herein.

Retroviral particles (e.g. lentiviral particles) included in various aspects of the present invention are in illustrative embodiments, replication incompetent, especially for safety reasons for embodiments that include introducing cells transduced with such retroviral particles into a subject. When replication incompetent retroviral particles are used to transduce a cell, retroviral particles are not produced from the transduced cell. Modifications to the retroviral genome are known in the art to assure that retroviral particles that include the genome are replication incompetent. However, it will be understood that in some embodiments for any of the aspects provided herein, replication competent recombinant retroviral particles can be used.

A skilled artisan will recognize that the functional elements discussed herein can be delivered to packaging cells and/or to T cells using different types of vectors, such as expression vectors. Illustrative aspects of the invention utilize retroviral vectors, and in some particularly illustrative embodiments lentiviral vectors. Other suitable expression vectors can be used to achieve certain embodiments herein. Such expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus), for example a gamma retrovirus; or human immunodeficiency virus (see, e.g., Miyoshi et al., PNAS 94:10319 23, 1997; Takahashi et al., J Virol 73:7812 7816, 1999); and the like.

As disclosed herein, replication incompetent recombinant retroviral particles are a common tool for gene delivery (Miller, Nature (1992) 357:455-460). The ability of replication incompetent recombinant retroviral particles to deliver an unrearranged nucleic acid sequence into a broad range of rodent, primate and human somatic cells makes replication incompetent recombinant retroviral particles well suited for transferring genes to a cell. In some embodiments, the replication incompetent recombinant retroviral particles can be derived from the Alpharetrovirus genus, the Betaretrovirus genus, the Gammaretrovirus genus, the Deltaretrovirus genus, the Epsilonretrovirus genus, the Lentivirus genus, or the Spumavirus genus. There are many retroviruses suitable for use in the methods disclosed herein. For example, murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) can be used. A detailed list of retroviruses may be found in Coffin et al (“Retroviruses” 1997 Cold Spring Harbor Laboratory Press Eds: J M Coffin, S M Hughes, H E Varmus pp 758-763). Details on the genomic structure of some retroviruses may be found in the art. By way of example, details on HIV may be found from the NCBI Genbank (i.e. Genome Accession No. AF033819).

In illustrative embodiments, the replication incompetent recombinant retroviral particles can be derived from the Lentivirus genus. In some embodiments, the replication incompetent recombinant retroviral particles can be derived from HIV, SIV, or FIV. In further illustrative embodiments, the replication incompetent recombinant retroviral particles can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. In vivo, HIV can infect terminally differentiated cells that rarely divide, such as lymphocytes and macrophages.

In illustrative embodiments, replication incompetent recombinant retroviral particles provided herein contain Vpx polypeptide.

In some embodiments, replication incompetent recombinant retroviral particles provided herein comprise and/or contain Vpu polypeptide.

In illustrative embodiments, a retroviral particle is a lentiviral particle. Such retroviral particle typically includes a retroviral genome within a capsid which is located within a viral envelope.

In some embodiments, DNA-containing viral particles are utilized instead of recombinant retroviral particles. Such viral particles can be adenoviruses, adeno-associated viruses, herpesviruses, cytomegaloviruses, poxviruses, avipox viruses, influenza viruses, vesicular stomatitis virus (VSV), or Sindbis virus. A skilled artisan will appreciate how to modify the methods disclosed herein for use with different viruses and retroviruses, or retroviral particles. Where viral particles are used that include a DNA genome, a skilled artisan will appreciate that functional units can be included in such genomes to induce integration of all or a portion of the DNA genome of the viral particle into the genome of a T cell transduced with such virus.

In some embodiments, the HIV RREs and the polynucleotide region encoding HIV Rev can be replaced with N-terminal RGG box RNA binding motifs and a polynucleotide region encoding ICP27. In some embodiments, the polynucleotide region encoding HIV Rev can be replaced with one or more polynucleotide regions encoding adenovirus E1B 55-kDa and E4 Orf6.

Provided herein in one aspect is a container, such as a commercial container or package, or a kit comprising the same, comprising isolated replication incompetent recombinant retroviral particles according to any of the replication incompetent recombinant retroviral particle aspects provided herein. Furthermore, provided herein in another aspect is a container, such as a commercial container or package, or a kit comprising the same, comprising isolated packaging cells, in illustrative embodiments isolated packaging cells from a packaging cell line, according to any of the packaging cell and/or packaging cell line aspects provided herein. In some embodiments, the kit includes additional containers that include additional reagents such as buffers or reagents used in methods provided herein. Furthermore provided herein in certain aspects are use of any replication incompetent recombinant retroviral particle provided herein in any aspect, in the manufacture of a kit for genetically modifying a T cell or NK cell according to any aspect provided herein. Furthermore provided herein in certain aspects are use of any packaging cell or packaging cell line provided herein in any aspect, in the manufacture of a kit for producing the replication incompetent recombinant retroviral particles according to any aspect provided herein.

Provided herein in one aspect is a commercial container containing a replication incompetent recombinant retroviral particle and instructions for the use thereof to treat tumor growth in a subject, wherein the replication incompetent recombinant retroviral particle comprises in its genome a polynucleotide comprising one or more nucleic acid sequences operatively linked to a promoter active in T cells and/or NK cells. In some embodiments, a nucleic acid sequence of the one or more nucleic acid sequences can encode a chimeric antigen receptor (CAR) comprising an antigen-specific targeting region (ASTR), a transmembrane domain, and an intracellular activating domain. In some embodiments, a nucleic acid sequence of the one or more nucleic acid sequences can encode two or more inhibitory RNA molecules directed against one or more RNA targets.

The container that contains the recombinant retroviral particles can be a tube, vial, well of a plate, or other vessel for storage of a recombinant retroviral particle. The kit can include two or more containers wherein a second or other container can include, for example, a solution or media for transduction of T cells and/or NK cells, and/or the second or other container can include a pH-modulating pharmacologic agent. Any of these containers can be of industrial strength and grade. The replication incompetent recombinant retroviral particle in such aspects that include a kit and a nucleic acid encoding an inhibitory RNA molecule, can be any of the embodiments for such replication incompetent recombinant retroviral particles provided herein, which include any of the embodiments for inhibitory RNA provided herein.

In another aspect, provided herein is the use of a replication incompetent recombinant retroviral particle in the manufacture of a kit for genetically modifying a T cell or NK cell, wherein the use of the kit includes: contacting the T cell or NK cell ex vivo with the replication incompetent recombinant retroviral particle, wherein the replication incompetent recombinant retroviral particle includes a pseudotyping element on a surface and a T cell activation element on the surface, wherein said contacting facilitates transduction of the T cell or NK cell by the replication incompetent recombinant retroviral particle, thereby producing a genetically modified T cell or NK cell. In some embodiments, the T cell or NK cell can be from a subject. In some embodiments, the T cell activation element can be membrane-bound. In some embodiments, the contacting can be performed for between 1, 2, 3, 4, 5, 6, 7, or 8 hours on the low end of the range and 4, 5, 6, 7, 8, 10, 12, 15, 18, 21, and 24 hours on the high end of the range, for example, between 1 and 12 hours. The replication incompetent recombinant retroviral particle for use in the manufacture of a kit can include any of the aspects, embodiments, or subembodiments discussed elsewhere herein.

In another aspect, provided herein is a pharmaceutical composition for treating or preventing cancer or tumor growth comprising a replication incompetent recombinant retroviral particle as an active ingredient. In another aspect, provided herein is an infusion composition or other delivery solution for treating or preventing cancer or tumor growth comprising a replication incompetent recombinant retroviral particle. The replication incompetent recombinant retroviral particle of the pharmaceutical composition or infusion composition can include any of the aspects, embodiments, or subembodiments discussed above or elsewhere herein.

### Genetically Modified T Cells and NK Cells

In embodiments of the methods and compositions herein, genetically modified lymphocytes are produced, which themselves are a separate aspect of the invention. Such genetically modified lymphocytes can be genetically modified and/or transduced lymphocytes. In one aspect, provided herein a genetically modified T cell or NK cell is made using a method according to any aspect for genetically modifying T cells and/or NK cells in blood or a component thereof, provided herein. For example, in some embodiments, the T cell or NK cell has been genetically modified to express a first engineered signaling polypeptide. In illustrative embodiments, the first engineered signaling polypeptide can be a lymphoproliferative element or a CAR that includes an antigen-specific targeting region (ASTR), a transmembrane domain, and an intracellular activating domain. In some embodiments, the T cell or NK cell can further include a second engineered signaling polypeptide that can be a CAR or a lymphoproliferative element. In some embodiments, the lymphoproliferative element can be a chimeric lymphoproliferative element. In some embodiments, the T cell or NK cell can further include a pseudotyping element on a surface. In some embodiments, the T cell or NK cell can further include an activation element on a surface. The CAR, lymphoproliferative element, pseudotyping element, and activation element of the genetically modified T cell or NK cell can include any of the aspects, embodiments, or subembodiments disclosed herein. In illustrative embodiments, the activation element can be anti-CD3 antibody, such as an anti-CD3 scFvFc.

In some embodiments, genetically modified lymphocytes are lymphocytes such as T cells or NK cells that have been genetically modified to express a first engineered signaling polypeptide comprising at least one lymphoproliferative element and/or a second engineered signaling polypeptide comprising a chimeric antigen receptor, which includes an antigen-specific targeting region (ASTR), a transmembrane domain, and an intracellular activating domain. In some embodiments of any of the aspects herein, the NK cells are NKT cells. NKT cells are a subset of T cells that express CD3 and typically coexpress an αβ T-cell receptor, but also express a variety of molecular markers that are typically associated with NK cells (such as NK1.1 or CD56).

Genetically modified lymphocytes of the present disclosure possess a heterologous nucleic acid sequence that has been introduced into the lymphocyte by a recombinant DNA method. For example, the heterologous sequence in illustrative embodiments is inserted into the lymphocyte during a method for transducing the lymphocyte provided herein. The heterologous nucleic acid is found within the lymphocyte and in some embodiments is or is not integrated into the genome of the genetically modified lymphocyte.

In illustrative embodiments, the heterologous nucleic acid is integrated into the genome of the genetically modified lymphocyte. Such lymphocytes are produced, in illustrative embodiments, using a method for transducing lymphocytes provided herein, that utilizes a recombinant retroviral particle. Such recombinant retroviral particle can include a polynucleotide that encodes a chimeric antigen receptor that typically includes at least an antigen-specific targeting region (ASTR), a transmembrane domain, and an intracellular activating domain. Provided herein in other sections of this disclosure are various embodiments of replication incompetent recombinant retroviral particles and polynucleotides encoded in a genome of the replication incompetent retroviral particle, that can be used to produce genetically modified lymphocytes that themselves form another aspect of the present disclosure.

Genetically modified lymphocytes of the present disclosure can be isolated outside the body. For example, such lymphocytes can be found in media and other solutions that are used for ex vivo transduction as provided herein. The lymphocytes can be present in a genetically unmodified form in blood that is collected from a subject in methods provided herein, and then genetically modified during method of transduction. The genetically modified lymphocytes can be found inside a subject after they are introduced or reintroduced into the subject after they have been genetically modified. The genetically modified lymphocytes can be a resting T cell or a resting NK cell, or the genetically modified T cell or NK cell can be actively dividing, especially after it expresses some of the functional elements provided in nucleic acids that are inserted into the T cell or NK cell after transduction as disclosed herein.

Provided herein in one aspect is a transduced and/or genetically modified T cell or NK cell, comprising a recombinant polynucleotide comprising one or more transcriptional units operatively linked to a promoter active in T cells and/or NK cells, in its genome.

In some embodiments, provided herein are genetically modified lymphocytes, in illustrative embodiments T cells and/or NK cells, that relate to either aspects for transduction of T cells and/or NK cells in blood or a component thereof, that include transcription units that encode one, two, or more (e.g. 1-10, 2-10, 4-10, 1-6, 2-6, 3-6, 4-6, 1-4, 2-4, 3-4) inhibitory RNA molecules. In some embodiments, such inhibitory RNA molecules are lymphoproliferative elements and therefore, can be included in any aspect or embodiment disclosed herein as the lymphoproliferative element as long as they induce proliferation of a T cell and/or an NK cell, or otherwise meet a test for a lymphoproliferative element provided herein.

Inhibitory RNA molecules directed against a variety of target RNAs can be used in embodiments of any of the aspects provided herein. For example, one, most or all of the one (e.g. two) or more inhibitory RNA molecules decrease expression of an endogenous TCR. In some embodiments, the RNA target is mRNA transcribed from a gene selected from the group consisting of: PD-1, CTLA4, TCR alpha, TCR beta, CD3 zeta, SOCS, SMAD2, a miR-155 target, IFN gamma, cCBL, TRAIL2, PP2A, and ABCG1. In some embodiments of this aspect at least one of the one (e.g. two) or more inhibitory RNA molecules is miR-155.

In some embodiments of the aspect immediately above where the T cell or NK cell comprises one or more (e.g. two or more) inhibitory RNA molecules and the CAR, or nucleic acids encoding the same, the ASTR of the CAR is an MRB ASTR and/or the ASTR of the CAR binds to a tumor associated antigen. Furthermore, in some embodiments of the above aspect, the first nucleic acid sequence is operably linked to a riboswitch, which for example is capable of binding a nucleoside analog, and in illustrative embodiments is an antiviral drug such as acyclovir.

In the methods and compositions disclosed herein, expression of engineered signaling polypeptides is regulated by a control element, and in some embodiments, the control element is a polynucleotide comprising a riboswitch. In certain embodiments, the riboswitch is capable of binding a nucleoside analog and when the nucleoside analog is present, one or both of the engineered signaling polypeptides are expressed.

### Nucleic Acids

The present disclosure provides nucleic acid encoding polypeptides of the present disclosure. A nucleic acid will in some embodiments be DNA, including, e.g., a recombinant expression vector. A nucleic acid will in some embodiments be RNA, e.g., in vitro synthesized RNA.

In some embodiments, a nucleic acid provides for production of a polypeptide of the present disclosure, e.g., in a mammalian cell. In other cases, a subject nucleic acid provides for amplification of the nucleic acid encoding a polypeptide of the present disclosure.

A nucleotide sequence encoding a polypeptide of the present disclosure can be operably linked to a transcriptional control element, e.g., a promoter, and enhancer, etc.

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some instances, the locus or construct or trans gene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.), the disclosures of which are incorporated herein by reference.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Neri (p46) promoter; see, e.g., Eckelhart et al. (2011) Blood 117:1565.

In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GALI promoter, a GALIO promoter, an ADH2 promoter, a PH05 promoter, a CUP1 promoter, a GAL7 promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacterial., 1991: 173(1): 86-93; Alpuche-Aranda et al., PNAS, 1992; 89(21): 10079-83), a nirB promoter (Harborne et al. (1992) Mal. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mal. Microbial. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Laci repressor protein changes conformation when contacted with lactose, thereby preventing the Laci repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

A nucleotide sequence encoding a polypeptide of the disclosure can be present in an expression vector and/or a cloning vector. Nucleotide sequences encoding two separate polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating subject recombinant constructs. The following bacterial vectors are provided by way of example: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). The following eukaryotic vectors are provided by way of example: pWLneo, pSV2cat, pOG44, PXR1, pSG (Stratagene) pSVK3, pBPV, pMSG, and pSVL (Pharmacia).

Expression vectors generally have convenient restriction sites located near the promoter sequence to provide for the insertion of nucleic acid sequences encoding heterologous proteins. A selectable marker operative in the expression host may be present.

As noted above, in some embodiments, a nucleic acid encoding a polypeptide of the present disclosure will in some embodiments be RNA, e.g., in vitro synthesized RNA. Methods for in vitro synthesis of RNA are known in the art; any known method can be used to synthesize RNA including a nucleotide sequence encoding a polypeptide of the present disclosure. Methods for introducing RNA into a host cell are known in the art. See, e.g., Zhao et al. (2010) Cancer Res. 15:9053. Introducing RNA including a nucleotide sequence encoding a polypeptide of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

Various aspects and embodiments that include a polynucleotide, a nucleic acid sequence, and/or a transcriptional unit, and/or a vector including the same, further include one or more of a Kozak-type sequence (also called a Kozak-related sequence herein), a woodchuck hepatitis virus post-transcriptional regulatory element (WPRE), and a double stop codon or a triple stop codon, wherein one or more stop codons of the double stop codon or the triple stop codon define a termination of a reading from of at least one of the one or more transcriptional units. In certain embodiments, a polynucleotide a nucleic acid sequence, and/or a transcriptional unit, and/or a vector including the same, further includes a Kozak-type sequence having a 5′ nucleotide within 10 nucleotides upstream of a start codon of at least one of the one or more transcriptional units. Kozak determined the Kozak consensus sequence, (GCC)GCCRCCATG (SEQ ID NO:327), for 699 vertebrate mRNAs, where R is a purine (A or G) (Kozak. Nucleic Acids Res. 1987 Oct. 26; 15(20):8125-48). In one embodiment the Kozak-type sequence is or includes CCACCAT/UG(G) (SEQ ID NO:328), CCGCCAT/UG(G) (SEQ ID NO:329), GCCGCCGCCAT/UG(G) (SEQ ID NO:330), or GCCGCCACCAT/UG(G) (SEQ ID NO:331) (with nucleotides in parenthesis representing optional nucleotides and nucleotides separated by a slash indicated different possible nucleotides at that position, for example depending on whether the nucleic acid is DNA or RNA. In these embodiments that include the AU/TG start codon, the A can be considered position 0. In certain illustrative embodiments, the nucleotides at −3 and +4 are identical, for example the −3 and +4 nucleotides can be G. In another embodiment the Kozak-type sequence includes an A or G in the 3rd position upstream of ATG where ATG is the start codon. In another embodiment the Kozak-type sequence includes an A or G in the 3rd position upstream of AUG where AUG is the start codon. In an illustrative embodiment, the Kozak sequence is (GCC)GCCRCCATG (SEQ ID NO:327), where R is a purine (A or G). In an illustrative embodiment, the Kozak-type sequence is GCCGCCACCAUG (SEQ ID NO:332). In another embodiment, which can be combined with the preceding embodiment that includes a Kozak-type sequence and/or the following embodiment that includes triple stop codon, the polynucleotide includes a WPRE element. WPREs have been characterized in the art (See e.g., (Higashimoto et al., Gene Ther. 2007; 14: 1298)) and as illustrated in WO2019/055946. In some embodiments, the WPRE element is located 3′ of a stop codon of the one or more transcriptional units and 5′ to a 3′ LTR of the polynucleotide. In another embodiment, which can be combined with either or both of the preceding embodiments (i.e. an embodiment wherein the polynucleotide includes a Kozak-type sequence and/or an embodiment wherein the polynucleotide includes a WPRE), the one or more transcriptional units terminates with one or more stop codons of a double stop codon or a triple stop codon, wherein the double stop codon includes a first stop codon in a first reading frame and a second stop codon in a second reading frame, or a first stop codon in frame with a second stop codon, and wherein the triple stop codon includes a first stop codon in a first reading frame, a second stop codon in a second reading frame, and a third stop codon in a third reading frame, or a first stop codon in frame with a second stop codon and a third stop codon.

A triple stop codon herein includes three stop codons, one in each reading frame, within 10 nucleotides of each other, and preferably having overlapping sequence, or three stop codons in the same reading frame, preferably at consecutive codons. A double stop codon means two stop codons, each in a different reading frame, within 10 nucleotides of each other, and preferably having overlapping sequences, or two stop codons in the same reading frame, preferably at consecutive codons.

In some of the methods and compositions disclosed herein, the introduction of DNA into PBMCs, B cells, T cells and/or NK cells and optionally the incorporation of the DNA into the host cell genome, is performed using methods that do not utilize replication incompetent recombinant retroviral particles. For example, other viral vectors can be utilized, such as those derived from adenovirus, adeno-associated virus, or herpes simplex virus-1, as non-limiting examples.

In some embodiments, methods provided herein can include transfecting target cells with non-viral vectors. In any of the embodiments disclosed herein that utilize non-viral vectors to transfect target cells, the non-viral vectors, including naked DNA, can be introduced into the target cells, such as for example, PBMCs, B cells, T cells and/or NK cells using methods that include electroporation, nucleofection, liposomal formulations, lipids, dendrimers, cationic polymers such as poly(ethylenimine) (PEI) and poly(l-lysine) (PLL), nanoparticles, cell-penetrating peptides, microinjection, and/or non-integrating lentiviral vectors. In some embodiments, DNA can be introduced into target cells, such as PBMCs, B cells, T cells and/or NK cells in a complex with liposomes and protamine. Other methods for transfecting T cells and/or NK cells ex vivo that can be used in embodiments of methods provided herein, are known in the art (see e.g., Morgan and Boyerinas, Biomedicines. 2016 Apr. 20; 4(2). pii: E9, incorporated by reference herein in its entirety).

In some embodiments of method provided herein, DNA can be integrated into the genome using transposon-based carrier systems by co-transfection, co-nucleofection or co-electroporation of target DNA as plasmid containing the transposon ITR fragments in 5′ and 3′ ends of the gene of interest and transposase carrier system as DNA or mRNA or protein or site specific serine recombinases such as phiC31 that integrates the gene of interest in pseudo attP sites in the human genome, in this instance the DNA vector contains a 34 to 40 bp attB site that is the recognition sequence for the recombinase enzyme (Bhaskar Thyagarajan et al. Site-Specific Genomic Integration in Mammalian Cells Mediated by Phage φC31 Integrase, Mol Cell Biol. 2001 June; 21(12): 3926-3934) and co transfected with the recombinase. For T cells and/or NK cells, transposon-based systems that can be used in certain methods provided herein utilize the Sleeping Beauty DNA carrier system (see e.g., U.S. Pat. No. 6,489,458 and U.S. patent application Ser. No. 15/434,595, incorporated by reference herein in their entireties), the PiggyBac DNA carrier system (see e.g., Manuri et al., Hum Gene Ther. 2010 April; 21(4):427-37, incorporated by reference herein in its entirety), or the Tol2 transposon system (see e.g., Tsukahara et al., Gene Ther. 2015 February; 22(2): 209-215, incorporated by reference herein in its entirety) in DNA, mRNA, or protein form. In some embodiments, the transposon and/or transposase of the transposon-based vector systems can be produced as a minicircle DNA vector before introduction into T cells and/or NK cells (see e.g., Hudecek et al., Recent Results Cancer Res. 2016; 209:37-50 and Monjezi et al., Leukemia. 2017 January; 31(1):186-194, incorporated by reference herein in their entireties). The CAR or lymphoproliferative element can also be integrated into the defined and specific sites in the genome using CRISPR or TALEN mediated integration, by adding 50-1000 bp homology arms homologous to the integration 5′ and 3′ of the target site (Jae Seong Lee et al. Scientific Reports 5, Article number: 8572 (2015), Site-specific integration in CHO cells mediated by CRISPR/Cas9 and homology-directed DNA repair pathway). CRISPR or TALEN provide specificity and genomic-targeted cleavage and the construct will be integrated via homology-mediated end joining (Yao X at al. Cell Res. 2017 June; 27(6):801-814. doi: 10.1038/cr.2017.76. Epub 2017 May 19). The CRISPR or TALEN can be co-transfected with target plasmid as DNA, mRNA, or protein.

### Inhibitory RNA Molecules

Embodiments of any of the aspects provided herein can include recombinant retroviral particles whose genomes are constructed to induce expression of one or more, and in illustrative embodiments two or more, inhibitory RNA molecules, such as for example, a miRNA or shRNA, after integration into a host cell, such as a lymphocyte (e.g. a T cell and/or an NK cell). Such inhibitory RNA molecules can be encoded within introns, including for example, an EF1-a intron. This takes advantage of the present teachings of methods to maximize the functional elements that can be included in a packageable retroviral genome to overcome shortcomings of prior teachings and maximize the effectiveness of such recombinant retroviral particles in adoptive T cell therapy.

In some embodiments, the inhibitory RNA molecule includes a 5′ strand and a 3′ strand (in some examples, sense strand and antisense strand) that are partially or fully complementary to one another such that the two strands are capable of forming a 18-25 nucleotide RNA duplex within a cellular environment. The 5′ strand can be 18, 19, 20, 21, 22, 23, 24, or 25 nucleotides in length, and the 3′ strand can be 18, 19, 20, 21, 22, 23, 24, or 25 nucleotides in length. The 5′ strand and the 3′ strand can be the same or different lengths, and the RNA duplex can include one or more mismatches. Alternatively, the RNA duplex has no mismatches.

The inhibitory RNA molecules included in compositions and methods provided herein, in certain illustrative examples, do not exist and/or are not expressed naturally in T cells into whose genome they are inserted. In some embodiments, the inhibitory RNA molecule is a miRNA or an shRNA. In some embodiments, where reference is made herein or in priority filings, to a nucleic acid encoding an siRNA, especially in a context where the nucleic acid is part of a genome, it will be understood that such nucleic acid is capable of forming an siRNA precursor such as miRNA or shRNA in a cell that is processed by DICER to form a double stranded RNA that typically interacts with, or becomes part of a RISK complex. In some embodiments, an inhibitory molecule of an embodiment of the present disclosure is a precursor of a miRNA, such as for example, a Pri-miRNA or a Pre-miRNA, or a precursor of an shRNA. In some embodiments, the miRNA or shRNA are artificially derived (i.e. artificial miRNAs or siRNAs). In other embodiments, the inhibitory RNA molecule is a dsRNA (either transcribed or artificially introduced) that is processed into an siRNA or the siRNA itself. In some embodiments, the miRNA or shRNA has a sequence that is not found in nature, or has at least one functional segment that is not found in nature, or has a combination of functional segments that are not found in nature.

In some embodiments, inhibitory RNA molecules are positioned in the first nucleic acid molecule in a series or multiplex arrangement such that multiple miRNA sequences are simultaneously expressed from a single polycistronic miRNA transcript. In some embodiments, the inhibitory RNA molecules can be adjoined to one another either directly or indirectly by non-functional linker sequence(s). The linker sequence in some embodiments, is between 5 and 120 nucleotides in length, and in some embodiments can be between 10 and 40 nucleotides in length, as non-limiting examples. In illustrative embodiments the first nucleic acid sequence encoding one or more (e.g. two or more) inhibitory RNAs and the second nucleic acid sequence encoding a CAR (e.g. an MRB-CAR) are operably linked to a promoter that is active constitutively or that can be induced in a T cell or NK cell. As such, the inhibitory RNA molecule(s) (e.g. miRNAs) as well as the CAR are expressed in a polycistronic manner. Additionally, functional sequences can be expressed from the same transcript. For example, any of the lymphoproliferative elements provided herein that are not inhibitory RNA molecules, can be expressed from the same transcript as the CAR and the one or more (e.g. two or more) inhibitory RNA molecules.

In some embodiments, the inhibitory RNA molecule is a naturally occurring miRNA such as but not limited to miR-155. Alternatively, artificial miRNAs can be produced in which sequences capable of forming a hybridizing/complementary stem structure and directed against a target RNA, are placed in a miRNA framework that includes microRNA flanking sequences for microRNA processing and a loop, which can optionally be derived from the same naturally occurring miRNA as the flanking sequences, between the stem sequences. Thus, in some embodiments, an inhibitory RNA molecule includes from 5′ to 3′ orientation: a 5′ microRNA flanking sequence, a 5′ stem, a loop, a 3′ stem that is partially or fully complementary to said 5′ stem, and a 3′ microRNA flanking sequence. In some embodiments, the 5′ stem (also called a 5′ arm herein) is 18, 19, 20, 21, 22, 23, 24 or 25 nucleotides in length. In some embodiments, the 3′ stem (also called a 3′ arm herein) is 18, 19, 20, 21, 22, 23, 24, or 25 nucleotides in length. In some embodiments, the loop is 3 to 40, 10 to 40, 20 to 40, or 20 to 30 nucleotides in length, and in illustrative embodiments the loop can be 18, 19, 20, 21, or 22 nucleotides in length. In some embodiments, one stem is two nucleotides longer than the other stem. The longer stem can be the 5′ or the 3′ stem.

In some embodiments, the 5′ microRNA flanking sequence, 3′ microRNA flanking sequence, or both, are derived from a naturally occurring miRNA, such as but not limited to miR-155, miR-30, miR-17-92, miR-122, and miR-21. In certain embodiments, the 5′ microRNA flanking sequence, 3′ microRNA flanking sequence, or both, are derived from a miR-155, such as, e.g., the miR-155 from Mus musculus or Homo sapiens. Inserting a synthetic miRNA stem-loop into a miR-155 framework (i.e. the 5′ microRNA flanking sequence, the 3′ microRNA flanking sequence, and the loop between the miRNA 5′ and 3′ stems) is known to one of ordinary skill in the art (Chung, K. et al. 2006. Nucleic Acids Research. 34(7):e53; U.S. Pat. No. 7,387,896). The SIBR (synthetic inhibitory BIC-derived RNA) sequence (Chung et al. 2006 supra), for example, has a 5′ microRNA flanking sequence consisting of nucleotides 134-161 (SEQ ID NO:333) of the Mus musculus BIC noncoding mRNA (Genbank ID AY096003.1) and a 3′ microRNA flanking sequence consisting of nucleotides 223-283 of the Mus musculus BIC noncoding mRNA (Genbank ID AY096003.1). In one study, the SIBR sequence was modified (eSIBR) to enhance expression of miRNAs (Fowler, D. K. et al. 2015. Nucleic acids Research 44(5):e48). In some embodiments of the present disclosure, miRNAs can be placed in the SIBR or eSIBR miR-155 framework. In illustrative embodiments herein, miRNAs are placed in a miR-155 framework that includes the 5′ microRNA flanking sequence of miR-155 represented by SEQ ID NO:333, the 3′ microRNA flanking sequence represented by SEQ ID NO:334 (nucleotides 221-265 of the Mus musculus BIC noncoding mRNA); and a modified miR-155 loop (SEQ ID NO:335). Thus, in some embodiments, the 5′ microRNA flanking sequence of miR-155 is SEQ ID NO:333 or a functional variant thereof, such as, for example, a sequence that is the same length as SEQ ID NO:333, or 95%, 90%, 85%, 80%, 75%, or 50% as long as SEQ ID NO: 333 or is 100 nucleotides or less, 95 nucleotides or less, 90 nucleotides or less, 85 nucleotides or less, 80 nucleotides or less, 75 nucleotides or less, 70 nucleotides or less, 65 nucleotides or less, 60 nucleotides or less, 55 nucleotides or less, 50 nucleotides or less, 45 nucleotides or less, 40 nucleotides or less, 35 nucleotides or less, 30 nucleotides or less, or 25 nucleotides or less; and is at least 50%, 55%, 60%, 65%, 70%, 75%, 80%, 85%, 90%, or 95% identical to SEQ ID NO:333. In some embodiments, the 3′ microRNA flanking sequence of miR-155 is SEQ ID NO:334 or a functional variant thereof, such as, for example, the same length as SEQ ID NO:334, or 95%, 90%, 85%, 80%, 75%, or 50% as long as SEQ ID NO:334 or is a sequence that is 100 nucleotides or less, 95 nucleotides or less, 90 nucleotides or less, 85 nucleotides or less, 80 nucleotides or less, 75 nucleotides or less, 70 nucleotides or less, 65 nucleotides or less, 60 nucleotides or less, 55 nucleotides or less, 50 nucleotides or less, 45 nucleotides or less, 40 nucleotides or less, 35 nucleotides or less, 30 nucleotides or less, or 25 nucleotides or less; and is at least 50%, 55%, 60%, 65%, 70%, 75%, 80%, 85%, 90%, or 95% identical to SEQ ID NO:334. However, any known microRNA framework that is functional to provide proper processing within a cell of miRNAs inserted therein to form mature miRNA capable of inhibiting expression of a target mRNA to which they bind, is contemplated within the present disclosure.

In some embodiments, at least one, at least two, at least three, or at least four of the inhibitory RNA molecules encoded by a nucleic acid sequence in a polynucleotide of a replication incompetent recombinant retroviral particle has the following arrangement in the 5′ to 3′ orientation: a 5′ microRNA flanking sequence, a 5′ stem, a loop, a 3′ stem that is partially or fully complementary to said 5′ stem, and a 3′ microRNA flanking sequence. In some embodiments, all of the inhibitory RNA molecules have the following arrangement in the 5′ to 3′ orientation: a 5′ microRNA flanking sequence, a 5′ stem, a loop, a 3′ stem that is partially or fully complementary to said 5′ stem, and a 3′ microRNA flanking sequence. As disclosed herein, the inhibitory RNA molecules can be separated by one or more linker sequences, which in some embodiments have no function except to act as spacers between inhibitory RNA molecules.

In some embodiments, where two or more inhibitory RNA molecules (in some examples, 1, 2, 3, 4, 5, 6, 7, 8, 9, or 10 inhibitory RNA molecules) are included, these inhibitory RNA molecules are directed against the same or different RNA targets (such as e.g. mRNAs transcribed from genes of interest). In illustrative embodiments, between 2 and 10, 2 and 8, 2 and 6, 2 and 5, 3 and 5, 3 and 6, or 4 inhibitory RNA molecules are included in the first nucleic acid sequence. In an illustrative embodiment, four inhibitory RNA molecules are included in the first nucleic acid sequence.

In some embodiments, the one or more inhibitor RNA molecules are one or more lymphoproliferative elements, accordingly, in any aspect or embodiment provided herein that includes a lymphoproliferative element, unless incompatible therewith (e.g. a polypeptide lymphoproliferative element), or already state therein. In some embodiments, the RNA targets are mRNAs transcribed from genes that are expressed by T cells such as but not limited to PD-1 (prevent inactivation); CTLA4 (prevent inactivation); TCRα (safety—prevent autoimmunity); TCRb (safety—prevent autoimmunity); CD3Z (safety—prevent autoimmunity); SOCS1 (prevent inactivation); SMAD2 (prevent inactivation); a miR-155 target (promote activation); IFN gamma (reduce CRS); cCBL (prolong signaling); TRAIL2 (prevent death); PP2A (prolong signaling); ABCG1 (increase cholesterol microdomain content by limiting clearance of cholesterol). In illustrative examples, miRNAs inserted into the genome of T cells in methods provided herein, are directed at targets such that proliferation of the T cells is induced and/or enhanced and/or apoptosis is suppressed.

In some embodiments, the RNA targets include mRNAs that encode components of the T cell receptor (TCR) complex. Such components can include components for generation and/or formation of a T cell receptor complex and/or components for proper functioning of a T cell receptor complex. Accordingly, in one embodiment at least one of the two or more of inhibitory RNA molecules causes a decrease in the formation and/or function of a TCR complex, in illustrative embodiments one or more endogenous TCR complexes of a T cell. The T cell receptor complex includes TCRa, TCRb, CD3d, CD3e, CD3g, and CD3z. It is known that there is a complex interdependency of these components such that a decrease in the expression of any one subunit will result in a decrease in the expression and function of the complex. Accordingly, in one embodiment the RNA target is an mRNA expressing one or more of TCRa, TCRb, CD3d, CD3e, CD3g, and CD3z endogenous to a transduced T cell. In certain embodiments, the RNA target is mRNA transcribed from the endogenous TCRα or TCRβ gene of the T cell whose genome comprises the first nucleic acid sequence encoding the one or more miRNAs. In illustrative embodiments, the RNA target is mRNA transcribed from the TCRα gene. In certain embodiments, inhibitory RNA molecules directed against mRNAs transcribed from target genes with similar expected utilities can be combined. In other embodiments, inhibitory RNA molecules directed against target mRNAs transcribed from target genes with complementary utilities can be combined. In some embodiments, the two or more inhibitory RNA molecules are directed against the mRNAs transcribed from the target genes CD3Z, PD1, SOCS1, and/or IFN gamma.

In some embodiments, the inhibitory RNA, for example miRNA, targets mRNA encoding Cb1 Proto-Oncogene (RNF55) (also known as cCBL and RNF55) (HGNC: 1541, Entrez Gene: 867, OMIM: 165360), T-Cell Receptor T3 Zeta Chain (CD3z) (HGNC: 1677, Entrez Gene: 919, OMIM: 186780), PD1, CTLA4, T Cell Immunoglobulin Mucin 3 (TIM3) (also known as Hepatitis A Virus Cellular Receptor 2) (HGNC: 18437 Entrez Gene: 84868, OMIM: 606652), Lymphocyte Activating 3 (LAG3) (HGNC: 6476, Entrez Gene: 3902, OMIM: 153337), SMAD2, TNF Receptor Superfamily Member 10b (TNFRSF10B) (HGNC: 11905, Entrez Gene: 8795, OMIM: 603612), Protein Phosphatase 2 Catalytic Subunit Alpha (PPP2CA) (HGNC: 9299, Entrez Gene: 5515, OMIM: 176915), Tumor Necrosis Factor Receptor Superfamily Member 6 (TNFRSF6) (also known as Fas Cell Surface Death Receptor (FAS)) (HGNC: 11920, Entrez Gene: 355, OMIM: 134637), B And T Lymphocyte Associated (BTLA) (HGNC: 21087, Entrez Gene: 151888, OMIM: 607925), T Cell Immunoreceptor With Ig And ITIM Domains (TIGIT) (HGNC: 26838, Entrez Gene: 201633, OMIM: 612859), Adenosine A2a Receptor (ADORA2A or A2AR) (HGNC: 263, Entrez Gene: 135, OMIM: 102776), Aryl Hydrocarbon Receptor (AHR) (HGNC: 348, Entrez Gene: 196, OMIM: 600253), Eomesodermin (EOMES) (HGNC: 3372, Entrez Gene: 8320, OMIM: 604615), SMAD Family Member 3 (SMAD3) (HGNC: 6769, Entrez Gene: 4088, OMIM: 603109), SMAD Family Member 4 (SMAD4) (GNC: 6770, Entrez Gene: 4089, OMIM: 600993), TGFBR2, Protein Phosphatase 2 Regulatory Subunit B delta (PPP2R2D) (HGNC: 23732, Entrez Gene: 55844, OMIM: 613992), Tumor Necrosis Factor Ligand Superfamily Member 6 (TNFSF6) (also known as FASL) (HGNC: 11936, Entrez Gene: 356, OMIM: 134638), Caspase 3 (CASP3) HGNC: 1504, Entrez Gene: 836, OMIM: 600636), Suppressor Of Cytokine Signaling 2 (SOCS2) (HGNC: 19382, Entrez Gene: 8835, OMIM: 605117), Kruppel Like Factor 10 (KLF10) (also known as TGFB-Inducible Early Growth Response Protein 1 (TIEG1)) (HGNC: 11810, Entrez Gene: 7071, OMIM: 601878), JunB Proto-Oncogene, AP-1 Transcription Factor Subunit (JunB) (HGNC: 6205, Entrez Gene: 3726, OMIM: 165161), Cbx3, Tet Methylcytosine Dioxygenase 2 (Tet2) (HGNC: 25941, Entrez Gene: 54790, OMIM: 612839), Hexokinase 2 (HK2) (HGNC: 4923, Entrez Gene: 3099, OMIM: 601125), Src homology region 2 domain-containing phosphatase-1 (SHP1) (HGNC: 9658, Entrez Gene: 5777, OMIM: 176883) Src homology region 2 domain-containing phosphatase-2 (SHP2) (HGNC: 9644, Entrez Gene: 5781, OMIM: 176876); or in some embodiments encoding TIM3, LAG3, TNFRSF10B, PPP2CA, TNFRSF6 (FAS), BTLA, TIGIT, A2AR, AHR, EOMES, SMAD3, SMAD4, PPP2R2D, TNFSF6 (FASL), CASP3, SOCS2, TIEG1, JunB, Cbx3, Tet2, HK2, SHP1, or SHP2. In some illustrative embodiments, the inhibitory RNA, for example miRNA, targets mRNA encoding FAS, AHR, CD3z, cCBL, Chromobox 1 (Cbx) (HGNC: 1551, Entrez Gene: 10951, OMIM: 604511), HK2, FASL, SMAD4, or EOMES; or in some illustrative embodiments, the inhibitory RNA, for example miRNA, targets mRNA encoding FAS, AHR, Cbx3, HK2, FASL, SMAD4, or EOMES; or in some illustrative embodiments, the inhibitory RNA, for example miRNA, targets mRNA encoding AHR, Cbx3, HK2, SMAD4, or EOMES.

In some further illustrative embodiments, a vector or genome herein, includes 2 or more, 2-10, 2-8, 2-6, 3-5, 2, 3, 4, 5, 6, 7, or 8 of the inhibitory RNA (e.g. miRNA) identified herein, for example in the paragraph immediately above. In some further illustrative embodiments, a vector or genome herein, includes 2 or more, 2-10, 2-8, 2-6, 3-5, 2, 3, 4, 5, 6, 7, or 8 inhibitory RNA (e.g. miRNA) that target mRNA encoding FAS, cCBL, AHR, CD3z, Cbx, EOMES, or HK2, or a combination of 1 or more inhibitory RNA that target such mRNA. In some further illustrative embodiments, a vector or genome herein, includes 2 or more, 2-10, 2-8, 2-6, 3-5, 2, 3, 4, 5, 6, 7, or 8 inhibitory RNA (e.g. miRNA) that target mRNA encoding AHR, Cbx3, EOMES, or HK2, or a combination of 1 or more inhibitory RNA that target such mRNA.

In some embodiments provided herein, the two or more inhibitory RNA molecules can be delivered in a single intron, such as but not limited to EF1-a intron A. Intron sequences that can be used to harbor miRNAs for the present disclosure include any intron that is processed within a T cell. As indicated herein, one advantage of such an arrangement is that this helps to maximize the ability to include miRNA sequences within the size constraints of a retroviral genome used to deliver such sequences to a T cell in methods provided herein. This is especially true where an intron of the first nucleic acid sequence includes all or a portion of a promoter sequence used to express that intron, a CAR sequence, and other functional sequences provided herein, such as lymphoproliferative element(s) that are not inhibitory RNA molecules, in a polycistronic manner. Sequence requirements for introns are known in the art. In some embodiments, such intron processing is operably linked to a riboswitch, such as any riboswitch disclosed herein. Thus, the riboswitch can provide a regulatory element for control of expression of the one or more miRNA sequences on the first nucleic acid sequence. Accordingly, in illustrative embodiments provided herein is a combination of an miRNA directed against an endogenous T cell receptor subunit, wherein the expression of the miRNA is regulated by a riboswitch, which can be any of the riboswitches discussed herein.

In some embodiments, inhibitory RNA molecules can be provided on multiple nucleic acid sequences that can be included on the same or a different transcriptional unit. For example, a first nucleic acid sequence can encode one or more inhibitory RNA molecules and be expressed from a first promoter and a second nucleic acid sequence can encode one or more inhibitory RNA molecules and be expressed from a second promoter. In illustrative embodiments, two or more inhibitory RNA molecules are located on a first nucleic acid sequence that is expressed from a single promoter. The promoter used to express such miRNAs, are typically promoters that are inactive in a packaging cell used to express a retroviral particle that will deliver the miRNAs in its genome to a target T cell, but such promoter is active, either constitutively or in an inducible manner, within a T cell. The promoter can be a Pol I, Pol II, or Pol III promoter. In some illustrative embodiments, the promoter is a Pol II promoter.

### Characterization and Commercial Production Methods

The present disclosure provides various methods and compositions that can be used as research reagents in scientific experimentation and for commercial production. Such scientific experimentation can include methods for characterization of lymphocytes, such as NK cells and in illustrative embodiments, T cells using methods for genetically modifying, for example transducing lymphocytes provided herein. Such methods for example, can be used to study activation of lymphocytes and the detailed molecular mechanisms by which activation makes such cells transducible. Furthermore, provided herein are genetically modified lymphocytes that will have utility for example, as research tools to better understand factors that influence T cell proliferation and survival. Such genetically modified lymphocytes, such as NK cells and in illustrative embodiments T cells, can furthermore be used for commercial production, for example for the production of certain factors, such as growth factors and immunomodulatory agents, that can be harvested and tested or used in the production of commercial products.

The scientific experiments and/or the characterization of lymphocytes can include any of the aspects, embodiments, or subembodiments provided herein useful for analyzing or comparing lymphocytes. In some embodiments, T cells and/or NK cells can be transduced with the replication incompetent recombinant retroviral particles provided herein that include polynucleotides. In some embodiments, transduction of the T cells and/or NK cells can include polynucleotides that include polynucleotides encoding polypeptides of the present disclosure, for example, CARs, lymphoproliferative elements, and/or activation elements. In some embodiments, the polynucleotides can include inhibitory RNA molecules as discussed elsewhere herein. In some embodiments, the lymphoproliferative elements can be chimeric lymphoproliferative elements.

### Exemplary Embodiments

Provided in this Exemplary Embodiments section are exemplary aspects and embodiments provided herein and further discussed throughout this specification. For the sake of brevity and convenience, all of the disclosed aspects and embodiments and all of the possible combinations of the disclosed aspects and embodiments are not listed in this section. It will be understood that embodiments are provided that are specific embodiments for many aspects, as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are discussed more specifically in other sections of this detailed description.

Unless incompatible with, or already stated in an aspect or embodiment, for any of the methods for genetically modifying and/or transducing lymphocytes (e.g. PBMCs, or T cells and/or NK cells), or uses that include such methods, or genetically modified cells produced using such methods, and any other method or product by process, provided herein, including but not limited to in this Exemplary Embodiments section that includes a contacting step of contacting retroviral particles with lymphocytes (e.g. PBMCs, or T cells and/or NK cells), in certain embodiments, the contacting step can be performed (or can occur) for between 30 seconds and 72 hours, for example, between 1 minute and 12 hours, or between 5 minutes and 12 hours, 10 hours, 8 hours, 6 hours, 4 hours, 2 hours, 1 hour, 30 minutes, or 15 minutes. In some embodiments, the contacting can be performed for less than 24 hours, for example, less than 12 hours, less than 8 hours, less than 4 hours, and in illustrative embodiments less than 2 hours, less than 1 hour, less than 30 minutes or less than 15 minutes, but in each case there is at least an initial contacting step in which retroviral particles and cells are brought into contact in suspension in a transduction reaction mixture. Such suspension can include allowing cells and retroviral particles to settle or causing such settling through application of a force, such as a centrifugal force, to the bottom of a vessel or chamber. However, in certain illustrative embodiments, such force is less than that used for spinoculation, as discussed in more detail herein. After such initial contacting, there can be an additional optional incubating in the reaction mixture containing cells and retroviral particles in suspension in the reaction mixture for the time periods specified without removing retroviral particles that remain free in solution and not associated with cells. In illustrative embodiments, the contacting can be performed (or can occur) for between 30 seconds or 1, 2, 5, 10, 15, 30 or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. In certain illustrative embodiments, the contacting step can be performed for between 30 seconds, 1 minute, 5 minutes, 10 minutes, 15 minutes, or 30 minutes on the low end of the range and 1 hour, 2 hours, 4 hours, 6 hours, 8 hours, 10 hours, or 12 hours on the high end of the range. In some embodiments, the contacting step is performed for between 30 seconds, 1 minute, and 5 minutes on the low end of the range, and 10 minutes, 15 minutes, 30 minutes, 45 minutes, or 60 minutes on the high end of the range. In another illustrative embodiment, the contacting is performed for between an initial contacting step only (without any further incubating in the reaction mixture including the retroviral particles free in suspension and cells in suspension) without any further incubation in the reaction mixture, or a 5 minute or less, 10 minute or less, 15 minute or less, 30 minute or less, or 1 hour or less incubation in the reaction mixture. In some embodiments, the replication incompetent recombinant retroviral particles can be immediately washed out after adding them to the cell(s) to be genetically modified and/or transduced such that the contacting time is carried out for the length of time it takes to wash out the replication incompetent recombinant retroviral particles. Accordingly, typically the contacting includes at least an in initial contacting step in which a retroviral particle(s) and a cell(s) are brought into contact in suspension in a transduction reaction mixture. Such methods can be performed without prior activation.

In any of the aspects and embodiments provided herein that include, or optionally include, a nucleic acid sequence encoding an inhibitory RNA molecule, including, but not limited to, aspects and embodiments provided in this Exemplary Embodiments section, unless already stated therein, or incompatible therewith, such nucleic acid sequence is included and such inhibitory RNA molecule, in certain embodiments, targets any of the gene (e.g. mRNAs encoding) targets identified for example in the Inhibitory RNA Molecules section herein; or in certain embodiments targets TCRa, TCRb, SOCS1, miR155 target, IFN gamma, cCBL, TRAIL2, PP2A, ABCG1, cCBL, CD3z, CD3z, PD1, CTLA4, TIM3, LAG3, SMAD2, TNFRSF10B, PPP2CA, TNFRSF6 (FAS), BTLA, TIGIT, A2AR, AHR, EOMES, SMAD3, SMAD4, TGFBR2, PPP2R2D, TNFSF6 (FASL), CASP3, SOCS2, TIEG1, JunB, Cbx3, Tet2, HK2, SHP1, or SHP2; or in certain embodiments targets cCBL, CD3z, CD3z, PD1, CTLA4, TIM3, LAG3, SMAD2, TNFRSF10B, PPP2CA, TNFRSF6 (FAS), BTLA, TIGIT, A2AR, AHR, EOMES, SMAD3, SMAD4, TGFBR2, PPP2R2D, TNFSF6 (FASL), CASP3, SOCS2, TIEG1, JunB, Cbx3, Tet2, HK2, SHP1, or SHP2; or in certain embodiments targets mRNA encoding TIM3, LAG3, TNFRSF10B, PPP2CA, TNFRSF6 (FAS), BTLA, TIGIT, A2AR, AHR, EOMES, SMAD3, SMAD4, PPP2R2D, TNFSF6 (FASL), CASP3, SOCS2, TIEG1, JunB, Cbx3, Tet2, HK2, SHP1, or SHP2; or in certain illustrative embodiments, targets mRNA encoding FAS, AHR, CD3z, cCBL, Cbx, HK2, FASL, SMAD4, or EOMES; or in certain illustrative embodiments targets mRNA encoding FAS, AHR, Cbx3, HK2, FASL, SMAD4, or EOMES; or in further illustrative embodiments targets mRNA encoding AHR, Cbx3, HK2, SMAD4, or EOMES. In some embodiments, the inhibitory RNA molecule includes at least one of the sequences of SEQ ID NOs:342-449. In some embodiments, the inhibitory RNA molecule includes at least one of the sequences of SEQ ID NOs:394-401, 406-409, 438-441, or 446-449.

In any of the aspects and embodiments provided herein that include, or optionally include, a nucleic acid sequence encoding an inhibitory RNA molecule, including, but not limited to, aspects and embodiments provided in this Exemplary Embodiments section, unless already stated therein, or incompatible therewith, such nucleic acid sequence is included and such inhibitory RNA molecule, in certain embodiments, include 2 or more, 2-10, 2-8, 2-6, 3-5, 2, 3, 4, 5, 6, 7, or 8 inhibitory RNA, or of the targeted inhibitory RNA (e.g. miRNA) identified herein, for example in the paragraph immediately above; or in certain embodiments such polynucleotide includes 2 or more, 2-10, 2-8, 2-6, 3-5, 2, 3, 4, 5, 6, 7, or 8 inhibitory RNA (e.g. miRNA) that target mRNA encoding FAS, cCBL, AHR, CD3z, Cbx, EOMES, or HK2, or a combination of 1 or more inhibitory RNA that target such mRNA; or in certain further illustrative embodiments, such polynucleotide includes 2 or more, 2-10, 2-8, 2-6, 3-5, 2, 3, 4, 5, 6, 7, or 8 inhibitory RNA (e.g. miRNA) that target mRNA encoding FAS, AHR, Cbx3, EOMES, or HK2, or a combination of 1 or more inhibitory RNA that target such mRNA. Such aspects and embodiments provided herein that include a nucleic acid that encodes an inhibitory RNA molecule, include, but are not limited to, aspects and embodiments provided herein that are directed to polynucleotides or vectors, for example replication incompetent retroviral particles, or aspects comprising a genome, such as isolated cells or replication incompetent retroviral particles.

Provided herein in one aspect is a method for genetically modifying and/or transducing a lymphocyte (e.g. a T cell or an NK cell) or a population thereof, comprising contacting blood cells comprising the lymphocyte (e.g. the T cell or NK cell) or the population thereof, ex vivo with a replication incompetent recombinant retroviral particle comprising in its genome a polynucleotide comprising one or more nucleic acid sequences operatively linked to a promoter active in lymphocytes (e.g. T cells and/or NK cells), wherein a first nucleic acid sequence of the one or more nucleic acid sequences encodes a chimeric antigen receptor (CAR) comprising an antigen-specific targeting region (ASTR), a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification and/or transduction of the lymphocyte (e.g. T cell or NK cell), or at least some of the lymphocytes (e.g. T cells and/or NK cells) by the replication incompetent recombinant retroviral particle, thereby producing a genetically modified and/or transduced lymphocyte (e.g. T cell and/or NK cell). In such method, the contacting is typically performed in a reaction mixture, sometimes referred to herein as a transduction reaction mixture, comprising a population of lymphocytes (e.g. T cells and/or NK cells) and contacted with a population of replication incompetent recombinant retroviral particles. Various contacting times are provided herein, including, but not limited to, in this Exemplary Embodiments section, that can be used in this aspect to facilitate membrane association, and eventual membrane fusion of the lymphocytes (e.g. T cells and/or the NK cells) to the replication incompetent recombinant retroviral particles. In an illustrative embodiment, contacting is performed for less than 15 minutes.

Provided herein in one aspect, is use of replication incompetent recombinant retroviral particles in the manufacture of a kit for genetically modifying lymphocytes (e.g. T cells or NK cells) of a subject, wherein the use of the kit comprises: contacting blood cells comprising the lymphocytes (e.g. T cells and/or the NK cells) ex vivo in a reaction mixture, with the replication incompetent recombinant retroviral particles, wherein the replication incompetent recombinant retroviral particles comprise a pseudotyping element on their surface, wherein the replication incompetent recombinant retroviral particles comprise a polynucleotide comprising one or more nucleic acid sequences, typically transcriptional units operatively linked to a promoter active in lymphocytes (e.g. T cells and/or NK cells), wherein the one or more transcriptional units encode a first polypeptide comprising a chimeric antigen receptor (CAR), a first polypeptide comprising a lymphoproliferative element (LE), or a first polypeptide comprising an LE and a second polypeptide comprising a CAR, thereby producing the genetically modified lymphocytes (e.g. the genetically modified T cells and/or the genetically modified NK cells). Various contacting times are provided herein, including, but not limited to, in this Exemplary Embodiments section, that can be used in this aspect to facilitate membrane association, and eventual membrane fusion of the lymphocytes (e.g. T cells and/or the NK cells) to the replication incompetent recombinant retroviral particles. In an illustrative embodiment, contacting is performed for less than 15 minutes.

In another aspect, provided herein is a genetically modified lymphocyte (e.g. T cell or NK cell) made by genetically modifying lymphocytes (e.g. T cells and/or NK cells) according to a method comprising contacting blood cells comprising the T cells or NK cells ex vivo in a reaction mixture, with replication incompetent recombinant retroviral particles, wherein the replication incompetent recombinant retroviral particles comprise a pseudotyping element on their surface, wherein the replication incompetent recombinant retroviral particles comprise a polynucleotide comprising one or more nucleic acid sequences, typically transcriptional units operatively linked to a promoter active in lymphocytes (e.g. T cells and/or NK cells), wherein the one or more transcriptional units encode a first polypeptide comprising a chimeric antigen receptor (CAR), a first polypeptide comprising a lymphoproliferative element (LE), or a first polypeptide comprising an LE and a second polypeptide comprising a CAR, thereby producing the genetically modified lymphocytes (e.g. T cells and/or the genetically modified NK cells). Various contacting times are provided herein, including, but not limited to, in this Exemplary Embodiments section, that can be used in this aspect to facilitate membrane association, and eventual membrane fusion of the lymphocytes (e.g. T cells and/or the NK cells) to the replication incompetent recombinant retroviral particles. In an illustrative embodiment, contacting is performed for less than 15 minutes.

Provided herein in another aspect is a replication incompetent recombinant retroviral particle for use in a method for genetically modifying lymphocyte, for example a T cell and/or NK cell, wherein the method comprises contacting blood cells comprising the lymphocyte, for example T cell and/or NK cell, of the subject in a reaction mixture, ex vivo, with a replication incompetent recombinant retroviral particle comprising in its genome a polynucleotide comprising one or more nucleic acid sequences operatively linked to a promoter active in T cells and/or NK cells, wherein a first nucleic acid sequence of the one or more nucleic acid sequences encodes a chimeric antigen receptor (CAR) comprising an antigen-specific targeting region (ASTR), a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates transduction of at least some of the resting T cells and/or NK cells by the replication incompetent recombinant retroviral particles, thereby producing a genetically modified T cell and/or NK cell. Various contacting times are provided herein, including, but not limited to, in this Exemplary Embodiments section, that can be used in this aspect to facilitate membrane association, and eventual membrane fusion of the lymphocytes (e.g. T cells and/or the NK cells) to the replication incompetent recombinant retroviral particles. In an illustrative embodiment, contacting is performed for less than 15 minutes. In some embodiments the method can further include introducing the genetically modified T cell and/or NK cell into a subject. In illustrative embodiments, the blood cells comprising the lymphocyte (e.g. the T cell and/or NK cell) are from the subject, and thus the introducing is a reintroducing. In this aspect, in some embodiments, a population of lymphocytes (e.g. T cells and/or NK cells) are contacted in the contacting step, genetically modified and/or transduced, and introduced into the subject in the introducing step.

Provided herein in another aspect is the use of a replication incompetent recombinant retroviral particle in the manufacture of a kit for genetically modifying a lymphocyte, for example a T cell and/or NK cell of a subject, wherein the use of the kit comprises contacting blood cells comprising the lymphocyte, for example the T cell and/or the NK cell of the subject ex vivo in a reaction mixture, with replication incompetent recombinant retroviral particles comprising in their genome a polynucleotide comprising one or more nucleic acid sequences operatively linked to a promoter active in T cells and/or NK cells, wherein a first nucleic acid sequence of the one or more nucleic acid sequences encodes a chimeric antigen receptor (CAR) comprising an antigen-specific targeting region (ASTR), a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification of at least some of the T cells and/or NK cells by the replication incompetent recombinant retroviral particles, thereby producing a genetically modified T cell and/or NK cell. As indicated herein, various contacting times are provided herein, that can be used in this aspect to facilitate membrane association, and eventual membrane fusion of the lymphocyte (e.g. T cell and/or the NK cell) to the replication incompetent recombinant retroviral particles. In an illustrative embodiment, contacting is performed for less than 15 minutes. In illustrative embodiments, the blood cells comprising the lymphocyte (e.g. the T cell and/or NK cell) are from the subject, and thus the introducing is a reintroducing. In this aspect, in some embodiments, a population of T cells and/or NK cells are contacted in the contacting step, genetically modified and/or transduced, and introduced into the subject in the introducing step.

Provided herein in another aspect is the use of replication incompetent recombinant retroviral particles in the manufacture of a medicament for genetically modifying lymphocytes, for example T cells and/or NK cells of a subject, wherein the use of the medicament comprises:
A) contacting blood cells comprising the T cells and/or NK cells of the subject ex vivo in a reaction mixture, with the replication incompetent recombinant retroviral particles comprising in their genome a polynucleotide comprising one or more nucleic acid sequences operatively linked to a promoter active in T cells and/or NK cells, wherein a first nucleic acid sequence of the one or more nucleic acid sequences encodes a chimeric antigen receptor (CAR) comprising an antigen-specific targeting region (ASTR), a transmembrane domain, and an intracellular activating domain, and optionally another of the one or more nucleic acid sequences encodes one or more (e.g. two or more) inhibitory RNA molecules directed against one or more RNA targets, and further optionally another of the one or more nucleic acid sequences encodes a polypeptide lymphoproliferative element, wherein said contacting facilitates genetic modification of at least some of the lymphocytes (for example, T cells and/or NK cells) by the replication incompetent recombinant retroviral particles, thereby producing genetically modified T cells and/or NK cells; and optionally
B) introducing the genetically modified T cell and/or NK cell into the subject, thereby genetically modifying the lymphocytes, for example T cells and/or NK cells of the subject.

In such aspects in the immediately above paragraph, as indicated herein, various contacting times are provided herein, that can be used in this aspect to facilitate membrane association, and eventual membrane fusion of the lymphocytes (e.g. T cells and/or the NK cells) to the replication incompetent recombinant retroviral particles. In an illustrative embodiment, contacting is performed for less than 15 minutes. In some embodiments of such method, the blood cells, lymphocyte(s) (e.g. T cell(s) and/or NK cell(s)) are from a subject, typically in such embodiments from blood collected from the subject. In some embodiments of the method aspect provided in this paragraph, the genetically modified and/or transduced lymphocyte (e.g. T cell and/or NK cell) or population thereof, is introduced or reintroduced into a subject.

In any of the use aspects herein, genetically modified lymphocyte(s) (e.g. T cell(s) or NK(s) cell) aspects herein, or methods aspects for genetically modifying and/or transducing a lymphocyte(s) (e.g. T cell(s) or an NK cell(s)) according to any embodiment herein, including but not limited to, any embodiment in this Exemplary Embodiments section, including those above, unless incompatible with, or already stated, the reaction mixture comprises at least 10%, 20%, 25%, 50%, 75%, 80%, 90%, 95%, or 99% whole blood and optionally an effective amount of an anticoagulant, or the reaction mixture further comprises at least one additional blood or blood preparation component that is not a PBMC, and in further illustrative embodiments such blood or blood preparation component is one or more of the Noteworthy Non-PBMC Blood or Blood Preparation Components provided herein.

In another aspect, provided herein is a reaction mixture, comprising replication incompetent recombinant retroviral particles, a T cell activation element, and blood cells, wherein the recombinant retroviral particles comprise a pseudotyping element on their surface, wherein the blood cells comprise T cells and/or NK cells, wherein the replication incompetent recombinant retroviral particles comprise a polynucleotide comprising one or more nucleic acid sequences, typically transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), and/or one or more inhibitory RNA molecules, and wherein the reaction mixture comprises at least 10%, 20%, 25%, 50%, 75%, 80%, 90%, 95%, or 99% whole blood. The one or more inhibitory RNA molecule(s) can be directed against any target provided herein, including, but not limited to, in this Exemplary Embodiments section.

In one aspect, provided herein is a reaction mixture, comprising replication incompetent recombinant retroviral particles, and blood cells, wherein the recombinant retroviral particles comprise a pseudotyping element on their surface, wherein the blood cells comprise T cells and/or NK cells, and wherein the reaction mixture comprises at least 10%, 20%, 25%, 50%, 60%, 70%, 75%, 80%, 90%, 95%, or 99% whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein.

In another aspect, provided herein is a reaction mixture, comprising replication incompetent recombinant retroviral particles, a T cell activation element, and blood cells, wherein the recombinant retroviral particles comprise a pseudotyping element on their surface, wherein the blood cells comprise T cells and/or NK cells, wherein the replication incompetent recombinant retroviral particles comprise a polynucleotide comprising one or more nucleic acid sequences, typically transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), and/or one or more inhibitory RNA molecules, and wherein the reaction mixture comprises at least 10%, 20%, 25%, 50%, 75%, 80%, 90%, 95%, or 99% whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein. The one or more inhibitory RNA molecule(s) can be directed against any target provided herein, including, but not limited to, in this Exemplary Embodiments section.

In another aspect, provided herein is a method for genetically modifying T cells and/or NK cells in blood or a component thereof, comprising contacting blood cells comprising the T cells and/or NK cells ex vivo, with replication incompetent recombinant retroviral particles in a reaction mixture, wherein the replication incompetent recombinant retroviral particles comprise a pseudotyping element on their surface, wherein said contacting facilitates association of the T cells and/or NK cells with the replication incompetent recombinant retroviral particles, wherein the recombinant retroviral particles genetically modify and/or transduce the T cells and/or NK cells, and wherein the reaction mixture comprises at least 10% 10%, 20%, 25%, 50%, 60%, 70%, 75%, 80%, 90%, 95%, or 99% whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein

In another aspect, provided herein is use of replication incompetent recombinant retroviral particles in the manufacture of a kit for genetically modifying T cells and/or NK cells of a subject, wherein the use of the kit comprises: contacting blood cells comprising the T cells and/or NKs cell ex vivo in a reaction mixture, with the replication incompetent recombinant retroviral particles, wherein the replication incompetent recombinant retroviral particles comprise a pseudotyping element on their surface, wherein said contacting facilitates association of the T cells or NK cells with the replication incompetent recombinant retroviral particles, wherein the recombinant retroviral particles genetically modify and/or transduce the T cells and/or NK cells, and wherein the blood cells comprise T cells, NK cells, and wherein the reaction mixture comprises at least 10%, 20%, 25%, 50%, 60%, 70%, 75%, 80%, 90%, 95%, or 99% whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein.

In another aspect, provided herein is a genetically modified T cell or NK cell made by genetically modifying T cells and/or NK cells according to a method comprising, contacting blood cells comprising the T cells and/or NK cells ex vivo, with replication incompetent recombinant retroviral particles in a reaction mixture, wherein the replication incompetent recombinant retroviral particles comprise a pseudotyping element on their surface, wherein said contacting facilitates association of the T cells and/or NK cells with the replication incompetent recombinant retroviral particles, wherein the recombinant retroviral particles genetically modify and/or transduce the T cells and/or NK cells, and wherein the reaction mixture comprises at least 10%, 20%, 25%, 50%, 60%, 70%, 75%, 80%, 90%, 95%, or 99% whole blood and optionally an effective amount of an anticoagulant, or wherein the reaction mixture further comprises at least one additional blood or blood preparation component that is not a PBMC, and in illustrative embodiments such blood or blood preparation component is one or more of the Noteworthy Non-PBMC Blood or Blood Preparation Components provided herein.

The one or more Noteworthy Non-PBMC Blood or Blood Preparation Components are present in certain illustrative embodiments of any of the reaction mixture, use, genetically modified T cell or NK cell, or method for genetically modifying T cells and/or NK cells provided herein, including but not limited to those provided in this Exemplary Embodiments section, because in these certain illustrative embodiments, the reaction mixture comprises at least 10% whole blood. In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiment, the reaction mixture comprises between 10%, 15%, 20%, 25%, 30%, 40%, 50%, 60%, 70%, and 75% on the low end of the range, and 80%, 90%, 95%, 96%, 97%, 98%, 99%, 99.9%, or 99.99% on the high end of the range of whole blood, or at least 10%, 15%, 20%, 25%, 30%, 40%, 50%, 60%, 70%, 75%, 80%, 90%, 95%, 96%, 97%, 98%, 99%, 99.9%, or 99.99% whole blood.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the blood cells in the reaction mixture comprise at least 10% neutrophils and at least 0.5% eosinophils, as a percent of the white blood cells in the reaction mixture.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture comprises at least 20%, 25%, 30%, or 40% neutrophils as a percent of white blood cells in the reaction mixture, or between 20% and 80%, 25% and 75%, or 40% and 60% neutrophils as a percent of white blood cells in the reaction mixture.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture comprises at least 0.1% eosinophils, or between 0.25% and 8% eosinophils, or between 0.5% and 4% as a percent of white blood cells in the reaction mixture.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the blood cells in the reaction mixture are not subjected to a PBMC enrichment procedure before the contacting.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is formed by adding the recombinant retroviral particles to whole blood.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is formed by adding the recombinant retroviral particles to substantially whole blood comprising an effective amount of an anti-coagulant.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is in a closed cell processing system. In certain embodiments of such a reaction mixture, use, genetically modified T cell or NK cell, or method for genetically modifying T cells and/or NK cells, the blood cells in a reaction mixture are PBMCs and the reaction mixture is in contact with a leukodepletion filter assembly in the closed cell processing system, and in optional further embodiments the leukodepletion filter assembly comprises a HemaTrate filter.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture comprises an anti-coagulant. For example, in certain embodiments, the anti-coagulant is selected from the group consisting of acid citrate dextrose, EDTA, or heparin. In certain embodiments, the anti-coagulant is other than acid citrate dextrose. In certain embodiments, the anti-coagulant comprises an effective amount of heparin.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is in a blood bag during the contacting.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is in contact with a T lymphocyte and/or NK cell-enriching filter in the closed cell processing system before the contacting, and wherein the reaction mixture comprises granulocytes, wherein the granulocytes comprise at least 10% of the white blood cells in the reaction mixture, or wherein the reaction mixture comprises at least 10% as many granulocytes as T cells, wherein the genetically modified lymphocytes (e.g. T cells or NK cells) are subject to a PBMC enrichment process after the contacting.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, blood cells in the reaction mixture are PBMCs and wherein the reaction mixture is in contact with a leukodepletion filter assembly in the closed cell processing system after the contacting comprising an optional incubating in the reaction mixture.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the whole blood is other than cord blood.

In certain embodiments of any of the reaction mixture, use, genetically modified T cell or NK cell, or method for genetically modifying T cells and/or NK cells provided herein, included but not limited to those provided in this Exemplary Embodiments section, unless incompatible with, or already stated in an aspect or embodiments, the reaction mixture is in contact with a leukodepletion filter assembly in a closed cell processing system before the contacting, at the time the recombinant retroviral particles and the blood cells are contacted, during the contacting comprising an optional incubating in the reaction mixture, and/or after the contacting comprising the optional incubating in the reaction mixture, wherein the T cells and/or NK cells, or the genetically modified T cells and/or NK cells are further subjected to a PBMC enrichment procedure.

In one aspect, provided herein is a replication incompetent recombinant retroviral particle comprising in its genome a polynucleotide comprising one or more nucleic acid sequences operatively linked to a promoter active in T cells and/or NK cells, wherein:

a. a first nucleic acid sequence of the one or more nucleic acid sequences encodes one or more (e.g. two or more) inhibitory RNA molecules directed against one or more RNA targets, and
b. a second nucleic acid sequence of the one or more nucleic acid sequences encodes a chimeric antigen receptor (CAR) comprising an antigen-specific targeting region (ASTR), a transmembrane domain, and an intracellular activating domain. The one or more inhibitory RNA molecule(s) can be directed against any target provided herein, including, but not limited to, in this Exemplary Embodiments section.

Provided in another aspect herein is a mammalian packaging cell line comprising a packageable RNA genome for a replication incompetent retroviral particle, wherein said packageable RNA genome comprises:

a. a 5′ long terminal repeat, or active fragment thereof;
b. a nucleic acid sequence encoding a retroviral cis-acting RNA packaging element;
c. a polynucleotide comprising one or more nucleic acid sequences operatively linked to a promoter active in T cells and/or NK cells, wherein a first nucleic acid sequence of the one or more nucleic acids encodes one or more (e.g. two or more) inhibitory RNA molecules directed against one or more RNA targets and a second nucleic acid sequence of the one or more nucleic acid sequences encodes a chimeric antigen receptor (CAR) comprising an antigen-specific targeting region (ASTR), a transmembrane domain, and an intracellular activating domain; and
d. a 3′ long terminal repeat, or active fragment thereof. The one or more inhibitory RNA molecule(s) can be directed against any target provided herein, including, but not limited to, in this Exemplary Embodiments section. Provided in another aspect herein is a retroviral vector comprising a packageable RNA genome for a replication incompetent retroviral particle, wherein said packageable RNA genome comprises:
a. a 5′ long terminal repeat, or active fragment thereof;
b. a nucleic acid sequence encoding a retroviral cis-acting RNA packaging element;
c. a polynucleotide comprising one or more nucleic acid sequences operatively linked to a promoter active in T cells and/or NK cells, wherein a first nucleic acid sequence of the one or more nucleic acids encodes one or more (e.g. two or more) inhibitory RNA molecules directed against one or more RNA targets and a second nucleic acid sequence of the one or more nucleic acid sequences encodes a chimeric antigen receptor (CAR) comprising an antigen-specific targeting region (ASTR), a transmembrane domain, and an intracellular activating domain; and
a 3′ long terminal repeat, or active fragment thereof. The one or more inhibitory RNA molecule(s) can be directed against any target provided herein, including, but not limited to, in this Exemplary Embodiments section.

In some embodiments of the retroviral vector aspect, or the mammalian packaging cell line aspect, the polynucleotide of (c) can be in reverse orientation to the nucleic acid sequence encoding the retroviral cis-acting RNA packaging element (b), the 5′ long terminal repeat (a), and/or the 3′ long terminal repeat (d).

In some embodiments of the retroviral vector aspect or the mammalian packaging cell line aspect, expression of the packageable RNA genome is driven by an inducible promoter active in the mammalian packaging cell line.

In some embodiments of the retroviral vector aspect or the mammalian packaging cell line aspect, the retroviral cis-acting RNA packaging element can comprise a central polypurine tract (cPPT)/central termination sequence, an HIV Psi, or a combination thereof. The retroviral vector can optionally include an antibiotic resistance gene and/or a detectable marker.

Provided herein in another aspect is a genetically modified T cell and/or NK cell comprising:

a. one or more (e.g. two or more) inhibitory RNA molecules directed against one or more RNA targets; and
b. a chimeric antigen receptor (CAR) comprising an antigen-specific targeting region (ASTR), a transmembrane domain, and an intracellular activating domain, wherein said one or more (e.g. two or more) inhibitory RNA molecules and the CAR are encoded by nucleic acid sequences that are genetic modifications of the T cell and/or NK cell. The one or more inhibitory RNA molecule(s) can be directed against any target provided herein, including, but not limited to, in this Exemplary Embodiments section.

In some embodiments of the genetically modified T cell and/or NK cell aspect, the genetically modified T cell and/or NK cell also comprises at least one lymphoproliferative element that is not an inhibitory RNA molecule, typically a polypeptide lymphoproliferative element, wherein said lymphoproliferative element is encoded by a nucleic acid that is a genetic modification of the T cell and/or NK cell. In some embodiments, the inhibitory RNA molecules, the CAR, and/or the at least one polypeptide lymphoproliferative element are expressed in a polycistronic matter. In illustrative embodiments, the inhibitory RNA molecules are expressed from a single polycistronic transcript.

Provided herein in another aspect is a replication incompetent recombinant retroviral particle, wherein the replication incompetent recombinant retroviral particle comprises in its genome a polynucleotide comprising one or more nucleic acid sequences operatively linked to a promoter active in T cells and/or NK cells, wherein a first nucleic acid sequence of the one or more nucleic acid sequences encodes one or more (e.g. two or more) inhibitory RNA molecules directed against one or more RNA targets and a second nucleic acid sequence of the one or more nucleic acid sequences encodes a chimeric antigen receptor (CAR) comprising an antigen-specific targeting region (ASTR), a transmembrane domain, and an intracellular activating domain, wherein the method comprises contacting a T cell and/or NK cell of the subject ex vivo, and said contacting facilitates transduction of at least some of the resting T cells and/or NK cells by the replication incompetent recombinant retroviral particles, thereby producing a genetically modified T cell and/or NK cell. The one or more inhibitory RNA molecule(s) can be directed against any target provided herein, including, but not limited to, in this Exemplary Embodiments section.

Provided herein in another aspect is a commercial container containing a replication incompetent recombinant retroviral particle and optionally instructions for the use thereof to treat tumor growth in a subject, wherein the replication incompetent recombinant retroviral particle comprises in its genome a polynucleotide comprising one or more nucleic acid sequences operatively linked to a promoter active in T cells and/or NK cells, wherein a first nucleic acid sequence of the one or more nucleic acid sequences encodes one or more (e.g. two or more) inhibitory RNA molecules directed against one or more RNA targets and a second nucleic acid sequence of the one or more nucleic acid sequences encodes a chimeric antigen receptor (CAR) comprising an antigen-specific targeting region (ASTR), a transmembrane domain, and an intracellular activating domain. The one or more inhibitory RNA molecule(s) can be directed against any target provided herein, including, but not limited to, in this Exemplary Embodiments section.

In any of the aspects provided immediately above that include a polynucleotide comprising one or more nucleic acid sequences operatively linked to a promoter active in T cells and/or NK cells, wherein a first nucleic acid sequence of the one or more nucleic acid sequences encodes one or more (e.g. two or more) inhibitory RNA molecules directed against one or more RNA targets, and a second nucleic acid sequence of the one or more nucleic acid sequences encodes a chimeric antigen receptor (CAR) comprising an antigen-specific targeting region (ASTR), a transmembrane domain, and an intracellular activating domain, the polynucleotide may further include a third nucleic acid sequence that encodes at least one lymphoproliferative element that is not an inhibitory RNA molecule, and in illustrative embodiments is a polypeptide, for example any of the polypeptide lymphoproliferative elements disclosed herein.

In any of the aspects provided immediately above that include a polynucleotide comprising one or more nucleic acid sequences operatively linked to a promoter active in T cells and/or NK cells, wherein a first nucleic acid sequence of the one or more nucleic acid sequences encodes one or more (e.g. two or more) inhibitory RNA molecules directed against one or more RNA targets, the inhibitory RNA molecule can have any of the structures and/or be any of the embodiments provided herein in the Inhibitory RNA Molecules section. For example, the inhibitory RNA can in some embodiments include a 5′ strand and a 3′ strand that are partially or fully complementary to one another, wherein said 5′ strand and said 3′ strand are capable of forming an 18-25 nucleotide RNA duplex. Furthermore, the inhibitory RNA molecule can be a miRNA or an shRNA and in certain embodiments, at least one or all of the inhibitory RNA molecules comprise a 5′ arm, 3′ arm, or both, derived from a naturally occurring miRNA. For example, such as a naturally occurring miRNA can be selected from the group consisting of: miR-155, miR-30, miR-17-92, miR-122, and miR-21, and in illustrative embodiments miR-155.

In any of the aspects provided immediately above that include a polynucleotide comprising one or more nucleic acid sequences operatively linked to a promoter active in T cells and/or NK cells, wherein a first nucleic acid sequence of the one or more nucleic acid sequences encodes two or more inhibitory RNA molecules directed against one or more RNA targets, in some embodiments, the first nucleic acid sequence encodes two to four inhibitory RNA molecules. In illustrative embodiments, between 2 and 10, 2 and 8, 2 and 6, 2 and 5, 2 and 4, 3 and 5, or 3 and 6 inhibitory RNA molecules are included in the first nucleic acid sequence. In an illustrative embodiment, four inhibitory RNA molecules are included in the first nucleic acid sequence.

In any of the aspects provided immediately above that include a polynucleotide comprising one or more nucleic acid sequences operatively linked to a promoter active in T cells and/or NK cells, wherein a first nucleic acid sequence of the one or more nucleic acid sequences encodes one or more (e.g. two or more) inhibitory RNA molecules directed against one or more RNA targets, the one or more (e.g. two or more) inhibitory RNA molecules can be in an intron. In some embodiments, the intron is in a promoter. In illustrative embodiments, the intron is EF-1alpha intron A. In some embodiments, the intron is adjacent to and downstream of a promoter, which in illustrative embodiments, is inactive in a packaging cell used to produce the replication incompetent recombinant retroviral particle.

In any of the reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated at least 10%, 20%, 25%, 30%, 40%, 50%, most, 60%, 70%, 75%, 80%, 90%, 95%, or 99% of the T cells are resting T cells, or of the NK cells are resting NK cells, when they are combined with the replication incompetent retroviral particles to form the reaction mixture.

In any of the use, genetically modified T cell or NK cell, or method for genetically modifying T cells and/or NK cells aspects and embodiments provided herein, including, but not limited to, in this Exemplary Embodiments section, unless incompatible with, or otherwise stated, the cell or cells are not subjected to a spinoculation procedure, for example not subjected to a spinoculation of at least 800 g for at least 30 minutes.

In some embodiments of any of the use, genetically modified T cell or NK cell, or method for genetically modifying T cells and/or NK cells aspects and embodiments provided herein, including, but not limited to, in this Exemplary Embodiments section, unless incompatible with, or otherwise stated, the method further comprises administering the genetically modified T cells and/or NK cells to a subject, optionally wherein the subject is the source of the blood cells. In some subembodiments of these and embodiments of any of the methods and uses herein, including those in this Exemplary Embodiments section, provided that it is not incompatible with, or already stated, the genetically modified and/or transduced lymphocyte (e.g. T cell and/or NK cell) or population thereof, undergoes 4 or fewer cell divisions ex vivo prior to being introduced or reintroduced into the subject. In some embodiments, no more than 8 hours, 6 hours, 4 hours, 2 hours, or 1 hour pass(es) between the time blood is collected from the subject and the time the genetically modified T cells and/or NK cells are reintroduced into the subject. In some embodiments, all steps after the blood is collected and before the blood is reintroduced, are performed in a closed system, optionally in which a person monitors the closed system throughout the processing.

In any of the replication incompetent recombinant retroviral particle, reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the replication incompetent recombinant retroviral particle(s) comprise a membrane-bound T cell activation element on their surface. In some subembodiments of these and embodiments of any of the aspects provided herein, including those in this Exemplary Embodiments section, provided that it is not incompatible with, or already stated, the T cell activation element can be one or more of an anti-CD3 antibody or an anti-CD28 antibody. In some embodiments of these and embodiments of any of the aspects provided herein, including, but not limited to, in this Exemplary Embodiments section, unless incompatible with, or otherwise stated, the T cell activation element is one or more polypeptides, in illustrative embodiments membrane-bound polypeptides capable of binding CD28, OX40, 4-1BB, ICOS, CD9, CD53, CD63, CD81, and/or CD82. In some embodiments, a membrane-bound polypeptide capable of binding to CD3 is fused to a heterologous GPI anchor attachment sequence and/or a membrane-bound polypeptide capable of binding to CD28 is fused to a heterologous GPI anchor attachment sequence. In illustrative embodiments, the membrane-bound polypeptide capable of binding to CD28 is CD80, or an extra-cellular domain thereof, bound to a CD16B GPI anchor attachment sequence. In some embodiments, the T cell activation element further includes one or more polypeptides capable of binding CD3. In some subembodiments of these and embodiments of any of the aspects provided herein, including those in this Exemplary Embodiments section, provided that it is not incompatible with, or already stated, the T cell activation element is a membrane-bound anti-CD3 antibody, wherein the anti-CD3 antibody is bound to the membrane of the recombinant retroviral particles. In some embodiments, the membrane-bound anti-CD3 antibody is anti-CD3 scFv or an anti-CD3 scFvFc. In some embodiments, the membrane-bound anti-CD3 antibody is bound to the membrane by a heterologous GPI anchor. In some embodiments, the anti-CD3 antibody is a recombinant fusion protein with a viral envelope protein. In some embodiments, the anti-CD3 antibody is a recombinant fusion protein with the viral envelope protein from MuLV. In some embodiments, the anti-CD3 is a recombinant fusion protein with the viral envelope protein of MulV which is mutated at a furin cleavage site.

In any of the use, genetically modified T cell or NK cell, or method for genetically modifying T cells and/or NK cells aspects and embodiments provided herein, including, but not limited to, in this Exemplary Embodiments section, unless incompatible with, or otherwise stated, an ABC transporter inhibitor and/or substrate, in further subembodiments an exogenous ABC transporter inhibitor and/or substrate, is not present before, during, or both before and during the genetic modification and/or transduction.

In any of the reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the recombinant retroviral particles are present in the reaction mixture at an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15 or are present in the reaction mixture at an MOI of at least 0.1, 0.5, 1, 2, 2.5, 3, 5, 10 or 15.

In any of the reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, at least 5%, at least 10%, at least 15%, or at least 20% of the T cells and/or NK cells are genetically modified, or between 5%, 10%, 15%, 20%, or 25% on the low end of the range, and 20%, 25%, 50%, 60%, 70%, 80%, or 85% on the high end of the range.

In any of the polynucleotide, replication incompetent recombinant retroviral particle, reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the one or more transcriptional units can encode a polypeptide comprising a lymphoproliferative element (LE). Any of the polypeptide lymphoproliferative elements disclosed herein, for example, but not limited to those disclosed in the “Lymphoproliferative elements” section herein, or functional mutants and/or fragments thereof, can be encoded. In some embodiments, the LE comprises an intracellular domain from CD2, CD3D, CD3E, CD3G, CD4, CD8A, CD8B, CD27, mutated Delta Lck CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof.

In any of the replication incompetent recombinant retroviral particle, reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the replication incompetent recombinant retroviral particles are lentiviral particles. In further illustrative embodiments, the genetically modified cell is a genetically modified T cell or a genetically modified NKT cell.

In any of the polynucleotide, replication incompetent recombinant retroviral particle, reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the one or more transcriptional units can encode a polypeptide comprising a CAR. In some embodiments, the CAR is a microenvironment restricted biologic (MRB)-CAR. In other embodiments, the ASTR of the CAR binds to a tumor associated antigen. In other embodiments, the ASTR of the CAR is a microenvironment-restricted biologic (MRB)-ASTR.

In certain embodiments, any of the aspects and embodiments provided herein that include a polynucleotide, in some instances in the genome of a replication incompetent recombinant retroviral particle or a genetically modified T cell and/or NK cell, that comprises a nucleic acid sequences operatively linked to a promoter active in T cells and/or NK cells, that encodes at least one polypeptide lymphoproliferative element. In illustrative embodiments, the polypeptide lymphoproliferative element is any of the polypeptide lymphoproliferative elements disclosed herein. In some embodiments, any or all of the nucleic acid sequences provided herein can be operably linked to a riboswitch. In some embodiments, the riboswitch is capable of binding a nucleoside analog. In some embodiments, the nucleoside analog is an antiviral drug.

In any of the aspects and embodiments provided herein that include a replication incompetent recombinant retroviral particle, including, but not limited to aspects and embodiments in this Exemplary Embodiments section, unless incompatible with, or already stated in an aspect or embodiment, in illustrative embodiments, the replication incompetent recombinant retroviral particle comprises a pseudotyping element on its surface that is capable of binding to a T cell and/or NK cell and facilitating membrane fusion of the replication incompetent recombinant retroviral particle thereto. In some embodiments, the pseudotyping element is a viral envelope protein. In some embodiments, the viral envelope protein is one or more of the feline endogenous virus (RD114) envelope protein, the oncoretroviral amphotropic envelope protein, the oncoretroviral ecotropic envelope protein, the vesicular stomatitis virus envelope protein (VSV-G), the baboon retroviral envelope glycoprotein (BaEV), the murine leukemia envelope protein (MuLV), and/or the paramyxovirus Measles envelope proteins H and F, or a fragment of any thereof that retains the ability to bind to resting T cells and/or resting NK cells. In illustrative embodiments, the pseudotyping element is VSV-G. As discussed elsewhere herein, the pseudotyping element can include a fusion with a T cell activation element, which in illustrative embodiments, can be a fusion with any of the envelope protein pseudotyping elements, for example MuLV or VSV-G, with an anti-CD3 antibody. In further illustrative embodiments, the pseudotyping elements include both a VSV-G and a fusion of an antiCD3scFv to MuLV.

In any of the aspects provided herein that include a replication incompetent recombinant retroviral particle, in some embodiments, the replication incompetent recombinant retroviral particle comprises on its surface a nucleic acid encoding a domain recognized by a monoclonal antibody approved biologic.

In certain illustrative embodiments of any of the reaction mixture, use, genetically modified T cell or NK cell, or method for genetically modifying T cells and/or NK cells aspects and embodiments provided herein, including, but not limited to, in this Exemplary Embodiments section, unless incompatible with, or otherwise stated, the blood cells in the reaction mixture are blood cells that were produced by a PBMC enrichment procedure and comprise PBMCs, or the blood cells in illustrative embodiments are PBMCs. In illustrative embodiments, such embodiments including PMBC enrichment are not combined with an embodiment where the reaction mixture includes at least 10% whole blood. Thus, in certain illustrative embodiments herein, the blood cells in a reaction mixture are the PBMC cell fraction from a PBMC enrichment procedure to which retroviral particles are added to form the reaction mixture, and in other illustrative embodiments, the blood cells in a reaction mixture are from whole blood to which retroviral particles are added to form the reaction mixture.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. Materials and Methods for Transduction Experiments

This Example provides materials and methods used in experiments disclosed in subsequent Examples herein.

### Recombinant Lentiviral Particle Production by Transient Transfection.

293T cells (Lenti-X™ 293T, Clontech) were adapted to suspension culture by serial growth in Freestyle™ 293 Expression Medium (ThermoFisher Scientific), named F1XT cells, and were used as the packaging cells for experiments herein unless noted otherwise.

Where noted, a typical 4 vector packaging system included 3 packaging plasmids that encoded (i) gag/pol, (ii) rev, and (iii) a pseudotyping element such as VSV-G. The 4^{th} vector of this packaging system is the genomic plasmid, a third generation lentiviral expression vector (containing a deletion in the 3′ LTR leading to self-inactivation) that encoded 1 or more genes of interest. For transfections using 4 plasmids, the total DNA used (1 μg/mL of culture volume) was a mixture of the 4 plasmids at the following molar ratios: 1× gag/pol-containing plasmid, 1× Rev-containing plasmid, lx viral envelope containing plasmid (VSV-G unless noted otherwise), and 2× genomic plasmid unless noted otherwise. Where noted, a typical 5 vector packaging system was used in which a 5^{th} vector encoding, for example, a T cell activation element such as antiCD3-scFvFc-GPI, was added to the otherwise 4 vector packaging system. For transfections using 5 plasmids, the total DNA used (1 μg/mL of culture volume) was a mixture of the 5 plasmids at the following molar ratios: 1× gag/pol-containing plasmid, lx Rev-containing plasmid, lx VSV-G containing plasmid, 2× genomic plasmid, and 1× of the 5^{th} vector unless noted otherwise.

Plasmid DNA was dissolved in 1.5 ml Gibco™ Opti-MEM™ growth media for every 30 mL of culture containing packaging cells. Polyethylenimine (PEI) (Polysciences) (dissolved in weak acid) was diluted in 1.5 ml Gibco™ Opti-MEM™ to 2 μg/mL. A 3 ml mixture of PEI and DNA was made by combining the two prepared reagents at a ratio of 2ug of PEI to 1 ug of DNA. After a 5-minute room temperature incubation, the two solutions were mixed together thoroughly, and incubated at room temperature for 20 more minutes. The final volume (3 ml) was added to 30 ml of packaging cells in suspension at 1×10⁶ cells/mL in a 125 mL Erlenmeyer flask. The cells were then incubated at 37° C. for 72 hours with rotation at 125 rpm and with 8% CO₂ for transfection.

After 72 hours, the supernatants were harvested and clarified by centrifugation at 1,200 g for 10 minutes. The clarified supernatants were decanted to a new tube. Virus was purified from the clarified supernatants by centrifugation, polyethylene glycol (PEG) precipitation, or depth filtration. For purification by centrifugation, the lentiviral particles were precipitated by overnight centrifugation at 3,300 g, at 4° C. The supernatant was discarded, and the lentiviral particle pellets were resuspended in 1:100 of the initial volume of packaging cell culture. For purification by PEG precipitation, ¼ volume PEG was added to the clarified supernatant and incubated overnight at 4° C. The mixture was then centrifuged at 1600 g for 1 hour (for 50 ml conical tubes) or 1800 g for 1.5 hours (for 500 ml conical tubes). The supernatant was discarded, and the lentiviral particle pellets were resuspended in 1:100 of the initial volume of packaging cell culture. For purification by depth filtration, the clarified supernatants were concentrated by tangential flow filtration (TFF) and benzonase digested. The virus was then purified and buffer exchanged by diafiltration into the final formulation (PBS with 2% lactose).

Lentiviral particles were titered by serial dilution and analysis of transgene expression, by transduction into 293T and/or Jurkat cells and analysis of transgene expression by FACS or qPCR for lentiviral genome using Lenti-X™ qRT-PCR Titration Kit (#631235) or p24 assay ELISA kit from Takara (Lenti-X™ p24 Rapid Titer Kit #632200).

### Genomic Plasmids Used in Examples.

The following lentiviral genomic vectors encode genes and features of interest as indicated:

F1-3-23 encodes a CD19 CAR comprised of an anti-CD19scFv, a CD8 stalk and transmembrane region, and an intracellular domain from CD3z followed by T2A and an eTag (aCD19:CD8:CD3z-T2A-eTag).

Additional lentiviral genomic vectors are described in specific examples.

### Example 2. Transduction Efficiency of Unstimulated PBMCs Exposed for 4 Hours to Retroviral Particles Pseudotyped VSV-G or Influenza HA and NA and Optionally Copseudotyped with Envelopes Derived from VSV-G, MV, or MuLV, and Further, Optionally, Displaying an Anti-CD3 scFv on their Surfaces

In this example, lentiviral particles pseudotyped or cospeudotyped with various different envelope proteins and optionally displaying a T cell activation element, were exposed to unstimulated human PBMCs for 4 hours and transduction efficiency was assessed.

Recombinant lentiviral particles were produced in F1XT cells. The cells were transiently transfected using PEI with a genomic plasmid and separate packaging plasmids encoding gag/pol, rev, and an envelope plasmid. For certain samples, the transfection reaction mixture also included a plasmid encoding UCHT1scFvFc-GPI, a copseudotyping envelope, or a copseudotyping envelope fused to an antiCD3scFv. The genomic plasmid used for samples in this example was F1-0-03 as disclosed in other examples herein. The pseudotyping and copseudotyping plasmids used for samples in this example encoded envelope proteins from VSV-G (SEQ ID NO:336), U-VSV-G (SEQ ID NO: 455) in which the anti-CD3 scFv from UCHT1 was fused to the amino terminus of the VSV-G envelope, influenza HA from HIN1 PR8 1934 (SEQ ID NO: 311) and NA from H10N7-HKWF446C-07 (SEQ ID NO:312), U-MuLV (SEQ ID NO:341) in which the anti-CD3 scFv from UCHT1 was fused to the amino terminus of the MuLV envelope, U-MuLV variants in which 8 to 31 C-terminal amino acids were deleted from the cytoplasmic tail, U-MuLVSUx (SEQ ID NO: 454) in which the furin-mediated cleavage site Lys-Tyr-Lys-Arg in U-MuLV was replaced with the Ile-Glu-Gly-Arg peptide, or MVHΔ24 (SEQ ID NO: 315) in which the C-terminal 24 amino acids of the measles virus H protein were removed.

In certain samples the U-MuLV envelope protein was endcoded on the rev packaging plasmid in tandem in the format U-MuLV-IRES2-rev (MuLVIR) or in the format U-MuLV-T2A-rev (MuLV2R). By putting the copseudotyping element on a packaging vector such as rev, 4 rather than 5 separate plasmids were used to transfect packaging cells. It was observed herein that transfecting with 4 rather than 5 plasmids resulted in higher viral titers.

On Day 0, PBMCs were prepared from buffy coats from 2 donors as described in Example 1 without any additional steps to remove monocytes. After isolation, 1×10⁶ unstimulated PBMCs in 1 ml of X-Vivo15 were seeded into each well of a 96 deep-well plates. Viral particles were added at an MOI of 1 or 10 as indicated, and the plates were incubated for 4 hours at 37° C. and 5% CO2. After the 4 hour exposure, the cells were pelleted for 5 minutes at 400 g and washed 3 times by resuspending the cells in 2 mls of DPBS+2% HSA and centrifuging for 5 minutes at 400 g, before the cells in each well were resuspended in 1 ml X-Vivo15 and incubated at 37° C. and 5% CO2. No exogenous cytokines were added to the samples at any time. Each sample was run in duplicate using PBMCs from each of the 2 donors. Samples were collected at Day 6 to determine transduction efficiencies based on eTAG, and CD3 expression as determined by FACs analysis using a lymphocyte gate based on forward and side scatter.

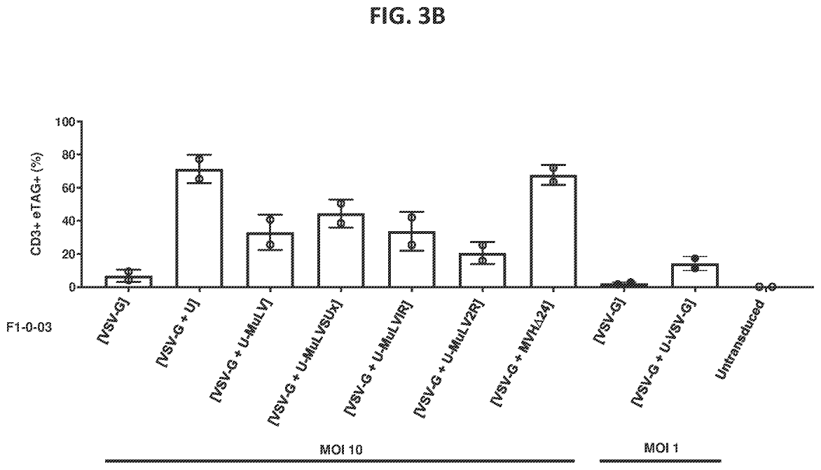

FIG. 3A shows the total number of live cells per well on Day 6 following transduction. Compared to samples exposed to viral particles pseudotyped with VSV-G alone, samples exposed to viral particles pseudotyped with VSV-G and also displaying UCHT1 had a greater number of cells per well. This was observed both when UCHT1scFv was displayed as a GPI-linked scFvFc and when the scFv was fused to either the VSV-G or MuLV viral envelopes. Not to be limited by theory, the stimulation of CD3+T and NK cells by the antiCD3 scFv is believed to lead to proliferation and survival which can account for at least a portion of this increase in cell number.

FIG. 3B shows the percent of CD3+ cells transduced as measured by eTAG expression. Samples exposed to viral particles pseudotyped with VSV-G that also either displayed UCHT1ScFvFc-GPI or were copseudotyped with U-MuLV, U-MuLVSUx, U-VSV-G, or MVHΔ24 had higher transduction efficiencies than samples exposed to viral particles pseudotyped with VSV-G alone that didn't display an antiCD3 antibody. Among the 4 samples tested in this experiment at an MOI of 10, the efficiency by which VSV-G+UCHT1scFvFc-GPI viral particles transduced CD3+ unstimulated PBMCs was 64.3%, 66.3%, 78.0%, and 76.7%. Among the 4 samples tested in this experiment at an MOI of 10, the efficiency by which VSV-G+U-MuLV viral particles transduced CD3+ unstimulated PBMCs was 37.6%, 43.8%, 20.5%, and 30.8%. When copseudotyped with VSV-G, individual variants of U-MuLV in which the 4, 8, 12, 16, 20, 24, 28, and 31 C-terminal amino acids were deleted, transduced CD3+ unstimulated PBMCs in 4 hours similar to full length U-MuLV (not shown). Similarly, when copseudotyped with VSV-G, individual variants of U-MuLVSUx in which the Factor X cleavage site (AAAIEGR) between the transmembrane (TM) and surface (SU) units was replaced with (G4S)3 or “AAAIAGA”, transduced CD3+ unstimulated PBMCs in 4 hours similar to U-MuLVSUx (not shown). Among the 4 samples tested in this experiment at an MOI of 10, the efficiency by which VSV-G+MVHΔ24 viral particles transduced CD3+ unstimulated PBMCs was 64.5%, 62.4%, 72.3%, and 71.5%. In a separate experiment, viral particles pseudotyped with influenza HA from H1N1 PR8 1934 and NA from H10N7-HKWF446C-07 transduced CD3+ unstimulated PBMCs with comparable efficiency to viral particles copseudotyped with VSV-G+U-MuLV.

### Example 3. Efficient Genetic Modification of Resting Lymphocytes by Exposure of Whole Blood to Recombinant Retroviral Particles for 4 Hours Followed by a PBMC Enrichment Procedure

In this example, unstimulated human T cells and NKT cells were effectively genetically modified by a 4 hour incubation of a reaction mixture that included whole blood and retroviral particles that were pseudotyped with VSV-G and displayed a T cell activation element on their surface. PBMCs were subsequently isolated from the transduction reaction mixture using a traditional density gradient centrifugation-based PBMC enrichment procedure. Transduction of CD3+ cells was assessed by expression of the eTag transgene using flow cytometry.

Depth filtration was used to purify the following lentiviral particles used in this Example: F1-3-23 pseudotyped with VSV-G (F1-3-23G); and F1-3-23 pseudotyped with VSV-G and displaying the T cell activation element, UCHT1-scFvFc-GPI (F1-3-23GU).

10 ml samples of whole fresh blood in Vacutainer tubes containing anticoagulants were purchased. (StemExpress, San Diego). The anticoagulant in individual samples was either EDTA 1.8 mg/ml or Na-Heparin 16 USP units per mL of blood. Recombinant lentiviral particles were added directly to the Vacutainer tubes of whole blood at an MOI of 5 (assuming 1×10⁶ PBMCs/ml of blood) to initiate contacting of the lentiviral particles to lymphocytes in the whole blood, and incubated for 4 hours, at 37° C., 5% CO₂ with gentle mixing every hour to disrupt any sedimentation. After the 4 hour incubation, PBMCs from each whole blood sample were isolated individually using SepMate50 tubes (STEMCELL Technologies) according to the manufacturer's protocol. PBMCs were collected in 15 ml conical tubes and washed by resuspending the cells in 10 ms DPBS+2% HSA, and centrifuging them for 5 minutes at 400 g. This wash procedure was repeated 3 times before the cells were resuspended in 10 ml X-Vivo15 and cultured upright in T75 flasks at 37° C. and 5% CO₂. No exogenous cytokines were added to the samples at any time. Samples were collected at Day 6 to determine transduction efficiencies based on eTag and CD3 expression on live cells as determined by FACs analysis using a lymphocyte gate based on forward and side scatter.

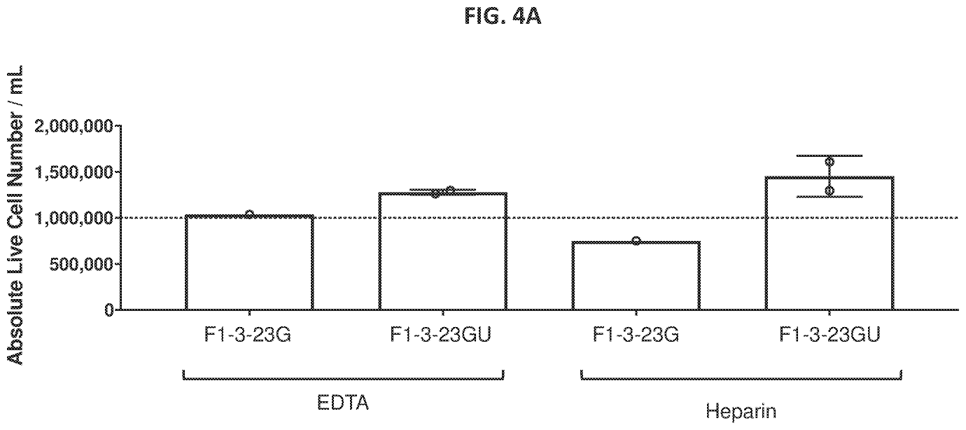

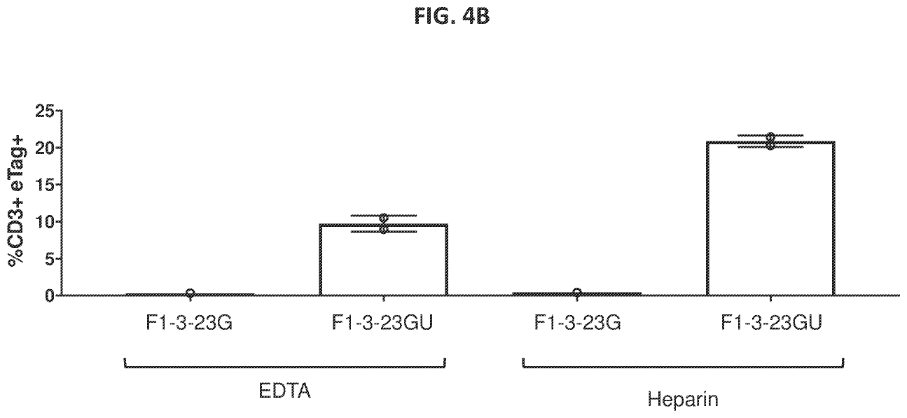

FIGS. 4A and 4B show histograms of the absolute live cell count per ml (FIG. 4A) and the percentage of CD3+eTag+ cells (i.e. transduced T cells) (FIG. 4B) at Day 6 after transduction of whole blood. Consistent with our previous results and the results of others studying transduction of isolated PBMCs, we see in this Example that recombinant retroviral particles pseudotyped with VSV-G alone are extremely inefficient at transducing PBMCs in whole blood. We have seen previously, however, that recombinant retroviral particles pseudotyped with VSV-G and displaying a T cell activation element, are capable of efficiently transducing isolated PBMCs. Surprisingly, these histograms show that a PBMC enrichment step is not required for retroviral particles to efficiently transduce PBMCs present in whole blood. Rather, retroviral particles pseudotyped with VSV-G and displaying antiCD3-scFvFc when added directly to whole blood containing an anticoagulant can effectively genetically modify and transduce PBMCs therein. Genetic modification can be achieved by a contacting and incubation that is as brief as 4 hours before the cells are washed to remove free recombinant retroviral particles. After the cells are genetically modified, they can be effectively isolated using a PBMC enrichment procedure. As shown in this Example, the anticoagulant can be EDTA or Na-Heparin. Similar results were obtained using ACD as the anticoagulant in other experiments.

### Example 4. Time Course of Retroviral Transduction of Unstimulated PBMCs by Exposure Times of 4 Hours to Less than 1 Minute

In this experiment, recombinant lentiviral particles were contacted and incubated with unstimulated PBMCs for between 4 hours and less than 1 minute, and were examined for their ability to transduce the PBMCs and promote their survival and/or proliferation in vitro in the absence of any exogenous cytokines.

### Methods

Recombinant lentiviral particles were produced in 293T cells (Lenti-X™ 293T, Clontech) that were adapted to suspension culture in Freestyle™ 293 Expression Medium (Thermo Fisher Scientific). The cells were transiently transfected using PEI with a genomic plasmid and separate packaging plasmids encoding gag/pol, rev, and a pseudotyping plasmid encoding VSV-G as described in Example 3 of WO 2019/055946. For certain samples, the transfection reaction mixture also included a plasmid encoding UCHT1scFvFc-GPI as further described in Example 3 of WO 2019/055946. Two genomic plasmids were used in this example. The first plasmid included a Kozak sequence, a CD8a signal peptide, a FLAG tag, and an anti-CD19:CD8:CD3z CAR followed by a triple stop sequence (F1-3-253). The second plasmid included a Kozak sequence, a CD8a signal peptide, a FLAG tag, an anti-CD19:CD8:CD3z CAR, T2A, and the CLE DL3A-4 (E013-T041-S186-S051) followed by a triple stop sequence (F1-3-451).

On Day 0, PBMCs were enriched from buffy coats (San Diego Blood Bank) from 2 donors by density gradient centrifugation with Ficoll-Paque PREMIUM® (GE Healthcare Life Sciences) and SepMate™-50 (Stemcell™ Technologies) according to the manufacturer's instructions. No additional steps were taken to remove monocytes. After isolation, the PBMCs were diluted to 1×10⁶ PBMCs per 1 ml of X-Vivo15 (LONZA) and 1 ml was seeded into each well of 96 deep-well plates. Cells from each donor were also set aside for phenotype analysis by FACS. No anti-CD3, anti-CD28, IL-2, IL-7, or other exogenous cytokine was added to activate or otherwise stimulate the lymphocytes prior to transduction. Lentiviral particles were added directly to the non-stimulated PBMCs at an MOI of 1. The transductions were incubated at 37° C. and 5% CO₂ for either 4 hours, 2 hours, 30 minutes, 15 minutes, 7.5 minutes, 5 minutes, 2.5 minutes or not incubated at all before the cells were spun down using a 5 minute centrifugation at 400 g, and then washed 3 times in 1 ml of DPBS+2% HSA, using 5 minute centrifugations at 400 g. Thus, for a calculation of combined transduction and incubation times, 5 minutes could be added to account for the first centrifugation, in which it is believed that the vast majority of lentiviral particles not associated with cells, were separate away from the cells. The cells in each well were then resuspended in 1 ml X-Vivo15 and incubated at 37° C. and 5% CO₂. For samples treated with antiviral drugs, dapivirine or dolutegravir was added to a final concentration of 10 μM during the transduction and the transduction reaction was incubated at 37° C. and 5% CO₂ for 4 hours. The drugs were replenished at the same concentrations in the recovery medium after the three washes. No exogenous cytokines were added to the samples at any time. Samples were collected at Day 6 and transduction efficiencies based on FLAG expression was determined by FACS analysis using a lymphocyte gate based on forward and side scatter.

Results

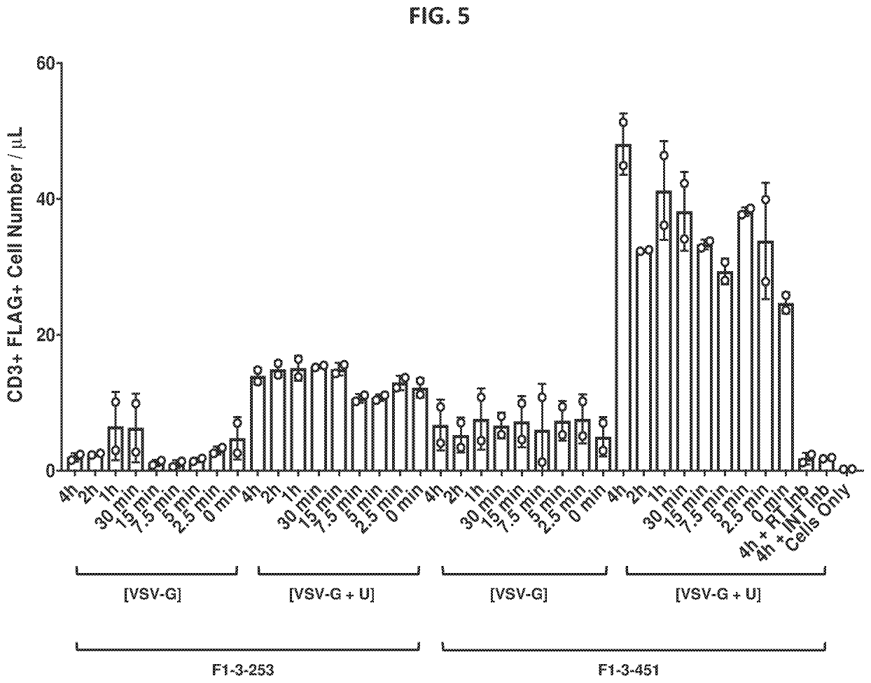

In this example, an incubation period of less than 1 minute was found to be as effective at promoting the transduction of unstimulated PBMCs by recombinant lentiviral particles as was an incubation period of 4 hours. FIG. 5 shows the CD3+FLAG+ absolute cell count (per ul) at Day 6 after transduction of unstimulated PBMCs from 1 Donor by the different recombinant lentiviral particles for the indicated period of time. The ability of each of the recombinant lentiviral particles to transduce PBMCs was similar across all incubation periods. This is particularly evident for the lentiviral particles that express anti-CD3scFvFc-GPI and had higher transduction efficiencies than their non anti-CD3scFvFc-GPI expressing counterparts. For all incubation times examined, the total number of transduced PBMCs was greater in those samples transduced by [F1-3-451GU] than by [F1-3-253GU]indicating that the DL3A CLE encoded in F1-3-451 is promoting the survival and/or proliferation of these cells. The inhibition of transduction by dapivirine, a reverse transcriptase, and dolutegravir, an integrase inhibitor, as shown in FIG. 5 demonstrate that genetic modification and transgene expression by these PBMCs is not pseudotransduction, but rather is the result of transduction in which the viral transgene RNA is reverse transcribed, integrated into the genomes of PBMCs, and expressed. Similar results were observed using PBMCs from the second Donor.

### Example 5. miRNA Expression Increased In Vivo Survival and/or Proliferation of Transduced Cells Expressing a CAR

In this example, two miRNA libraries (Library 314 and Library 315) of candidate (putative) blocks of 4 miRNA precursors were assembled in series from pools of individual miRNA precursors. The miRNA blocks were inserted into the EF-1 alpha intron of lentiviral constructs encoding an EF-1 alpha promoter driving expression of a CAR. Human PBMCs were transduced with lentiviral particles encoding these libraries, and injected into tumor-bearing mice. After 20 days, the tumors were harvested and the identity of the miRNA blocks in the PBMCs from the tumors was determined by PCR followed by Sanger Sequencing. Thus, the screen identified miRNA blocks that are able to promote the proliferation and/or survival of transduced PBMCs in a tumor.

### Methods

### Library Preparation

108 gBlocks® Gene Fragments were used to generate a library of constructs each containing 4 miRNA precursors in series in positions 1 (P1), 2 (P2), 3 (P3), and 4 (P4). Each gBlock® was specific to P1, P2, P3, or P4 and contained a miR-155 framework (SEQ ID NO:457), including a 5′arm and a 3′arm as described in Example 17 of WO 2019/055946, in which a unique miRNA fragment targeting an mRNA transcript corresponding to 1 of 27 different genes was used to replace the miR-155 stem-loop precursor. For clarity, the sequences of miRNA fragments differed for each position P1-P4 even among miRNA fragments that targeted mRNA transcripts corresponding to the same gene. The gBlocks® for each position contained a unique 40 bp overlap sequence and the type IIs assembly method was used to assemble combinations of four gBlocks® in their prescribed order, to generate the library. By these methods, a total diversity of 531,441 unique constructs (27 miRNA at P1×27 miRNA at P2×27 miRNA at P3×27 miRNA at P4) was possible.

The library of miRNA constructs was separately cloned into the EF-1 alpha intron A of F1-1-315 and F1-2-314 to generate Library 315 and Library 314, respectively. In addition to the EF-1 alpha promoter, F1-1-315 included a CD8a signal peptide, an anti-ROR2:CD28:CD3z CAR, T2A, and an eTag. Similarly, in addition to the EF-1 alpha promoter, F1-2-314 included a CD8a signal peptide, an anti-Axl:CD8:CD3z CAR, T2A, and an eTag. FIGS. 26A and 26B of WO 2019/055946 include a similar lentiviral vector with an EF-1 alpha promoter, including intron A with 4 miRNA precursors, that drove expression of GFP instead of either CAR.

The 27 gene targets in this example and the sequence identification numbers for DNA sequences corresponding to the miRNAs in each position are shown in Table 2 below.

TABLE 2
SEQ ID NOs. of DNA sequences corresponding
to miRNA at each position for each target.

| Gene Target | Position 1 | Position 2 |
|---|---|---|
| cCBL | SEQ ID NO: 342 | SEQ ID NO: 343 |
| CD3z | SEQ ID NO: 346 | SEQ ID NO: 347 |
| PD1 | SEQ ID NO: 350 | SEQ ID NO: 351 |
| CTLA4 | SEQ ID NO: 354 | SEQ ID NO: 355 |
| TIM3 | SEQ ID NO: 358 | SEQ ID NO: 359 |
| LAG3 | SEQ ID NO: 362 | SEQ ID NO: 363 |
| SMAD2 | SEQ ID NO: 366 | SEQ ID NO: 367 |
| TNFRSF10B | SEQ ID NO: 370 | SEQ ID NO: 371 |
| PPP2CA | SEQ ID NO: 374 | SEQ ID NO: 375 |
| TNFRSF6 | SEQ ID NO: 378 | SEQ ID NO: 379 |
| BTLA | SEQ ID NO: 382 | SEQ ID NO: 383 |
| TIGIT | SEQ ID NO: 386 | SEQ ID NO: 387 |
| A2AR | SEQ ID NO: 390 | SEQ ID NO: 391 |
| AHR | SEQ ID NO: 394 | SEQ ID NO: 395 |
| EOMES | SEQ ID NO: 398 | SEQ ID NO: 399 |
| SMAD3 | SEQ ID NO: 402 | SEQ ID NO: 403 |
| SMAD4 | SEQ ID NO: 406 | SEQ ID NO: 407 |
| TGFBR2 | SEQ ID NO: 410 | SEQ ID NO: 411 |
| PPP2R2D | SEQ ID NO: 414 | SEQ ID NO: 415 |
| TNFSF6 | SEQ ID NO: 418 | SEQ ID NO: 419 |
| CASP3 | SEQ ID NO: 422 | SEQ ID NO: 423 |
| SOCS2 | SEQ ID NO: 426 | SEQ ID NO: 427 |
| TIEG1 | SEQ ID NO: 430 | SEQ ID NO: 431 |
| JunB | SEQ ID NO: 434 | SEQ ID NO: 435 |
| Cbx3 | SEQ ID NO: 438 | SEQ ID NO: 439 |
| Tet2 | SEQ ID NO: 442 | SEQ ID NO: 443 |
| HK2 | SEQ ID NO: 446 | SEQ ID NO: 447 |
| Gene Target | Position 3 | Position 4 |
| cCBL | SEQ ID NO: 344 | SEQ ID NO: 345 |
| CD3z | SEQ ID NO: 348 | SEQ ID NO: 349 |
| PD1 | SEQ ID NO: 352 | SEQ ID NO: 353 |
| CTLA4 | SEQ ID NO: 356 | SEQ ID NO: 357 |
| TIM3 | SEQ ID NO: 360 | SEQ ID NO: 361 |
| LAG3 | SEQ ID NO: 364 | SEQ ID NO: 365 |
| SMAD2 | SEQ ID NO: 368 | SEQ ID NO: 369 |
| TNFRSF10B | SEQ ID NO: 372 | SEQ ID NO: 373 |
| PPP2CA | SEQ ID NO: 376 | SEQ ID NO: 377 |
| TNFRSF6 | SEQ ID NO: 380 | SEQ ID NO: 381 |
| BTLA | SEQ ID NO: 384 | SEQ ID NO: 385 |
| TIGIT | SEQ ID NO: 388 | SEQ ID NO: 389 |
| A2AR | SEQ ID NO: 392 | SEQ ID NO: 393 |
| AHR | SEQ ID NO: 396 | SEQ ID NO: 397 |
| EOMES | SEQ ID NO: 400 | SEQ ID NO: 401 |
| SMAD3 | SEQ ID NO: 404 | SEQ ID NO: 405 |
| SMAD4 | SEQ ID NO: 408 | SEQ ID NO: 409 |
| TGFBR2 | SEQ ID NO: 412 | SEQ ID NO: 413 |
| PPP2R2D | SEQ ID NO: 416 | SEQ ID NO: 417 |
| TNFSF6 | SEQ ID NO: 420 | SEQ ID NO: 421 |
| CASP3 | SEQ ID NO: 424 | SEQ ID NO: 425 |
| SOCS2 | SEQ ID NO: 428 | SEQ ID NO: 429 |
| TIEG1 | SEQ ID NO: 432 | SEQ ID NO: 433 |
| JunB | SEQ ID NO: 436 | SEQ ID NO: 437 |
| Cbx3 | SEQ ID NO: 440 | SEQ ID NO: 441 |
| Tet2 | SEQ ID NO: 444 | SEQ ID NO: 445 |
| HK2 | SEQ ID NO: 448 | SEQ ID NO: 449 |

### Lentiviral Particle Production

Library 315 and Library 314 were separately used to produce lentiviral particles in 30 ml suspension cultures of 293T cells. The lentiviral particles were harvested and concentrated by PEG precipitation. Other details regarding lentiviral particle production are provided in Example 17 of WO 2019/055946.

### Transduction

On Day 0, PBMCs were isolated from ACD peripheral blood and 5.0×10⁷ viable PBMCs were seeded into each of two 1L G-Rex devices in 100 ml with Complete OpTmizer™ CTS™ T-Cell Expansion SFM supplemented with 100 IU/ml IL-2 (Novoprotein, GMP-CD66), 10 ng/ml IL-7 (Novoprotein, GMP-CD47), and 50 ng/ml anti-CD3 antibody (Novoprotein, GMP-A018) to activate the PBMCs, which included T cells and NK cells, for viral transduction. Lentiviral particles were added directly to the activated PBMCs in 1 G-Rex for Library 315 and the other G-Rex for Library 314 at an MOI of 5, and incubated overnight. The G-Rex devices were incubated in a standard humidified tissue culture incubator at 37° C. and 5% CO₂ with additions of 100 IU/ml recombinant human IL-2 and 10 ng/ml recombinant human IL-7 solution every 48 hours and the cultures were expanded until day 12 at which time the cells are predominantly T cells. Other details regarding PBMC enrichment, transduction, and ex vivo expansion are provided in Example 16 of WO 2019/055946.

### Tumor Inoculation and Administration of Transduced Cells

A xenograft model using NOD Scid Gamma (NSG) mice was chosen to probe the ability of human PBMCs transduced with lentiviral particles of Library 315 or Library 314 to survive and/or proliferate in vivo, where the tumors expressed or did not express the antigen recognized by the CAR encoded in the genomes of these lentiviral particles. Mice were handled in accordance with Institutional Animal Care and Use Committee approved protocols. Subcutaneous (sc) tumor xenografts were established in the hind flank of 12 week old female NOD-Prkdc^{scid}II2rg^{tm1}/Begen (B-NSG) mice (Beijing Biocytogen Co. Ltd.). Briefly, cultured CHO cells, cultured CHO cells transfected to stably express human ROR2 (CHO-ROR2) or human AXL (CHO-AXL) were separately washed in DPBS (Thermo Fisher), counted, resuspended in cold DPBS and mixed with an appropriate volume of Matrigel ECM (Corning; final concentration 5 mg/mL) at a concentration of 0.47×10⁶ cells/200 μl on ice. Animals were prepared for injection using standard approved anesthesia with hair removal (Nair) prior to injection. 200 μl of either cell suspension in ECM was injected sc into the rear flanks for CHO cells (n=2), CHO-ROR2 cells (n=1), and CHO-AXL cells (n=1), respectively.

5 days after tumor inoculation, 1 mouse bearing a CHO tumor and 1 mouse bearing a CHO-ROR2 tumor were dosed intravenously (IV) by tail vein injection with 200 μl DPBS containing 1×10⁷ PBMCs transduced with lentiviral particles from Library 315 after 12 days of ex vivo culture. Similarly, 5 days after tumor inoculation, 1 mouse bearing a CHO tumor and 1 mouse bearing a CHO-Axl tumor were dosed intravenously (IV) by tail vein injection with 200 μl DPBS containing 1×10⁷ PBMCs transduced with lentiviral particles from Library 314.

### Tumor Harvesting and DNA Sequencing

On day 20 after dosing with transduced PBMCs, the tumors were excised. DNA from half of each tumor was extracted and 4 ug from each tumor was used as a template in a PCR reaction for 25 cycles to amplify the EF-1alpha intron. The amplicons were cloned into a sequencing vector, transformed into bacteria, and streaked onto plates. 18 total colonies (˜5 per mouse) were selected and DNA was prepared and analyzed using Sanger sequencing to determine the sequences of a sample of the miRNA constructs present in the tumor.

### Results

A mouse xenograft model was used to determine whether miRNA targeting specific gene transcripts were able to increase the proliferation and/or survival of transduced PBMCs expressing CARs in vivo, where the xenografts were tumors with or without expression of the target antigen of the CARs. For this analysis, a library of miRNA constructs was generated consisting of miRNAs directed against 27 distinct targets. The miRNA constructs analyzed contained 4 positions for 4 separate miRNAs, as shown in FIG. 26B and Example 17 and Example 18 of WO 2019/055946. Tumor DNA was analyzed by sequencing the EF-1alpha intron to identify which miRNA constructs were present 20 days after injection of transduced PBMCs, and therefore which miRNA constructs increased proliferation and/or survival.

531,441 different combinations of 4 miRNAs in series were possible. Of the 18 EF-1alpha introns sequenced, 13 contained a miRNA construct where all 4 miRNA in the construct were directed against one target, and 2 contained miRNA constructs directed to more than 1 target. Table 3 below shows the miRNA species recovered from each of the 4 tumors examined in this example.

TABLE 3
Identity of miRNA target at each position of the
miRNA constructs that were sequenced.

| Library | Tumor | Position 1 | Position 2 | Position 3 | Position 4 |
|---|---|---|---|---|---|
| Library | CHO | FAS | FAS | FAS | FAS |
| 315 | | FAS | FAS | FAS | FAS |
| | | AHR | AHR | AHR | AHR |
| | | FAS | FAS | FAS | FAS |
| | | CD3z | CD3z | CD3z | CD3z |
| Library | CHO-ROR2 | NA | NA | NA | NA |
| 315 | | FAS | FAS | FAS | FAS |
| | | FAS | FAS | FAS | FAS |
| | | cCBL | cCBL | cCBL | cCBL |
| Library | CHO | Cbx | Cbx | Cbx | Cbx |
| 314 | | cCBL | cCBL | cCBL | cCBL |
| | | HK2 | HK2 | HK2 | HK2 |
| | | NA | NA | NA | NA |
| Library | CHO-AXL | NA | NA | NA | NA |
| 314 | | FAS | FAS | FAS | FAS |
| | | FAS | FASL | SMAD4 | HK2 |
| | | SMAD4 | SMAD4 | SMAD4 | SMAD4 |
| | | EOMES | NA | EOMES | AHR |

Notably, 6 EF-1alpha introns contained a miRNA construct with all 4 miRNA directed against TNFRSF6 (FAS). 2 EF-1alpha introns contained a miRNA construct with all 4 miRNA directed against cCBL. For each of AHR, CD3z, Cbx, and HK2, 1 EF-1alpha intron was identified that contained an miRNA construct with all 4 miRNA directed against that gene transcript. “NA” indicated that no miRNA block was identified in that position. Together, these results indicate that knocking down transcripts encoding FAS, cCBL, CD3z, Cbx, HK2, FASL, SMAD4, EOMES, and AHR can promote the survival and/or proliferation of T cells in the tumor microenvironment. The identification of 4 miRNA in series to FAS under each condition in 6 of the 18 samples examined indicates that knocking down FAS transcripts confers a particular advantage for survival and/or proliferation. Furthermore, this data suggests that there is a dosage effect such that 4 species of miRNA directed to FAS, cCBL, AHR, CD3z, Cbx, and HK2, leads to greater knockdown of transcripts encoding these genes than does 1, 2, or 3 species, and that this increased knockdown confers a survival and/or proliferation advantage.

### Example 6. Identification of Candidate Chimeric Polypeptide Lymphoproliferative Elements Using an In Vivo Assay

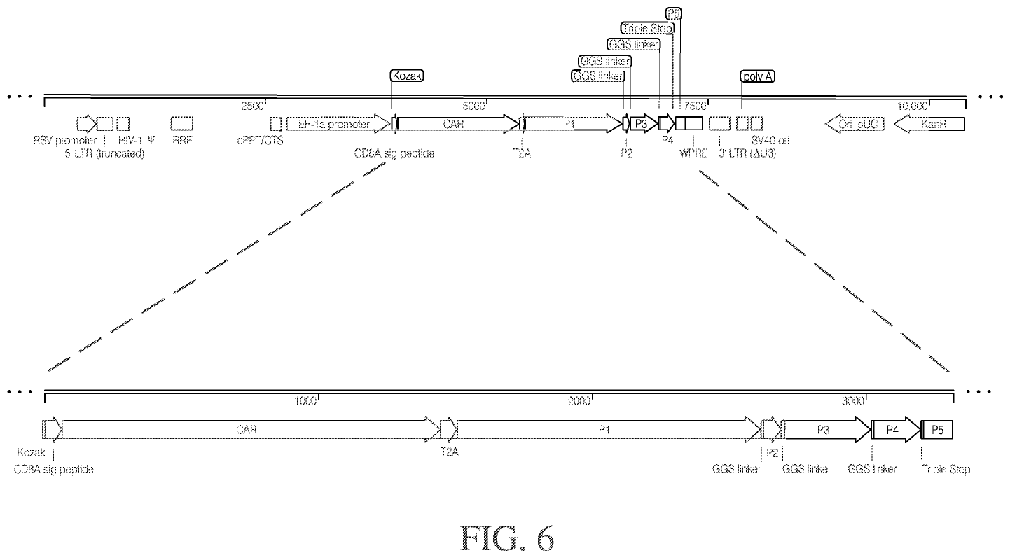

In this example, two chimeric polypeptide libraries (Library 6 and Library 8) of candidate (putative) chimeric lymphoproliferative elements (CLEs) were assembled into viral vectors from pools of extracellular-transmembrane block sequences, intracellular block sequences, and a barcode library according to the chimeric polypeptide-encoding construct provided in FIG. 6. The chimeric library candidates (putative CLEs) were screened for the ability of the candidate chimeric polypeptides to promote expansion of T cells in vivo.

### Library Constructs

Two libraries were made and analyzed in this study; Library 6 and Library 8. The libraries shared a common structure, which is shown in FIG. 6. FIG. 6 provides a schematic of a non-limiting, exemplary transgene expression cassette containing a polynucleotide sequence encoding a CAR and a candidate CLE from a library having 4 modules driven by an EF-1 alpha promoter and a Kozak-type sequence (GCCGCCACC(SEQ ID NO:450)), in a lentiviral vector backbone. Each candidate lymphoproliferative element included 4 modules; an extracellular module (P1), a transmembrane module (P2), and 2 intracellular modules (P3 and P4). The P1 module encoded an eTAG at the 5′ terminus of a c-Jun domain. A triple stop sequence (TAATAGTGA (SEQ ID NO:451)) separated P4 from a DNA barcode (P5). A WPRE (CTCCTTTCCATGCCTGCTCGCCTCTGTTGCCACCTGCATTCTGCGCGGGACGTCCTTCTGCTA CGTCCCTTCGGCCCTCAATCCAGCGGACCTTCCTTCCCGCGGCCTGCTGCCGGCTCTGCGGCC TCTTCCGCGTCTTCGCCTTCGCCCTCAGACGAGTCGGATCTCCCTTTGGGCCGCCTCCCCGCC TG (SEQ ID NO:452)) was present between the last stop codon (starting 4 bp after the last nucleotide of P5) and the 3′ LTR (which started 83 nucleotides after the last nucleotide of the WPRE).

The CAR and P1 were separated by a polynucleotide sequence encoding a T2A ribosomal skip sequence. The general design and construction of the library, including the barcode, was as disclosed in Example 11 of WO 2019/055946, except for the P1 and P2 domains, as set out in more detail later in this Example.

The design of Library 6 and Library 8 differed only in the polynucleotide encoding the CAR. The CAR of Library 6 encoded an MRB-ASTR that has an scFv that recognizes human AXL, a CD28 stalk and transmembrane sequence (SEQ ID NO:25), a CD28 intracellular domain deleted for Lck binding (ICΔ) (SEQ ID NO:55), and an intracellular activating domain from CD3z (SEQ ID NO: 28). The CAR of Library 8 encoded a FLAG-tagged MRB-ASTR that has an scFv that recognizes human ROR2, a CD8 stalk and transmembrane sequence (SEQ ID NO:24), a CD137 intracellular domain (SEQ ID NO:53), and an intracellular activating domain from CD3z (SEQ ID NO: 28).

### Synthesis of Viral Vectors and Lentiviral Production

Vectors were synthesized and lentiviral particles were produced for each library as disclosed in Example 11 of WO 2019/055946.

### Transduction and Culturing of PBMCs

Whole human blood from 2 healthy donors was collected and processed separately using a Sepax 2 S-100 device to obtain PBMCs as described in Example 12 of WO 2019/055946. 4.75e7 or 5e7 viable PBMCs for Libraries 6 and 8, respectively, were seeded into each of two 1L G-Rex devices in 100 ml and activated, transduced, and the cultures were expanded for 12 days as described in Example 5 above. 3.9e9 total cells were recovered (82-fold expansion) for Library 6, 9.71e7 of which were CD3+eTAG+ transduced T cells. 2.47e9 total cells were recovered (49-fold expansion) for Library 8, 2.44e8 of which were CD3+eTAG+ transduced T cells. 4e6 cells from each expansion were set aside and frozen for later analysis by next generation sequencing.

### Tumor Inoculation and Administration of Transduced Cells

A xenograft model using NSG mice was chosen to probe the ability of human PBMCs transduced with lentiviral particles of Library 6 or Library 8 to survive and/or proliferate in vivo, where the tumors expressed or did not express the antigen recognized by the CAR encoded in the genomes of these lentiviral particles. Subcutaneous (sc) CHO, CHO-ROR2, or CHO-AXL tumor xenografts were established in the hind flanks of B-NSG (Beijing Biocytogen Co. Ltd.) mice as described in Example 5.

5 days after tumor inoculation, 6 mice bearing CHO tumors and 5 mice bearing CHO-Axl tumors were dosed intravenously (IV) by tail vein injection with 200 μl DPBS containing 7×10⁷ PBMCs transduced with lentiviral particles from Library 6. Similarly, 5 days after tumor inoculation, 6 mice bearing CHO tumors and 5 mice bearing CHO-ROR2 tumors were dosed IV by tail vein injection with 200 μl DPBS containing 7×10⁷ PBMCs transduced with lentiviral particles from Library 8. Mice bearing CHO tumors, CHO-AXL tumors, or CHO-ROR2 tumors were also dosed with 200 μl DPBS alone as controls.

### Tissue Harvesting, Isolation of Human CD45+ Cells, and DNA Sequencing

Approximately 100 μl of blood was collected from each mouse on days 7, 14, and 21 (for Library 6) or days 7, 14, and 19 (for Library 8) after dosing with transduced PBMCs. Spleen and tumor was also collected when the mice were euthanized on day 21 or day 19. Half of each tissue was processed to isolate human CD45+ cells by mechanically disrupting the tissue, enzymatic digestion with collagenase IV and DNAse I, and magnetic isolation of cells using hCD45 antibody (Biolegend, 304004). Genomic DNA was prepared from these hCD45+ cells and corresponds to “purified spleen” and “purified tumor” samples. Genomic DNA was prepared directly from the other half of each tissue and corresponds to “non purified spleen” and “non purified tumor. Purified genomic DNA was sequenced using an Illumina HiSeq, generating paired-end 150 bp reads. Usually, a subset of 10 million reads was extracted from each indexed fastq file and processed for analysis using barcode reader, a custom R script engineered to extract barcode sequences based on the presence of a constant region. Purified genomic DNA was also sequenced on a PacBio sequencing system to obtain longer read lengths to associate barcodes with constructs.

qPCR

Genomic DNA (gDNA) isolated from tissue samples were evaluated for the presence of transduced lymphocytes by bioanalytical qPCR. Genomic DNA was isolated from the samples using the QIAamp DNA Blood Mini kit (Qiagen 51106) and the DNA was further cleaned using the QIAamp DNA Micro Kit (56304). A TaqMan assay (Thermo Fisher) was performed on the isolated genomic DNA using a primer and probe set specific for the 5′ LTR of lentivirus to quantitate lentivirus copy number per ug of tissue.

### Data Analysis

DNA barcodes were identified in a 20 million subset of Illumina HiSeq sequenced reads. Count data for all samples was assembled and barcodes present in less than 2 samples were considered artifactual and discarded. Count data from pre-injection PBMCs was used as a representation of the initial barcode population. Full length constructs were identified using an association table created by Long Read Sequencing of a few select samples. After summing up counts for barcodes mapping to the same construct, all data was scaled based on qPCR-quantified lentivirus copy number per ug of tissue. Samples with very low lentivirus copy numbers were removed from the analysis. Ranking of CAR/antigen signal-independent chimeric polypeptide candidates was obtained by calculating the total counts for each construct in each tissue of interest from mice bearing CHO tumors devoid of the cognate target antigen recognized by the CAR. Ranking for CAR/antigen signal-dependent drivers was obtained using the following formula: MR*-log 10(P), where the MR was the mean ratio between the count values in the mice bearing tumors with antigen (CHO-AXL or CHO-ROR2) and tumors without antigen (CHO) and P was the p value obtained from a one-sided Mann-Whitney-Wilcoxon test comparing the count values in the mice bearing tumors with or without antigen. One-sided Mann-Whitney-Wilcoxon tests were used to determine whether a particular part was enriched as compared with all other represented parts for a specific position. Individual tissue p values were aggregated using the Stouffer sumz method to obtain final rankings. Full construct rankings were obtained by averaging individual tissue ranks.

### Results

In this experiment, chimeric polypeptide candidates were designed to have 4 test domains, which included an extracellular domain (P1), a transmembrane domain (P2), a first intracellular domain (P3), and a second intracellular domain (P4) (FIG. 6). As explained in Examples 11 and 12 of WO 2019/055946, the constructs included a DNA barcode to aid in analysis and identification of the construct using next-generation sequencing. Additionally, all of the constructs included nucleic acid sequences encoding a recognition and/or elimination domain in frame with the extracellular domain. The constructs in this Example also encoded a CAR directed to human AXL or human ROR2 upstream of the chimeric polypeptide candidate (FIG. 6). The extracellular domains (P1), transmembrane domains (P2), first intracellular domains (P3), and second intracellular domains (P4) used to generate the chimeric polypeptide candidates in Library 6 and Library 8 were the same as in Example 12 of WO 2019/055946. The libraries did not include all of the possible combinations of P1-P4 domains.

The number of constructs present after transduction of PBMCs and 12 days of growth in culture in the presence of exogenous cytokines was determined for both Library 6 and Library 8 by counting the number of individual barcodes that were present in more than one read in the day 12 cultured sample. Of the 697,410 potential combinations, 219,649 and 127,634 different constructs were detected for Library 6 and Library 8, respectively. Detailed information about the top candidates analyzed can be determined from Table 1 and Tables 4-8. The coding system for constructs is the same as explained for Examples 11 and 12 of WO 2019/055946.

After culturing for 12 days, transduced PBMCs were injected into mice bearing tumors with or without antigen. PBMCs transduced with constructs from Library 6, which encoded the anti-AXL CAR, were injected into mice bearing CHO tumors or CHO-AXL tumors, and PBMCs transduced with constructs from Library 8, which encoded the anti-ROR2 CAR, were injected into mice bearing CHO tumors or CHO-ROR2 tumors. After 21 or 19 days of in vivo expansion (Library 6 and Library 8, respectively), samples from the blood, spleen, and tumor of each mouse were harvested. Half of each spleen and tumor was processed to isolate CD45+ cells and is referred to herein in this example as a “purified” sample. DNA from each sample from each mouse (blood, non-purified spleen, purified spleen, non-purified tumor, and purified tumor) was sequenced. The barcodes on each construct were used to identify and sum the number of sequencing reads for each construct in each sample.

A non-parametric analysis was used to identify constructs that promoted PBMC cell proliferation in vivo in either a CAR/antigen signal-independent or CAR/antigen signal-dependent manner. To identify chimeric polypeptide candidates that were CAR/antigen signal-independent, each sample of each construct was ranked based on the number of sequencing reads in mice bearing CHO tumors. The top constructs were identified as having the best average rank of the 5 tissue samples. The top 100 chimeric polypeptide candidates that were CAR/antigen signal-independent for Library 6 and Library 8 are shown in Tables 31 and 32, respectively.

To identify chimeric polypeptide candidates that were CAR/antigen signal-dependent, the ranking for each sample included the ratio of reads between mice bearing tumors with antigen (CHO-AXL or CHO-ROR2) and mice bearing tumors without antigen (CHO). The top 100 chimeric polypeptide candidates that were CAR/antigen signal-dependent for Library 6 and Library 8 are shown in Tables 33 and 34, respectively.

An additional analysis was run to identify noteworthy chimeric polypeptide candidates that were CAR/antigen signal-independent. For this analysis, 20 parts were first identified that performed the best for any P2, P3, or P4 position, based on a statistical test to determine whether a particular part was enriched as compared with all other represented parts for a specific position. In this combined analysis, from constructs that included at least one of these 20 parts, best-performing constructs from either Library 6 or Library 8 were identified based on the sum of the normalized counts in mice bearing CHO tumors. The 30 best-performing chimeric polypeptide candidates according to this analysis that were CAR/antigen signal-independent are shown in Table 8.

Several of the CLEs identified in the library screen and shown in Table 8 were generated as individual chimeric polypeptides in lentivirus constructs behind the anti-AXL CAR as configured in Library 6 and run in confirmatory in vitro screens. Frozen PBMCs from 3 donors were thawed and rested in Complete OpTmizer™ CTS™ T-Cell Expansion SFM supplemented with 100 IU/ml of IL-2 and 10 ng/ml IL-7 overnight in a standard humidified tissue culture incubator at 37° C. and 5% CO₂. The PBMCs were activated on Day 0 with 50 ng/ml anti-CD3 and transduced on Day 1 with viral particles at an MOI of 5. On Day 2 the PBMCs were transferred to the wells of a 24-well G-Rex plate and cultured in Complete OpTmizer™ CTS™ T-Cell Expansion SFM in the absence of any exogenous cytokines until Day 35 days. In replicate experiments performed using PBMCs from 3 donors, CLE's with P2, P3, and P4 configurations T001-S121-S212 and T044-S186-S053 showed particularly noteworthy expansion on Days 14, 21, 28, and 35.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

TABLE 1
Parts, names, and amino acid sequences for domains of lymphoproliferative parts P1-P2, P1, P2,
P3, and P4.

| Part | Name | Amino Acid Sequence |
|---|---|---|
| M001 | eTAG IL7RA Ins | MLLLVTSLLLCELPHPAFLLIPRKVCNGIGIGEFKDSLSINATNIKHFKNCTSISGDLHILPVAFRGDSFTHTPPLDPQELDILK |
| | PPCL (interleukin 7 | TVKEITGFLLIQAWPENRTDLHAFENLEIIRGRTKQHGQFSLAVVSLNITSLGLRSLKEISDGDVIISGNKNLCYANTINWK |
| | receptor) | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGPEINNSSGEMDPILLPPCLTISILSFFSVALLVILACVL (SEQ ID NO: 84) |
| M002 | eTAG IL7RA Ins | MLLLVTSLLLCELPHPAFLLIPRKVCNGIGIGEFKDSLSINATNIKHFKNCTSISGDLHILPVAFRGDSFTHTPPLDPQELDILK |
| | PPCL (interleukin 7 | TVKEITGFLLIQAWPENRTDLHAFENLEIIRGRTKQHGQFSLAVVSLNITSLGLRSLKEISDGDVIISGNKNLCYANTINWK |
| | receptor) | KLFGTSGQKTKIISNRGENSCKATGQPEINNSSGEMDPILLPPCLTISILSFFSVALLVILACVL (SEQ ID NO: 85) |
| M007 | Myc Tag LMP1 | MEQKLISEEDLEHDLERGPPGPRRPPRGPPLSSSLGLALLLLLLALLFWLYIVMSDWTGGALLVLYSFALMLIIIILIIFIFRRD |
| | NC_007605 1 | LLCPLGALCILLLMITLLLIALWNLHGQALFLGIVLFIFGCLLVLGIWIYLLEMLWRLGATIWQLLAFFLAFFLDLILLIIALYLQ |
| | | QNWWILLVDLLWLLLFLAILIWM (SEQ ID NO: 86) |
| M008 | Myc LMP1 | MEQKLISEEDLSSSLGLALLLLLLALLFWLYIVMSDWIGGALLVLYSFALMLIIIILIIFIFRRDLLCPLGALCILLLMITLLLIAL |
| | NC_007605_1 | WNLHGQALFLGIVLFIFGCLLVLGIWIYLLEMLWRLGATIWQLLAFFLAFFLDLILLIIALYLQQNWWTLLVDLLWLLLFLAI |
| | | LIWM (SEQ ID NO: 87) |
| M009 | LMP1 | MEHDLERGPPGPRRPPRGPPLSSSLGLALLLLLLALLFWLYIVMSDWTGGALLVLYSFALMLIIIILIIFIFRRDLLCPLGALCI |
| | NC_007605_1 | LLLMITLLLIALWNLHGQALFLGIVLFIFGCLLVLGIWIYLLEMLWRLGATIWQLLAFFLAFFLDLILLIIALYLQQNWWTLL |
| | | VDLLWLLLFLAILIWM (SEQ ID NO: 88) |
| M010 | LMP1 | MSLGLALLLLLLALLFWLYIVMSDWTGGALLVLYSFALMLIIIILIIFIFRRDLLCPLGALCILLLMITLLLIALWNLHGQALFLG |
| | NC_007605_1 | IVLFIFGCLLVLGIWIYLLEMLWRLGATIWOLLAFFLAFFLDLILLIIALYLQQNWWILLVDLLWLLLFLAILIWM (SEQ ID |
| | | NO: 89) |
| M012 | eTAG CRLF2 | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_022148_3 | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGAETPTPPKPKLSKCILISSLAILLMVSLLLLSLW (SEQ ID NO: 90) |
| M013 | eTAG CRLF2 | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_022148_3 | KLFGTSGQKTKIISNRGENSCKATGQAETPTPPKPKLSKCILISSLAILLMVSLLLLSLW (SEQ ID NO: 91) |
| M018 | eTAG CSF2RB | MLLLVTSLLLCELPHPAFLLIPRKVCNGIGIGEFKDSLSINATNIKHFKNCTSISGDLHILPVAFRGDSFTHTPPLDPQELDILK |
| | NM_000395_2 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGTESVLPMWVLALIEIFLTIAVLLAL (SEQ ID NO: 92) |
| M019 | eTAG CSF2RB | MLLLVTSLLLCELPHPAFLLIPRKVCNGIGIGEFKDSLSINATNIKHFKNCTSISGDLHILPVAFRGDSFTHTPPLDPQELDILK |
| | NM_000395_2 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQTESVLPMWVLALIEIFLTIAVLLAL (SEQ ID NO: 93) |
| M024 | eTAG CSF3R | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000760_3 | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGTPEGSELHIILGLFGLLLLLNCLCGTAWLCC (SEQ ID NO: 94) |
| M025 | eTAG CSF3R | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000760_3 | KLFGTSGQKTKIISNRGENSCKATGQTPEGSELHIILGLFGLLLLLNCLCGTAWLCC (SEQ ID NO: 95) |
| M030 | eTAG EPOR | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000121_3 | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGTPSDLDPCCLTLSLILVVILVLLTVLALLS (SEQ ID NO: 96) |
| M031 | eTAG EPOR | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000121_3 | KLFGTSGQKTKIISNRGENSCKATGQTPSDLDPCCLTLSLILVVILVLLTVLALLS (SEQ ID NO: 97) |
| M036 | eTAG GHR | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000163_4 | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGTLPQMSCIFTCCEDFYFPWLLCIIFGIFGLTVMLFVFLFS (SEQ ID NO: 98) |
| M037 | eTAG GHR | MLLLVTSLLLCELPHPAFLLIPRKVCNGIGIGEFKDSLSINATNIKHFKNCTSISGDLHILPVAFRGDSFTHTPPLDPQELDILK |
| | transcript variant | TVKEITGFLLIQAWPENRTDLHAFENLEIIRGRTKQHGQFSLAVVSLNITSLGLRSLKEISDGDVIISGNKNLCYANTINWK |
| | 1 NM_000163_4 | KLFGTSGQKTKIISNRGENSCKATGQTLPQMSQFTCCEDFYFPWLLCIIFGIFGLTVMLFVFLFS (SEQ ID NO: 99) |
| M042 | eTAG truncated | MLLLVTSLLLCELPHPAFLLIPRKVCNGIGIGEFKDSLSINATNIKHFKNCTSISGDLHILPVAFRGDSFTHTPPLDPQELDILK |
| | after Fn F523C | TVKEITGFLLIQAWPENRTDLHAFENLEIIRGRTKQHGQFSLAVVSLNITSLGLRSLKEISDGDVIISGNKNLCYANTINWK |
| | IL27RA | KLFGTSGQKTKIISNRGENSCKATGQVCHALCSPEGCWGPEPRDCVSCRNVSRGRECVDKCNLLEGEPREFVENSECIQ |
| | NM_004843_3 | CHPECLPQAMNITCTGRGPDNCIQCAHYIDGPHCVKTCPAGVMGENNTLVWKYADAGHVCHLCHPNCTYGCTGPGL |
| | | EGCPTNGHLPDNTLRWKVLPGILCLWGLFLLGCGLSLA (SEQ ID NO: 100) |
| M043 | eTAG truncated | MLLLVTSLLLCELPHPAFLLIPRKVCNGIGIGEFKDSLSINATNIKHFKNCTSISGDLHILPVAFRGDSFTHTPPLDPQELDILK |
| | after Fn F523C | TVKEITGFLLIQAWPENRTDLHAFENLEIIRGRTKQHGQFSLAVVSLNITSLGLRSLKEISDGDVIISGNKNLCYANTINWK |
| | IL27RA | KLFGTSGQKTKIISNRGENSCKATGQHLPDNTLRWKVLPGILCLWGLFLLGCGLSLA (SEQ ID NO: 101) |
| | NM_004843_3 | |
| M048 | eTAG truncated | MLLLVTSLLLCELPHPAFLLIPRKVCNGIGIGEFKDSLSINATNIKHFKNCTSISGDLHILPVAFRGDSFTHTPPLDPQELDILK |
| | after Fn S505N | TVKEITGFLLIQAWPENRTDLHAFENLEIIRGRTKQHGQFSLAVVSLNITSLGLRSLKEISDGDVIISGNKNLCYANTINWK |
| | MPL | KLFGTSGQKTKIISNRGENSCKATGQVCHALCSPEGCWGPEPRDCVSCRNVSRGRECVDKCNLLEGEPREFVENSECIQ |
| | NM_005373_2 | CHPECLPQAMNITCTGRGPDNCIQCAHYIDGPHCVKTCPAGVMGENNTLVWKYADAGHVCHLCHPNCTYGCTGPGL |
| | | EGCPTNGETATETAWISLVTALHLVLGLNAVLGLLLL (SEQ ID NO: 102) |
| M049 | eTAG truncated | MLLLVTSLLLCELPHPAFLLIPRKVCNGIGIGEFKDSLSINATNIKHFKNCTSISGDLHILPVAFRGDSFTHTPPLDPQELDILK |
| | after Fn S505N | TVKEITGFLLIQAWPENRTDLHAFENLEIIRGRTKQHGQFSLAVVSLNITSLGLRSLKEISDGDVIISGNKNLCYANTINWK |
| | MPL | KLFGTSGQKTKIISNRGENSCKATGQETATETAWISLVTALHLVLGLNAVLGLLLL (SEQ ID NO: 103) |
| | NM_005373_2 | |
| E006 | eTag 0A JUN | MLLLVTSLLLCELPHPAFLLIPRKVCNGIGIGEFKDSLSINATNIKHFKNCTSISGDLHILPVAFRGDSFTHTPPLDPQELDILK |
| | NM_002228_3 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKV (SEQ ID NO: 104) |
| E007 | eTag 1A JUN | MLLLVTSLLLCELPHPAFLLIPRKVCNGIGIGEFKDSLSINATNIKHFKNCTSISGDLHILPVAFRGDSFTHTPPLDPQELDILK |
| | NM_002228_3 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVA (SEQ ID NO: 105) |
| E008 | eTag 2A JUN | MLLLVTSLLLCELPHPAFLLIPRKVCNGIGIGEFKDSLSINATNIKHFKNCTSISGDLHILPVAFRGDSFTHTPPLDPQELDILK |
| | NM_002228_3 | TVKEITGFLLIQAWPENRTDLHAFENLEIIRGRTKQHGQFSLAVVSLNITSLGLRSLKEISDGDVIISGNKNLCYANTINWK |
| | | KFGTSGQKTKIISNRGENSCKATGQVCHALCSPEGCWGPEPRDCVSCRNVSRGRECVDKCNLLEGEPREFVENSECIQC |
| | | HPECLPQAMNITCTGRGPDNCIQCAHYIDGPHCVKTCPAGVMGENNTLVWKYADAGHVCHLCHPNCTYGCTGPGLE |
| | | GCPTNGLERIARLEEKVKTLKAQNSELASTANMLREQVAQLKQKVAA (SEQ ID NO: 106) |
| E009 | eTag 3A JUN | MLLLVTSLLLCELPHPAFLLIPRKVCNGIGIGEFKDSLSINATNIKHFKNCTSISGDLHILPVAFRGDSFTHTPPLDPQELDILK |
| | NM_002228_3 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVAAA (SEQ ID NO: 107) |
| E010 | eTag 4A JUN | MLLLVTSLLLCELPHPAFLLIPRKVCNGIGIGEFKDSLSINATNIKHFKNCTSISGDLHILPVAFRGDSFTHTPPLDPQELDILK |
| | NM_002228_3 | TVKEITGFLLIQAWPENRTDLHAFENLEIIRGRTKQHGQFSLAVVSLNITSLGLRSLKEISDGDVIISGNKNLCYANTINWK |
| | | KLFGTSGQKTKIISNRGENSCKATGQVCHALCSPEGCWGPEPRDCVSCRNVSRGRECVDKCNLLEGEPREFVENSECIQ |
| | | CHPECLPQAMNITCTGRGPDNCIQCAHYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVAAAA (SEQ ID NO: 108) |
| E011 | Myc Tag 0A JUN | MTILGTTFGMVFSLLQVVSGEQKLISEEDLLERIARLEEKVKTLKAQNSELASTANMLREQVAQLKQKV (SEQ ID |
| | NM_002228_3 | NO: 109) |
| E012 | Myc Tag 1A JUN | MTILGTTFGMVFSLLQVVSGEQKLISEEDLLERIARLEEKVKTLKAQNSELASTANMLREQVAQLKQKVA (SEQ ID |
| | NM_002228_3 | NO: 110) |
| E013 | Myc Tag 2A JUN | MTILGTTFGMVFSLLQVVSGEQKLISEEDLLERIARLEEKVKTLKAQNSELASTANMLREQVAQLKQKVAA (SEQ ID |
| | NM_002228_3 | NO: 111) |
| E014 | Myc Tag 3A JUN | MTILGTTFGMVFSLLQVVSGEQKLISEEDLLERIARLEEKVKTLKAQNSELASTANMLREQVAQLKQKVAAA (SEQ ID |
| | NM_002228_3 | NO: 112) |
| E015 | Myc Tag 4A JUN | MTILGTTFGMVFSLLQVVSGEQKLISEEDLLERIARLEEKVKTLKAQNSELASTANMLREQVAQLKQKVAAAA (SEQ ID |
| | NM_002228_3 | NO: 113) |
| T001 | CD2 transcript | LIIGICGGGSLLMVFVALLVFYI (SEQ ID NO: 114) |
| | variant 1 | |
| | NM_001328609_1 | |
| T002 | CD3D transcript | GIIVTDVIATLLLALGVFCFA (SEQ ID NO: 115) |
| | variant 1 | |
| | NM_000732_4 | |
| T003 | CD3E | VMSVATIVIVDICITGGLLLLVYYWS (SEQ ID NO: 116) |
| | NM_000733_3 | |
| T004 | CD3G | GFLFAEIVSIFVLAVGVYFIA (SEQ ID NO: 117) |
| | NM_000073_2 | |
| T005 | CD3Z CD247 | LCYLLDGILFIYGVILTALFL (SEQ ID NO: 118) |
| | transcript variant | |
| | 1 NM_198053_2 | |
| T006 | CD4 transcript | MALIVLGGVAGLLLFIGFIGLGIFF (SEQ ID NO: 119) |
| | variant 1 and 2 | |
| | NM_000616_4 | |
| T007 | CD8A transcript | IYIWAPLAGTCGVLLLSLVIT (SEQ ID NO: 120) |
| | variant 1 | |
| | NM_001768_6 | |
| T008 | CD8B transcript | LGLLVAGVLVLLVSLGVAIHLCC (SEQ ID NO: 121) |
| | variant 2 | |
| | NM_172213_3 | |
| T009 | CD27 | ILVIFSGMFLVFTLAGALFLH (SEQ ID NO: 122) |
| | NM_001242_4 | |
| T010 | CD28 transcript | FWVLVVVGGVLACYSLLVTVAFIIFWV (SEQ ID NO: 123) |
| | variant 1 | |
| | NM_006139_3 | |
| T011 | CD40 transcript | ALVVIPIIFGILFAILLVLVFI (SEQ ID NO: 124) |
| | variant 1 and 6 | |
| | NM_001250_5 | |
| T012 | CD79A transcript | IITAEGIILLFCAVVPGTLLLF (SEQ ID NO: 125) |
| | variant 1 | |
| | NM_001783_3 | |
| T013 | CD796 transcript | GIIMIQTLLIILFIIVPIFLLL (SEQ ID NO: 126) |
| | variant 3 | |
| | NM_001039933_2 | |
| T014 | CRLF2 transcript | FILISSLAILLMVSLLLLSLW (SEQ ID NO: 127) |
| | variant 1 | |
| | NM_022148_3 | |
| T015 | CRLF2 transcript | CILISSLAILLMVSLLLLSLW (SEQ ID NO: 128) |
| | variant 1 | |
| | NM_022148_3 | |
| T016 | CSF2RA transcript | NLGSVYIYVLLIVGTLVCGIVLGFLF (SEQ ID NO: 129) |
| | variant 7 and 8 | |
| | NM_001161529_1 | |
| T017 | CSF2RB | MWVLALIVIFLTIAVLLAL (SEQ ID NO: 130) |
| | NM_000395_2 | |
| T018 | CSF2RB | MWVLALIEIFLTIAVLLAL (SEQ ID NO: 131) |
| | NM_000395_2 | |
| T019 | CSF3R transcript | IILGLFGLLLLLTCLCGTAWLCC (SEQ ID NO: 132) |
| | variant 1 | |
| | NM_000760_3 | |
| T020 | CSF3R transcript | IILGLFGLLLLLNCLCGTAWLCC (SEQ ID NO: 133) |
| | variant 1 | |
| | NM_000760_3 | |
| T021 | EPOR transcript | LILTLSLILVVILVLLTVLALLS (SEQ ID NO: 134) |
| | variant 1 | |
| | NM_000121_3 | |
| T022 | EPOR transcript | CCLTLSLILVVILVLLTVLALLS (SEQ ID NO: 135) |
| | variant 1 | |
| | NM_000121_3 | |
| T023 | FCER1G | LCYILDAILFLYGIVLTLLYC (SEQ ID NO: 136) |
| | NM_004106_1 | |
| T024 | FCGR2C | IIVAVVTGIAVAAIVAAVVALIY (SEQ ID NO: 137) |
| | NM_201563_5 | |
| T025 | FCGRA2 transcript | IIVAVVIATAVAAIVAAVVALIY (SEQ ID NO: 138) |
| | variant 1 | |
| | NM_001136219_1 | |
| T026 | GHR transcript | FPWLLIIIFGIFGLTVMLFVFLFS (SEQ ID NO: 139) |
| | variant 1 | |
| | NM_000163_4 | |
| T027 | GHR transcript | FPWLLCIIFGIFGLTVMLFVFLFS (SEQ ID NO: 140) |
| | variant 1 | |
| | NM_000163_4 | |
| T028 | ICOS | FWLPIGCAAFVVVCILGCILI (SEQ ID NO: 141) |
| | NM_012092.3 | |
| T029 | IFNAR1 | IWLIVGICIALFALPFVIYAA (SEQ ID NO: 142) |
| | NM_000629_2 | |
| T030 | IFNAR2 transcript | IGGIITVFLIALVLTSTIVTL (SEQ ID NO: 143) |
| | variant 1 | |
| | NM_207585_2 | |
| T031 | IFNGR1 | SLWIPVVAALLLFLVLSLVFI (SEQ ID NO: 144) |
| | NM_000416_2 | |
| T032 | IFNGR2 transcript | VILISVGTFSLLSVLAGACFF (SEQ ID NO: 145) |
| | variant 1 | |
| | NM_001329128_1 | |
| T033 | IFNLR1 | FLVLPSLLILLLVIAAGGVIW (SEQ ID NO: 146) |
| | NM_170743_3 | |
| T034 | IL1R1 transcript | HMIGICVTLTVIIVCSVFIYKIF (SEQ ID NO: 147) |
| | variant 2 | |
| | NM_001288706_1 | |
| T035 | IL1RAP transcript | VLLVVILIVVYHVYWLEMVLF (SEQ ID NO: 148) |
| | variant 1 | |
| | NM_002182_3 | |
| T036 | IL1RL1 transcript | IYCIIAVCSVFLMLINVLVII (SEQ ID NO: 149) |
| | variant 1 | |
| | NM_016232.4 | |
| T037 | IL1RL2 | AYLIGGLIALVAVAVSVVYIY (SEQ ID NO: 150) |
| | NM_003854.2 | |
| T038 | IL2RA transcript | VAVAGCVFLLISVLLLSGL (SEQ ID NO: 151) |
| | variant 1 | |
| | NM_000417_2 | |
| T039 | IL2RB transcript | IPWLGHLLVGLSGAFGFIILVYLLI (SEQ ID NO: 152) |
| | variant 1 | |
| | NM_000878_4 | |
| T040 | IL2RG | VVISVGSMGLIISLLCVYFWL (SEQ ID NO: 153) |
| | NM_000206_2 | |
| T041 | IL3RA transcript | TSLLIALGTLLALVCVFVIC (SEQ ID NO: 154) |
| | variant 1 and 2 | |
| | NM_002183_3 | |
| T042 | IL4R transcript | LLLGVSVSCIVILAVCLLCYVSIT (SEQ ID NO: 155) |
| | variant 1 | |
| | NM_000418_3 | |
| T043 | IL5RA transcript | FVIVIMATICFILLILSLIC (SEQ ID NO: 156) |
| | variant 1 | |
| | NM_000564_4 | |
| T044 | IL6R transcript | TFLVAGGSLAFGTLLCIAIVL (SEQ ID NO: 157) |
| | variant 1 | |
| | NM_000565_3 | |
| T045 | IL6ST transcript | AIVVPVCLAFLLTTLLGVLFCF (SEQ ID NO: 158) |
| | variant land 3 | |
| | NM_002184_3 | |
| T046 | IL7RA | ILLTISILSFFSVALLVILACVL (SEQ ID NO: 159) |
| | NM_002185_3 | |
| T047 | IL7RA Ins PPCL | ILLPPCLTISILSFFSVALLVILACVL (SEQ ID NO: 160) |
| | (interleukin 7 | |
| | receptor) | |
| T048 | IL9R transcript | GNTLVAVSIFLLLTGPTYLLF (SEQ ID NO: 161) |
| | variant 1 | |
| | NM_002186_2 | |
| T049 | IL10RA transcript | VIIFFAFVLLLSGALAYCLAL (SEQ ID NO: 162) |
| | variant 1 | |
| | NM_001558_3 | |
| T050 | IL10RB | WMVAVILMASVFMVCLALLGCF (SEQ ID NO: 163) |
| | NM_000628_4 | |
| T051 | IL11RA | SLGILSFLGLVAGALALGLWL (SEQ ID NO: 164) |
| | NM_001142784_2 | |
| T052 | IL12RB1 transcript | WLIFFASLGSFLSILLVGVLGYLGL (SEQ ID NO: 165) |
| | variant 1 and 4 | |
| | NM_005535_2 | |
| T053 | IL12RB2 transcript | WMAFVAPSICIAIIMVGIFST (SEQ ID NO: 166) |
| | variant 1 and 3 | |
| | NM_001559_2 | |
| T054 | IL13RA1 | LYITMLLIVPVIVAGAIIVLLLYL (SEQ ID NO: 167) |
| | NM_001560_2 | |
| T055 | IL13RA2 | FWLPFGFILILVIFVTGLLL (SEQ ID NO: 168) |
| | NM_000640_2 | |
| T056 | IL15RA transcript | VAISTSTVLLCGLSAVSLLACYL (SEQ ID NO: 169) |
| | variant 4 | |
| | NM_001256765_1 | |
| T057 | IL17RA | VYWFITGISILLVGSVILLIV (SEQ ID NO: 170) |
| | NM_014339_6 | |
| T058 | IL17RB | LLLLSLLVATWVLVAGIYLMW (SEQ ID NO: 171) |
| | NM_018725_3 | |
| T059 | IL17RC transcript | WALVWLACLLFAAALSLILLL (SEQ ID NO: 172) |
| | variant 1 | |
| | NM_153460_3 | |
| T060 | IL17RD transcript | AVAITVPLVVISAFATLFTVM (SEQ ID NO: 173) |
| | variant 2 | |
| | NM_017563_4 | |
| T061 | IL17RE transcript | LGLLILALLALLTLLGVVLAL (SEQ ID NO: 174) |
| | variant 1 | |
| | NM_153480_1 | |
| T062 | IL18R1 transcript | GMHAVLILVAVVCLVTVCV1 (SEQ ID NO: 175) |
| | variant 1 | |
| | NM_003855_3 | |
| T063 | IL18RAP | GVVLLYILLGTIGTLVAVLAA (SEQ ID NO: 176) |
| | NM_003853_3 | |
| T064 | IL20RA transcript | IIFWYVLPISITVFLFSVMGY (SEQ ID NO: 177) |
| | variant 1 | |
| | NM_014432_3 | |
| T065 | IL20RB | VLALFAFVGFMLILVVVPLFV (SEQ ID NO: 178) |
| | NM_144717_3 | |
| T066 | IL21R transcript | GWNPHLLLLLLLVIVFIPAFW (SEQ ID NO: 179) |
| | variant 2 | |
| | NM_181078_2 | |
| T067 | IL22RA1 | YSFSGAFLFSMGFLVAVLCYL (SEQ ID NO: 180) |
| | NM_021258_3 | |
| T068 | IL23R | LLLGMIVFAVMLSILSLIGIF (SEQ ID NO: 181) |
| | NM_144701_2 | |
| T069 | IL27RA | VLPGILFLWGLFLLGCGLSLA (SEQ ID NO: 182) |
| | NM_004843_3 | |
| T070 | IL27RA | VLPGILCLWGLFLLGCGLSLA (SEQ ID NO: 183) |
| | NM_004843_3 | |
| T071 | IL31RA transcript | IILITSLIGGGLLILIILTVAYGL (SEQ ID NO: 184) |
| | variant 1 | |
| | NM_139017_5 | |
| T072 | LEPR transcript | AGLYVIVPVIISSSILLLGTLLI (SEQ ID NO: 185) |
| | variant 1 | |
| | NM_002303_5 | |
| T073 | LIFR | VGLIIAILIPVAVAVIVGVVTSILC (SEQ ID NO: 186) |
| | NM_001127671_1 | |
| T074 | MPL | ISLVTALHLVLGLSAVLGLLLL (SEQ ID NO: 187) |
| | NM_005373_2 | |
| T075 | MPL | ISLVTALHLVLGLNAVLGLLLL (SEQ ID NO: 188) |
| | NM_005373_2 | |
| T076 | OSMR transcript | LIHILLPMVFCVLLIMVMCYL (SEQ ID NO: 189) |
| | variant 4 | |
| | NM_001323505_1 | |
| T077 | PRLR transcript | TTVWISVAVLSAVICLIIVWAVAL (SEQ ID NO: 190) |
| | variant 1 | |
| | NM_000949_6 | |
| T078 | TNFRSF4 | VAAILGLGLVLGLLGPLAILL (SEQ ID NO: 191) |
| | NM_003327_3 | |
| T079 | TNFRSF8 | PVLDAGPVLFWVILVLVVVVGSSAFLLC (SEQ ID NO: 192) |
| | transcript variant | |
| | 1 NM_001243_4 | |
| T080 | TNFRSF9 | IISFFLALTSTALLFLLFFLTLRFSVV (SEQ ID NO: 193) |
| | NM_001561_5 | |
| T081 | TNFRSF14 | WWFLSGSLVIVIVCSTVGLII (SEQ ID NO: 194) |
| | transcript variant | |
| | 1 NM_003820_3 | |
| T082 | TNFRSF18 | LGWLTVVLLAVAACVLLLTSA (SEQ ID NO: 195) |
| | transcript variant | |
| | 1 NM_004195_2 | |
| S036 | CD2 transcript | TKRKKQRSRRNDEELETRAHRVATEERGRKPHQIPASTPQNPATSQHPPPPPGHRSQAPSHRPPPPGHRVQHQPQKR |
| | variant 1 | PPAPSGTQVHQQKGPPLPRPRVQPKPPHGAAENSLSPSSN (SEQ ID NO: 196) |
| | NM_001328609_1 | |
| S037 | CD3D transcript | GHETGRLSGAADTQALLRNDQVYQPLRDRDDAQYSHLGGNWARNK (SEQ ID NO: 197) |
| | variant 1 | |
| | NM_000732_4 | |
| S038 | CD3E | KNRKAKAKPVTRGAGAGGRQRGQNKERPPPVPNPDYEPIRKGQRDLYSGLNQRRI (SEQ ID NO: 198) |
| | NM_000733_3 | |
| S039 | CD3G | GQDGVRQSRASDKQTLLPNDQLYQPLKDREDDQYSHLQGNQLRRN (SEQ ID NO: 199) |
| | NM_000073_2 | |
| S042 | CD4 transcript | CVRCRHRRRQAERMSQIKRLLSEKKTCQCPHRFQKTCSPI (SEQ ID NO: 200) |
| | variant 1 and 2 | |
| | NM_000616_4 | |
| S043 | CD8A transcript | LYCNHRNRRRVCKCPRPVVKSGDKPSLSARYV (SEQ ID NO: 201) |
| | variant 1 | |
| | NM_001768_6 | |
| S044 | CD8B transcript | RRRRARLRFMKQPQGEGISGTFVPQCLHGYYSNTTTSQKLLNPWILKT (SEQ ID NO: 202) |
| | variant 2 | |
| | NM_172213_3 | |
| S045 | CD8B transcript | RRRRARLRFMKQLRLHPLEKCSRMDY (SEQ ID NO: 203) |
| | variant 3 | |
| | NM_172101_3 | |
| S046 | CD8B transcript | RRRRARLRFMKQFYK (SEQ ID NO: 204) |
| | variant 5 | |
| | NM_004931_4 | |
| S047 | CD27 | QRRKYRSNKGESPVEPAEPCRYSCPREEEGSTIPIQEDYRKPEPACSP (SEQ ID NO: 205) |
| | NM_001242_4 | |
| S048 | mutated Delta Lck | RSKRSRLLHSDYMNMTPRRPGPTRKHYQAYAAARDFAAYRS (SEQ ID NO: 206) |
| | CD28 transcript | |
| | variant 1 | |
| | NM_006139_3 | |
| S049 | CD28 transcript | RSKRSRLLHSDYMNMTPRRPGPTRKHYQPYAPPRDFAAYRS (SEQ ID NO: 207) |
| | variant 1 | |
| | NM_006139_3 | |
| S050 | CD40 transcript | KKVAKKPTNKAPHPKQEPQEINFPDDLPGSNTAAPVQETLHGCQPVTQEDGKESRISVQERQ (SEQ ID NO: 208) |
| | variant land 6 | |
| | NM_001250_5 | |
| S051 | CD40 transcript | SESSEKVAKKPTNKAPHPKQEPQEINFPDDLPGSNTAAPVQETLHGCQPVTQEDGKESRISVQERQ (SEQ ID |
| | variant 5 | NO: 209) |
| | NM_001322421_1 | |
| S052 | CD79A transcript | RKRWCINEKLGLDAGDEYEDENLYEGLNLDDCSMYEDISRGLQGTYQDVGSLNIGDVQLEKP (SEQ ID NO: 210) |
| | variant 1 | |
| | NM_001783_3 | |
| S053 | CD796 transcript | LDKDDSKAGMEEDHTYEGLDIDQTATYEDIVTLRTGEVKWSVGEHPGQE (SEQ ID NO: 211) |
| | variant 3 | |
| | NM_001039933_2 | |
| S054 | CRLF2 transcript | KLWRVKKFLIPSVPDPKSIFPGLFEIHQGNFQEWITDTQNVAHLHKMAGAEQESGPEEPLVVOLAKTEAESPRMLDPQ |
| | variant 1 | TEEKEASGGSLQLPHOPLOGGDVVTIGGFTFVMNDRSYVAL (SEQ ID NO: 212) |
| | NM_022148_3 | |
| S057 | CSF2RB | RFCGIYGYRLRRKWEEKIPNPSKSHLFQNGSAELWPPGSMSAFTSGSPPHQGPWGSRFPELEGVFPVGFGDSEVSPLTI |
| | NM_000395_2 | EDPKHVCDPPSGPDTTPAASDLPTEQPPSPQPGPPAASHTPEKQASSFDFNGPYLGPPHSRSLPDILGQPEPPQEGGSQ |
| | | KSPPPGSLEYLCLPAGGQVQLVPLAQAMGPGQAVEVERRPSQGAAGSPSLESGGGPAPPALGPRVGGQDQKDSPVAI |
| | | PMSSGDTEDPGVASGYVSSADLVFTPNSGASSVSLVPSLGLPSDQTPSLCPGLASGPPGAPGPVKSGFEGYVELPPIEGR |
| | | SPRSPRNNPVPPEAKSPVLNPGERPADVSPTSPCIPEGLLVLQQVGDYCFLPGLGPGPLSLRSKPSSPGPGPEIKNLDQAF |
| | | QVKKPPGQAVPQVPVIQLFKALKQQDYLSLPPWEVNKPGEVC (SEQ ID NO: 213) |
| S058 | CSF2RA transcript | KRFLRIQRLFPPVPQIKDKLNDNHEVEDEIIWEEFTPEEGKGYREEVLTVKEIT (SEQ ID NO: 214) |
| | variant 7 and 8 | |
| | NM_001161529_1 | |
| S059 | CSF2RA transcript | KRFLRIQRLFPPVPQIKDKLNDNHEVEDEMGPQRHHRCGWNLYPTPGPSPGSGSSPRLGSESSL (SEQ ID NO: 215) |
| | variant 9 | |
| | NM_001161531_1 | |
| S062 | CSF3R transcript | SPNRKNPLWPSVPDPAHSSLGSWVPTIMEEDAFQLPGLGTPPITKLTVLEEDEKKPVPWESHNSSETCGLPTLVQTYVL |
| | variant 1 | QGDPRAVSTQPQSQSGTSDQVLYGOLLGSPTSPGPGHYLRCDSTQPLLAGLTPSPKSYENLWFQASPLGTLVTPAPSQ |
| | NM_000760_3 | EDDCVFGPLLNFPLLQGIRVHGMEALGSF (SEQ ID NO: 216) |
| S063 | CSF3R transcript | SPNRKNPLWPSVPDPAHSSLGSWVPTIMEELPGPRQGQWLGQTSEMSRALTPHPCVQDAFQLPGLGTPPITKLTVLE |
| | variant 3 | EDEKKPVPWESHNSSETCGLPTLVQTYVLQGDPRAVSTQPQSQSGTSDQVLYGOLLGSPTSPGPGHYLRCDSTQPLLA |
| | NM_156039_3 | GLTPSPKSYENLWFQASPLGTLVTPAPSQEDDCVFGPLLNFPLLQGIRVHGMEALGSF (SEQ ID NO: 217) |
| S064 | CSF3R transcript | SPNRKNPLWPSVPDPAHSSLGSWVPTIMEEDAFQLPGLGTPPITKLTVLEEDEKKPVPWESHNSSETCGLPTLVQTYVL |
| | variant 4 | QGDPRAVSTQPQSQSGTSDQAGPPRRSAYFKDQIMLHPAPPNGLLCLFPITSVL (SEQ ID NO: 218) |
| | NM_172313_2 | |
| S069 | EPOR transcript | HRRALKQKIWPGIPSPESEFEGLFTTHKGNFQLWLYQNDGCLWWSPCTPFTEDPPASLEVLSERCWGTMQAVEPGTD |
| | variant 1 | DEGPLLEPVGSEHAQDTYLVLDKWLLPRNPPSEDLPGPGGSVDIVAMDEGSEASSCSSALASKPSPEGASAASFEYTILD |
| | NM_000121_3 | PSSQLLRPWTLCPELPPTPPHLKYLYLVVSDSGISTDYSSGDSQGAQGGLSDGPYSNPYENSLIPAAEPLPPSYVACS |
| | | (SEQ ID NO: 219) |
| S072 | EPOR transcript | HRRALKQKIWPGIPSPESEFEGLFTTHKGNFQLWLYQNDGCLWWSPCTPFTEDPPASLEVLSERCWGTMQAVEPGTD |
| | variant 1 | DEGPLLEPVGSEHAQDTYLVLDKWLLPRNPPSEDLPGPGGSVDIVAMDEGSEASSCSSALASKPSPEGASAASFEYTILD |
| | NM_000121_3 | PSSQLLRPWTLCPELPPTPPHLKFLFLVVSDSGISTDYSSGDSQGAQGGLSDGPYSNPYENSLIPAAEPLPPSYVACS |
| | | (SEQ ID NO: 220) |
| S074 | FCER1G | RLKIQVRKAAITSYEKSDGVYTGLSTRNQETYETLKHEKPPQ (SEQ ID NO: 221) |
| | NM_004106_1 | |
| S075 | FCGR2C | CRKKRISANSTDPVKAAQFEPPGRQMIAIRKROPEETNNDYETADGGYMTLNPRAPTDDDKNIYLTLPPNDHVNSNN |
| | NM_201563_5 | (SEQ ID NO: 222) |
| S076 | FCGRA2 transcript | CRKKRISANSTDPVKAAQFEPPGRQMIAIRKRQLEETNNDYETADGGYMTLNPRAPTDDDKNIYLTLPPNDHVNSNN |
| | variant 1 | (SEQ ID NO: 223) |
| | NM_001136219_1 | |
| S077 | GHR transcript | KQQRIKMLILPPVPVPKIKGIDPDLLKEGKLEEVNTILAIHDSYKPEFHSDDSWVEFIELDIDEPDEKTEESDTDRLLSSDHE |
| | variant 1 | KSFISNLGVKDGDSGRTSCCEPDILETDFNANDIHEGTSEVAQPQRLKGEADLLCLDQKNONNSPYHDACPATQQPSVI |
| | NM_000163_4 | QAEKNKPQPLPTEGAESTHQAAHIQLSNPSSLSNIDFYAQVSDITPAGSVVLSPGQKNKAGMSQCDMHPEMVSLCQE |
| | | NFLMDNAYFCEADAKKCIPVAPHIKVESHIQPSLNQEDIYITTESLTTAAGRPGTGEHVPGSEMPVPDYTSIHIVQSPQGL |
| | | ILNATALPLPDKEFLSSCGYVSTDQLNKIMP (SEQ ID NO: 224) |
| S080 | ICOS | CWLTKKKYSSSVHDPNGEYMFMRAVNTAKKSRLTDVTL (SEQ ID NO: 225) |
| | NM_012092.3 | |
| S081 | IFNAR1 | KVFLRCINYVFFPSLKPSSSIDEYFSEQPLKNLLLSTSEEQIEKCFIIENISTIATVEETNQTDEDHKKYSSQTSQDSGNYSNED |
| | NM_000629_2 | ESESKTSEELQQDFV (SEQ ID NO: 226) |
| S082 | IFNAR2 transcript | KWIGYICLRNSLPKVLNFHNFLAWPFPNLPPLEAMDMVEVIYINRKKKVWDYNYDDESDSDTEAAPRTSGGGYTMHG |
| | variant 1 | LTVRPLGOASATSTESQLIDPESEEEPDLPEVDVELPTMPKDSPQQLELLSGPCERRKSPLQDPFPEEDYSSTEGSGGRITF |
| | NM_207585_2 | NVDLNSVFLRVLDDEDSDDLEAPLMLSSHLEEMVDPEDPDNVQSNHLLASGEGTQPTFPSPSSEGLWSEDAPSDQSDT |
| | | SESDVDLGDGYIMR (SEQ ID NO: 227) |
| S083 | IFNAR2 transcript | KWIGYICLRNSLPKVLRQGLAKGWNAVAIHRCSHNALQSETPELKQSSCLSFPSSWDYKRASLCPSD (SEQ ID |
| | variant 2 | NO: 228) |
| | NM_000874_4 | |
| S084 | IFNGR1 | CFYIKKINPLKEKSIILPKSLISVVRSATLETKPESKYVSLITSYQPFSLEKEVVCEEPLSPATVPGMHTEDNPGKVEHTEELSSI |
| | NM_000416_2 | TEVVTTEENIPDVVPGSHLTPIERESSSPLSSNQSEPGSIALNSYHSRNCSESDHSRNGFDTDSSCLESHSSLSDSEFPPNN |
| | | KGEIKTEGQELITVIKAPTSFGYDKPHVLVDLLVDDSGKESLIGYRPTEDSKEFS (SEQ ID NO: 229) |
| S085 | IFNGR2 transcript | LVLKYRGLIKYWFHTPPSIPLQIEEYLKDPTQPILEALDKDSSPKDDVWDSVSIISFPEKEQEDVLQTL (SEQ ID NO: 230) |
| | variant 1 | |
| | NM_001329128_1 | |
| S086 | IFNLR1 | KTLMGNPWFQRAKMPRALDFSGHTHPVATFQPSRPESVNDLFLCPQKELTRGVRPTPRVRAPATQQTRWKKDLAED |
| | NM_170743_3 | EEEEDEEDTEDGVSFQPYIEPPSFLGQEHQAPGHSEAGGVDSGRPRAPLVPSEGSSAWDSSDRSWASTVDSSWDRAG |
| | | SSGYLAEKGPGQGPGGDGHQESLPPPEFSKDSGFLEELPEDNLSSWATWGTLPPEPNLVPGGPPVSLQTLTFCWESSP |
| | | EEEEEARESEIEDSDAGSWGAESTQRTEDRGRTLGHYMAR (SEQ ID NO: 231) |
| S087 | IFNLR1 transcript | KTLMGNPWFQRAKMPRALELTRGVRPTPRVRAPATQQTRWKKDLAEDEEEEDEEDTEDGVSFQPYIEPPSFLGQEHQ |
| | variant 2 | APGHSEAGGVDSGRPRAPLVPSEGSSAWDSSDRSWASTVDSSWDRAGSSGYLAEKGPGQGPGGDGHQESLPPPEFS |
| | NM_173064_2 | KDSGFLEELPEDNLSSWATWGTLPPEPNLVPGGPPVSLQTLTFCWESSPEEEEEARESEIEDSDAGSWGAESTQRTEDR |
| | | GRTLGHYMAR (SEQ ID NO: 232) |
| S098 | IL1R1 transcript | KIDIVLWYRDSCYDFLPIKVLPEVLEKQCGYKLFIYGRDDYVGEDIVEVINENVKKSRRLIIILVRETSGFSWLGGSSEEQIA |
| | variant 2 | MYNALVQDGIKVVLLELEKIQDYEKMPESIKFIKQKHGAIRWSGDFTQGPQSAKTRFWKNVRYHMPVQRRSPSSKHQ |
| | NM_001288706_1 | LLSPATKEKLQREAHVPLG (SEQ ID NO: 233) |
| S099 | IL1R1 transcript | KIDIVLWYRDSCYDFLPIKASDGKTYDAYILYPKTVGEGSTSDCDIFVFKVLPEVLEKQCGYKLFIYGRDDYVGEDIVEVINE |
| | variant 3 | NVKKSRRLIIILVRETSGFSWLGGSSEEQIAMYNALVQDGIKVVLLELEKIQDYEKMPESIKFIKQKHGAIRWSGDFTQGP |
| | NM_001320978_1 | QSAKTRFWKNVRYHMPVQRRSPSSKHOLLSPATKEKLQREAHVPLG (SEQ ID NO: 234) |
| S100 | IL1RAP transcript | YRAHFGTDETILDGKEYDIYVSYARNAEEEEFVLLTLRGVLENEFGYKLCIFDRDSLPGGIVTDETLSFIQKSRRLLVVLSPNY |
| | variant 1 | VLQGTQALLELKAGLENMASRGNINVILVQYKAVKETKVKELKRAKTVLTVIKWKGEKSKYPQGRFWKQLQVAMPVKK |
| | NM_002182_3 | SPRRSSSDEQGLSYSSLKNV (SEQ ID NO: 235) |
| S101 | IL1RAP transcript | YRAHFGTDETILDGKEYDIYVSYARNAEEEEFVLLTLRGVLENEFGYKLCIFDRDSLPGGNTVEAVFDFIQRSRRMIVVLSP |
| | variant 6 | DYVTEKSISMLEFKLGVMCQNSIATKLIVVEYRPLEHPHPGILQLKESVSFVSWKGEKSKHSGSKFWKALRLALPLRSLSA |
| | NM_001167931_1 | SSGWNESCSSQSDISLDHVQRRRSRLKEPPELQSSERAAGSPPAPGTMSKHRGKSSATCRCCVTYCEGENHLRNKSRAE |
| | | IHNQPQWETHLCKPVPQESETQWIQNGTRLEPPAPQISALALHHFTDLSNNNDFYIL (SEQ ID NO: 236) |
| S102 | IL1RL1 transcript | LKMFWIEATLLWRDIAKPYKTRNDGKLYDAYVVYPRNYKSSTDGASRVEHFVHQILPDVLENKCGYTLCIYGRDMLPGE |
| | variant 1 | DVVTAVETNIRKSRRHIFILTPQITHNKEFAYEQEVALHCALIQNDAKVILIEMEALSELDMLQAEALQDSLQHLMKVQG |
| | NM_016232.4 | TIKWREDHIANKRSLNSKFWKHVRYQMPVPSKIPRKASSLTPLAAQKQ (SEQ ID NO: 237) |
| S103 | IL1RL2 | NIFKIDIVLWYRSAFHSTETIVDGKLYDAYVLYPKPHKESQRHAVDALVLNILPEVLERQCGYKLFIFGRDEFPGQAVANVI |
| | NM_003854.2 | DENVKLCRRLIVIVVPESLGFGLLKNLSEEQIAVYSALIQDGMKVILIELEKIEDYTVMPESIQYIKQKHGAIRWHGDFTEQS |
| | | QCMKTKFWKTVRYHMPPRRCRPFPPVQLLQHTPCYRTAGPELGSRRKKCTLTTG (SEQ ID NO: 238) |
| S104 | IL2RA transcript | TWQRRQRKSRRTI (SEQ ID NO: 239) |
| | variant 1 | |
| | NM_000417_2 | |
| S105 | IL2RB transcript | NCRNTGPWLKKVLKCNTPDPSKFFSQLSSEHGGDVQKWLSSPFPSSSFSPGGLAPEISPLEVLERDKVTQLLLQQDKVPE |
| | variant 1 | PASLSSNHSLTSCFTNQGYFFFHLPDALEIEACQVYFTYDPYSEEDPDEGVAGAPTGSSPQPLQPLSGEDDAYCTFPSRD |
| | NM_000878_4 | DLLLFSPSLLGGPSPPSTAPGGSGAGEERMPPSLQERVPRDWDPQPLGPPTPGVPDLVDFQPPPELVLREAGEEVPDA |
| | | GPREGVSFPWSRPPGQGEFRALNARLPLNTDAYLSLQELQGQDPTHLV (SEQ ID NO: 240) |
| S106 | IL2RG | ERTMPRIPTLKNLEDLVTEYHGNFSAWSGVSKGLAESLQPDYSERLCLVSEIPPKGGALGEGPGASPCNQHSPYWAPPC |
| | NM_000206_2 | YTLKPET (SEQ ID NO: 241) |
| S109 | IL3RA transcript | RRYLVMQRLFPRIPHMKDPIGDSFQNDKLVVWEAGKAGLEECLVTEVQVVQKT (SEQ ID NO: 242) |
| | variant 1 and 2 | |
| | NM_002183_3 | |
| S110 | IL4R transcript | KIKKEWWDQIPNPARSRLVAIIIQDAQGSQWEKRSRGQEPAKCPHWKNCLTKLLPCFLEHNMKRDEDPHKAAKEMPF |
| | variant 1 | QGSGKSAWCPVEISKTVLWPESISVVRCVELFEAPVECEEEEEVEEEKGSFCASPESSRDDFQEGREGIVARLTESLFLDLL |
| | NM_000418_3 | GEENGGFCQQDMGESCLLPPSGSTSAHMPWDEFPSAGPKEAPPWGKEQPLHLEPSPPASPTQSPDNLTCTETPLVIA |
| | | GNPAYRSFSNSLSQSPCPRELGPDPLLARHLEEVEPEMPCVPQLSEPTTVPQPEPETWEQILRRNVLQHGAAAAPVSAP |
| | | TSGYQEFVHAVEQGGTQASAVVGLGPPGEAGYKAFSSLLASSAVSPEKCGFGASSGEEGYKPFQDLIPGCPGDPAPVPV |
| | | PLFTFGLDREPPRSPOSSHLPSSSPEHLGLEPGEKVEDMPKPPLPQEQATDPLVDSLGSGIVYSALTCHLCGHLKQCHGQ |
| | | EDGGQTPVMASPCCGCCCGDRSSPPTTPLRAPDPSPGGVPLEASLCPASLAPSGISEKSKSSSSFHPAPGNAQS5SQTPK |
| | | IVNFVSVGPTYMRVS (SEQ ID NO: 243) |
| S113 | IL4R transcript | KIKKEWWDQIPNPARSRLVAIIIQDAQGSQWEKRSRGQEPAKCPHWKNCLTKUPCFLEHNMKRDEDPHKAAKEMPF |
| | variant 1 | QGSGKSAWCPVEISKTVLWPESISVVRCVELFEAPVECEEEEEVEEEKGSFCASPESSRDDFQEGREGIVARLTESLFLDLL |
| | NM_000418_3 | GEENGGFCQQDMGESCLLPPSGSTSAHMPWDEFPSAGPKEAPPWGKEQPLHLEPSPPASPTQSPDNLTCTETPLVIA |
| | | GNPAYRSFSNSLSQSPCPRELGPDPLLARHLEEVEPEMPCVPQLSEPTTVPQPEPETWEQILRRNVLQHGAAAAPVSAP |
| | | TSGYQEFVHAVEQGGTQASAVVGLGPPGEAGYKAFSSLLASSAVSPEKCGFGASSGEEGYKPFQDLIPGCPGDPAPVPV |
| | | PLFTFGLDREPPRSPOSSHLPSSSPEHLGLEPGEKVEDMPKPPLPQEQATDPLVDSLGSGIVFSALTCHLCGHLKQCHGQ |
| | | EDGGQTPVMASPCCGCCCGDRSSPPTTPLRAPDPSPGGVPLEASLCPASLAPSGISEKSKSSSSFHPAPGNAQS5SQTPK |
| | | IVNFVSVGPTYMRVS (SEQ ID NO: 244) |
| S115 | IL5RA transcript | KICHLWIKLFPPIPAPKSNIKDLFVTTNYEKAGSSETEIEVICYIEKPGVETLEDSVF (SEQ ID NO: 245) |
| | variant 1 | |
| | NM_000564_4 | |
| S116 | IL6R transcript | RFKKTWKLRALKEGKTSMHPPYSLGQLVPERPRPTPVLVPLISPPVSPSSLGSDNTSSHNRPDARDPRSPYDISNTDYFFP |
| | variant 1 | R (SEQ ID NO: 246) |
| | NM_000565_3 | |
| S117 | IL6ST transcript | NKRDLIKKHIWPNVPDPSKSHIAQWSPHTPPRHNFNSKDQMYSDGNFTDVSVVEIEANDKKPFPEDLKSLDLFKKEKIN |
| | variant 1 and 3 | TEGHSSGIGGSSCMSSSRPSISSSDENESSQNTSSTVQYSTVVHSGYRHQVPSVQVFSRSESTQPLLDSEERPEDLQLVD |
| | NM_002184_3 | HVDGGDGILPRQQYFKONCSQHESSPDISHFERSKQVSSVNEEDFVRLKQQ1SDHISQ5CGSGQMKMFQEVSAADAF |
| | | GPGTEGOVERFETVGMEAATDEGMPKSYLPQTVRQGGYMPQ (SEQ ID NO: 247) |
| S120 | IL7RA Isoform 1 | WKKRIKPIVWPSLPDHKKTLEHLCKKPRKNLNVSFNPESFLDCQIHRVDDIQARDEVEGFLQDTFPQQLEESEKQRLGG |
| | NM_002185.4 | DVQSPNCPSEDVVITPESFGRDSSLTCLAGNVSACDAPILSSSRSLDCRESGKNGPHVYQDLLLSLGTTNSTLPPPFSLQS |
| | | GILTLNPVAQGQPILTSLGSNQEEAYVTMSSFYQNQ (SEQ ID NO: 248) |
| S121 | IL7RA Isoform 3 | WKKRIKPIVWPSLPDHKKTLEHLCKKPRKVSVFGA (SEQ ID NO: 249) |
| | (C-term deletion) | |
| | (interleukin 7 | |
| | receptor) | |
| S126 | IL9R transcript | KLSPRVKRIFYQNVPSPAMFFQPLYSVHNGNFQTWMGAHGAGVLLSQDCAGTPQGALEPCVQEATALLTCGPARPW |
| | variant 1 | KSVALEEEQEGPGTRLPGNLSSEDVLPAGCTEWRVQTLAYLPQEDWAPTSLTRPAPPDSEGSR55555555NNNNYCAL |
| | NM_002186_2 | GCYGGWHLSALPGNTQSSGPIPALACGLSCDHQGLETQQGVAWVLAGHCQRPGLHEDLQGMLLPSVLSKARSWTF |
| | | (SEQ ID NO: 250) |
| S129 | IL10RA transcript | QLYVRRRKKLPSVLLFKKPSPFIFISQRPSPETQDTIHPLDEEAFLKVSPELKNLDLHGSTDSGFGSTKPSLQTEEPQFLLPD |
| | variant 1 | PHPQADRTLGNREPPVLGDSCSSGSSNSTDSGICLQEPSLSPSTGPTWEQQVGSNSRGQDDSGIDLVQNSEGRAGDT |
| | NM_001558_3 | QGGSALGHHSPPEPEVPGEEDPAAVAFQGYLRQTRCAEEKATKTGCLEEESPLTDGLGPKFGRCLVDEAGLHPPALAK |
| | | GYLKQDPLEMTLASSGAPTGQWNQPTEEWSLLALSSCSDLGISDWSFAHDLAPLGCVAAPGGLLGSFNSDLVTLPLISS |
| | | LQSSE (SEQ ID NO: 251) |
| S130 | IL10RB | ALLWCVYKKTKYAFSPRNSLPQHLKEFLGHPHHNTLLFFSFPLSDENDVFDKLSVIAEDSESGKQNPGDSCSLGTPPGQG |
| | NM_000628_4 | PQS (SEQ ID NO: 252) |
| S135 | IL11RA | RLRRGGKDGSPKPGFLASVIPVDRRPGAPNL (SEQ ID NO: 253) |
| | NM_001142784_2 | |
| S136 | IL12RB1 transcript | NRAARHLCPPLPTPCASSAIEFPGGKETWQWINPVDFQEEASLQEALVVEMSWDKGERTEPLEKTELPEGAPELALDTE |
| | variant 1 and 4 | LSLEDGDRCKAKM (SEQ ID NO: 254) |
| | NM_005535_2 | |
| S137 | IL12RB1 transcript | NRAARHLCPPLPTPCASSAIEFPGGKETWQWINPVDFQEEASLQEALVVEMSWDKGERTEPLEKTELPEGAPELALDTE |
| | variant 3 | LSLEDGDRCDR (SEQ ID NO: 255) |
| | NM_001290023_1 | |
| S138 | IL12RB2 transcript | HYFQQKVFVLLAALRPQWCSREIPDPANSTCAKKYPIAEEKTQLPLDRLLIDWPTPEDPEPLVISEVLHQVTPVFRHPPCS |
| | variant 1 and 3 | NWPQREKGIQGHQASEKDMMHSASSPPPPRALQAESRQLVDLYKVLESRGSDPKPENPACPWTVLPAGDLPTHDGY |
| | NM_001559_2 | LPSNIDDLPSHEAPLADSLEELEPQHISLSVFP5SSLHPLTFSCGDKLTLDQLKMRCDSLML (SEQ ID NO: 256) |
| S141 | IL13RA1 | KRLKIIIFPPIPDPGKIFKEMFGDQNDDTLHWKKYDIYEKQTKEETDSVVLIENLKKASQ (SEQ ID NO: 257) |
| | NM_001560_2 | |
| S142 | IL13RA2 | RKPNTYPKMIPEFFCDT (SEQ ID NO: 258) |
| | NM_000640_2 | |
| S143 | IL15RA transcript | KSRQTPPLASVEMEAMEALPVTWGTSSRDEDLENCSHHL (SEQ ID NO: 259) |
| | variant 4 | |
| | NM_001256765_1 | |
| S144 | IL17RA | CMTWRLAGPGSEKYSDDTKYTDGLPAADLIPPPLKPRKVWIIYSADHPLYVDVVLKFAQFLLTACGTEVALDLLEEQAISE |
| | NM_014339_6 | AGVMTWVGRQKQEMVESNSKIIVLCSRGTRAKWQALLGRGAPVRLRCDHGKPVGDLFTAAMNMILPDFKRPACFG |
| | | TYVVCYFSEVSCDGDVPDLFGAAPRYPLMDRFEEVYFRIQDLEMFQPGRMHRVGELSGDNYLRSPGGRQLRAALDRF |
| | | RDWQVRCPDWFECENLYSADDQDAPSLDEEVFEEPLLPPGTGIVKRAPLVREPGSQACLAIDPLVGEEGGAAVAKLEP |
| | | HLQPRGQPAPQPLHTLVLAAEEGALVAAVEPGPLADGAAVRLALAGEGEACPLLGSPGAGRNSVLFLPVDPEDSPLGSS |
| | | TPMASPDLLPEDVREHLEGLMLSLFEQSLSCQAQGGCSRPAMVLTDPHTPYEEEQRQSVCISDQGYISRSSPQPPEGLT |
| | | EMEEEEEEEQDPGKPALPLSPEDLESLRSLQRQLLFRQLQKNSGWDTMGSESEGPSA (SEQ ID NO: 260) |
| S145 | IL17RB | RHERIKKTSFSTTTLLPPIKVLVVYPSEICFHHTICYFTEFLQNHCRSEVILEKWQKKKIAEMGPVQWLATQKKAADKVVFL |
| | NM_018725_3 | LSNDVNSVCDGTCGKSEGSPSENSQDLFPLAFNLFCSDLRSQIHLHKYVVVYFREIDTKDDYNALSVCPKYHLMKDATAF |
| | | CAELLHVKQQVSAGKRSQACHDGCCSL (SEQ ID NO: 261) |
| S146 | IL17RC transcript | KKDHAKGWLRLLKQDVRSGAAARGRAALLLYSADDSGFERLVGALASALCQLPLRVAVDLWSRRELSAQGPVAWFHA |
| | variant 1 | QRRQTLQEGGVVVLLFSPGAVALCSEWLQDGVSGPGAHGPHDAFRASLSCVLPDFLQGRAPGSYVGACFDRLLHPDA |
| | NM_153460_3 | VPALFRTVPVFTLPSQLPDFLGALQQPRAPRSGRLQERAEQVSRALQPALDSYFHPPGTPAPGRGVGPGAGPGAGDGT |
| | | (SEQ ID NO: 262) |
| S147 | IL17RC transcript | KKDHAKAAARGRAALLLYSADDSGFERLVGALASALCQLPLRVAVDLWSRRELSAQGPVAWFHAQRRQTLQEGGVVV |
| | variant 4 | LLFSPGAVALCSEWLQDGVSGPGAHGPHDAFRASLSCVLPDFLQGRAPGSYVGACFDRLLHPDAVPALFRTVPVFTLPS |
| | NM_001203263_1 | QLPDFLGALQQPRAPRSGRLQERAEQVSRALQPALDSYFHPPGTPAPGRGVGPGAGPGAGDGT (SEQ ID NO: 263) |
| S148 | IL17RD transcript | CRKKQQENIYSHLDEESSESSTYTAALPRERLRPRPKVFLCYSSKDGQNHMNVVQCFAYFLQDFCGCEVALDLWEDFSL |
| | variant 2 | CREGQREWVIQKIHESQFIIVVCSKGMKYFVDKKNYKHKGGGRGSGKGELFLVAVSAIAEKLRQAKQSSSAALSKFIAVY |
| | NM_017563_4 | FDYSCEGDVPGILDLSTKYRLMDNLPQLCSHLHSRDHGLQEPGQHTRQGSRRNYFRSKSGRSLYVAICNMHQFIDEEPD |
| | | WFEKQFVPFHPPPLRYREPVLEKFDSGLVLNDVMCKPGPESDFCLKVEAAVLGATGPADSQHESQHGGLDQDGEARP |
| | | ALDGSAALQPLLHTVKAGSPSDMPRDSGIYDSSVPSSELSLPLMEGLSTDQTETSSLTESVSSSSGLGEEEPPALPSKLLSS |
| | | GSCKADLGCRSYTDELHAVAPL (SEQ ID NO: 264) |
| S149 | IL17RE transcript | TCRRPQSGPGPARPVLLLHAADSEAQRRLVGALAELLRAALGGGRDVIVDLWEGRHVARVGPLPWLWAARTRVARE |
| | variant 1 | QGTVLLLWSGADLRPVSGPDPRAAPLLALLHAAPRPLLLLAYFSRLCAKGDIPPPLRALPRYRLLRDLPRLLRALDARPFAE |
| | NM_153480_1 | ATSWGRLGARQRRQSRLELCSRLEREAARLADLG (SEQ ID NO: 265) |
| S154 | IL18R1 transcript | YRVDLVLFYRHLTRRDETLTDGKTYDAFVSYLKECRPENGEEHTFAVEILPRVLEKHFGYKLCIFERDVVPGGAVVDEIHSL |
| | variant 1 | IEKSRRLIIVLSKSYMSNEVRYELESGLHEALVERKIKIILIEFTPVTDFTFLPQSLKLLKSHRVLKWKADKSLSYNSRFWKNLL |
| | NM_003855_3 | YLMPAKTVKPGRDEPEVLPVLSES (SEQ ID NO: 266) |
| S155 | IL18RAP | SALLYRHWIEIVLLYRTYQSKDQTLGDKKDFDAFVSYAKWSSFPSEATSSLSEEHLALSLFPDVLENKYGYSLCLLERDVAP |
| | NM_003853_3 | GGVYAEDIVSIIKRSRRGIFILSPNYVNGPSIFELQAAVNLALDDQTLKLILIKFCYFQEPESLPHLVKKALRVLPTVTWRGLK |
| | | SVPPNSRFWAKMRYHMPVKNSQGFTWNQLRITSRIFQWKGLSRTETTGRSSQPKEW (SEQ ID NO: 267) |
| S156 | IL20RA transcript | SIYRYIHVGKEKHPANLILIYGNEFDKRFFVPAEKIVINFITLNISDDSKISHQDMSLLGKSSDVSSLNDPQPSGNLRPPQEE |
| | variant 1 | EEVKHLGYASHLMEIFCDSEENTEGTSLTQQESLSRTIPPDKTVIEYEYDVRTTDICAGPEEQELSLQEEVSTQGTLLESQA |
| | NM_014432_3 | ALAVLGPQTLQYSYTPQLQDLDPLAQEHTDSEEGPEEEPSTTLVDWDPQTGRLCIPSLSSFDQDSEGCEPSEGDGLGEE |
| | | GLLSRLYEEPAPDRPPGENETYLMQFMEEWGLYVQMEN (SEQ ID NO: 268) |
| S157 | IL20RB | WKMGRLLQYSCCPVVVLPDTLKITNSPQKLISCRREEVDACATAVMSPEELLRAWIS (SEQ ID NO: 269) |
| | NM_144717_3 | |
| S158 | IL21R transcript | SLKTHPLWRLWKKIWAVPSPERFFMPLYKGCSGDFKKWVGAPFTGSSLELGPWSPEVPSTLEVYSCHPPRSPAKRLQLT |
| | variant 2 | ELQEPAELVESDGVPKPSFWPTAQNSGGSAYSEERDRPYGLVSIDTVTVLDAEGPCTWPCSCEDDGYPALDLDAGLEPS |
| | NM_181078_2 | PGLEDPLLDAGTTVLSCGCVSAGSPGLGGPLGSLLDRLKPPLADGEDWAGGLPWGGRSPGGVSESEAGSPLAGLDMD |
| | | TFDSGFVGSDCSSPVECDFTSPGDEGPPRSYLRQWVVIPPPLSSPGPQAS (SEQ ID NO: 270) |
| S161 | IL22RA1 | SYRYVTKPPAPPNSLNVQRVLTFQPLRFIQEHVLIPVFDLSGPSSLAQPVQYSQIRVSGPREPAGAPQRHSLSEITYLGQP |
| | NM_021258_3 | DISILQPSNVPPPOILSPLSYAPNAAPEVGPPSYAPQVTPEAQFPFYAPQAISKVQPSSYAPQATPDSWPPSYGVCMEGS |
| | | GKDSPTGTLSSPKHLRPKGQLQKEPPAGSCMLGGLSLQEVTSLAMEESQEAKSLHQPLGICTDRTSDPNVLHSGEEGTP |
| | | QYLKGQLPLLSSVQIEGHPMSLPLQPPSRPCSPSDQGPSPWGLLESLVCPKDEAKSPAPETSDLEQPTELDSLFRGLALTV |
| | | QWES (SEQ ID NO: 271) |
| S165 | IL23R | NRSFRTGIKRRILLLIPKWLYEDIPNMKNSNVVKMLQENSELMNNNSSEQVLYVDPMITEIKEIFIPEHKPTDYKKENTGP |
| | NM_144701_2 | LETRDYPQNSLFDNTTVVYIPDLNTGYKPOISNFLPEGSHLSNNNEITSLTLKPPVDSLDSGNNPRLQKHPNFAFSVSSVN |
| | | SLSNTIFLGELSLILNQGECSSPDIQNSVEEETTMLLENDSPSETIPEQTLLPDEFVSCLGIVNEELPSINTYFPQNILESHFNR |
| | | ISLLEK (SEQ ID NO: 272) |
| S168 | IL27RA | TSGRCYHLRHKVLPRWVWEKVPDPANSSSGQPHMEQVPEAQPLGDLPILEVEEMEPPPVMESSQPAQATAPLDSGY |
| | NM_004843_3 | EKHFLPTPEELGLLGPPRPQVLA (SEQ ID NO: 273) |
| S169 | IL27RA | TSWVWEKVPDPANSSSGQPHMEQVPEAQPLGDLPILEVEEMEPPPVMESSQPAQATAPLDSGYEKHFLPTPEELGLL |
| | NM_004843_3 | GPPRPQVLA (SEQ ID NO: 274) |
| S170 | IL31RA transcript | KKPNKLTHLCWPTVPNPAESSIATWHGDDFKDKLNLKESDDSVNTEDRILKPCSTPSDKLVIDKLVVNFGNVLQEIFTDE |
| | variant 1 | ARTGQENNLGGEKNGYVTCPFRPDCPLGKSFEELPVSPEIPPRKSQYLRSRMPEGTRPEAKEQLLFSGQ5LVPDHLCEEG |
| | NM_139017_5 | APNPYLKNSVTAREFLVSEKLPEHTKGEV (SEQ ID NO: 275) |
| S171 | IL31RA transcript | KKPNKLTHLCWPTVPNPAESSIATWHGDDFKDKLNLKESDDSVNTEDRILKPCSTPSDKLVIDKLVVNFGNVLQEIFTDE |
| | variant 4 | ARTGQENNLGGEKNGTRILSSCPTSI (SEQ ID NO: 276) |
| | NM_001242638_1 | |
| S174 | LEPR transcript | SHQRMKKLFWEDVPNPKNCSWAQGLNFQKPETFEHLFIKHTASVTCGPLLLEPETISEDISVDTSWKNKDEMMPTTVV |
| | variant 1 | SLLSTTDLEKGSVCISDQFNSVNFSEAEGTEVTYEDESQRQPFVKYATLISNSKPSETGEEQGLINSSVTKCFSSKNSPLKDS |
| | NM_002303_5 | FSNSSWEIEAQAFFILSDQHPNIISPHLTFSEGLDELLKLEGNFPEENNDKKSIYYLGVTSIKKRESGVLLTDKSRVSCPFPAP |
| | | CLFTDIRVLQDSCSHFVENNINLGTSSKKTFASYMPQFQTCSTQTHKIMENKMCDLTV (SEQ ID NO: 277) |
| S175 | LEPR transcript | SHQRMKKLFWEDVPNPKNCSWAQGLNFQKMLEGSMFVKSHHHSLISSTQGHKHCGRPQGPLHRKTRDLCSLVYLLT |
| | variant 2 | LPPLLSYDPAKSPSVRNTQE (SEQ ID NO: 278) |
| | NM_001003680_3 | |
| S176 | LEPR transcript | SHORMKKLFWEDVPNPKNCSWAQGLNFQKRTDIL (SEQ ID NO: 279) |
| | variant 3 | |
| | NM_001003679_3 | |
| S177 | LEPR transcript | SHORMKKLFWEDVPNPKNCSWAQGLNFQKKMPGTKELLGGGWLT (SEQ ID NO: 280) |
| | variant 5 | |
| | NM_001198688_1 | |
| S180 | LIFR | YRKREWIKETFYPDIPNPENCKALQFQKSVCEGSSALKTLEMNPCTPNNVEVLETRSAFPKIEDTEIISPVAERPEDRSDAE |
| | NM_001127671_1 | PENHVVVSYCPPIIEEEIPNPAADEAGGTAQVIYIDVQSMYQPQAKPEEEQENDPVGGAGYKPQMHLPINSTVEDIAAE |
| | | EDLDKTAGYRPQANVNTWNLVSPDSPRSIDSNSEIVSFGSPCSINSRQFLIPPKDEDSPKSNGGGWSFTNFFQNKPND |
| | | (SEQ ID NO: 281) |
| S183 | LMP1 | YYHGQRHSDEHHHDDSLPHPQQATDDSGHESDSNSNEGRHHLLVSGAGDGPPLCSONLGAPGGGPDNGPQDPDN |
| | NC_007605_1 | TDDNGPQDPDNTDDNGPHDPLPQDPDNTDDNGPQDPDNTDDNGPHDPLPHSPSDSAGNDGGPPQLTEEVENKG |
| | | GDQGPPLMTDGGGGHSHDSGHGGGDPHLPTLLLGSSGSGGDDDDPHGPVQLSYYD (SEQ ID NO: 282) |
| S186 | MPL | RWQFPAHYRRLRHALWPSLPDLHRVLGQYLRDTAALSPPKATVSDTCEEVEPSLLEILPKSSERTPLPLCSSQAQMDYRR |
| | NM_005373_2 | LQPSCLGTMPLSVCPPMAESGSCCTTHIANHSYLPLSYWQQP (SEQ ID NO: 283) |
| S189 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 1 | WQGRPGASVGRLLELLTKLGRDDVLLELGPSIEEDCQKYILKQQQEEAEKPLQVAAVDSSVPRTAELAGITTLDDPLGH |
| | NM_001172567_1 | MPERFDAFICYCPSDIQFVQEMIRQLEQTNYRLKLCVSDRDVLPGTCVWSIASELIEKRLARRPRGGCRRMVVVVSDDY |
| | | LQSKECDFQTKFALSLSPGAHQKRLIPIKYKAMKKEFPSILRFITVCDYTNPCTKSWFWTRLAKALSLP (SEQ ID |
| | | NO: 284) |
| S190 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 2 | WQGRPGASVGRLLELLTKLGRDDVLLELGPSIEEDCQKYILKQQQEEAEKPLQVAAVDSSVPRTAELAGITTLDDPLGH |
| | NM_002468_4 | MPERFDAFICYCPSDIQFVQEMIRQLEQTNYRLKLCVSDRDVLPGTCVWSIASELIEKRCRRMVVVVSDDYLQSKECDF |
| | | QTKFALSLSPGAHQKRLIPIKYKAMKKEFPSILRFITVCDYTNPCTKSWFWTRLAKALSLP (SEQ ID NO: 285) |
| S191 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 3 | WQGRPGASVGRLLELLTKLGRDDVLLELGPSIGHMPERFDAFICYCPSDIQFVQEMIRQLEQTNYRLKLCVSDRDVLPG |
| | NM_001172568_1 | TCVWSIASELIEKRCRRMVVVVSDDYLQSKECDFQTKFALSLSPGAHQKRLIPIKYKAMKKEFPSILRFITVCDYTNPCTKS |
| | | WFWTRLAKALSLP (SEQ ID NO: 286) |
| S192 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 4 | WQGRPGASVGRLLELLTKLGRDDVLLELGPSIEEDCQKYILKQQQEEAEKPLQVAAVDSSVPRTAELAGITTLDDPLGAA |
| | NM_001172569_1 | GWWWLSLMITCRARNVTSRPNLHSASLQVPIRSD (SEQ ID NO: 287) |
| S193 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 5 | WQGRPGASVGRLLELLTKLGRDDVLLELGPSIGAAGWWWLSLMITCRARNVTSRPNLHSASLQVPIRSD (SEQ ID |
| | NM_001172566_1 | NO: 288) |
| S194 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 1 | WQGRPGASVGRLLELLTKLGRDDVLLELGPSIEEDCQKYILKQQQEEAEKPLQVAAVDSSVPRTAELAGITTLDDPLGH |
| | NM_001172567_1 | MPERFDAFICYCPSDI (SEQ ID NO: 289) |
| S195 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 3 | WQGRPGASVGRLLELLTKLGRDDVLLELGPSIGHMPERFDAFICYCPSDI (SEQ ID NO: 290) |
| | NM_001172568_1 | |
| S196 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 1 | WQGRPGASVGRLLELLTKLGRDDVLLELGPSIEEDCQKYILKQQQEEAEKPLQVAAVDSSVPRTAELAGITTLDDPLGH |
| | NM_001172567_1 | MPERFDAFICYCPSDIQFVQEMIRQLEQTNYRLKLCVSDRDVLPGTCVWSIASELIEKRLARRPRGGCRRMVVVVSDDY |
| | | LQSKECDFQTKFALSLSPGAHQKRPIPIKYKAMKKEFPSILRFITVCDYTNPCTKSWFWTRLAKALSLP (SEQ ID |
| | | NO: 291) |
| S197 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 2 | WQGRPGASVGRLLELLTKLGRDDVLLELGPSIEEDCQKYILKQQQEEAEKPLQVAAVDSSVPRTAELAGITTLDDPLGH |
| | NM_002468_4 | MPERFDAFICYCPSDIQFVQEMIRQLEQTNYRLKLCVSDRDVLPGTCVWSIASELIEKRCRRMVVVVSDDYLQSKECDF |
| | | QTKFALSLSPGAHQKRPIPIKYKAMKKEFPSILRFITVCDYTNPCTKSWFWTRLAKALSLP (SEQ ID NO: 292) |
| S198 | MYD88 transcript | MAAGGPGAGSAAPVSSTSSLPLAALNMRVRRRLSLFLNVRTQVAADVVTALAEEMDFEYLEIRQLETQADPTGRLLDA |
| | variant 3 | WQGRPGASVGRLLELLTKLGRDDVLLELGPSIGHMPERFDAFICYCPSDIQFVQEMIRQLEQTNYRLKLCVSDRDVLPG |
| | NM_001172568_1 | TCVWSIASELIEKRCRRMVVVVSDDYLQSKECDFQTKFALSLSPGAHQKRPIPIKYKAMKKEFPSILRFITVCDYTNPCTKS |
| | | WFWTRLAKALSLP (SEQ ID NO: 293) |
| S199 | OSMR transcript | KSQWIKETCYPDIPDPYKSSILSLIKFKENPHLIIMNVSDCIPDAIEVVSKPEGTKIQFLGTRKSLTETELTKPNYLYLLPTEKN |
| | variant 4 | HSGPGPCICFENLTYNQAASDSGSCGHVPVSPKAPSMLGLMTSPENVLKALEKNYMNSLGEIPAGETSLNYVSQLASP |
| | NM_001323505_1 | MFGDKDSLPTNPVEAPHCSEYKMQMAVSLRLALPPPTENSSLSSITLLDPGEHYC (SEQ ID NO: 294) |
| S202 | PRLR transcript | KGYSMVTCIFPPVPGPKIKGFDAHLLEKGKSEELLSALGCQDFPPTSDYEDLLVEYLEVDDSEDQHLMSVHSKEHPSQG |
| | variant 1 | MKPTYLDPDTDSGRGSCDSPSLLSEKCEEPQANPSTFYDPEVIEKPENPETTHTWDPQCISMEGKIPYFHAGGSKCSTW |
| | NM_000949_6 | PLPQPSQHNPRSSYHNITDVCELAVGPAGAPATLLNEAGKDALKSSQTIKSREEGKATQQREVESFHSETDQDTPWLLP |
| | | QEKTPFGSAKPLDYVEIHKVNKDGALSLLPKCIRENSGKPKKPGTPENNKEYAKVSGVMDNNILVLVPDPHAKNVACFE |
| | | ESAKEAPPSLEQNQAEKALANFTATSSKCRLQLGGLDYLDPACFTHSFH (SEQ ID NO: 295) |
| S211 | TNFRSF4 | ALYLLRRDQRLPPDAHKPPGGGSFRTPIQEEQADAHSTLAKI (SEQ ID NO: 296) |
| | NM_003327_3 | |
| S212 | TNFRSF8 | HRRACRKRIRQKLHLCYPVQTSQPKLELVDSRPRRSSTQLRSGASVTEPVAEERGLMSQPLMETCHSVGAAYLESLPLQ |
| | transcript variant | DASPAGGPSSPRDLPEPRVSTEHTNNKIEKIYIMKADTVIVGTVKAELPEGRGLAGPAEPELEEELEADHTPHYPEQETEP |
| | 1 NM_001243_4 | PLGSCSDVMLSVEEEGKEDPLPTAASGK (SEQ ID NO: 297) |
| S213 | TNFRSF9 | KRGRKKLLYIFKQPFMRPVQTTQEEDGCSCRFPEEEEGGCEL (SEQ ID NO: 298) |
| | NM_001561_5 | |
| S214 | TNFRSF14 | CVKRRKPRGDVVKVIVSVQRKRQEAEGEATVIEALQAPPDVTTVAVEETIPSFTGRSPNH (SEQ ID NO: 299) |
| | transcript variant | |
| | 1 NM_003820_3 | |
| S215 | TNFRSF18 | QLGLHIWQLRSQCMWPRETQLLLEVPPSTEDARSCQFPEEERGERSAEEKGRLGDLWV (SEQ ID NO: 300) |
| | transcript variant | |
| | 1 NM_004195_2 | |
| S216 | TNFRSF18 | QLGLHIWQLRKTQLLLEVPPSTEDARSCQFPEEERGERSAEEKGRLGDLWV (SEQ ID NO: 301) |
| | transcript variant | |
| | 3_NM_148902_1 | |
| X001 | Linker | GSGGSEGGGSEGGAATAGSGSGS (SEQ ID NO: 302) |

TABLE 4
Constructs present in Library 6 Top 100
in vivo, antigen independent.

| Ranking | Block Sequence |
|---|---|
| 1 | E006-T030-S129-S047 |
| 2 | E006-T023-S171-S211 |
| 3 | E008-T001-S121-S212 |
| 4 | E006-T064-S190-S080 |
| 5 | E006-T047-S141-S050 |
| 6 | E008-T001-S064-S047 |
| 7 | E006-T048-X001-S211 |
| 8 | E009-T073-S062-S053 |
| 9 | E010-T035-S190-S047 |
| 10 | E010-T055-S192-S051 |
| 11 | E006-T071-S165-S076 |
| 12 | E009-T075-S165-S050 |
| 13 | E010-T027-S117-S053 |
| 14 | E007-T054-S197-S212 |
| 15 | E007-T056-S170-S050 |
| 16 | E007-T050-S190-S051 |
| 17 | E008-T060-S190-S074 |
| 18 | E007-T080-S059-S080 |
| 19 | E007-T057-S059-S075 |
| 20 | E006-T045-S177-S216 |
| 21 | E010-T077-S058-S053 |
| 22 | E006-T073-S120-S048 |
| 23 | E009-T063-S192-S053 |
| 24 | E008-T067-S190-S074 |
| 25 | E009-T057-S117-S074 |
| 26 | E007-T045-S190-S211 |
| 27 | E009-T068-S083-S212 |
| 28 | E007-T039-S197-S080 |
| 29 | E010-T036-S058-S048 |
| 30 | E008-T056-S190-S050 |
| 31 | E010-T026-S120-S038 |
| 32 | E006-T017-S062-S039 |
| 33 | E009-T073-S142-X002 |
| 34 | E009-T077-S192-S212 |
| 35 | E007-T052-S199-S049 |
| 36 | E007-T061-S186-S211 |
| 37 | E009-T009-S197-S038 |
| 38 | E008-T029-S161-S216 |
| 39 | E010-T006-S190-X002 |
| 40 | E010-T081-S190-X002 |
| 41 | E008-T045-S062-S211 |
| 42 | E008-T049-S116-S076 |
| 43 | E009-T029-S190-S211 |
| 44 | E008-T068-S158-S076 |
| 45 | E007-T058-S194-S037 |
| 46 | E010-T024-S115-S039 |
| 47 | E010-T070-S190-S216 |
| 48 | E010-T049-S115-S074 |
| 49 | E006-T059-S190-S051 |
| 50 | E006-T035-S197-S039 |
| 51 | E009-T076-S190-S051 |
| 52 | E007-T032-S117-S051 |
| 53 | E010-T078-S197-S051 |
| 54 | E006-T026-S165-S037 |
| 55 | E007-T081-S194-S047 |
| 56 | E010-T003-S194-S215 |
| 57 | E009-T069-S194-S050 |
| 58 | E010-T057-S190-S211 |
| 59 | E008-T006-S129-S216 |
| 60 | E008-T078-S190-S211 |
| 61 | E006-T065-S194-S080 |
| 62 | E009-T012-S171-S074 |
| 63 | E009-T041-S165-S038 |
| 64 | E006-T057-S194-S038 |
| 65 | E006-T012-S176-S076 |
| 66 | E008-T052-S197-S050 |
| 67 | E007-T016-S135-S212 |
| 68 | E009-T007-S192-S051 |
| 69 | E006-T065-S165-S047 |
| 70 | E008-T011-S135-S075 |
| 71 | E007-T002-S190-S051 |
| 72 | E006-T037-S165-S053 |
| 73 | E007-T021-S130-S212 |
| 74 | E010-T071-S194-S211 |
| 75 | E007-T023-S158-S080 |
| 76 | E008-T078-S177-S215 |
| 77 | E010-T008-S196-S049 |
| 78 | E006-T026-S199-S053 |
| 79 | E006-T027-S084-S037 |
| 80 | E007-T034-S189-S212 |
| 81 | E010-T074-S130-S212 |
| 82 | E008-T072-S192-S075 |
| 83 | E008-T021-S109-S039 |
| 84 | E006-T065-S135-S214 |
| 85 | E006-T073-X001-S074 |
| 86 | E008-T032-S175-X002 |
| 87 | E010-T072-S192-S050 |
| 88 | E008-T067-S189-S050 |
| 89 | E008-T073-S192-S074 |
| 90 | E006-T023-S183-S076 |
| 91 | E010-T041-S147-S076 |
| 92 | E010-T067-S130-S074 |
| 93 | E008-T023-S194-S212 |
| 94 | E006-T063-S190-S211 |
| 95 | E006-T053-S194-X002 |
| 96 | E008-T019-S194-S211 |
| 97 | E007-T020-S109-S050 |
| 98 | E006-T024-S194-S074 |
| 99 | E009-T049-S194-S050 |
| 100 | E008-T027-S126-S053 |

TABLE 5
Constructs present in Library 8 Top 100
in vivo, antigen independent.

| Ranking | Block Sequence |
|---|---|
| 1 | E006-T032-S197-S075 |
| 2 | E006-T013-S196-S048 |
| 3 | E008-T030-S057-S037 |
| 4 | E006-T069-S177-S080 |
| 5 | E009-T056-S104-S080 |
| 6 | E006-T006-S171-S215 |
| 7 | E006-T023-S117-S080 |
| 8 | E006-T057-S180-S051 |
| 9 | E007-T032-S064-S052 |
| 10 | E006-T044-S186-S053 |
| 11 | E009-T020-S121-S037 |
| 12 | E009-T012-S154-X002 |
| 13 | E010-T042-S194-S050 |
| 14 | E009-T062-S190-S074 |
| 15 | E006-T018-S141-S213 |
| 16 | E009-T026-S100-S047 |
| 17 | E006-T053-S186-S074 |
| 18 | E010-T021-S197-S049 |
| 19 | E007-T005-S143-S211 |
| 20 | E009-T005-S157-S216 |
| 21 | E006-T038-S192-S039 |
| 22 | E007-T005-S170-S076 |
| 23 | E009-T069-S143-S049 |
| 24 | E006-T057-S189-S038 |
| 25 | E008-T065-S069-S053 |
| 26 | E009-T042-S058-S074 |
| 27 | E006-T045-S072-S051 |
| 28 | E010-T011-S121-S038 |
| 29 | E009-T072-S154-X002 |
| 30 | E010-T072-S194-S047 |
| 31 | E008-T038-S165-S052 |
| 32 | E010-T057-S141-S050 |
| 33 | E006-T056-S196-S212 |
| 34 | E010-T066-S197-S051 |
| 35 | E008-T031-S083-S212 |
| 36 | E009-T006-S062-S053 |
| 37 | E010-T043-S186-S075 |
| 38 | E008-T003-S138-S039 |
| 39 | E008-T057-S141-S049 |
| 40 | E008-T056-S192-S039 |
| 41 | E009-T049-S199-S037 |
| 42 | E006-T045-S197-S053 |
| 43 | E007-T012-S130-S052 |
| 44 | E007-T015-S069-S038 |
| 45 | E009-T065-S062-X002 |
| 46 | E008-T014-X001-S051 |
| 47 | E008-T026-S058-S050 |
| 48 | E008-T048-S161-S050 |
| 49 | E006-T067-S145-S052 |
| 50 | E009-T049-S135-S052 |
| 51 | E006-T080-S121-S074 |
| 52 | E009-T044-S130-S037 |
| 53 | E007-T016-S165-S037 |
| 54 | E008-T047-S194-X002 |
| 55 | E006-T050-S186-S039 |
| 56 | E008-T055-X001-S216 |
| 57 | E008-T013-S197-S216 |
| 58 | E010-T072-S192-S212 |
| 59 | E007-T001-S064-S215 |
| 60 | E007-T065-S197-S075 |
| 61 | E010-T040-S189-S047 |
| 62 | E009-T039-S117-S074 |
| 63 | E007-T042-S177-S048 |
| 64 | E010-T061-S175-S213 |
| 65 | E008-T063-S069-S075 |
| 66 | E008-T070-S165-S212 |
| 67 | E009-T012-S064-S211 |
| 68 | E006-T006-S194-S211 |
| 69 | E010-T035-S121-S214 |
| 70 | E006-T011-S170-S211 |
| 71 | E006-T048-S058-S053 |
| 72 | E009-T040-S058-S214 |
| 73 | E009-T019-S146-S050 |
| 74 | E010-T045-S135-S075 |
| 75 | E006-T071-S058-S049 |
| 76 | E008-T031-S170-S211 |
| 77 | E007-T030-S176-S048 |
| 78 | E008-T007-S192-S213 |
| 79 | E006-T035-S121-S075 |
| 80 | E008-T060-S064-S214 |
| 81 | E010-T077-S117-S037 |
| 82 | E007-T066-S054-X002 |
| 83 | E008-T023-S194-S214 |
| 84 | E009-T044-S083-S038 |
| 85 | E007-T077-S062-S074 |
| 86 | E006-T063-S130-S052 |
| 87 | E009-T010-S170-S074 |
| 88 | E010-T072-S192-S038 |
| 89 | E010-T016-S168-S037 |
| 90 | E010-T036-S197-S074 |
| 91 | E010-T004-S194-S216 |
| 92 | E009-T049-S085-S075 |
| 93 | E009-T059-S193-S039 |
| 94 | E007-T042-S099-S053 |
| 95 | E008-T031-S104-S076 |
| 96 | E006-T039-S115-S080 |
| 97 | E006-T073-S117-S053 |
| 98 | E010-T032-X001-S049 |
| 99 | E007-T029-S104-S049 |
| 100 | E006-T072-S158-S047 |

TABLE 6
Constructs present in Library 6 Top 100
in vivo, antigen dependent

| Ranking | Block Sequence |
|---|---|
| 1 | E006-T066-S109-X002 |
| 2 | E010-T012-S192-S214 |
| 3 | E009-T028-S130-S212 |
| 4 | E010-T032-S186-S050 |
| 5 | E007-T052-S197-S075 |
| 6 | E007-T052-S102-S049 |
| 7 | E009-T023-S190-S050 |
| 8 | E008-T008-S194-S215 |
| 9 | E010-T058-S121-S080 |
| 10 | E009-T019-S194-S049 |
| 11 | E008-T004-S142-S212 |
| 12 | E007-T012-S054-S076 |
| 13 | E010-T077-S192-S074 |
| 14 | E006-T073-X001-S074 |
| 15 | E006-T070-S197-S037 |
| 16 | E006-T069-S197-S053 |
| 17 | E006-T061-S190-S080 |
| 18 | E008-T032-S190-S213 |
| 19 | E008-T022-S109-S052 |
| 20 | E009-T078-S190-S047 |
| 21 | E009-T015-S083-S053 |
| 22 | E010-T072-S146-S047 |
| 23 | E010-T078-S197-S051 |
| 24 | E007-T063-S196-S050 |
| 25 | E010-T055-S192-S051 |
| 26 | E006-T059-S190-S051 |
| 27 | E006-T026-S199-S053 |
| 28 | E010-T002-S194-S050 |
| 29 | E009-T075-S165-S050 |
| 30 | E010-T082-X001-S052 |
| 31 | E008-T032-S083-S074 |
| 32 | E007-T040-S192-S049 |
| 33 | E007-T045-S192-S051 |
| 34 | E010-T025-S194-S047 |
| 35 | E006-T078-S082-S048 |
| 36 | E010-T082-S186-S047 |
| 37 | E010-T072-S192-S050 |
| 38 | E007-T039-S197-S080 |
| 39 | E010-T072-S186-S037 |
| 40 | E008-T035-S176-S038 |
| 41 | E008-T056-S190-S050 |
| 42 | E007-T021-S130-S212 |
| 43 | E009-T049-S194-S050 |
| 44 | E007-T032-S117-S051 |
| 45 | E009-T052-S102-S049 |
| 46 | E010-T005-S192-S214 |
| 47 | E007-T061-S186-S211 |
| 48 | E009-T057-S117-S074 |
| 49 | E007-T016-S135-S212 |
| 50 | E009-T073-S062-S053 |
| 51 | E008-T049-S116-S076 |
| 52 | E006-T023-S171-S211 |
| 53 | E006-T048-X001-S211 |
| 54 | E010-T035-S190-S047 |
| 55 | E007-T054-S197-S212 |
| 56 | E006-T045-S177-S216 |
| 57 | E006-T071-S165-S076 |
| 58 | E009-T049-S194-S051 |
| 59 | E009-T049-S197-S051 |
| 60 | E010-T072-S176-S074 |
| 61 | E009-T049-S190-S051 |
| 62 | E008-T067-S197-S074 |
| 63 | E009-T049-S196-S051 |
| 64 | E006-T026-S165-S037 |
| 65 | E009-T049-S189-S051 |
| 66 | E006-T035-S197-S039 |
| 67 | E006-T012-S176-S076 |
| 68 | E006-T065-S194-S080 |
| 69 | E009-T073-S142-X002 |
| 70 | E009-T069-S194-S050 |
| 71 | E007-T045-S190-S211 |
| 72 | E009-T076-S190-S051 |
| 73 | E010-T070-S190-S216 |
| 74 | E010-T049-S115-S074 |
| 75 | E010-T024-S115-S039 |
| 76 | E007-T081-S194-S047 |
| 77 | E007-T058-S194-S037 |
| 78 | E009-T029-S190-S211 |
| 79 | E006-T073-S120-S048 |
| 80 | E008-T045-S062-S211 |
| 81 | E009-T063-S192-S053 |
| 82 | E008-T067-S190-S074 |
| 83 | E010-T026-S120-S038 |
| 84 | E009-T068-S083-S212 |
| 85 | E010-T081-S190-X002 |
| 86 | E008-T060-S190-S074 |
| 87 | E007-T080-S059-S080 |
| 88 | E010-T077-S058-S053 |
| 89 | E006-T047-S141-S050 |
| 90 | E009-T009-S197-S038 |
| 91 | E008-T001-S121-S212 |
| 92 | E007-T056-S170-S050 |
| 93 | E008-T068-S158-S076 |
| 94 | E006-T064-S190-S080 |
| 95 | E007-T050-S190-S051 |
| 96 | E006-T030-S129-S047 |
| 97 | E008-T001-S064-S047 |
| 98 | E007-T052-S199-S049 |
| 99 | E010-T027-S117-S053 |
| 100 | E010-T036-S058-S048 |

TABLE 7
Constructs present in Library 8 Top 100
in vivo, antigen dependent

| Ranking | Block Sequence |
|---|---|
| 1 | E006-T077-S129-X002 |
| 2 | E006-T031-S109-S216 |
| 3 | E007-T057-S195-S213 |
| 4 | E006-T006-S062-S052 |
| 5 | E008-T033-S197-S216 |
| 6 | E009-T010-S177-S037 |
| 7 | E006-T049-S109-S074 |
| 8 | E007-T029-S069-S076 |
| 9 | E006-T044-S062-S053 |
| 10 | E007-T048-S186-S053 |
| 11 | E009-T032-X001-S211 |
| 12 | E010-T018-S165-S051 |
| 13 | E006-T038-S154-X002 |
| 14 | E007-T021-S194-S211 |
| 15 | E009-T005-S142-S076 |
| 16 | E008-T012-S157-S216 |
| 17 | E009-T005-S197-S051 |
| 18 | E007-T021-S190-S047 |
| 19 | E010-T066-S129-S039 |
| 20 | E010-T033-S149-S215 |
| 21 | E006-T070-S085-S076 |
| 22 | E009-T041-S190-S214 |
| 23 | E007-T031-S130-S047 |
| 24 | E008-T073-S165-X002 |
| 25 | E010-T068-S194-S050 |
| 26 | E008-T006-S197-S213 |
| 27 | E010-T072-S104-S215 |
| 28 | E008-T045-S165-S080 |
| 29 | E008-T041-S104-S048 |
| 30 | E008-T001-S165-S048 |
| 31 | E009-T046-S155-S038 |
| 32 | E006-T026-S146-S212 |
| 33 | E010-T002-S192-S039 |
| 34 | E007-T052-S135-S074 |
| 35 | E006-T001-S158-S215 |
| 36 | E008-T031-S117-S215 |
| 37 | E007-T082-S142-S211 |
| 38 | E008-T044-X001-S211 |
| 39 | E007-T029-S197-S038 |
| 40 | E010-T032-X001-S049 |
| 41 | E009-T070-S161-X002 |
| 42 | E008-T011-S135-S213 |
| 43 | E007-T009-S059-S076 |
| 44 | E007-T037-S141-S216 |
| 45 | E010-T072-S192-S038 |
| 46 | E006-T015-S085-X002 |
| 47 | E008-T012-S146-S052 |
| 48 | E008-T068-S165-S050 |
| 49 | E006-T044-S192-S038 |
| 50 | E006-T026-S135-S074 |
| 51 | E007-T042-S169-X002 |
| 52 | E006-T007-S192-S049 |
| 53 | E008-T025-S121-S076 |
| 54 | E008-T065-S192-S213 |
| 55 | E008-T073-S069-S080 |
| 56 | E008-T073-S192-S214 |
| 57 | E010-T026-S064-S074 |
| 58 | E007-T001-S197-S216 |
| 59 | E009-T001-S109-S212 |
| 60 | E007-T063-S192-S047 |
| 61 | E009-T031-S063-S215 |
| 62 | E006-T044-S186-S053 |
| 63 | E008-T040-S069-S050 |
| 64 | E006-T005-S064-S213 |
| 65 | E007-T063-S069-S074 |
| 66 | E009-T078-S192-S214 |
| 67 | E007-T004-S194-S047 |
| 68 | E006-T057-S180-S051 |
| 69 | E009-T012-S154-X002 |
| 70 | E008-T073-S069-S076 |
| 71 | E010-T073-S189-S038 |
| 72 | E009-T073-S062-S211 |
| 73 | E009-T049-S142-S038 |
| 74 | E009-T078-S165-S074 |
| 75 | E009-T078-S197-S080 |
| 76 | E010-T044-S104-S048 |
| 77 | E009-T013-S175-S211 |
| 78 | E007-T029-S197-S211 |
| 79 | E006-T038-S192-S039 |
| 80 | E006-T048-S115-S216 |
| 81 | E010-T043-S117-S048 |
| 82 | E007-T012-S142-S211 |
| 83 | E010-T065-S130-S075 |
| 84 | E007-T016-S106-S037 |
| 85 | E006-T032-S138-S053 |
| 86 | E007-T022-S121-S076 |
| 87 | E007-T070-S054-S074 |
| 88 | E010-T051-S115-S051 |
| 89 | E010-T079-S072-S039 |
| 90 | E007-T003-S142-S080 |
| 91 | E009-T008-S062-S037 |
| 92 | E007-T063-S142-S075 |
| 93 | E007-T024-S135-S074 |
| 94 | E010-T057-S197-S211 |
| 95 | E009-T065-S145-S051 |
| 96 | E008-T012-S141-S213 |
| 97 | E007-T025-S202-S214 |
| 98 | E009-T036-S138-S047 |
| 99 | E009-T032-S141-S213 |
| 100 | E009-T058-S195-S048 |

TABLE 8
Constructs present in combined Library 6
and Library 8 sum of means analysis,
Top 30 in vivo, antigen independent

| Rank | Block | Sum |
|---|---|---|
| 1 | E006-T006-S171-S215 | 370,424 |
| 2 | E008-T001-S121-S212 | 320,942 |
| 3 | E009-T056-S104-S080 | 169,035 |
| 4 | E008-T030-S057-S037 | 167,467 |
| 5 | E006-T023-S117-S080 | 139,222 |
| 6 | E006-T032-S197-S075 | 120,909 |
| 7 | E009-T062-S190-S074 | 97,498 |
| 8 | E007-T032-S064-S052 | 93,519 |
| 9 | E010-T072-S192-S212 | 84,725 |
| 10 | E006-T044-S186-S053 | 71,737 |
| 11 | E006-T064-S190-S080 | 69,102 |
| 12 | E009-T006-S062-S053 | 53,397 |
| 13 | E008-T003-S138-S039 | 52,634 |
| 14 | E006-T038-S192-S039 | 49,701 |
| 15 | E009-T073-S062-S053 | 40,515 |
| 16 | E009-T032-S170-S074 | 35,245 |
| 17 | E010-T021-S197-S049 | 33,588 |
| 18 | E007-T005-S170-S076 | 22,931 |
| 19 | E007-T054-S197-S212 | 22,916 |
| 20 | E007-T039-S197-S080 | 19,845 |
| 21 | E008-T038-S165-S052 | 17,583 |
| 22 | E008-T078-S190-S211 | 16,857 |
| 23 | E008-T031-S083-S212 | 16,809 |
| 24 | E010-T066-S197-S051 | 16,457 |
| 25 | E006-T056-S196-S212 | 15,881 |
| 26 | E008-T065-S069-S053 | 15,512 |
| 27 | E008-T001-S064-S047 | 15,240 |
| 28 | E009-T010-S170-S074 | 14,526 |
| 29 | E006-T006-S194-S211 | 13,077 |
| 30 | E006-T045-S072-S051 | 12,177 |

What is claimed is:

1. Use of replication incompetent recombinant retroviral particles in the manufacture of a kit for genetically modifying T cells or NK cells of a subject, wherein the use of the kit comprises: contacting blood cells comprising the T cells and/or the NK cells ex vivo in a reaction mixture, with the replication incompetent recombinant retroviral particles, wherein the replication incompetent recombinant retroviral particles comprise a pseudotyping element on their surface, wherein the replication incompetent recombinant retroviral particles comprise a polynucleotide comprising one or more transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), or a first polypeptide comprising an LE and a second polypeptide comprising a CAR, and wherein said contacting is performed for less than 15 minutes to facilitate membrane fusion of the T cells and/or the NK cells to the replication incompetent recombinant retroviral particles, thereby producing the genetically modified T cells and/or the genetically modified NK cells.

2. A genetically modified T cell or NK cell made by genetically modifying T cells and/or NK cells according to a method comprising contacting blood cells comprising the T cells or NK cells ex vivo in a reaction mixture, with replication incompetent recombinant retroviral particles, wherein the replication incompetent recombinant retroviral particles comprise a pseudotyping element on their surface, wherein the replication incompetent recombinant retroviral particles comprise a polynucleotide comprising one or more transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), or a first polypeptide comprising a LE and a second polypeptide comprising a CAR, and wherein said contacting is performed for less than 15 minutes to facilitate membrane fusion of the T cells and/or NK cells to the replication incompetent recombinant retroviral particles, thereby producing the genetically modified T cell or the genetically modified NK cell.

3. A method for genetically modifying a T cell or an NK cell, comprising contacting blood cells comprising T cells and/or NK cells ex vivo in a reaction mixture, with replication incompetent recombinant retroviral particles comprising a pseudotyping element on their surface, wherein the replication incompetent recombinant retroviral particles comprise a polynucleotide comprising one or more transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), or a first polypeptide comprising an LE and a second polypeptide comprising a CAR, and wherein said contacting is performed for less than 15 minutes to facilitate membrane fusion of the T cells and/or the NK cells to the replication incompetent recombinant retroviral particles, thereby producing the genetically modified T cell or the genetically modified NK cell.

4. A use, genetically modified T cell or NK cell, or method for genetically modifying and/or transducing a T cell or an NK cell according to any of the preceding claims, wherein the reaction mixture comprises at least 25% whole blood.

5. A reaction mixture, comprising replication incompetent recombinant retroviral particles, a T cell activation element, and blood cells, wherein the recombinant retroviral particles comprise a pseudotyping element on their surface, wherein the blood cells comprise T cells and/or NK cells, wherein the replication incompetent recombinant retroviral particles comprise a polynucleotide comprising one or more transcriptional units operatively linked to a promoter active in T cells and/or NK cells, wherein the one or more transcriptional units encode a first polypeptide comprising a chimeric antigen receptor (CAR), a first polypeptide comprising a lymphoproliferative element (LE), or a first polypeptide comprising an LE and a second polypeptide comprising a CAR, and wherein the reaction mixture comprises at least 25% whole blood.

6. A method for genetically modifying T cells and/or NK cells in blood or a component thereof, comprising contacting blood cells comprising the T cells and/or NK cells ex vivo, with replication incompetent recombinant retroviral particles in a reaction mixture comprising a T cell activation element, wherein the replication incompetent recombinant retroviral particles comprise a pseudotyping element on their surface, wherein said contacting facilitates association of the T cells and/or NK cells with the replication incompetent recombinant retroviral particles, wherein the recombinant retroviral particles genetically modify and/or transduce the T cells and/or NK cells, and wherein the reaction mixture comprises at least 25% whole blood.

7. A reaction mixture, use, genetically modified T cell or NK cell, or method for genetically modifying T cells and/or NK cells according to any of the preceding claims, wherein most of the T cells are resting T cells or the NK cells are resting NK cells when they are combined with the replication incompetent retroviral particles to form the reaction mixture.

8. A reaction mixture, use, genetically modified T cell or NK cell, or method for genetically modifying T cells and/or NK cells according to any of the preceding claims, wherein the replication incompetent recombinant retroviral particles comprise a membrane-bound T cell activation element on their surface.

9. A reaction mixture, use, genetically modified T cell or NK cell, or method for genetically modifying T cells and/or NK cells according to any of the preceding claims, wherein the one or more transcriptional units encode the first polypeptide comprising the CAR, and wherein the genetically modified T cell or NK cell is the genetically modified T cell, and optionally wherein the CAR is a microenvironment restricted biologic CAR.

10. A reaction mixture, use, genetically modified T cell or NK cell, or method for genetically modifying T cells and/or NK cells according to any of the preceding claims, wherein said one or more transcriptional units further encode an inhibitory RNA molecule that targets TCRa, TCRb, SOCS1, miR155 target, IFN gamma, cCBL, TRAIL2, PP2A, ABCG1, CD3z, PD1, CTLA4, TIM3, LAG3, SMAD2, TNFRSF10B, PPP2CA, TNFRSF6 (FAS), BTLA, TIGIT, A2AR, AHR, EOMES, SMAD3, SMAD4, TGFBR2, PPP2R2D, TNFSF6 (FASL), CASP3, SOCS2, TIEG1, JunB, Cbx3, Tet2, or HK2.

11. A reaction mixture, use, genetically modified T cell or NK cell, or method for genetically modifying T cells and/or NK cells according to any of the preceding claims, wherein said one or more transcriptional units further encode an inhibitory RNA molecule that targets TIM3, LAG3, TNFRSF10B, PPP2CA, TNFRSF6 (FAS), BTLA, TIGI, A2AR, AHR, EOMES, SMAD3, SMAD4, PPP2R2D, TNFSF6 (FASL), CASP3, SOCS2, TIEG1, JunB, Cbx3, Tet2, and HK2.

12. A reaction mixture, use, genetically modified T cell or NK cell, or method for genetically modifying T cells and/or NK cells according to any of the preceding claims, wherein said one or more transcriptional units further encode an inhibitory RNA molecule that targets FAS, AHR, CD3z, cCBL, Cbx, HK2, FASL, SMAD4, or EOMES.

13. A reaction mixture, use, genetically modified T cell or NK cell, or method for genetically modifying T cells and/or NK cells according to any of the preceding claims, wherein said one or more transcriptional units further encode an inhibitory RNA molecule that targets FAS, AHR, Cbx3, HK2, FASL, SMAD4, or EOMES.

14. A reaction mixture, use, genetically modified T cell or NK cell, or method for genetically modifying T cells and/or NK cells according to any of the preceding claims, wherein said one or more transcriptional units further encode at least one inhibitory RNA molecule that targets an mRNA encoding AHR, Cbx3, HK2, SMAD4, or EOMES.

15. A reaction mixture, use, genetically modified T cell or NK cell, or method for genetically modifying T cells and/or NK cells according to any of the preceding claims, wherein the blood cells are not subjected to a PBMC enrichment procedure before the contacting.

16. A reaction mixture, use, genetically modified T cell or NK cell, or method for genetically modifying T cells and/or NK cells according to any of the preceding claims, wherein the reaction mixture comprises at least 50% whole blood.

17. A reaction mixture, use, genetically modified T cell or NK cell, or method for genetically modifying T cells and/or NK cells according to any of the preceding claims, wherein the reaction mixture is formed by adding the recombinant retroviral particles to whole blood.

18. A reaction mixture, use, genetically modified T cell or NK cell, or method for genetically modifying T cells and/or NK cells according to any of the preceding claims, wherein the reaction mixture is formed by adding the recombinant retroviral particles to substantially whole blood comprising an effective amount of an anti-coagulant.

19. A reaction mixture, use, genetically modified T cell or NK cell, or method for genetically modifying T cells and/or NK cells according to any of the preceding claims, wherein the reaction mixture is in a closed cell processing system.

20. A reaction mixture, use, genetically modified T cell or NK cell, or method for genetically modifying T cells and/or NK cells according to claim 18, wherein the reaction mixture is in contact with a leukodepletion filter assembly in the closed cell processing system.

21. A reaction mixture, use, genetically modified T cell or NK cell, or method for genetically modifying T cells and/or NK cells according to any of the preceding claims, wherein the reaction mixture comprises an anti-coagulant.

22. A reaction mixture, use, genetically modified T cell or NK cell, or method for genetically modifying T cells and/or NK cells according claim 21, wherein the anti-coagulant is selected from the group consisting of acid citrate dextrose, EDTA, or heparin.

23. A reaction mixture, use, genetically modified T cell or NK cell, or method for genetically modifying T cells and/or NK cells according claim 21, wherein the anti-coagulant is other than acid citrate dextrose.

24. A reaction mixture, use, genetically modified T cell or NK cell, or method for genetically modifying T cells and/or NK cells according claim 21, wherein the anti-coagulant comprises an effective amount of heparin.

25. A use or method for genetically modifying T cells and/or NK cells according to any one of claims 1, 3, 4, or 6-24, wherein the method further comprises administering the genetically modified T cells and/or NK cells to a subject, wherein the subject is the source of the blood cells.

26. A reaction mixture, use, genetically modified T cell or NK cell, or method for genetically modifying T cells and/or NK cells according to any of the preceding claims, wherein the T cell activation element is one or more polypeptides capable of binding CD3, CD28, OX40, 4-1BB, ICOS, CD9, CD53, CD63, CD81, and CD82.

27. A reaction mixture, use, genetically modified T cell or NK cell, or method for genetically modifying T cells and/or NK cells according to claim 26, wherein the T cell activation element is an anti-CD3 antibody, wherein the anti-CD3 antibody is bound to the membrane of the recombinant retroviral particles, optionally wherein the membrane-bound anti-CD3 is anti-CD3 scFv or an anti-CD3 scFvFc, and optionally wherein the anti-CD3 is bound to the membrane by a GPI anchor, and optionally wherein the anti-CD3 is a recombinant fusion protein with a viral envelope protein, and optionally wherein the anti-CD3 is a recombinant fusion protein with the viral envelope protein from MuLV, and optionally wherein the anti-CD3 is a recombinant fusion protein with the viral envelope protein of MuLV which is mutated at a furin cleavage site.

28. A reaction mixture, use, genetically modified T cell or NK cell, or method for genetically modifying T cells and/or NK cells according to any preceding claim, wherein an ABC transporter inhibitor and/or substrate, optionally an exogenous ABC transporter inhibitor and/or substrate, is not present before, during, or both before and during the genetic modification and/or transduction.

29. A reaction mixture, use, genetically modified T cell or NK cell, or method for genetically modifying T cells and/or NK cells according to any preceding claim, wherein the recombinant retroviral particles are present in the reaction mixture at an MOI of at least 1.

30. A use, genetically modified T cell or NK cell, or method for genetically modifying T cells and/or NK cells according to any one of claims 1-4, or 6-29, wherein at least 5% of the T cells and/or NK cells are genetically modified.

31. A use, genetically modified T cell or NK cell, or method for genetically modifying T cells and/or NK cells according to any one of claims 1-4, or 6-30, wherein the cell or cells are not subjected to a spinoculation of at least 800 g for at least 30 minutes.

32. A reaction mixture, use, genetically modified T cell or NK cell, or method for genetically modifying T cells and/or NK cells according to any preceding claim, wherein the reaction mixture is in a blood bag during the contacting.

33. A reaction mixture, use, genetically modified T cell or NK cell, or method for genetically modifying T cells and/or NK cells according to any preceding claim, wherein the reaction mixture is in contact with a leukodepletion filter assembly in a closed cell processing system before the contacting, at the time the recombinant retroviral particles and the blood cells are contacted, during the contacting comprising an optional incubating in the reaction mixture, and/or after the contacting comprising the optional incubating in the reaction mixture, wherein the T cells and/or NK cells, or the genetically modified T cells and/or NK cells are further subjected to a PBMC enrichment procedure after the contacting.

34. A reaction mixture, use, genetically modified T cell or NK cell, or method for genetically modifying T cells and/or NK cells according to any preceding claim, wherein the reaction mixture is in contact with a T lymphocyte and/or NK cell-enriching filter in the closed cell processing system before the contacting, and wherein the reaction mixture comprises granulocytes, wherein the granulocytes comprise at least 10% of the white blood cells in the reaction mixture, or wherein the reaction mixture comprises at least 10% as many granulocytes as T cells.

35. A reaction mixture, use, genetically modified T cell or NK cell, or method for genetically modifying T cells and/or NK cells according to any preceding claim, wherein the reaction mixture is in contact with a leukodepletion filter assembly in the closed cell processing system after the contacting comprising an optional incubating in the reaction mixture.

36. A reaction mixture, use, genetically modified T cell or NK cell, or method for genetically modifying T cells and/or NK cells according to any of the preceding claims, wherein the whole blood is other than cord blood.

37. A reaction mixture, use, genetically modified T cell or NK cell, or method for genetically modifying T cells and/or NK cells according to any of the preceding claims, wherein the one or more transcriptional units encode a first polypeptide comprising a lymphoproliferative element.

38. A reaction mixture, use, genetically modified T cell or NK cell, or method for genetically modifying T cells and/or NK cells according to claim 37, wherein the lymphoproliferative element comprises Box1 and Box2 Janus kinase (JAK)-binding motifs and a Signal Transducer and Activator of Transcription (STAT) binding motif comprising a tyrosine residue.

39. A reaction mixture, use, genetically modified T cell or NK cell, or method for genetically modifying T cells and/or NK cells according to claim 38, wherein the LE comprises an intracellular signaling domain from CD2, CD3D, CD3E, CD3G, CD4, CD8A, CD8B, CD27, mutated Delta Lck CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof.

40. A reaction mixture, use, genetically modified T cell or NK cell, or method for genetically modifying T cells and/or NK cells according to any one of claims 1-4, or 6-39, wherein the replication incompetent recombinant retroviral particles are lentiviral particles, and wherein the genetically modified cell is a genetically modified T cell or a genetically modified NKT cell.

41. A reaction mixture, use, genetically modified T cell or NK cell, or method for genetically modifying T cells or NK cells according to any one of claims 1-3, or 6-14, or 19-20, or 25-33, or 35, or 37-40, wherein said blood cells are PBMCs.

42. A reaction mixture, use, genetically modified T cell or NK cell, or method for genetically modifying T cells and/or NK cells according to claim 14, wherein the inhibitory RNA molecule comprises at least one of the sequences of SEQ ID NOs: 394-401, 406-409, 438-441, or 446-449.
